(19) United States
(12) Patent Application Publication
(10) Pub. No.: US 2024/0384227 A1
(43) Pub. Date: Nov. 21, 2024

(54) MATERIALS CHEMISTRIES AND MICROTOPOGRAPHIES AND USES THEREOF

(71) Applicant: THE UNIVERSITY OF NOTTINGHAM, Nottingham, Nottinghamshire (GB)

(72) Inventors: Morgan Alexander, Nottingham Nottinghamshire (GB); Amir Ghaemmaghami, Nottingham, Nottinghamshire (GB); Paul Williams, Nottingham, Nottinghamshire (GB); Simon Avery, Nottingham, Nottinghamshire (GB); Andrew Hook, Nottingham, Nottinghamshire (GB); Felicity Rose, Nottingham, Nottinghamshire (GB); Chris Denning, Nottingham, Nottinghamshire (GB)

(73) Assignee: THE UNIVERSITY OF NOTTINGHAM, Nottingham, Nottinghamshire (GB)

(21) Appl. No.: 17/799,914

(22) PCT Filed: Feb. 15, 2021
(86) PCT No.: PCT/IB2021/051274

§ 371 Date: Nov. 30, 2022

(30) Foreign Application Priority Data
Feb. 13, 2020 (GB) 2002011.1

(51) Int. Cl. C12N 5/00; A61L 27/34; A61L 27/50; A61L 31/04; A61L 31/14

(52) U.S. Cl. CPC C12N 5/0062; A61L 27/34; A61L 27/50; A61L 31/048; A61L 31/14; A61L 2400/18; A61L 2420/08; C12N 2533/30; C12N 2535/00

(57) ABSTRACT

A microtopography system for modulating one or more cellular processes on a surface is described. In particular a microtopography system comprising: a repeated microtopographic pattern and a polymer coating, said microtopographic pattern comprising: an array of repeated micropillars applied to a surface of a product, said micropillars being formed of surface structures between 1-100 μm in height, and 1-50 μm in width; and said polymer coating comprising one of a (meth)acrylate or (meth)acrylamide monomer, or mixture of two (meth)acrylate or (meth)acrylamide monomers. The microtopographic pattern and said polymer coating act to modulate one or more cellular processes on the surface.

### FIELD

The present invention relates to surface chemistries and combinations of surface chemistries with microtopographies which modulate cellular processes, uses of such chemistries and combinations, and products comprising them on their surface.

The developments made in the biomaterials field over the past 25 years (Bhat & Kumar, 2013) and the rise of newly available materials have prompted their use as an essential component of modern medicine and industrial activity utilising biological processes. Used for modulating cell proliferation and healing in regenerative medicine, for drug delivery, to fabricate a broad range of medical devices, and for producing molecules and products of interest on an industrial scale, biomaterials play an important role in disease management and healthcare improvement in day to day practice as well as in a variety of industrial areas. Moreover, there is a growing demand to improve the levels of wellbeing in rapidly expanding and ageing populations (Holzapfel et al., 2013), as well as to find new, cheaper ways to exploit biological processes for industrial purposes. Despite the progress made to date, a poor understanding of the complex cellular mechanisms and environment at a surface-cell interface has hampered the rational design of biomaterials. It remains important to identify relationships between the surface of biomaterials and any influence on biological processes in order to exploit these for human benefit.

For example, when given a suitable environment for adhesion, cells such as immune cells can attach to surfaces and increase or decrease their metabolic and/or proliferative activities, as well as influence differentiation potential of cells surrounding them and eventual cell fate. In the context of immunomodulation, the local environment of an implanted material may be able to influence immune rejection the implanted material via influencing the polarisation of immune cells surrounding or attached to the surface of the implanted material. Control of responses of immune cells to materials has applications as diverse as in vivo reprogramming of cells for use as cancer vaccines and controlling the foreign body response to medical devices and engineered implants. Similarly, controlling responses and differentiation of stem cells are attractive for regenerative medicine applications due to their multi-potency, ability to facilitate neovascularisation, and immunomodulatory effects. In the context of materials implanted into a subject, pathogenic bacteria may attach to a surface of the implanted material and form a biofilm which leads to clinical infection (Davies, 2003). In other industries, food spoilage and contamination of the surface and local area may occur upon bacterial attachment.

However, although synthetic biomaterials are ubiquitous, as medical devices they often fail. When used as cell carriers in regenerative medicine or in cell factories, control over the desired cell phenotype is limited. Critically, our understanding of mechanisms by which simple materials influence cell response is limited, making it difficult to improve them by design. Most attempts to manage the issue of the unwanted proliferation of microbes have focussed on the incorporation of antimicrobials to materials with the aim of killing or biologically inhibiting the growth of microbes (Zhao et al., 2009; Goodman et al., 2013). However, the main drawbacks of these strategies are their limited long-term efficacy and the enhancement of antimicrobial resistance (Swearingen et al., 2016). Similarly, pharmaceutically induced polarisation of immune cells relies on expensive-to-research and produce small molecules or biologics, which have many drawbacks such as toxic off-target effects, low efficacy, difficulty in applying to target sites, regulatory hurdles, and in the field of oncology the promotion of tumour resistance.

There is thus a need in the industry to develop alternative or improved methods to modulate cellular processes such influencing cell fate and reprogramming, or immune cell polarisation, which have a long-term efficacy, which are relatively cheap to make, which have a low toxicity profile and which do not force selective pressures on organisms.

### SUMMARY

### Microtopography System

According to an aspect, the invention provides a microtopography system for modulating one or more cellular processes on a surface, said microtopography system comprising a repeated microtopographic pattern and a polymer coating, said microtopographic pattern comprising an array of repeated micropillars applied to a surface of a product, said micropillars being formed of surface structures between 1-50 μm in height, and 1-50 μm in width, and said polymer coating comprising one of a (meth)acrylate or (meth)acrylamide monomer, and wherein said microtopographic pattern and said polymer coating act to modulate one or more cellular processes on the surface. Said microtopographic pattern and said polymer coating may act synergistically to modulate said one or more cellular processes.

In an embodiment, the micropillar may be about 1-100 μm in height (vertical), such as about between 5-45 μm, 10-40 μm, 15-35 μm, 20-30 μm, 25 μm, or 50-100 μm in height. In one preferred embodiment the micro-pillar may be approximately 10 μm in height.

Similarly, the micropillars may be between 1-100 μm in width (lateral), such as 2-45 μm, 3-40 μm, 4-35 μm, 5-30 μm, 10-25 μm, or 15-20 μm, or 50-100 μm in width. In one embodiment the micropillars are approximately 3+/−0.6 μm in width. Suitably, a micro-pillar may be 3-23 μm wide laterally and about 10 μm in height, such as 9.1+/−0.6 μm in height and 3+/−0.6 μm in width.

In one embodiment the microtopography of the micropillars above the underlying surface may have a mean area below 50 μm². In other embodiments, the micropillars have an eccentricity of <1, and preferably less than 0.5, preferably between 0.01-0.49, more preferable between 0.1-0.4, most preferably between 0.2-0.3.

Typically the micropillars are shaped according to a topography determined using a screening technique of possible primitive shape combinations. Said primitive combinations may comprise one or more of rectangles (including square), circles, triangles or other primitive shapes. Said shapes may be combined using a computational algorithm to generate a hybrid shape or micropillar that does not resemble the original primitives. It can be appreciated that such a hybrid shape may be a single conjoined shape, or may be a collection of shapes, in which case the micropillar is considered to include all shapes in the collection. The micropillars are then arranged on the surface in a repeating patterned array. Accordingly, in addition to interaction between the shapes or morphology of a single micropillar, cellular processes may be influenced by adjacent micropillars.

A microtopography may be assembled in periodical repetitions of a specific micro-pillar in a defined space, for example in a micro-well. Such a micro-well (also referred to herein as a TopoUnit) may have pre-defined dimensions, and may be present on a chip which comprises multiple micro-wells. Suitable dimensions may be about 500×500 μm, about 300 μm by 300 μm, or about 290 μm×290 μm. Each micro-well may be surrounded by a wall, for example which is about 40 μm tall. Each chip may comprise about 66 by about 66 wells of the same dimensions.

A microtopography may be constructed using a silicon mould using photolithography and etching to produce the ‘negative’ (inverse of the desired topography) master of the topographies. The desired ‘positive’ may be produced by injecting a 1:2 mixture of monomers trimethylolpropane tri(3-mercaptopropionate):tetra(ethylene glycol) diacrylate (1:2 TMPMP:TEGDA) containing the photoinitiator 2,2-dimethoxy-2-phenylacetophenone (DMPA) between a methacrylate-functionalised glass slide and the silicon master. This is herein referred to as the substrate, and may undergo UV curing and solvent washing. A specific (meth)acrylate or (meth)acrylamide monomer solution (50% w/v or 75% v/v monomer solutions in N,N-dimethylformamide (DMF) containing 0.05% w/v DMPA) may then be deposited onto each microtopography in each TopoUnit, before a further step of UV curing to polymerise to monomers and further washing steps.

The microtopography applied to the surface of a TopoUnit may be subjected to oxygen plasma etching to reduce the hydrophobicity of the material.

The features, including microtopography and surface chemistry, may be confirmed using a variety of techniques known to the skilled person, for example spectrometric and/or spectroscopic techniques, such as time-of-flight secondary ion mass spectrometry (ToF-SIMS), in situ mass spectrometer and X-ray photoelectron spectroscopy (XPS). A combination of techniques may be used to confirm the surface chemistry of the microtopography.

A microtopography may be applied to a pre-existing surface, or a surface may be constructed to comprise a given microtopography as a principle of its construction.

In an embodiment, the repeated microtopographic pattern and the polymer coating of the microtopography system may have been identified as suitable for modulating said one or more cellular processes according to a method of screening described herein.

### Polymers

Polymers used in any aspect of the invention may be formed from (meth)acrylate and (meth)acrylamide monomers.

Polymers may be embossed onto a microtopography which has been applied to a surface, and may be formed by in situ photopolymerisation of the respective monomer(s) drop cast on top of TPMP-co-TEGDA moulded microtopographical features. Alternatively, polymers may be applied to a flat or smooth surface on which no microtopography has been applied.

In a second aspect, the invention provides a polymer system for modulating cellular processes on a surface, said polymer system comprising a surface with a polymer coating applied to it, said polymer coating comprising one of a (meth)acrylate or (meth)acrylamide monomer, or mixture two (meth)acrylate or (meth)acrylamide monomers, and wherein the polymer coating acts to modulate a cellular process on the surface.

In an embodiment of any aspect of the invention, the polymer or mixture of polymers is identified as suitable for modulating said one or more cellular processes according to the method of screening the invention. The microtopography and/or polymer may be identified as modulating the one or more cellular process either positively or negatively.

The present invention provides microtopography and polymer systems that can be applied to surfaces such as existing biomaterials, clinical devices and tools including those for surgical and dental use, as well as industrial materials and those used in food storage and preparation as well as food products themselves, to modulate cellular activities. Surfaces with such combinations of surface materials chemistries and microtopographies applied possess a low toxicity profile, and can provide a more effective, sometimes synergistic, way of modulating cellular processes than using a single factor surface modification such as materials chemistries and microtopographies alone. Such systems may be used to prevent biofilm formation, promote wound healing, prevent infection and promote bone formation in regenerative medicine, for example.

### Combinations

The combinations of microtopographies and polymers screened may be classified into groups, for example by collating the features of a defined number of microtopographies or polymers which give a desired outcome on the modulation of a cellular process of interest, to create a predictive model to suggest microtopographies which provide the desired modulation of the cellular process of interest. For example the top 50, top 100, or top 200 microtopographies which increase or decrease the level of cellular process of interest, and the top 50, top 100, or top 200 polymers which increase or decrease the level of cellular process of interest may be collated and used in machine learning methods to create such predictive models.

Computational tools may be applied to identify key surface parameters, for example size and organisation of the primitive features in a micro-pillar. The information can then be used to create a predictive model to suggest microtopographies which provide the desired modulation of the cellular process of interest.

Such combinations may provide unexpected and powerful synergistic effects on modulating the one or more cellular reprocesses of the aspects of the invention.

### Cellular Processes

In an embodiment of any of the aspects of the invention, the one or more cellular processes comprises or consists of cell attachment, cell differentiation, cell proliferation, cell viability, cell pluripotency, protein expression and/or immune cell modulation.

The cell attachment may be prokaryote or eukaryote attachment. For example, the cell attachment can be one or more of Gram positive bacterial cell attachment; Gram negative bacterial cell attachment; fungal cell attachment, Antigen Presenting Cell (APC) attachment such as macrophage or dendritic cell attachment; neutrophil attachment; fibroblast attachment and/or proliferation; stem cell attachment such as human mesenchymal stem cell, or embryonic stem cell attachment.

In a system, method or product (or use of a product) described herein, wherein to induce a n increase hPSC attachment, the polymer comprises a hyperbranching solution of TCDMDA-containing polymer.

The cell differentiation may be stem cell differentiation such as mesenchymal stem cell differentiation to an osteoblast, or monocyte differentiation into dendritic cells or macrophages, or differentiation of fibroblasts to myofibroblasts. Cell differentiation may also be from stem cells to to cardiomyocytes, neurons, adipocytes, hepatocytes, chondrocytes. A stem cell may be an induced pluripotent stem cell (iPSC).

The immune cell modulation may comprise or consist of immune activity. The immune activity may be pro-inflammatory or anti-inflammatory. The immune activity may be one or more of the activation and/or polarisation of macrophages to an M0, M1 or M2 phenotype; the maturation and/or activation or suppression of dendritic cells; the activation or suppression of neutrophils; the production of cytokines from APCs. In an embodiment of any of the aspects of the invention, the microtopographic pattern and/or polymer coating modulate multiple cellular processes on a surface such as cell attachment, cell differentiation, cell proliferation, protein expression, and/or immune cell modulation, or a mixture thereof. For example, the microtopographic pattern and polymer coating may both reduce bacterial cell attachment and increase M2 macrophage polarisation or dendritic cell activation at the surface.

The cell proliferation may comprise or consist of fibroblast proliferation.

The protein expression may be smooth muscle actin (SMA) expression. Suitably, the SMA expression is increased on differentiation of fibroblasts to myofibroblasts. Suitably, the SMA expression and proliferation of fibroblasts are modulated.

The skilled person will understand that a cellular process measured, detected or modulated can relate to any cellular activity which can be measured or observed, for example directly or indirectly, visually, or numerically. Such cellular processes may include one or more of cell attachment, cell proliferation, cell differentiation, cell motility, cell viability, cell pluripotency, cell metabolism, enzymatic activity, production of specific compounds or metabolites, protein expression, cellular proliferation, DNA replication, cell signalling, cell morphology, immune activity (interchangeably used with the word ‘immunomodulation’)

The skilled person will also understand that a number of methods, readily available at their disposal using common general knowledge, may be utilised in order to measure or detect a cellular process of interest. Such methods may include fluorescence microscopy such as confocal microscopy, other fluorescence based techniques such as FACS and spectroscopy, qRT-PCR, single cell RNA seq, mass spectrometry or other protein quantification methods, western blotting, ELISA, assays to determine the metabolic activity of a cell such as glucose metabolism and respiratory burst, biological assays such as cell survival assays, cell adhesion and protein/particle uptake assays.

The modulation of a cellular process may refer to the increase or decrease of the level of that cellular process measured or detected when compared to the level of that cellular process measured or detected of a control condition, such as a reference surface as described above. The modulation may be determined to be increased or decreased only when a threshold value relative to the control condition is reached. One or a number of parameters may be considered when establishing a relevant threshold value. Thus, a threshold value may be in the units corresponding to the method used to measure or detect the cellular process. Where multiple parameters are measured and considered to establish the threshold value, arbitrary units may be given. The threshold value may be subject to statistical analysis. The threshold value may be dependent upon the exact cellular process measured or detected. The skilled person will readily understand that the nature of the cellular process measured or detected will influence both the method of measurement or detection and any threshold required to make a determination as to whether the cellular process is modulated relative to the control condition.

### Cells

The one or more cells in which a cellular process is modulated, or forming the first and/or second set of cells according to aspects of the invention, may be prokaryotic or eukaryotic cells.

Eukaryotic cells may be fungal cells or mammalian cells such as cancer cells, immune cells, skin cells, fibroblasts, stem cells. Immune cells may be monocytes, Antigen Presenting Cells (APCs) such as macrophages or dendritic cells, CD4+ T-cells, CD8+ T-cells, B-Lymphocytes, Natural Killer (NK) cells, neutrophils. Stem cells may be human mesenchymal stem cells (hMSCs), or induced pluripotent stem cells.

Cells cultured in the methods of screening according to the invention will be cultured in their preferred culture medium and conditions. The skilled person will readily be able to derive the required conditions from the common general knowledge.

### Products

In an embodiment of any aspect of the invention, the product may be one or more of the following: an implantable medical device, prosthetic, surgical tool, dental tool or dental device. For example, the product may be a catheter, dental screw, knee joint replacement, hip joint replacement, heart valve replacement, a stent, pacemaker, glucose sensor, contraceptive implant, breast implant, Implantable Cardioverter Defibrillators, spinal screws/rods/artificial discs, contact lenses, different types of shunts and stents prone to fibrosis and infection (e.g. nasolacrimal stents), wound care products.

In an embodiment, the product may be one or more of the following: cell culture dish or other research laboratory equipment, shower curtains, drainage pipes, food packaging, food processing tools or machinery including vats, food products, ship hulls, marine sensors, anti-fouling paint for subsea and maritime applications offshore wind foundations, bouyancy modules, oil rig structures, marine sensors; food processing equipment (Vats, Pipework), food preparation areas; water systems (food manufacture, healthcare water loop systems, water containers (i.e., domestic/industrial plumbing, waste water management). The product may also be applied to products in the beverage industry such as beer lines. The product may also have application to glass for use in products including (touch-screen displays and windows). The product may be a crop or crop product.

According to a third aspect of the present invention, there is provided a method of screening for a microtopography system according to the invention, wherein the method comprises:
i. Applying at least one microtopography to a surface;
ii. Applying a polymer to at least a substantial portion of the surface;
iii. Culturing one or more of a first set of cells on the surface with said microtopography and said polymer applied to it, and culturing a matching number and type of cells of a second set of cells on a reference surface;
iv. Measuring or detecting the level of one or more cellular processes of the first and second set of cells;
v. Comparing the level of the one or more measured or detected cellular processes of the first and second set of cells; and
vi. Determining whether the level of each of the one or more measured or detected cellular processes between the first and second set of cells is modulated either positively or negatively on the surface with said microtopography and said polymer applied to it compared to the reference surface.

In an embodiment, the polymer is formed from a (meth)acrylate or (meth)acrylamide monomer.

Advantageously, the invention allows the identification of combinations of surface materials chemistries and microtopographies which can be applied to surfaces such as existing biomaterials, clinical devices and tools including those for surgical and dental use, as well as industrial materials and those used in food storage and preparation, as well as food products themselves, or crops or crop products, to modulate cellular activities. This approach reduces costs and can provide a more effective way of modulating cellular processes than approaches using a single factor surface modification, such as microtopographies alone.

In a fourth aspect, there of the present invention, there is provided a method of screening for a polymer system according to the invention, wherein the method comprises:
i. Applying a polymer or mixture of polymers to at least a substantial portion of the surface;
ii. Culturing one or more of a first set of cells on the surface with said microtopography and said polymer applied to it, and culturing a matching number and type of cells of a second set of cells on a reference surface;
iii. Measuring or detecting the level of one or more cellular processes of the first and second set of cells;
iv. Comparing the level of the one or more measured or detected cellular processes of the first and second set of cells; and
v. Determining whether the level of each of the one or more measured or detected cellular processes between the first and second set of cells is modulated either positively or negatively on the surface with said polymer or mixture of polymers applied to it compared to the reference surface.

In an embodiment, the polymer or mixture of polymers is formed from a (meth)acrylate or (meth)acrylamide monomer or a mixture of two (meth)acrylate or (meth)acrylamide monomers.

In a fifth aspect, the invention provides a method of modulating one or more cellular processes at a surface of a product, wherein the method comprises applying a microtopography to said surface, and applying a polymer to at least a substantial portion of said surface. Suitably, the polymer is applied to the microtopography which has been applied to said surface.

In a sixth aspect, the invention provides a method of modulating one or more cellular processes at a surface of a product, wherein the method comprises applying a polymer to at least a substantial portion of said surface. Suitably, the polymer is applied to the microtopography which has been applied to said surface.

In a seventh aspect, the invention provides a product with a surface on which a microtopography has been applied, and on which a polymer has been applied to at least a substantial portion of, for use in modulating one or more cellular processes at said surface. Suitably, the polymer is applied to the microtopography which has been applied to said surface.

In an eighth aspect, the invention provides a product with a surface on which a polymer or mixture of polymers has been applied to at least a substantial portion of, for use in modulating one or more cellular processes at said surface.

In an embodiment of the sixth or seventh aspects, the product is for use in preventing rust formation, preventing food spoilage, preventing crop disease, tissue culture and research product coating such as cell culture dishes and plasticsware, glassware, anti-fouling paint, food processing equipment and preparation areas, food products, water systems and containers.

In a ninth aspect, there is provided a product of the invention for use in treating or preventing a disease or disorder in a subject. The disease or disorder may be selected from: a bacterial infection, fungal infection, an inflammatory disease or disorder, a bone disorder, fibrosis, wound healing.

In an embodiment, a bacterial infection may be caused by one or more of Pseudomonas spp., Staphylococcus spp., Bacillus spp., Lactobacillus sp., proteus spp., Enterobacter spp., Escherichia Coli, Klebsiella spp., Salmonella spp., Listeria spp., Yersinia spp., Legionella spp, Clostridium spp., Acinetobacter spp. For example, a bacterial infection may be caused by one or more of Pseudomonas aeruginosa, Staphylococcus aureus, Proteus mirabilis, Acinetobacter baumannii. Suitably, the bacterial infection may be the result of biofilm formation.

In an embodiment, an inflammatory disease or disorder may be selected from the group consisting of transplant rejection, Graft Versus Host Disease (GVHD), psoriasis, eczema, rheumatoid arthritis, a cancer, ulcerative colitis, Crohn's disease, diabetic/chronic wounds, non-healing fractures, an autoimmune disease.

In an embodiment, the bone disorder may be osteoporosis, rheumatoid arthritis, a bone cancer. In an embodiment, the fungal infection may be caused by one or more of Candida albicans, Botrytis cinerea, Zymosteptoria.tritici, Aspergillus brasiliensis, Candida auris and Colletotrichum gloeosporioides.

In a tenth aspect, there is provided a method of treating or preventing a disease or disorder in a subject, comprising:
i. Applying a microtopography to the surface of an implantable medical or dental product;
ii. applying a polymer to at least a substantial portion of said surface.
iii. applying said product the subject.

In an eleventh aspect, there is provided a method of treating or preventing a disease or disorder in a subject, comprising:
i. Applying a polymer or mixture of polymers to the surface of an implantable medical or dental product;
ii. applying said product the subject.

In an embodiment of the tenth or eleventh aspect, the disease or disorder may be selected from: a bacterial infection, fungal infection, an inflammatory disease or disorder, a bone disorder, fibrosis, non-healing/chronic wounds.

In an embodiment, a bacterial infection may be caused by one or more of Pseudomonas spp., Staphylococcus spp., Bacillus spp., Lactobacillus sp., proteus spp., Enterobacter spp., Escherichia Coli, Klebsiella spp., Salmonella spp., Listeria spp., Yersinia spp., Legionella spp, Clostridium spp., Acinetobacter spp., For example, a bacterial infection may be caused by one or more of Pseudomonas aeruginosa, Staphylococcus aureus, Proteus mirabilis, Acinetobacter baumannii. Suitably, the bacterial infection may be the result of biofilm formation.

In an embodiment, an inflammatory disease or disorder may be selected from the group consisting of transplant rejection, Graft Versus Host Disease (GVHD), psoriasis, eczema, rheumatoid arthritis, a cancer, ulcerative colitis, Crohn's disease, diabetic/chronic wounds, non-healing fractures, an autoimmune disease.

In an embodiment, the bone disorder may be osteoporosis, rheumatoid arthritis, a bone cancer.

In an embodiment of the tenth or eleventh aspects, the application of said product to the subject step is via surgical or non-surgical means, such as direct application to a wound.

In an embodiment, the product is an implantable medical device, prosthetic, surgical tool, dental tool or dental device. Suitably, the product may be a catheter, dental screw, knee joint replacement, hip joint replacement, heart valve replacement, a stent, pacemaker, glucose sensor, contraceptive implant, breast implant, Implantable Cardioverter Defibrillators, spinal screws/rods/artificial discs, contact lenses.

The invention therefore provides means to prevent or treat a variety of medical indications ranging from primary bacterial infection at the site of an implant or wound, and infection as result of biofilm formation on implanted medical devices and dental products. The invention provides achieves these effects by reducing foreign body reaction to devices, both initially when inserting a device (both short term such as a catheter, or long term such as a vascular graft). Other effects of the invention include reducing aseptic loosening of dental screws and other hard implants such as knee and hip joints, pace makers and glucose sensors where the electrical contact with the surroundings are impaired, or where the development of fibrous capsule increases the risk of complications (e.g. breast implants). Similarly the invention may provide a platform for the adherence of cells of interest to influence their differentiation and/or activity, for example to promote stem cell differentiation to osteoblasts in diseases resulting in the need for increased bone formation. The invention also achieves the described effects by skewing immune cell activity to promote wound healing (e.g. polarisation of M2 macrophages in after procedures), to promote inflammatory responses (e.g. by polarisation to M1 macrophages in cases of infection) or to promote an anti-inflammatory response in the case of transplants etc. (e.g by reducing dendritic cell activation).

The skilled person will recognise that an immune disorder/disease is any disease or disorder in a subject characterised by aberrant immune cell activity, including both over-active and suppressed immune activity compared to a healthy individual. Exemplary immune disorders/diseases may be an inflammatory disease, immunosuppression, transplant rejection, medical device rejection. Suitably, the immune disorder/disease may be one or more of rheumatoid arthritis, systemic lupus erythematosus, inflammatory bowel disease, Crohn's disease, multiple sclerosis, Type I diabetes, Guillain-Barre syndrome, psoriasis, cancer, eczema, fibrosis, chronic non-healing wounds.

In the case of preventing or reducing transplant or medical device (including prosthetics) rejection and/or GVHD, the surface of a product, such as a prosthetic, implantable medical device, cell culture dish coating, or biodegradable and/or porous protective sheet may be constructed with, or have applied to it, a microtopography and polymer which have been identified or predicted to downregulate monocyte and/or APC attachment and/or pro-inflammatory immune activity. This would reduce the inflammatory response of APCs which recognise the transplant or graft as foreign, and thus reduce the likelihood of rejection of the transplant or foreign material, or would induce the differentiation and polarisation of monocytes to a macrophage phenotype of interest. The microtopography and polymer may have been identified or predicted to have the desired properties using a method of screening of the invention.

In the case of promoting stem cell differentiation down a specific lineage, for example hMSCs to osteoblasts or fibroblasts to myofibroblasts, the surface of a product or of a product made from said polymer, for example a prosthetic, implantable medical device or cell culture dish coating, may be constructed with, or have applied to it, a microtopography which has been identified or predicted to downregulate or resist cell attachment. The microtopography may have been identified or predicted to have the desired properties using a method of screening of the invention.

In the context of bacterial and/or fungal attachment to surfaces, a community of microorganisms living may establish on a surface from a planktonic state to form a biofilm, leading to clinical infection. Once the microorganisms attach to the surface, a complex process initiates in which micro-colonies are formed, and cell-cell communication and quorum sensing occurs. Such biofilms are an issue in a variety of situations, particularly in the medical field, where implanted devices or prosthetics which are difficult to remove or exchange see the accumulation of pathogenic microorganisms on a biofilm and the progression of pathogenesis. Biofilms may also form on surfaces which come into contact with food, causing general hygiene issues. Typically, bacteria attach to surfaces using specialised structures such as flagella and pili which are formed of proteins such as adhesins, as well as by hydrodynamic and electrostatic interactions. Polysaccharides, lipopolysaccharide and glycoproteins may also contribute to the attachment. In the case of preventing or reducing the risk of biofilm formation, the surface of a product, for example a prosthetic or implantable medical device, may be constructed with, or have applied to it, a microtopography which has been identified or predicted to downregulate or resist cell attachment. The microtopography may have been identified or predicted to have the desired properties using a method of screening of the invention.

Similarly, in dental procedures, it is desirable to prevent infection of any open wound during any procedure, or to prevent the formation of a biofilm on any dental implant such as a screw. At the same time, differentiation of monocytes and the polarisation to M2 macrophages, known to be crucial in many aspects of healing and tissue regeneration, especially in dental applications such as to promote differentiation of human dental pulp cells, would be highly desirable. Thus, a combination of cellular processes may be modulated to achieve the desired outcome.

A subject may be a human or non-human mammal, such as a pig, dog, cat, horse, donkey.

### Stem Cell Attachment

In a system, method or product (or use of a product) described herein, to induce an increase in hPSC attachment, the polymer comprises or consitst of a hyperbranching solution of TCDMDA-containing polymer.

### Cardiomyocytes

In a system, method or product (or use of a product) described herein, to induce an increase in cardiomyocyte attachment, the polymer may be a nitrogen containing polymer.

In a system, method or product (or use of a product) described herein, to improve functionality of cardiomyocytes, the polymer may be an amine-containing polymer. Optionally, the functionality may be contraction, relaxation

### Cell Differentiation

Totipotent, multipotent and pluripotent stem cells have the ability to divide and to differentiate into a range of different cell types. Stem cell therapy is a promising approach to cure degenerative diseases, cancer, damaged tissues, or any disease for which there are very limited therapeutic options. Stem cell therapies could potentially improve the efficiency of the human body regenerative response following an injury or insult, in addition to being a source of powerful therapeutic compounds that hold the promise of the restoration of normal function of a given tissue. Additionally, being able to direct the differentiation of pluripotent or totipotent stem cell, or even near-terminal cells into a specific downstream or terminal cell of choice provides an opportunity to sculpt cellular responses and biological processes to give a desired outcome, in both medical and more general and wellbeing contexts.

However, current methods to induce the differentiation of cells down a certain pathway involve the use of extremely expensive soluble molecules such as cytokine cocktails, as well as complex media to culture said cells.

Very little is known about how a cells microenvironment, including the topography and surface chemistry of nearby structures, affects cell fate and differentiation. The ability to direct cells to differentiate down a certain lineage, at specific surface interfaces presents an extremely attractive premise in the field of regenerative medicine as well as immunology. For example, differentiation of hMSCs to osteoblasts may have application in bone repair following disease or trauma or ageing, as well as for use as an in vitro model of bone for drug discovery and development.

In an embodiment of any aspect of the invention, the one or more cellular processes comprises or consists of inducing (increasing) cell differentiation.

Suitably, the cell differentiation may be stem cell differentiation. The differentiation may be from human mesenchymal stem cells (hMSCs) to osteoblasts. The differentiation of hMSCs to osteoblasts may be identified by measuring or detecting the presence of alkaline phosphatase (ALP), amongst other markers known generally. The differentiation of hMSCs to osteoblasts may be confirmed when ALP expression is increased 20%, 30%, 50%, 80%, 100%, 200% in hMSCs contacted with said surface relative to hMSCs which are contacted with a reference surface.

In an embodiment, for inducing the differentiation of hMSCs to osteoblasts, the microtopography of an aspect of the invention has features with a radius of about 2-3 μm, preferably 2.5 μm, spacings of about 5-10 μm and the polymer is BzHPEA. In another embodiment, microtopography may have features with a radius of about 2.5-3.5 μm, preferably 3.5 μm, and the polymer is mMAOES. In another embodiment, microtopography may have features with a radius of about 2.5-3.5 μm, preferably 3.5 μm, and the polymer is MAPU.

### Immune Modulation

Modulation of immune activity is currently at the forefront of modern medicine, and is seen as the future of the industry to tackle treatment of infections, inflammatory diseases and cancer. Current strategies to control the activity of immune cells include the use of pharmacological small molecules, biologics and even genetic engineering of patient's cells to train them to recognise antigenic targets of interest. All these strategies are extremely expensive, can have toxic side effects and many force directed evolution of pathogens and cancers.

Depending on the circumstance or indication, either an upregulation of immune activity or a downregulation of immune activity can be desired. For example, in an inflammatory disease, the downregulation of inflammatory responses is desired, whilst in infectious scenarios, the upregulation of immune activity of certain cells, such of APCs is highly desirable.

Macrophages, either tissue resident or those which differentiate from peripheral blood monocytes, represent a heterogeneous population that are present in nearly all tissues of the body and as such, encounter a variety of environments and stimuli, both chemical and physical, and initiates specific inflammatory or healing responses to such stimuli. Similarly, Dendritic Cells, so called ‘sentinels of the immune system’ are specialised APCs which are able to uniquely undertake the process of cross presentation, whereby they ingest and process antigens to present to T-cells, thereby initiating an appropriate adaptive immune response. Upregulating such activities of immune cells such as APCs is clearly desirable in situations such as potential infection, whereas the activity of these cells largely contributes to inflammatory diseases and transplant/medical device rejection, so would preferably be downregulated in such circumstances. It is therefore extremely desirable to be able to modulate the activity of these cells in a given environment.

However, little is known about the effect of interplay between tissue microenvironment topography, surface chemistries and spatial arrangements on immune cell function and behaviour. The ability to modify or modulate the activation status of APCs, such as the polarisation status of macrophages and activation and maturation of dendritic cells, is emerging as an important new approach to tackle inflammatory diseases or to induce a targeted immune response to pathogens.

The skilled person will also appreciate that the immune activity of cells in the microenvironment of the surface on which a microtopography and polymer has been applied may be modulated, either directly as a result of sensing and signalling induced by attachment to the surface, or indirectly through cell-cell signalling initiated from cells which are either attached to the surface or which are in close proximity to the surface.

Immune activity may be measured by the expression of specific markers in a set of subset of cells, or the observable morphology or phenotype of specific cells (including differentiation status). The skilled person will understand that many biological methods, tools and markers are at their disposal to directly or indirectly measure the immune activity of cells, including soluble molecule production and secretion such as cytokine production, cell surface and intracellular protein expression, changes in morphology, cell adherence, mRNA levels and the oxidative state of the cells.

The skilled person will understand that some markers are inflammatory markers (increased immune activity), whilst some are anti-inflammatory or wound healing markers (decreased immune activity), and that the increase in an inflammatory marker would contribute to an increased up upregulated immune activity, whilst an increase in an anti-inflammatory marker would contribute to a decreased immune activity, and vice versa. Additionally, attachment of DCs may lead to their maturation, and the activation of such cells may require the presence of an antigen.

CD14+ monocytes may differentiate into macrophages. Macrophages may be classified as M0 (resting), M1 (which are pro-inflammatory), or M2 (which are anti-inflammatory).

Classically activated macrophages (pro-inflammatory) are classified as ‘M1’. A suitable marker for M1 macrophages is the expression and/or secretion of TNFα, or expression of calprotectin. Other markers may include CD86, MHCII, CD25.

Alternatively activated macrophages (anti-inflammatory) are classified as ‘M2’. A suitable marker for M2 macrophages is the expression and/or secretion of IL-10, or expression of the mannose receptor. M2 macrophages play a significant role in fibrotic encapsulation, and co-ordinating a reduced, localised immune response to a biomaterial surface.

Resting macrophages are classified at M0, and may be classified as such when compared to a polarised M1 or M2 macrophage. A mixture of markers may be used to determine the activation state of a macrophage. The ratio of M2 to M1 macrophages may also be used to determine the status (pro-inflammatory or anti-inflammatory) of a population of macrophages.

Mature DCs may be identified via upregulated cell surface expression of markers such as CD80, CD86 and MHC-II compared to naïve, non-mature dendritic cells. Similarly, activated DCs may be identified via upregulation of markers such as CD40. A mixture of markers may be used to determine the activation and/or maturation state of DCs.

The skilled person will understand that the marker used to identify the maturation/activation state of an APC will depend on the nature (subset) and location of the APC. A mixture of markers may be used to determine the immune activity of cells.

In an embodiment of any aspect of the invention, the one or more cellular processes comprise or consists of immune cell modulation.

In an embodiment, the immune cell modulation is inducing (increasing) the differentiation of human CD14+ monocytes into APCs.

In an embodiment, the APCs are macrophages and dendritic cells.

In an embodiment, the macrophages are polarised to an M2 or M1 phenotype. The differentiation and polarisation of human CD14+ monocytes into M1 and M2 macrophages may be identified by measuring or detecting the presence of Tumour Necrosis Factor (TNF) or interleukin-10 (IL-10) respectively.

In an embodiment, to induce the differentiation and polarisation of CD14+ monocytes to M1/M2 macrophages, the microtopography of an aspect of the invention has cylindrical pillars with a mean area below 50 μm², a maximum radii of about 1-3 μm, an eccentricity of below 0.5, preferably between 0.1-0.4, more preferably between 0.15-0.35, and the polymer is DMAm, BzHPEA or DEAEMA. It can be appreciated that by cylindrical, it is determined to be an elongated broadly cylindrically shaped object. Such object may have a substantially circular cross-section and may be considered to be broadly elliptical or the like.

In another embodiment, the CD14+ monocytes are differentiated into dendritic cells, which can be mature and/or activated or suppressed. The maturation of dendritic cells may be identified by measuring or detecting the presence of or increase in expression of (compared to non-mature dendritic cells or CD14+ monocytes) one or more of CD80, CD86 and MHC-II. The activation of dendritic cells may be identified by measuring or detecting the presence of or increase in expression of (compared to non-mature dendritic cells or CD14+ monocytes) CD40. The suppression of dendritic cells may be identified by measuring or detecting the absence of or decrease in expression of (compared to non-mature dendritic cells or CD14+ monocytes) CD40. The skilled person will be aware of multiple marker sint he art which can be used to identify dendritic cell activation and/or suppression. In an embodiment, to induce the differentiation of monocyte-derived dendritic cells (MoDCs) to activated dendritic cells, the polymer is any one of BADPODA, DEAEA, EaNiA, HFiPMA, COEA, F7BA, pEGMEMA, HEA, pEGDA or PhEA.

Preferably, in an embodiment, to induce the differentiation of monocyte-derived dendritic cells (MoDCs) to activated dendritic cells, the polymer is any one of BADPODA, DEAEA, HFiPMA, or pEGMEMA.

Preferably, in an embodiment, to induce the activation of monocyte-derived dendritic cells (MoDCs), the polymer is any one of BADPODA, DEAEA, HFiPMA, or pEGMEMA.

In an embodiment, to induce the differentiation of monocyte-derived dendritic cells (MoDCs) to suppressed dendritic cells, the polymer or mixture of polymers is any one of: COEA, THFuA, ZnA, PEDAM, PhMAm, MAPU, HDFHuA, (EDGMA about 66%+HDFDA about 33%), MTEMA.

Preferably, in an embodiment, to induce the differentiation of monocyte-derived dendritic cells (MoDCs) to suppressed dendritic cells, the polymer or mixture of polymers is any one of: COEA, THFuA, PhMAm or ZnA.

Preferably, in an embodiment, to induce the suppression of monocyte-derived dendritic cells (MoDCs), the polymer or mixture of polymers is any one of: COEA, THFuA, PhMAm or ZnA.

In an embodiment, to induce an increase in monocyte-derived dendritic cell viability and/or longevity, the polymer is any one of DFHA, MBMAm, SPAK, SPMAK, THFuMA, NpMA, PhEA, ZrCEA, DEGDMA, TEGDA

In an embodiment, to induce the differentiation of CD14+ monocytes to M0 macrophages, the polymer or mixture of polymers may be (EGDMA about 66%+HDFDMA about 33%), (BOEMA about 66%+DFFMOA about 33%), GPOTA, C398, C408

In an embodiment, to induce an increase in the differentiation of CD14+ monocytes to M1 macrophages, the polymer or mixture of polymers may be (CHMA about 66%+DMAEMA about 33%), tBCHMA, HDDMA, BDDA, DDDMA, TMOPTMA, H126, H98, H135, C176, C170, C240 In an embodiment, to induce an increase in the differentiation of CD14+ monocytes to M2 macrophages, the polymer or mixture of polymers may be (CHMA about 66%+iDMA about 33%), (PhMA about 66%+iDMA about 33%), IDMA, GDGDA, tBMA, TAlC, H47, H37, H9, C255, C140, C186

In an embodiment, to induce an increase in CD14+ monocyte or macrophage attachment to a surface, the polymer or mixture of polymers may be H133, H90, H103, H21, H94, H24, H69, H96, H92, H33, C56, C386, C32, C347, C295

In an embodiment, to induce a decrease in CD14+ monocyte or macrophage attachment to a surface, the polymer or mixture of polymers may be C358, C209, C434, C94, C48.

In an embodiment, to induce an increase in CD14+ monocyte attachment to a surface and increase in the differentiation of CD14+ monocytes to M1 macrophages, the polymer or mixture of polymers may be C170.

In an embodiment, to induce an increase in CD14+ monocyte attachment to a surface and increase in the differentiation of CD14+ monocytes to M2 macrophages, the polymer or mixture of polymers may be C162.

In an embodiment, to induce a decrease in CD14+ monocyte attachment to a surface and increase in the differentiation of CD14+ monocytes to M1 macrophages, the polymer or mixture of polymers may be C311.

In an embodiment, to induce a decrease in CD14+ monocyte attachment to a surface and increase in the differentiation of CD14+ monocytes to M1 macrophages, the polymer or mixture of polymers may be C164.

### Fibroblasts

In an embodiment of any aspect of the invention, the one or more cellular processes comprises or consists of cell proliferation and/or smooth muscle actin (SMA) expression. In an embodiment, the cell is a fibroblast.

In an embodiment, to induce an increase in SMA expression and increase in cell proliferation, the polymer is PhEA, THFuMA, CzEA or EGDA.

In an embodiment, to induce a decrease in SMA expression and decrease in cell proliferation, the polymer is PBPhMA, THFuA, pEGPHEA, EGDPEA, LMMA, NibMA, iDA, MAETA, or AODMBA.

In an embodiment, to induce a decrease in SMA expression and increase in cell proliferation, the polymer is NBnMA, TMPDAE, EGPEA, DMPMAm, THFuA or HFPDA.

In an embodiment, to induce an increase in SMA expression and decrease in cell proliferation, the polymer is PPDDA, 2EhMA, CIbMA or DVAd

In an embodiment of any aspect of the invention, the one or more cellular processes comprises or consists of fibroblast attachment to a surface.

In an embodiment, to induce a decrease in fibroblast attachment, the polymer is HEA, iPAM, AA, iBuMA, PPPDMA, MMaM, MAPU, HMAm or HEAm.

### Fungi

In an embodiment of any aspect of the invention, the one or more cellular processes comprises or consists of fungal cell attachment to one or more surface. The one or more surface may be a plant surface, biomedical device or other inanimate commercial material. The attachment may be of one or more of Candida albicans, Botrytis cinerea, Zymosteptoria.tritici Aspergillus brasiliensis, Candida auris and Colletotrichum gloeosporioides

In an embodiment, to induce a decrease in Candida spp., such as Candida albicans, attachment to a surface, the polymer is AODMBA, tBCHMA, tBCHA or IDMA.

In an embodiment, to induce a decrease in Botrytis cinerea and/or Colletotrichum gloeosporioides attachment to a surface, the polymer is mMAOES, DEGEEA or pEGPhEA.

In an embodiment, to induce a decrease in both Botrytis cinerea and Candida albicans attachment to a surface, the polymer is DEGMA or TEGMA.

In an embodiment, to induce a decrease in Botrytis cinerea, Zymoseptoria tritici or Aspergillus brasiliensis attachment to a surface, the polymer is mMAOES, DEGEEA or pEGPhEA.

In an embodiment, to induce a decrease in one or more of Botrytis cinerea, Zymoseptoria tritici, Aspergillus brasiliensis, Candida albicans, Colletotrichum gloeosporioides and Candida auris attachment to a surface, the polymer is DEGMA or TEGMA.

In an embodiment, to induce a decrease in one or more of B. cinerea, Z. tritici, A. brasiliensis, and/or Colletotrichum gloeosporioides attachment to a surface, the polymer is LaA.

In an embodiment, to induce a decrease in Candida albicans attachment to a surface, the polymer contains a carbonyl group.

In an embodiment, to induce an increase in Candida albicans attachment to a surface, the polymer contains a methylene nitrile group.

In an embodiment, to induce a decrease in fungal cell attachment to a surface, the polymer is hydrophilic, with a water contact angle (WCA) of 20-50° or 62-72°.

In an embodiment, to induce a decrease in Candida albicans attachment to a surface, the polymer is hydrophobic, with a water contact angle (WCA) of 62-96°.

In an embodiment, to induce a decrease in one or more of Botrytis cinerea, Zymoseptoria tritici, Aspergillus brasiliensis, Candida albicans, Colletotrichum gloeosporioides and Candida auris attachment to a surface, the polymer may comprise a co-polymer combining any of the homopolymers described herein.

### Neutrophils

In an embodiment of any aspect of the invention, the one or more cellular processes comprises or consists of neutrophil attachment.

In an embodiment, to induce an increase in neutrophil attachment to a surface, the polymer is DMPAm, AMPAm.C, MAEACI, DMEMAm, EGDA or AEMAm.C

### Stem Cell Pluripotency

In an embodiment of any aspect of the invention, the one or more cellular processes comprises or consists of retention of stem cell pluripotency after cell proliferation. Stem cell pluripotency can be measured using any or all of OCT4, NANOG, SOX2, TRA181 and/or SSEA4 expression, where a high expression corresponds with pluripotency.

In an embodiment, to induce an increase in retention of stem cell pluripotency after cell proliferation on a surface, the polymer or mixture of polymers may be poly tricyclodecane-dimethanol diacrylate-co-butyl acrylate (poly(TCDMDA-blend-BA)), suitably at a ratio of about 70:30, or 2:1, or neopentyl glycol diacrylate-co-2-hydroxyethyl methacrylate (poly(NGPDA-co-HEMA)) in a ratio of around 2:1 NGPDA:HEMA, tetraethylene glycol dimethacrylate-co-ethylene glycol dicyclopentenyl ether acrylate (poly(EG4DMA-co-EGDPEA)) in a ratio of around 2:1 EG4DMA:EGDPEA; or glycerol dimethacrylate-co-furfuryl methacrylate (poly(GDMA-co-FuMA)) in a ratio of around 2:1 GDMA:FuMA.

In an embodiment of any aspect of the invention, the microtopography and/or the polymer are identified as suitable for modulating said one or more cellular processes according to the methods of screening the invention. The microtopography and/or polymer may be identified as modulating the one or more cellular process either positively or negatively.

A reference surface referred to in relation to any of the above aspects is a surface in which no specific microtopography has been applied. Such a reference surface may be flat and/or smooth, and in relation to aspects referring to polymers, the reference surface may only have substrate applied to it (TMPMP-co-TEGDA).

A surface with a microtopography applied to it, and which has a polymer applied to at least a substantial portion of said surface, may refer to the scenario where the polymer is applied directly on top of the microtopography, and thus wherein the polymer is on the same side of the surface as the microtopography.

By eccentricity it means the measure of how close an ellipse is to being a circle. In the present description, microtopographies may be described as having an eccentricity. This is intended to capture the broad shape when viewed as a cross-section. An eccentricity of 0 defines a circular cross-section, whilst elliptical cross-sectional pillars of microtopographies would have an eccentricity between 0 and 1. It can be further appreciated that references to cylindrical micropillars or microtopographical features may be intended to describe both complete cylinders having a circular cross-section, elliptical cylinders, and non-circular or elliptical cylinders having rounded portioned cross-sections.

In any aspect of the invention, a surface coated with a polymer refers to the surface being coated with homopolymers. A surface coated with a mixture of polymers refers to the surface being coated with a mixture of two polymers (copolymers), or three polymers.

In an embodiment, the mixture of copolymers may be applied to the surface at a percentage of about 50%:50%, 75%:25%, 80%:20%, preferably 66%:33% In an embodiment of any aspect of the invention, said surface may be placed in a location where the modulation of the one or more cellular process is desired. This may be a location where the surface is likely to come into contact with a cell of interest. The cell of interest may be the same as the first and second set of cells the methods of screening of the invention. The cell of interest may be different to the first and second set of cells of the second aspect of the invention. The cell of interest may be any cell in which a given cellular process to be modulated is capable of being modulated.

At least a substantial portion of the surface of any aspect of the invention may refer to about 50%, about 60%, about 70%, about 80%, about 90%, about 95%, about 98%, about 99% of the surface, or 100% of the surface.

Any method of the invention may be an in vitro method, or an ex vivo, or in vivo method.

The term “about” as it relates to any value denotes that the value it refers to can be modified by 10% above and below said value. For example, “about 10” retains both 9 and 11 within its scope. Similarly 0.5 (or 0.50) can refer to 0.45 (or 0.445 and above when rounding) or 0.55 (or 0.555 and below when rounding).

A product described herein ‘for use’ in any method or purpose, may also refer to ‘use of’ that product for said method or purpose.

The skilled person will appreciate that preferred features of any one embodiment and/or aspect of the invention may be applied to all other embodiments and/or aspects of the invention.

### BRIEF DESCRIPTION OF THE FIGURES

FIG. 1—The ChemoTopoChip Combinatorial Screening Platform
a) Schematic showing ChemoTopoChip layout (walls of 30 μm height are used to separate each Topo unit); b) ChemoTopoChip ChemoTopo unit containing 35 topographies+flat area; c) Example Topo unit; d) ChemoTopoChip production process; e) Photographed ChemoTopoChip; f) TMPMP and TEGDA, used to mould ChemoTopoChip features.

FIG. 2—ChemoTopoChip Characterisation
a) Interference profilometer imaged ChemoTopoChip and example features from a ChemoTopo unit; b) ToF-SIMS images of functionalised surface showing the distribution of the thiol ion from the base and of 6 ions unique to specific functionlisation chemistries.

FIG. 3—hiMSC fate assessment on the ChemoTopChip a) Unfunctionalised ChemoTopo unit moulded from TMPMP-co-TEGDA base material, stained with α-tubulin; b) ChemoTopo unit surface functionalised with iBOMAm, stained with α-tubulin; c) Rank ordered hiMSC cell number showing distribution of hiMSC cell number across the ChemoTopoChip (N=3, n=3) ordered by chemistry 1-28, topography 1-36 and all combinations; d) Rank ordered ALP intensities normalised to flat TMPMP-co-TEGDA area showing distribution of hiMSC ALP intensity across the ChemoTopoChip (N=3, n=3) ordered by chemistry 1-28, topography 1-36 and all combinations; e) hiMSCs on flat TMPMP-co-TEGDA area (blue=ALP, yellow=α-tubulin); f) hiMSCs on flat mMAOES area (blue=ALP, yellow=α-tubulin); g) hiMSCs on TMPMP-co-TEGDA+Topo 3 area (blue=ALP, yellow=α-tubulin); h) hiMSCs on mMAOES+Topo 3 area (blue=ALP, yellow=α-tubulin).

FIG. 4—Human macrophage phenotype assessment on the ChemoTopoChip a) Unfunctionalised ChemoTopo units moulded from TMPMP-co-TEGDA base material cultured with human macrophages; b) ChemoTopo unit surface functionalised with iBOMAm cultured with human macrophages; c) Rank ordered human macrophage cell number normalised to flat TMPMP-co-TEGDA area showing distribution of human macrophage cell number across the ChemoTopoChip (N=2, n=3) ordered by chemistry 1-28, topography 1-36 and all combinations; d) Rank ordered human macrophage M2/M1 ratio normalised to flat TMPMP-co-TEGDA area showing distribution of human macrophage M2/M1 ratio across the ChemoTopoChip (N=2, n=3) ordered by chemistry 1-28, topography 1-36 and all combinations; e) macrophages on flat TMPMP-co-TEGDA area (blue=IL-10, yellow=TNFα); f) macrophages on flat 2-(4-benzoyl-3-hydroxyphenoxy)ethyl acrylate (BzHPEA) area (blue=IL-10, yellow=TNFα); g) macrophages on TMPMP-co-TEGDA+Topo 22 area (blue=IL-10, yellow=TNFα); h) macrophages on BzHPEA+Topo 22 area (blue=IL-10, yellow=TNFα).

FIG. 5—Chemistry-topography combination analysis a) SR plotted versus hiMSC cell number; b) SR plotted versus hiMSC ALP intensity (normalised); c) SR plotted versus human macrophage normalised cell number; d) SR plotted versus human macrophage M2/M1 ratio (normalised); e) Hit chemistries from macrophage and hiMSC datasets (see FIG. S1 for full list of chemistries); f) Hit topographies from macrophage and hiMSC datasets (see FIG. S2 for full list of topographies)

FIG. 6—Random Forest Modelling a) hiMSC ALP intensity random forest model using 1-hot descriptors for chemistries and topographical descriptors generated using CellProfiler; Error! Bookmark not defined, b) human macrophage polarisation random forest model using 1-hot descriptors for chemistries and topographical descriptors generated using CellProfiler; Error! Bookmark not defined, c) hiMSC ALP intensity random forest model top contributions; d) human macrophage polarisation random forest model top contributions.

FIG. 7—Chemistries Used in the ChemoTopoChip: nHDon, nHAcc and Log P refer to number of H-bond donors, number of H-bond acceptors and Log P (octanol/water partition coefficient) classified as high (H), medium (M) or low (L).

FIG. 8—Topographies Used on the ChemoTopoChip

FIG. 9—XPS of Base TMPMP-co-TEGDA and Example iBOMAm Functionalised Area

FIG. 10—Normalised hiMSC ALP Expression Levels Across ChemoTopoChip

FIG. 11—Normalised Macrophage M2/M1 Ratio Across ChemoTopoChip

FIG. 12—hiMSC ALP Intensity Topographical Descriptor Correlation Plots

FIG. 13—Human Macrophage M2/M1 Ratio Topographical Descriptor Correlation Plots

FIG. 14—Schematic of the high throughput screening approach used to identify hit polymers that drive macrophage phenotype towards a pro- or anti-inflammatory status, in-vitro and in-vivo. (a) High throughput printing of polymer arrays with different surface chemistries, (b) monocyte isolation from human buffy coats and seeding onto polymer arrays for 6 days, followed by macrophage phenotype assessment determined using a pro-inflammatory (M1) fluorescent marker in red (calprotectin) and an anti-inflammatory (M2) fluorescent marker in green (mannose receptor). (c) A selection of polymers that had high macrophage attachment and polarisation ability in-vitro were coated onto catheter segments and inserted subcutaneously into an in-vivo mouse model and assessed for their foreign body response. Example application of how polymer coatings could be used to encourage healing in dental and wound applications.

FIG. 15—Macrophage surface phenotype and adherence on homopolymer arrays. (a) Scatter plot showing M2/M1 cell number ratio for three biological samples. The M2/M1 cell number ratios above the upper green dashed line highlight M2 biased homopolymer hits and those below the lower red dashed line show polymers that induced M1 polarisation to a greater extent than in the cytokine reference populations. (b) The distribution of cell adherence on homopolymers. The large shaded area within each outlined rectangle indicates the mean value, and the mean±1s.d unit is presented in the narrow columns to the right (plus) and left (minus) of the mean. Data shown are mean values from 3 different biological replicates (donors) with a minimum of 2 repeats for each donor (donor 1:4 repeats, donor 2:3 repeats and donor 3:2 repeats). (c-g) Fluorescent images of cells stained for M1 marker calprotectin (red) and M2 marker mannose receptor (green) and nucleus (DAPI blue) on selected ‘hit’ polymers with either M2 (c and d) or M1 bias (e and f). (c) H47: poly(N-tris(hydroxymethyl) methyl acrylamide). (d) H37: poly(methacylamide). (e) H126: poly(isobutyl acrylate). (f) H98: poly(hydroxypropyl acrylate). Examples of (h) highly adhesive H42: poly(cyclohexyl methacrylate) and (i) poorly adhesive H39: poly(tridecafluorooctyl methacrylate). Scale bar=200 μm.

FIG. 16—Impact of co-polymers on macrophage polarisation and cell adherence. (a) Scatter plot showing M2/M1 cell number ratio of macrophages on co-polymers. Data shown are mean values from 3 different biological replicates (donors) including 3 technical repeats for each donor. Copolymers with M2/M1 cell number ratio ≥4 (upper green dashed line) and ≤0.4 (lower red dashed line) are considered M2 or M1 biased respectively. (b) Average number of adherent cells on co-polymer array. Numbers indicate the co-polymer identity. The large shaded area within each outlined area indicates the mean value, and the mean±1 SD unit is presented in the narrow columns to the right (plus) and left (minus) of the mean. (c-j) Fluorescent images of co-polymers that induce M1 (c-e), M2 (f-h) bias or similar number of M1 and M2 bias cells (M0) (i-j). Red shows calprotectin (M1 marker), green mannose receptor (M2 marker) and blue DAPI (nuclear stain). (k, l) Exemplar co-polymers with high (k) and low (l) cell attachment. Scale bar=200 μm.

FIG. 17—Histological analysis of tissue sections following 28-day implantation polymer coated catheter segments in a rodent model. Sections of tissue surrounding the foreign body site (*) were (a) H&E and (b) MTC stained 4 weeks post-implantation. Representative images show H&E and MTC stains with varying extents of foreign body response to each of the polymer coatings (no coating, M1 (24, 170), M2 (255, 301) and M0 (398, 408) like phenotypes from in-vitro studies) including; cell migration, macrophage, neutrophil and fibroblast infiltration and collagen thickness as a sign of fibrosis. (c) macrophage and (d) neutrophil infiltration counts from sites surrounding the foreign body and (e) collagen thickness measured from MTC stains as an indication of fibrosis. All data are presented as the mean with ±s.d (N=2 and n=5). Significance was calculated by one-way ANOVA with Tukey's post-hoc analysis: * P<0.05, ** P<0.01, *** <0.001. Scale bar=25 μm.

FIG. 18—Determining macrophage pro- or anti-inflammatory phenotype using fluorescence microscopy. (a) Determine fluorescent threshold diagram (b) M1 phenotype polarised with IFN-γ+GM-CSF (c) M2 phenotype polarised wit M-CSF+IL-4. (b,c). Fluorescent images of cells stained for calprotectin (27E10 antigen, red), and mannose receptor (MR, green). Scale bar=200 μm. (d,e) Scatter plot for number of M2/M1 polarised cells with cytokines on glass slide, X-axis average total cell number of the adherent cells, Y-axis is number of cells expressed MR⁺(M2-phenotype)/number of cell expressed calprotectin (M1-phenotype) on glass slide. n=3 (D, homo-polymer arrays experiment) and 2 (E, co-polymer arrays experiment) of different samples (M1 and M2) for each sample with 2 replicates.

FIG. 19—outlines the pipeline for polymer selection

FIG. 20—M2:M1 thresholds for macrophage polarisation. The thresholds are represented by the top and bottom horizontal dashed lines, corresponding to 4.0 and 0.3, respectively.

FIG. 21—Homo-polymer data clustering

FIG. 22—Machine Learning modelling results: (a) the structures of the molecules ranked by their average impact on model output, (b) the confusion matrices for the machine learning methods employed and (c) the chemical structures of the molecular entities that contributed most strongly to the attachment and polarisation.

FIG. 23—Macrophage functional analysis. (a-d) Cytokine secretion by cells cultured on different polymers, (a-d) show levels of TNF-alpha, IL-1β (pro-inflammatory cytokines), IL-10 and CCL18 (anti-inflammatory cytokines) respectively. Monocytes were cultured on co-polymer and TCPS surfaces for 6 days. Pro-inflammatory and anti-inflammatory cytokines levels in the culture supernatants were determined by ELISA. Data is presented as mean±S.D of 5 independent experiments using blood samples from 5 different donors. (e-m) Phagocytic activity of macrophages on polymer surfaces (f, g, i, j, l, m) or cytokine polarized macrophages (e, h, k). Monocytes were on surfaces coated with different polymers or in the presence of M1 and M2 polarising cytokines for 6 days. On Day 6 of culture, cells-were incubated with Alexa Fluor 488-labelled zymosan (green) particles (1 h at 37° C., 5% CO₂) then washed three times with PBS to remove non-phagocytosed particles before imaging. Images are representative of three independent experiments using 3 different donors each run in duplicates.

FIG. 24—Quantification of protein adsorbate thickness on polymer spots by XPS. To ensure precise measurements, the theoretical bulk and experimental bulk chemistry of the polymers pre- and post-incubation with serum containing media was compared. The nitrogen fraction was used to calculate protein thickness. The data showed significantly thicker protein layer on polymers H24 and C170 (M1 biased polymers) than C301 and C255 (M2 biased polymers) or C398 and C408 (naïve based polymers). All data shown as mean±s.d (n=5). Significance was calculated by one-way ANOVA with Tukey's post-hoc analysis: ** p<0.01, *** p<0.001.

FIG. 25. Macrophage phenotype analysis of tissue sections following 28-day implantation of catheter segments with (a) no coating and with polymer coatings (b) H24, (c) C170, (d) C255, (e) C301, (f) C398 and (g) C408 in a rodent model. The representative images show tissue sections stained for an M1-like marker (iNOS shown in green) and an M2-like marker (ARG-1 shown in magenta). A region of interest created around the foreign body sites (*) with background fluorescence subtraction was used to quantify the mean raw fluorescence intensity density (sum of all pixels in the given area). The ratio of M2-like macrophages to M1-like macrophages was calculated and is represented in image (h). All data are presented as the mean with ±s.d (N=2 and n=5). Significance was calculated by one-way ANOVA with Tukey's post-hoc analysis: ** p<0.01, *** p<0.001. Scale bar=25 μm

FIG. 26—representative human lung fibroblast marker staining.

FIG. 27—screening dtata depicting the attachment of polymers. The high throughput screening cell attachment graph shows the variation in fibroblast adhesion across the polymers. Representative images of TCP (control) and a low attachment polymer against high attachment polymer are depicted. A) Rank ordered graph of cell attachment on polymers. The dotted blue line intersects through TCP control. B) C) and D) Cells immunostained for nuclei (blue) and F-actin (green).

FIG. 28—expression of alpha smooth actin in the presence of polymers. The high throughput screening alpha—SMA expression graph shows the variation in alpha-SMA expression across the polymers. The black bars represent fibroblast culture on array without TGF-B1 while the gray bars represent the same order of polymers cultured with TGF-B1. The graph shows that the polymers are able to modulate alpha—SMA without the presence of TGF-B1 but even more so in the presence of TGF-B1 (as the same rank order of expression is not maintained with TGF-B1). Representative images of TCP (control) and alpha-SMA expression on polymers is shown.

FIG. 29—cell proliferation in the presence of polymers. The high throughput screening alpha-SMA expression graph shows the variation in alpha-SMA expression across the polymers. The black bars represent fibroblast culture on array without TGF-B1 while the gray bars represent the same order of polymers cultured with TGF-B1. The graph shows that the polymers are able to modulate alpha-SMA without the presence of TGF-B1 but even more so in the presence of TGF-B1 (as the same rank order of expression is not maintained with TGF-B1). Representative images of TCP (control) and alpha-SMA expression on polymers is shown. A) Cell proliferation of fibroblasts without presence of exogenous TGF-B1. B) Modulation of proliferation in the presence of TGF-B1. The dotted blue line intersects through TCP control.

FIG. 30—Selection process of polymers A) Shortlisting polymers based on cell attachment and cell size, the green ROI represents polymers with greater than 20 cells attached and 60 percent of cell size relative to cell size on TCP B) Shortlisted polymers based on ROI and 3SD criteria C) MFI of alpha-SMA of shortlisted polymers D) Polymer grouping relative to TCP control.

FIG. 31—homopolymer selection of FIG. 30D in detail. The grouping is as follows (relative to TCP control): 1—Increase in alpha-SMA expression and decrease in cell proliferation. 2—Increase in alpha—SMA and increase in cell proliferation. 3—Decrease in alpha—SMA expression and decrease in cell proliferation. 4—Decrease in alpha—SMA expression and increase in cell proliferation. Monomers chosen based on COV<25 percent (for CA, CP and ASMA). A high and low performing monomers (wrt CA, CP and ASMA) were chosen in each category. Decrease in SMA indicates a lack of differentiation towards myofibroblasts. Downstream this could translate to lower ECM secretion and modulation of fibrosis. Decrease in cell proliferation suggests a decrease in cell growth. This may be essential in certain medical conditions.

FIG. 32—List of fibroblast anti-attachment polymers.

FIG. 33—list of shortlisted polymers and their grouping based on FIG. 31. The highlighted polymers were selected for further studies.

FIG. 34—Polymer microarray screening for fungal attachment. (A) Fungal attachment assay procedure developed for C. albicans and B. cinerea. For detection, the C. albicans strain expressed yCherry, whereas B. cinerea was stained with Congo Red. (B) Microscopic images of B. cinerea spore adhesion on glass (top left) and three representative polymers from the microarray with differing attachment properties. The top right example indicates a polymer of interest. Polymer spots are diameter approx 300 μm. (C) Distribution of fungal-attachment results across the polymer arrays; percentage attachment values are relative to the median value (=100%) for each fungus. Values are means from at least three independent replicates. (D) Comparison of % attachment (relative to median) by B. cinerea and C. albicans to the different polymers. Eighty polymers giving the lowest attachment for each organism were selected for further study; 27 were common to B. cinerea and C. albicans.

FIG. 35—Biofilm formation on potential anti-attachment materials. Eighty polymers showing the lowest fungal attachment (from the preceding microarray-spot screen) were selected as materials for further study. These materials were scaled up to coat the 6.4-mm diameter wells of 96-well plates. Polymers showing surface cracking were excluded from the analysis. (A) Procedure for assessment of fungal biofilm formation on coated 96-well plates. (B) Extent of biofilm formation across the different polymers for C. albicans and B. cinerea. Materials of interest were designated as those showing <25% biofilm formation (dashed line), as compared with control microplate-wells that were not coated with polymer. The full data together with names and structures of the polymers of interest are listed in Tables 10 and 11. (C) These polymers of interest for B. cinerea were tested for biofilm formation also by the filamentous fungi Z. tritici and A. brasiliensis. Biofilm was again assessed after 24 h with XTT. The R² and p values for the Pearson correlations were 0.307 and 0.014 (Z. tritici and B. cinerea), 0.558 and 0.0002 (A. brasiliensis and B. cinerea), and 0.354 and 0.007 (Z. tritici and A. brasiliensis). The values are means±SEM from at least three independent experiments.

FIG. 35—Biofilm formation on potential anti-attachment materials. Eighty polymers showing the lowest fungal attachment (from the preceding microarray-spot screen) were selected as materials for further study. These materials were scaled up to coat the 6.4-mm diameter wells of 96-well plates. Polymers showing surface cracking were excluded from the analysis. (A) Procedure for assessment of fungal biofilm formation on coated 96-well plates. (B) Extent of biofilm formation across the different polymers for C. albicans and B. cinerea. Materials of interest were designated as those showing <25% biofilm formation (dashed line), as compared with control microplate-wells that were not coated with polymer. The full data together with names and structures of the polymers of interest are listed in Tables 10 and 11. (C) These polymers of interest for B. cinerea were tested for biofilm formation also by the filamentous fungi Z. tritici and A. brasiliensis. Biofilm was again assessed after 24 h with XTT. The R² and p values for the Pearson correlations were 0.307 and 0.014 (Z. tritici and B. cinerea), 0.558 and 0.0002 (A. brasiliensis and B. cinerea), and 0.354 and 0.007 (Z. tritici and A. brasiliensis). The values are means±SEM from at least three independent experiments.

FIG. 36—Anti-attachment versus growth inhibitory actions of selected materials. Materials of interest were tested for potential toxicity effects, alongside anti-attachment assays. Schematic summarising the procedures for assessment of attachment (A, left) or growth inhibition (A, right, B and C) (for chemical structures, see Tables 10-11). For attachment, C. albicans or B. cinerea were incubated for 2 h or 6 h, respectively, before the first wash and a further 22 h or 18 h before XTT assay. Growth inhibition by the coated materials was assayed either directly (A, right and B) or from release of toxic materials (C). (A) Attachment and toxicity results for C. albicans with the materials. Percentage values were calculated by comparison with control microplate-wells that were not coated with polymer. The differences between the polymers were not significant for either attachment or resistance. (B) B. cinerea cultivated for 15 days in PDB medium in 96-well plates coated with polymers. Scale bar, 1 cm. (C) Inhibition of B. cinerea growth by supernatant from pEGPhEA-coated wells (pEGPhEA was tested as the only material that exhibited toxicity in (B). Medium containing leached materials from the coated plate after 24 h was transferred to wells containing spores that had been pre-attached for 6 h before washing and addition of materials. Subsequent growth was compared with that from spores incubated with leached material-free medium, according to OD₆₀₀ determined after 24 h. *p<0.05 according to Student's t test, two tailed. Values are means±SEM from at least three replicate experiments.

FIG. 37—Resistance of AODMBA to Candida albicans biofilm formation on 3D-printed polymer forms and in drug resistant isolates. (A) AODMBA polymer 3D-printed into different forms including 3 mm-diameter coupons and 1.9 cm-length voice-prosthesis valve flap (bottom). (Photo credit: Yinfeng He, Univ. Nottingham). (B) Biofilm formation on the coupons (assayed as in FIG. 2A). PEG575DA (PEGDA) served as attachment positive-control. Values are means±SEM (n=3). **p≤0.01, unpaired t test. Microscopic images were taken just before XTT assay. Scale bar, 1.20 mm. (C) Candida albicans biofilm, stained with crystal violet (CV), on valve-flap samples 48 h post-inoculation. Biofilm that was evident with some AODMBA valve flaps detached when the form was moved or gently rinsed, unlike biofilms on commercial silicone-manufactured flaps (control). **p≤0.01, unpaired t test. Images are representative of ≥3 independent attachment assays. Scale bar, 0.37 cm. (Photo credit: Cindy Vallieres, Univ. Nottingham). (D) Percentage attachment to AODMBA by C. albicans CAF2-yCherry (WT) and azole-resistant isolates, C. albicans SCS119299X and J980280, relative to uncoated microplate-wells. Values are means±SEM from three independent experiments. There was no significant difference between strains according to Dunnett's multiple comparisons by two way ANOVA. Inset, azole resistances of the strains.

FIG. 38—Protection against fungal infection of plant leaves. Polymers were synthesised via free radical polymerisation using a thiol chain transfer agent; percentage of conversion, molecular weight (MN) and polydispersity (D) for each material were determined by ¹H-NMR and GPC analysis and shown in Table 12. (A) Materials of interest were prepared at 20% (w/v) (using 20% v/v isopropanol as solvent) and sprayed onto 1.5 cm dia. lettuce-leaf discs, before infection (right panel) or not (left panel) with B. cinerea (2,500 spores per leaf disc). Infection progress was examined daily up to 3 days post-infection (leaf samples deteriorated after that time). The box highlights polymers that gave the best protection from infection. Images are representative of at least three independent experiments, with 5 leaves infected per experiment. (B) Percentage of infected leaves at days 2 and 3 post-infection, corresponding to the right panel from (A); no infection was observed at day 1. The values are means±SEM. *p ≤0.05, ** p ≤0.01, *** p ≤0.001 according to multiple comparisons (Dunnett's multiple comparisons test) by two way ANOVA. EGMMA serves as a positive control. Data showing absence of toxicity to B. cinerea with these polymer preparations are presented in FIG. 46. (C) Resilience of coated polymer to rinsing with water, TEGMA was sprayed onto 1.5 cm dia. lettuce-leaf discs and leaves were washed with water or not before infection as in (B). Images taken at 3 days post-infection are representative of 5 leaves infected per condition.

FIG. 39—PLS regression correlating the natural log of fluorescence due to C. albicans attachment and surface chemistry as measured by ToF-SIMS. A) RMSECV for PLS regression conducted with varying numbers of latent variables. Three latent variables were selected for the final PLS model. B) The measured versus predicted fungal attachment values for the training (I) and test (x) datasets. The y=x line is drawn as a guide. R²=0.43. C) The regression coefficients for the X-variables after sparse selection. A total of 37 variables were used for the model. D) List of the top ions with the largest positive or negative regression coefficients used in the PLS model with possible chemical assignments.

FIG. 40—PLS regression correlating the natural log of fluorescence due to B. cinerea attachment and surface chemistry as measured by ToF-SIMS. A) RMSECV for PLS regression conducted with varying numbers of latent variables. Three latent variables were selected for the final PLS model. B) The measured versus predicted fungal attachment values for the training (I) and test (x) datasets. The y=x line is drawn as a guide. R²=0.20. C) The regression coefficients for the X-variables after sparse selection. A total of 24 variables were used for the model. D) List of the top ions with the largest positive or negative regression coefficients used in the PLS model with possible chemical assignments.

FIG. 41—ML model correlating the natural log of fluorescence due to C. albicans attachment and surface chemistry as measured by ToF-SIMS. A) The measured versus predicted fungal attachment values with standard deviation of measured and predicted values and prediction confidence levels. The y=x line is drawn as a guide. R²=0.47. B) Ions associated with high or low feature importance.

FIG. 42—ML model correlating the natural log of fluorescence due to B. cinerea attachment and surface chemistry as measured by ToF-SIMS. A) The measured versus predicted fungal attachment values with standard deviation of measured and predicted values and prediction confidence levels. The y=x line is drawn as a guide. R²=0.35. B) Ions associated with high or low feature importance.

FIG. 43—Machine learning results for C. albicans using signature molecular descriptors. A) Measured versus predicted attachment XGBoost regression results with standard deviation of measured and predicted values and prediction confidence levels. The y=x line is drawn as a guide. R²=0.70. B) Ranked feature importance, with the most relevant fragment descriptors selected for modelling. C) Molecular fragments most relevant to attachment.

FIG. 44—Machine learning results for B. cinerea using signature molecular descriptors. A) Measured versus predicted attachment XGBoost regression results with standard deviation of measured and predicted values and prediction confidence levels. The y=x line is drawn as a guide. R²=0.43. B) Ranked feature importance, with the most relevant fragment descriptors selected for modelling. C) Molecular fragments most relevant to attachment.

FIG. 45—Relationship between inoculum-size and subsequent biofilm detection with the XTT assay (metabolic activity measurement). As depicted in FIG. 35A, non-coated wells were inoculated with different concentrations of C. albicans cells or B. cinerea spores for 2 or 6 h, respectively. Non-adherent cells were washed away and fresh medium was added to the wells. After 24 h, wells were washed again and biofilm formation assessed using XTT salt. The values are means±SEM from at least three replicate experiments. In the screens performed during this work, materials-of-interest were designated as those yielding a biofilm (metabolic activity)<25% compared to the control. In the case of C. albicans, FIG. 41 shows that <25% is approximately equivalent to a biofilm that would be formed from a starting inoculum of ≤10 cells. That is, since the starting inoculum used in the main assays is ˜12,500 cells of C. albicans per well, ˜10 cells equates to a ˜99.9% reduction in attachment (bearing in mind that biofilm formation is saturated at a starting inoculum above ˜250 cells; top panel). In the case of B. cinerea, <25% is approximately equivalent to a biofilm that would be formed from a starting inoculum of ≤10,000 spores; as the starting inoculum used in the main assays is ˜250,000 B. cinerea spores/well, ˜10,000 spores equates to a ˜96% reduction in attachment.

FIG. 46—Absence of growth-inhibitory effects against B. cinerea by polymer preparations used for leaf infection assays. To assay potential growth-inhibitory effects of the relevant preparations, the bases of wells in 96-well plates were coated with the polymers (50 μl of the relevant polymer preparation were allowed to dry in each well) and then inoculated with the organism as described in FIG. 36B. The image shows growth after 15 d, in triplicate for each treatment.

FIG. 47—Retention of TEGMA on leaves after washing. (A) Leaf sections untreated (n) or coated with TEGMA (n) and either unwashed (dark) or washed (light) were assessed by ToF-SIMS using the same experimental conditions used to characterize polymer samples. Aliphatic carbon ions likely associated with wax on the leaf (C₄H₉⁺, C₆H₁₁⁺ and C₇H₁₃⁺) or ions likely associated with the oligoethylene glycol moiety (C₁₁H₂₀O₄⁺, C₁₂H₁₅O₆⁺ and C₁₃H₂₉O₇⁺) were quantified for each sample. Ion intensity was normalized to total ion count and then to the maximum intensity observed for each ion across the four sample sets. Error bars equal one standard deviation unit (N=3). Peaks associated with the oligoethylene moiety were significantly higher (p<0.025) on treated samples compared with untreated samples. No significant change was observed after washing. (B) Ion images for characteristic ions C₆H₁₁⁺ (leaf) and C₁₂H₁₅O₆⁺ (oligoethylene). The coating appears patchy on the TEGMA-treated sample, with regions associated with the polymer and other regions associated with the leaf both visible.

FIG. 48—workflow for neutrophil polymer array screening.

FIG. 49—Numerical and confocal assessment of neutrophil attachment to surfaces coated with different polymers.

FIG. 50—Classification of monomers which influence neutrophil attachment.

FIG. 51—Multi-generation microarray screen of polymeric substrates (a) A first-generation array of 284 chemically diverse monomers were screened for hPSC attachment with ReBI-PAT hiPSCs in E8 medium for 24 h. (b) Arrays were then fixed and stained for pluripotent marker OCT4, imaged using Imstar automated fluorescence microscopy and OCT4+ nuclei and total nuclei counts assessed with Cell-Profiler. Representative image shows a polymer spot (n) supporting high hPSC attachment. (c) Attachment on materials are ranked by OCT4+ nuclei count plotted against total cell number (DAPI). Nineteen materials selected for second-generation co-polymer screening (highlighted in red). Each point represents mean (n=9) and SEM for OCT4 count. (d) A total of 361 chemistries screened for 24 h included 19 selected monomers printed alone and mixed pairwise (2:1 v/v). (e) OCT4+hPSC attachment (n=9) was clustered by Euclidean distance measure (intensity scale represents OCT4+ nuclei count) and (f) ranked (high to low). Polymer D, (TCDMDA) containing polymers are denoted in red. All letter IDs mentioned are defined in FIG. 54.

FIG. 52—Screening polymers at scale-up. (a) Schematic view of polymer screening at scale up. ReBI-PAT hPSCs were seeded onto polymers (48 hr attachment hit materials from array screen, triplicate wells/polymer) and Matrigel™ at 4.5×10⁵ cells/cm² in E8 medium. All images (15 fields of view/polymer) were taken (Operetta, Perkin Elmer) and processed using Harmony image analysis software (Perkin Elmer). Brightfield images were processed at 24 hrs using scripts developed with PhenoLOGIC machine learning (script training (left centre panel) training: green dots=cells, red dots background, resultant overlays (left bottom panel)). hPSCs were fixed and stained for OCT4 expression and quantified by nuclei count (script right panel). (b) Representative brightfield images of REBI-PATs cultured on polymers (structures to letter IDs in FIG. 55) and Matrigel in E8 medium supplemented with ROCKi. (c) % cell coverage calculated relative to area imaged and Matrigel™ control and (d) mean colony sizes calculated from total colonies observed/field of view after 24 hrs culture. (e) Representative OCT4+ cell attachment on polymers remaining at 72 hrs (>85%). (e) Cell attachment calculated from total nuclei count of hPSCs attached after 72 hrs. All graphs represent mean (±SEM). One way ANOVA followed by Tukey's multiple comparison tests (*p<0.05, **p<0.01, p<0.001). Scale bars of all images presented are 200 μm.

FIG. 53—Characterisation of poly(TCDMDA-blend-BA) surface. Atomic force microscopy (a) Derjaguin-Muller-Toporov (DMT) modulus and (b) deformation micrographs of poly(TCDMDA-blend-BA) surface coated on poly(styrene) six well plates showing a nanoscale blend of poly-BA (˜50 nm islands of minor component, 30% v/v) in poly-TCD (background, major component, 70% v/v). (c) Representative brightfield images of ReBI-PAT attachment and maintenance at 24 h and 72 h of culture. (d) Growth curves of hESC: HUES7 and hiPSC AT1 and ReBI-PAT lines on poly(TCDMDA-blend-BA) and Matrigel™ presented as cumulative doubling time (for equation see methods). (e) LESA-MS/MS quantification of adsorbed proteins: FGF2, TGFβ1, insulin and transferrin from E8 medium on polymer surface after 1 h of incubation (N=3). Bar graph represents ±SEM. One-way Anova statistical tests were performed and compared between chemistries for each protein (*p<0.05). (f) Blocking of integrins and RGD-blocking peptides for 24 h on poly(TCDMDA-blend-BA) in E8. Bar graphs presented as mean and error bars represent ±STDEV. (N=3, n=3). One way ANOVA followed by Tukey's multiple comparison tests (*p<0.05, **p<0.01, ****p<0.0001) were performed. Statistical differences denoted with black asterisks represent comparisons between polymers and Matrigel unless indicated otherwise (g) Phosphokinase array blots were quantified using Image Studio Software for hESC line HUES7 (N=2, n=4) and hiPSC line AT1 (N=1, n=2). Heatmap represents total intensity values per phosphorylated kinase normalized to background intensity and HSP60 internal control. Graph shows Mean+STDEV. (h) Schematic to summarize identified hPSC and poly(TCDMDA-blend-BA) interactions. The upper panel are zoomed in single cell-polymer interactions from lower image showing hPSC colonies attached on a well of cultureware coated with poly(TCDMDA-blend-BA). In brief (left to right), adsorption of E8 proteins mediate initial phase of cell attachment (phase I) followed by integrin engagement (phase II) which subsequently promote key hPSC signaling pathways (phase Ill).

FIG. 54—Monomer structures of 23 materials selected for second generation co-polymer screen labelled A-W as referred to in main text.

FIG. 55—TOFSIMS analysis of poly(TCDMDA-blend-BA) surface on scaled up tissue culture wells. Ions characteristic to BA [C₄H₉]⁺ m/z=57.07 and TCDMDA (C₅H₇⁺ m/z=67.05) on polystyrene based tissue culture plastic ware [C₂H₇]⁺ m/z=91.06.

FIG. 56—Karyograms observed after 5 serial passages on poly (TCDMDA-blend-BA) for (a) hESC HUES7 (46,XY), (b) hiPSC AT1 (46, XX) and (c) hiPSC REBI-PAT (46, XY) cultured in E8 medium.

FIG. 57—hPSCs (hiPSC AT1 and REBI-PAT lines and hESC HUES7 line) were assessed for pluripotency markers after 18 days (5 serial passages) on poly(TCDMDA-blend-BA) and compared to Matrigel by (a) flow cytometry (b) quantitative real-time PCR, (c) and immunostaining (ReBI-PAT). Scale bar represents 200 μm.

FIG. 58—Protein expression integrin markers expression of AT1 cells cultured on Matrigel and D:Q (poly(TCDMDA-blend-BA) for at least three serial passages assessed by western blot analysis. (a) Representative images of Western Blotting bands for Integrins (αᵥ, α₅, β₁, β₄, β₅), stem cell marker Nanog, and house-keeping β-actin, (n=3) (b) Quantification of band intensity for integrin expression in AT-1 hiPSCs (n≥3), bars show Mean±STDEV; black bars shows Matrigel control and grey bars show AT-1 on the hit Polymer. Unpaired t-test were performed *P<0.033, **P<0.002, ***P<0.001.

FIG. 59—Viability assay of DCs cultured on polymers for 6 hours. 50,000 immature DCs were seeded in each well of 96-well plates in duplicate; data is presented as mean±range. Viability was assayed by CytoToxGlo (Promega). A box is set around the polymers that had more than 75% viability. A dotted line portrays the tissue culture plastic control.

FIG. 60—Polymer modulation of CD83 and CD86 expression. Polymers are investigated for their ability to activate/increase CD86 (A) and CD86 (B) expression levels after 24 hours of DC culture on polymers. Cells were stained for respective markers and analysed via flow cytometry. Data is from 3 independent experiments and show percentage of positive cells as mean±SD. Experimental conditions are compared to the negative immature DC (iDC) control. In a second set of experiments Polymer are investigated for their ability to suppress activation of DCs—decrease their CD83/CD86 expression levels. For this, DCs were conditioned on polymers for 6 hours, subsequently stimulated with 10 ng/mL LPS and cultured for 18 hours before being stained for CD86 (C) and CD83 (D). Data is from 3 independent experiments are presented; mean±SD. Experimental conditions are compared to the positive stimulated DC (mDC) control. ONE-way ANOVA with Bonferroni multiple comparisons test.

FIG. 61—Assessing modulated cytokine secretion of IL-10 (A) and IL-12 (B) after culturing iDCs for 24 hours on polymers. Polymers are investigated for their ability to increase IL-10 and IL-12 secretion. Experimental conditions are compared to the TCP iDC control. Data is from 3 independent experiments with 2 technical repeats each and represented as mean±SD. For assessing whether polymers can modulate IL-10 (C) and IL-12 (D) secretion in presence of a stimulus, DCs were conditioned on the different polymers for 6 hours, then stimulated with 10 ng/mL LPS and cultured for a further 18 hours. Data is from 3 independent experiments with 2 technical repeats each and represented as mean±SD. Experimental conditions are compared to the TCP stimulated DC (mDC) control. All data point have been normalised according to the viability these polymers effected from FIG. 59. ONE-way ANOVA with Bonferroni multiple comparisons test.

FIG. 62—Modulation of endocytosis capability A polymers that are characterised as activation inducing B Polymer that are characterised as activation suppressing A-B Data is from 3 independent experiments and % of uptake is presented, when compared to negative uptake (4° C.) control. Polymers are investigated for their ability to modulate particle uptake/endocytosis. Experimental conditions are compared to the stimulated DC (mDC) control. ONE-way ANOVA with Bonferroni multiple comparisons test.

FIG. 63—(a) Synergy of co-polymer combinations were quantified as a ratio of OCT4+ attachment for co-polymer to their corresponding homopolymer components (see supplementary information for methods) clustered by Euclidean distance measure. Synergy ratios (SR) >1 are synergistic combinations (denoted yellow—red), SR values=1 are additive combinations (denoted in white) and SR values <1 are antagonistic combinations (denoted in grey). All letter IDs mentioned are defined in FIG. S2. (b) SR scores were plotted against average total cell number (n=9, where n represents the no. of polymer spots). Data has been defined as synergistic (red), additive (grey) or antagonistic. Data points to the right of dotted line represent high attachment polymers. Highlighted data points (blue) are co-polymer candidates selected for scale-up experiments. All attachment data is summarized in table S3.

FIG. 64—Tri-lineage differentiation of REBI-PAT hPSCs cultured on poly (TCDMDA-blend-BA) for five passages. (a) Definitive endoderm differentiation induced early-stage marker expression of FOXA2 and SOX17 after 2 days. (b) Ectoderm differentiation induced neurogenesis marker expression after 5 days. (c) Mesoderm differentiation induced positive α-actinin expression after 8 days. Scale bars represent 100 μm.

FIG. 65—Phenotype modulation after polymer culture. A CD83 B CD86% of positive cells, C correlation of CD83 and CD86 modulation after 24 hours of DC culture on polymers, D CD83, E CD86% of positive cells, F correlation of stimulated cells on polymers. Data from 3 independent biological samples and show percentage of positive cells as mean (±SD) of all biological repeats. Experimental conditions are compared to the TCP cultured base control DCs (symbol: ⋄ stands for TCP control DCs, ♦ stands for TCP cultured DCs stimulated with 100 ng/mL LPS, □ symbolises polymers chosen for the next functional assay).

FIG. 66—Assessing stimulatory polymers for their effect on cytokine secretion of IL-10 (A), IL-12 (B) and endocytosis (uptake ability) (C). Experimental conditions are compared to the TCP iDC control. Data is from 3 independent experiments (donors are colour coded) with 2 technical repeats each and represented as mean±SD. Data is from 3 independent experiments and % of uptake is presented as mean±SD, when compared to TCP control. ONE-way ANOVA with Bonferroni multiple comparisons test. For significance: *<0.0332; **<0.0021; ***<0.0002; ****<0.0001.

FIG. 67—Assessing inhibitory polymers for their effect on cytokine secretion of IL-10 (A), IL-12 (B) and endocytosis (uptake ability) (C). Experimental conditions are compared to the TCP+LPS (activated DC) control. Data is from 3 independent experiments (donors are colour coded) with 2 technical repeats each and represented as mean±SD. Data is from 3 independent experiments and % of uptake is presented as mean±SD, when compared to TCP control. ONE-way ANOVA with Bonferroni multiple comparisons test. For significance: *<0.0332; **<0.0021; ***<0.0002; ****<0.0001

FIG. 68—Polymer treatment influences the outcome of DC-T cell interactions (A) T-cell proliferation assay using Pan T cells as responder cells after 8 days of co-culture; (B) IFN-γ production of Pan T cell-Polymer DC cultures. Data is from 3-4 independent experiments and presented as mean±SD, n=3-4, N=2-4, donors are colour coded. Conditions are compared to TCP control.

FIG. 69—Overview of further cytokine (A-C) and surface marker (D-G) modulation. Further analysis of ZnA's immunoregulatory properties (H-J). Data is from 3 independent experiments and presented as mean±SD, n=3, N=2 for the cytokines analysis, donors are colour coded.

FIG. 70—Schematic overview of the tumour killing assay. Human MCF7 breast cancer cells expressing HLA-A1 were first incubated with mitomycin-c (MMC) at 37° C. overnight to confirm they were rendered non-proliferative. Then DCs expressing HLA-A1 (MHC typed and MHC matching to the breast cancer cell line) were conditioned on the polymer coatings and cultured with MMC-treated MCF7 cells overnight at 37° C. before being cultured with MHC-matched naïve CD8+ T cells for 8 days in the presence of IL-2. Proliferated tumour-specific CTLs were then seeded on MCF7 monolayers for 6 hours in differing ratios (1:5 to 1:20).

FIG. 71—Specific tumour killing is increased in conditions where DCs were conditioned on polymers A (example images) and B specific lysis of different effector to target ratios. Specific lysis is measured by dividing dead/total cell numbers for each condition in at least 2 images per well (2 technical replicates each from n=1). Data is represented as mean±SD.

FIG. 72—Cytotoxicity after monomer addition to DC culture for 24 hours. Cytotoxicity assessed by two different assays (CytoToxGlo and tryphan blue); the red dotted concentration of monomer found via mass spectrometry analysis were found in polymer culture. Concentrations chosen up to 1000 more than measured concentration and one below. n=2 for tryphan blue, CytoToxGlo n=1.

FIG. 73—Phenotype after monomer addition into DC culture. Different amounts of monomer were added into DC culture, normalised per TCP unstimulated DC baseline per donor, n=2.

FIG. 74—Thickness of serum proteins adsorbed to polymer surface. n=2, with 2 areas each.

FIG. 75—shows that full thickness wounds (approx. 8 mm in diameter) were created in 10 week old diabetic mice with BKS.Cg-m Dock 7m+/+Lepr db/J background. The wounds received either non-functional particles or particles made of a ‘pro-healing’ polymer. Wound closure was monitored over 3 weeks followed by histological analysis of wounds in each group. On day 20 of the study, wound closure in the animal receiving the functionalised particles is almost complete with clear granulation tissue formation.

FIG. 76—demonstrates partial epithelialization in diabetic animal 20 days after icision.

FIG. 77—Shows more extensive granulation tissue formation, contraction & re-epithelialisation in wound in receipt of functional beads compared to non-functional beads. This is in line with wound images presented in slide 1. Similar numbers of beads present on the two wounds.

FIG. 78—Attachment of human pluripotent stem cells on “blend” and “co-polymer” TCDMDA containing solutions is comparable. (a) Growth comparison of HUES7 human pluripotent stem cells on poly(TCDMDA-blend-BA) UV polymerized and hyperbranched poly(TCDMDA-co-BMA) 6 well-plates. Plates produced for the hyperbranched condition were prepared with and without plasma treatment, whilst all plates produced by the UV polymerization methods have been plasma treated prior to well coating. (b) Attachment on poly(TCDMDA-co-BMA) after 24 hr on scaled-up 6 well-plates.

FIG. 79—shows monomer microarray screening results for human pluripotent derived cardiomyocytes (HPSC-CMs). High attachment hits are nitrogen containing polymers. Attachment of cardiomyocytes on monomer microarray screen of polymeric substrates (a) 284 chemically diverse monomers were screened for human pluripotent stem cell (REBI-PAT cell line) derived cardiomyocytes (REBI-PAT-CMs) cultured in serum-free medium for 7 days. Arrays were fixed and stained for cardiac marker alpha-actinin, imaged using Imstar automated fluorescence microscopy and ranked by alpha-actinin positive cell count. (b) High attachment polymer substrates (c) High attachment polymer substrates contain Nitrogen containing moieties ie. Amine-containing functional groups (NH2).

FIG. 80—Indicates that amine-containing polymers can improved cardiomyocyte functionality. Improved cardiomyocyte functionality on amine-based polymers coated on tissue culture plasticware (TCP). REBI-PAT-CMs were cultured on coated surfaces for 7 days before being analysed for spontaneous contraction using CELLOPTIQ-based optical imaging (100 frames/second) (a) Chemical structures of polymers assessed on TCP with identity of linker (BmAm) (b) Schematic shows the parameters measured for contractility analysis. The diagram shows one contraction peak. Contraction time represents the time taken for a peak to reach its maximum amplitude [contraction amplitude (0 (baseline)-100)]. Relaxation time is the time taken for a peak to return to the baseline (0). (c-e) Contraction parameters quantified: (c) contraction amplitude with a single representative contraction curve/condition (higher amplitude indicates improved functionality) (d) relaxation rate (contraction amplitude/relaxation time where a faster rate indicates improved functionality); and (e) contraction rate (contraction amplitude/contraction time where a faster rate indicates improved functionality). One-Way Anova with multiple comparisons were performed (* p<0.05, ** p<0.01, *** p<0.001, and **** p<0.0001). Comparisons are between Matrigel controls and polymer conditions unless otherwise indicated. N=3. n=15.

FIG. 81—Co-polymerization to optimize material properties (glass transition temperature, Tg) for fungal anti-attachment applications. (A) Examples of hit homo-polymers with high Tg versus TEGMA with low Tg. (B,C) the indicated co-polymers were prepared and compared for C. albicans (CA) attachment and for toxicity. Both co-polymers retained anti-attachment while offering improved Tg versus the parent homo-polymers.

FIG. 82—Adsorption of FGF2 and TGFβ proteins from incubation with E8 medium quantified by LESA-MS/MS. (n=3). Bar graph represent ±SEM. One-way Anova statistical tests were performed (*p<0.05).

FIG. 83—Phosphokinase kinase array (a) Array blots were quantified using Image Studio Software for HUES7 (n=2) and AT1 (n=1) cell lines. Individual total signal intensity was measured by manual gating. All intensity values were normalized to background intensity and HSP60 internal control. Spots were excluded from analysis if they were visually to weak/unclear. Samples per condition were obtained in parallel. Graph shows Mean+STDEV. Representative rray blots analysed per condition for (a) HUES7(b) AT1.

FIG. 84—shows a) Definitive endoderm differentiation on poly (TCDMDA-co-BA) induced early-stage marker expression of FOXA2 and SOX17 after 2 days. (b) Ectoderm differentiation on poly(TCDMDA-co-BA) induced neurogenesis marker expression after 5 days. (c) Mesoderm differentiation on poly(TCDMDA-co-BA) induced positive α-actinin expression after 12 days. All images represent 200 μm. Zoomed image of α-actnin staining represents 100 μm.

### METHODS

### Methods Relating to Analysis of ChemoTopo Combinations

### ChemoTopoChip Design

The ChemoTopoChip design comprised 36 Topo units of a 500×500 μm size, including one flat control, arranged in 3×3 mm ChemoTopo units repeated 27 times, each with a different chemical functionalisation. 1 The microtopographies used maximiseD the morphological differences of MSCs. The chemistries were chosen from libraries of (meth)acrylate and (meth)acylamide monomers to provide maximum chemical diversity. The monomers are used to functionalise the surface of topographically moulded chips, which minimises differences in material compliance between chemistries sensed by the attached cells.

### ChemoTopoChip Fabrication

A silicon mould was fabricated from the ChemoTopoChip design using photolithography and etching to produce the negative master of the topographies. The desired features were produced from this master by injecting a 1:2 mixture of monomers trimethylolpropane tri(3-mercaptopropionate):tetra(ethylene glycol) diacrylate (1:2 TMPMP:TEGDA) containing the photoinitiator 2,2-dimethoxy-2-phenylacetophenone (DMPA) between a methacrylate-functionalised glass slide and the silicon master (FIG. 1.f); UV curing and solvent washing then provided the moulded ChemoTopoChip substrate, chosen as similar photopolymerised thiol-ene systems have been reported as tough shape memory, flexible materials offering low shrinkage stress that are sufficiently transparent to allow transmission optical imaging. Functionalisation of the ChemoTopo units was carried out by deposition of 50% w/v or 75% v/v monomer solutions in N,N-dimethylformamide (DMF) containing 0.05% w/v DMPA onto each ChemoTopo unit; further UV curing and washing steps delivered the final ChemoTopoChip (FIG. 1.d, FIG. 1.e).

### ChemoTopoChip Characterisation

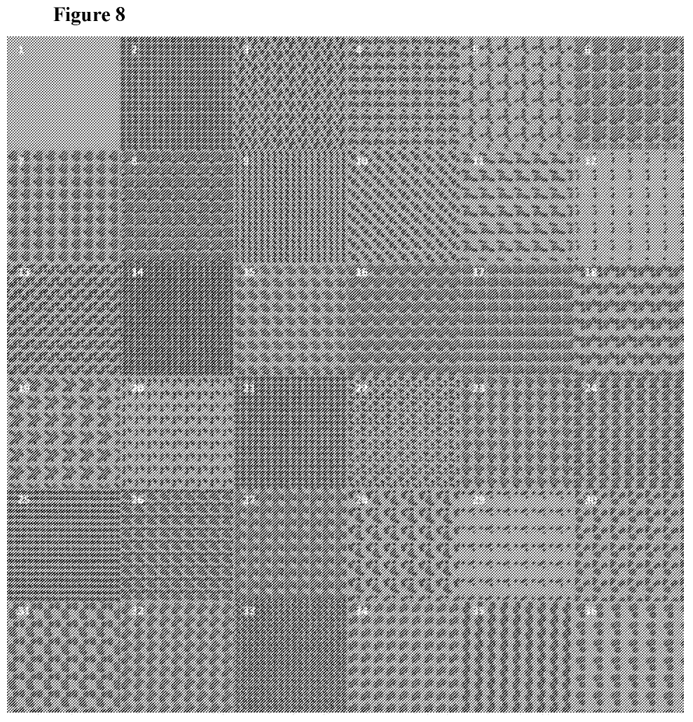

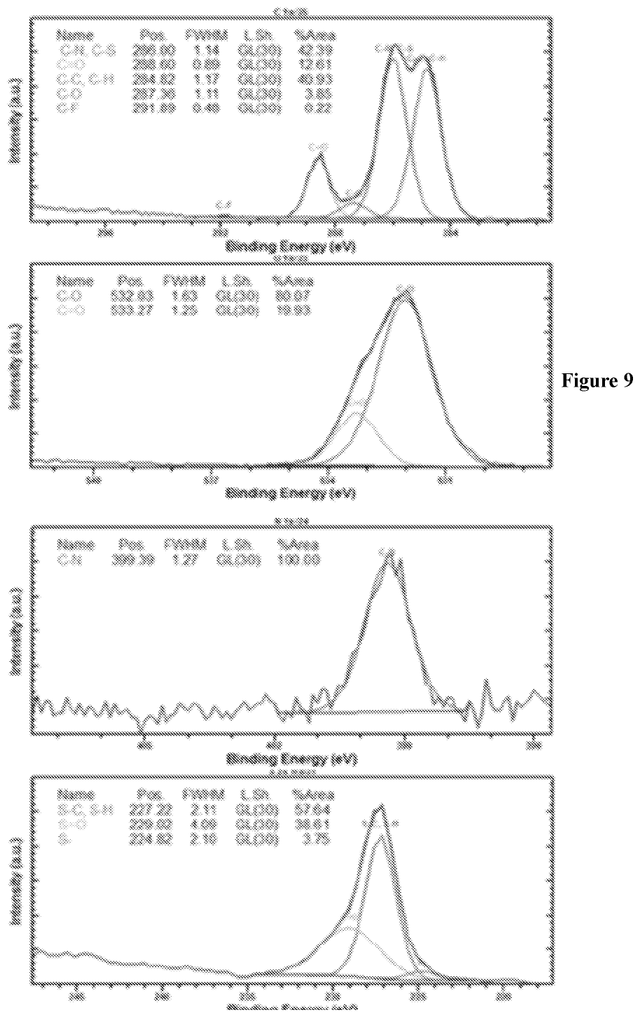

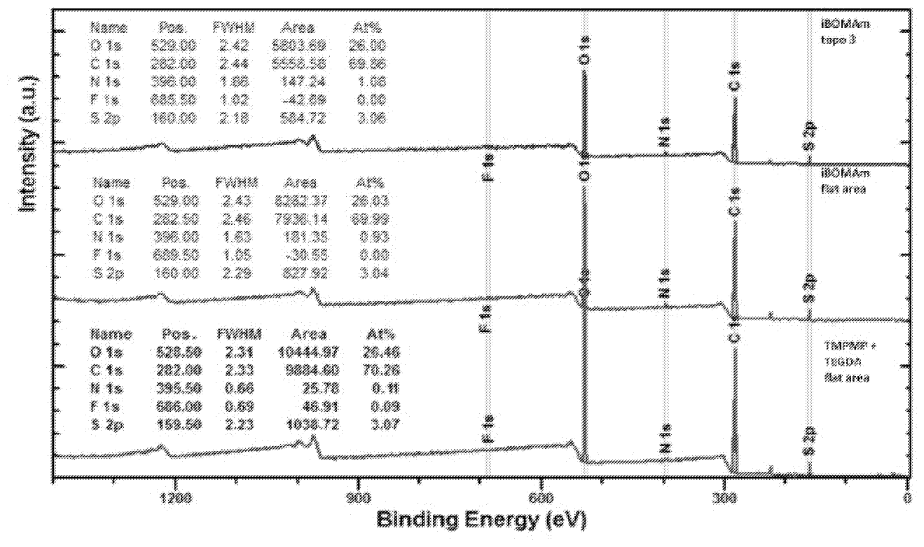

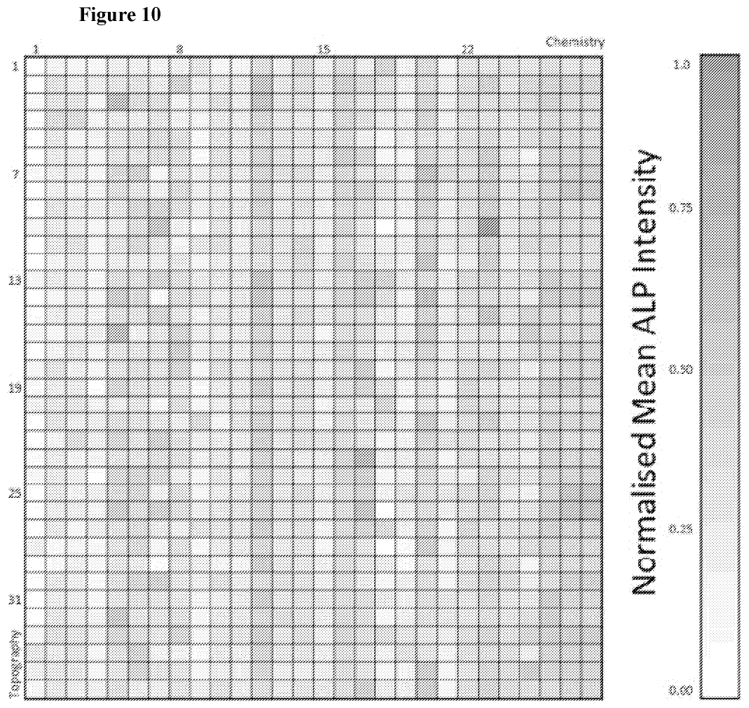

Surface chemical analysis is readily performed on this platform, allowing both the measured material surface chemistry and the biomolecular layer adsorbed from culture media to be probed for relationships with the observed cell response. The manufacture of the chip was optimised using surface chemical characterisation by time-of-flight secondary ion mass spectrometry (ToF-SIMS, FIG. 2.b) and X-ray photoelectron spectroscopy (XPS). ToF-SIMS showed that characteristic ions corresponding to the various surface chemistries were confined to the vicinity of their respective ChemoTopo units. Signals corresponding to the substrate [SH]− ion could be seen at similar intensities across the entire chip on both the base TMPMP-co-TEGDA material and the functionalised ChemoTopo units, indicating that this base material is also present at the surface. XPS analysis showed that the elemental compositions of equivalent chemistries on the flat area and topographically moulded areas of the chemically modified surfaces were approximately equal (FIG. 9). Incorporation of elements specific to the polymer overlayer could be detected, with the measured surface elemental composition consistent with the overlayer being thinner than the XPS analysis depth (ca. 10 nm). (FIG. 9)

The shape of the Topo units was characterised using optical interference profilometry which indicated that replication in the moulding process was effective, showing good feature reproduction after moulding and functionalisation (FIG. 2.a). Feature height was measured to be 9.1±0.6 μm across all Topo units compared to a feature height on the master of 10 μm, suggesting slight shrinkage on curing.

Substrate compliance, measured using AFM force measurement in peak force tapping mode, and found not to exhibit a difference for any materials across the chip when compared with the TMPMP-co-TEGDA base substrate modulus (see Table 1).

### Methacrylate Functionalisation of Glass Slides

Glass slides (26 mm×50 mm×0.40 mm) were activated using 02 plasma (pi=0.3 mbar, 100 W, 1 min) and immediately transferred into dry (4 Å MS) toluene (50 mL) under argon. 3-(trimethoxysilyl) propyl methacrylate (1 mL) was added, and the reaction mixture heated to 50° C. for 24 h. The slides were then cooled to room temperature and washed by sonication with 3×10 mL fresh toluene. The slides were then dried under vacuum in a silicone-free vacuum oven (50° C.) for 24 h.

### Moulding of TMPMP-Co-TEGDA Substrate

TEGDA (337 μL) and TMPMP (163 μL) were added together under argon with DMPA (16.9 mg). The mixture was then sonicated for 15 min to ensure mixing. Each ChemoTopoChip mould on the silicon wafer was framed on 3 sides with Scotch tape (3M) spacers, and a methacrylate silanised glass slide placed on top of each ChemoTopoChip to be moulded; standard glass microscope slides (25 mm×75 mm×1.0 mm) were placed on top as weights to hold the silanised slides in place. The TMPMP/TEGDA reaction mixture was transferred into an argon glove box (<2000 ppm 02) along with the silicon mould, and the monomer solution (60 μL) pipetted between the silicon wafer and silanised slides. The rate of pipetting was manually maintained at a similar rate to that of the capillary forces acting upon the solution. When all ChemoTopoChip positions were been pipetted (˜10 min per ChemoTopoChip) they were irradiated with UV light (368 nm, 2×15 W bulbs, 10 cm from source) for 10 min. Once complete, the entire moulding setup was removed from the glove box and the glass microscope slide weights removed. The silicon wafer was then placed on to a pre-heated (70° C.) hot plate; after 10 min, the moulded ChemoTopoChips were carefully removed using a scalpel (CAUTION: excessive force and speed will break the thin glass substrate). Once removed, the moulded ChemoTopoChips were cleaned by sonication in acetone (10 mL, 10 min) then isopropyl alcohol (10 mL, 10 min). Finally, the ChemoTopoChips were dried under vacuum (0.3 mbar) for 24 hours before functionalisation.

### Functionalisation of Moulded ChemoTopoChip Samples

Monomer solutions were made up as follows: 75% v/v in N,N-dimethylformamide (DMF) for oils; 50% w/v in DMF for solids. Next, 0.05% w/v photoinitiator DMPA was added to these solutions before degassing by sonication (10 min). The moulded ChemoTopoChip samples were then transferred into an argon glove box (<2000 ppm 02) along with these monomer solutions. A total of 3 μL of monomer solution was then applied to each respective ChemoTopo unit, taking care to evenly cover the entire area required for functionalisation. The ChemoTopoChips were then irradiated with UV light (368 nm, 2×15 W bulbs, 10 cm from source) for 15 min, before being removed from the argon glove box and sonicated in isopropanol for 10 min. Due to the lower bond dissociation energy of the acrylate π-bond compared with that of the thiol σ-bond, it was expected that these monomers would polymerise to the thiol moieties on the base TMPMP-co-TEGDA substrate after photoinitiation commences. The samples are then placed under vacuum (0.3 mbar) for 7 days before use.

### Mesenchymal Stem Cell Culture

Human immortalised mesenchymal stem cells (hiMSCs) were generated in-house by lentiviral transfection of E6/E7 and hTERT genes as previously described. Cells were cultured in Dulbecco's modified Eagle's medium supplemented with 10% (v/v) foetal bovine serum, 100 units/mL penicillin, 100 μg/mL streptomycin and non-essential amino acids. Positive controls were cultured in Human Mesenchymal Stem Cell (hMSC) Osteogenic Differentiation Medium (PT-3002; Lonza). All cells were maintained in a humidified incubator at 37° C. and 5% CO2 in air. Cells were re-suspended in the appropriate volume of media and seeded on 3 replicate ChemoTopoChips at 1×105 hiMSCs/chip (3 independent experiments using cells from 3 different passage numbers).

hiMSC Immunofluorescence Staining

For alkaline phosphatase (ALP) staining, cells were cultured on the ChemoTopoChips for five days in culture medium (at 37° C., 5% CO2 in air) then fixed using 70% (v/v) ethanol, permeabilised with 0.1% (v/v) Triton X-100 and incubated with a blocking solution of 3% (v/v) goat serum in 1% (v/v) BSA/PBS. Staining was carried out using human ALP antibody (Dilution 1:50; sc137213, Santa Cruz Biotech) and counterstained for α-tubulin (2 μg/mL; PA120988, Invitrogen) for 3 hours at room temperature. After washing, slides were incubated with the appropriate secondary antibodies in the green and red channels at room temperature (1:100 dilution). Nuclei were stained with NucBlue Fixed Cell ReadyProbes™ (Invitrogen).

### Monocyte Isolation and Culture for ChemoTopo

Buffy coats were obtained from the National Blood Service after obtaining written informed consent and approval from the ethics committee. Monocytes were isolated from peripheral blood mononuclear cells (PBMCs). A MACS magnetic cell separation system (CD14 MicroBeads positive selection with LS columns, Miltenyi Biotec) was used for the isolation as previously described. Isolated monocytes were prepared in RPMI-1640 medium containing 10% foetal bovine serum (FBS), 100 μg/ml streptomycin, 2 mM L-glutamine and 100 U/ml penicillin (Sigma-Aldrich). For assessment of cell attachment and phenotype characterisation, cells were re-suspended in the appropriate volume of media and seeded on the ChemoTopoChips at 2×10⁶ monocytes/chip and incubated at 37° C., 5% CO2 in a humidified incubator for 9 days.

### Macrophage Immunofluorescent Staining for ChemoTopo

On day 9, all adherent cells cultured on ChemoTopoChips were fixed in 4% paraformaldehyde (BioRad) in PBS, then blocked with 3% BSA (Sigma-Aldrich) and 1% Glycine (Fisher Scientific) in PBS. Subsequently, another blocking step was carried out using 5% goat serum (Sigma) in PBS. Adherent cells were stained with 2 μg/mL anti-human TNFα (IgG1) mAb (Abcam), and with 1 μg/ml anti-human IL-10 (IgG1) mAb (Abcam) followed by 1 h incubation at room temperature. After washing, cells were stained with 8 μg/ml Rhodamine-x goat anti-mouse IgG (H+L) secondary Ab (Invitrogen), and 8 μg/ml Alexa flour-647 goat anti-rabbit IgG (H+L) secondary antibody (Invitrogen) for another hour at room temperature. All samples were counterstained with 250 ng/ml DAPI (4′,6-Diamidino-2-Phenylindole) (Invitrogen) at room temperature.

### ChemoTopoChip Imaging

Imaging of all fixed and stained ChemoTopoChip samples was carried out using a widefield deconvolution-TIRF3 system (Zeiss, custom setup). Imaging was carried out in wide field mode using a 20×/0.5 NA air objective in the bright field and fluorescence channels with the excitation at 358 nm, 488 nm and 561 nm. The software used to capture was Zeiss Zen Blue, by using the “Sample Carrier Designer” wizard/module to manually create and calibrate the position list which was used to scan all the positions in the chip setup.

### CellProfiler Analysis

A custom CellProfiler pipeline was created to correct for uneven background illumination in each image, then each image cropped to within the Topo unit 30 μm wall. Nuclei were detected using an adaptive per-object algorithm in the blue channel images, followed by propagation from these primary detected objects to detect cell cytoskeleton and ALP staining (hiMSCs) or TNFα and IL-10 (human macrophages) in the green and red channel images. Intensity of detected objects was measured and exported, and images containing overlaid outlines of detected objects also saved to ensure correct operation of the pipeline.

### Random Forest Machine Learning

The raw dataset consisted of three technical repeats for each surface variable (topography, chemistry) within a chip, which were further replicated across multiple batches (biological repeats). Data set from repeats in a chip have been normalised against their correspondent flat values. Subsequently, replicate average values were calculated. The average between batches was then determined as the dependent variable for the predictive models. Macrophage polarisation and ALP intensity predictive models were generated.

Various topographies were encoded using descriptors generated by CellProfiler that relate directly to particular primitives in the topographical units. 1-hot descriptors were used for chemistries.

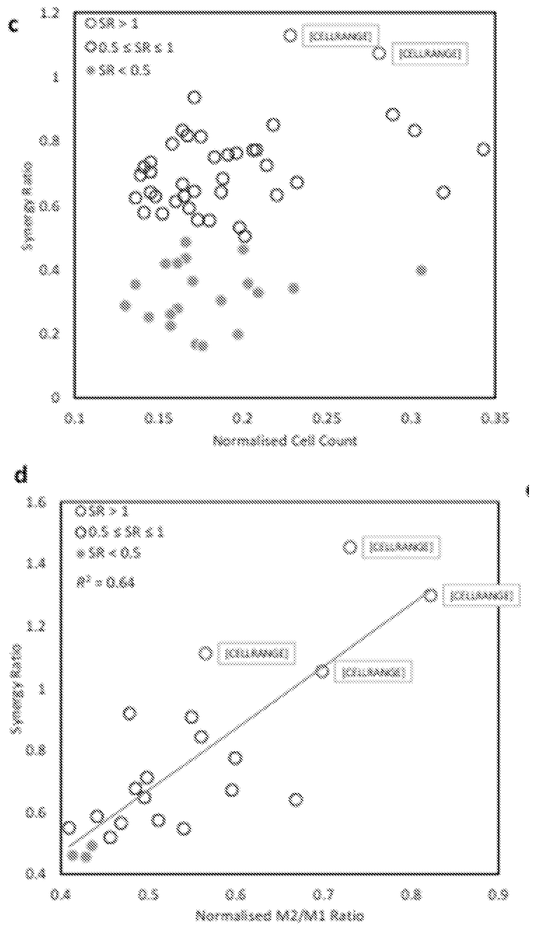

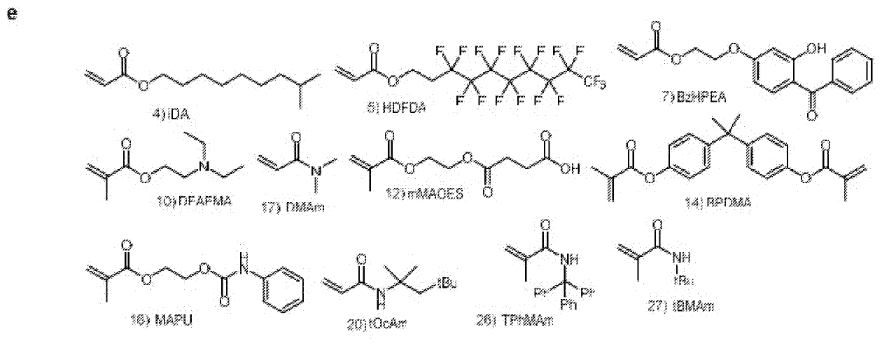

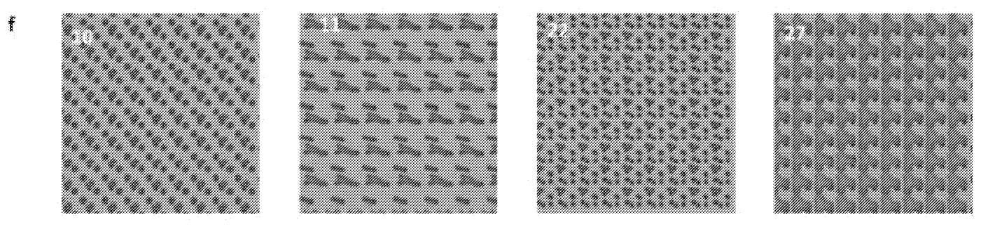

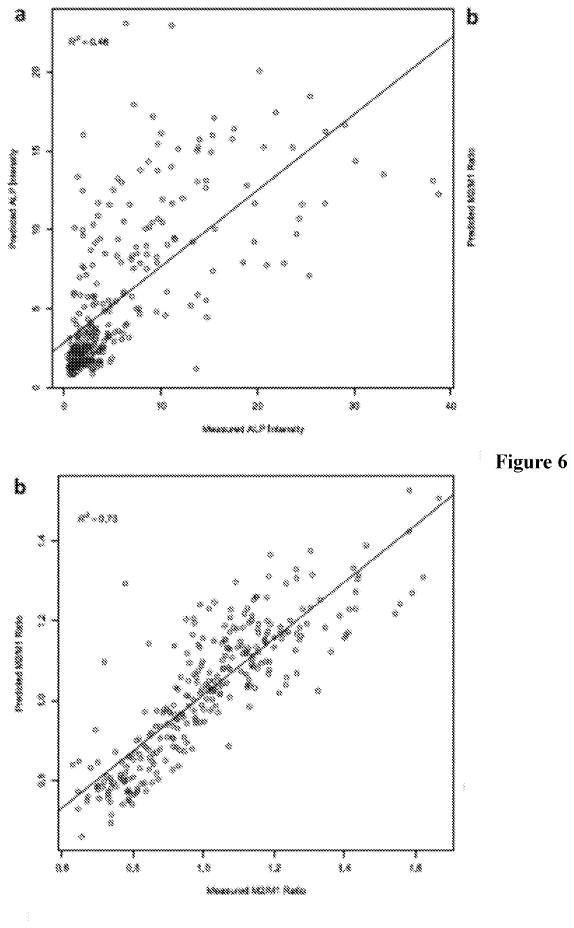

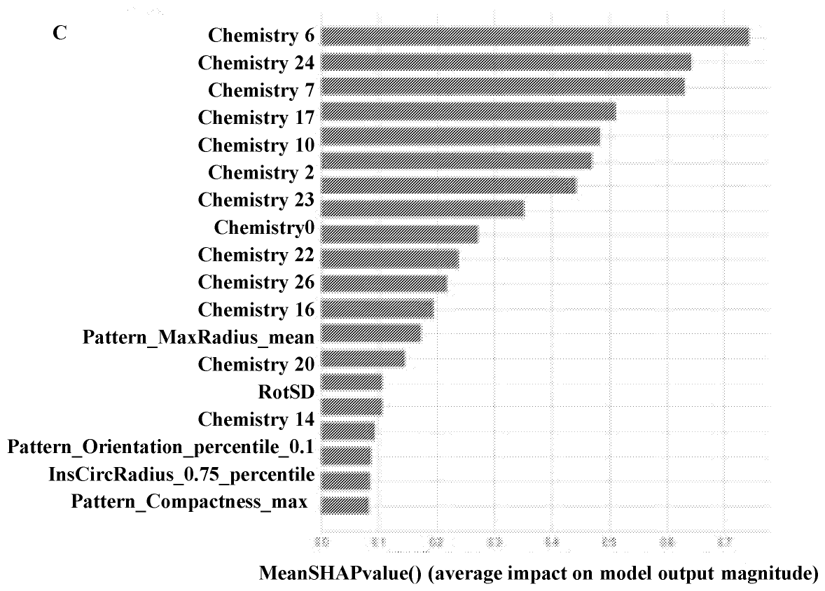

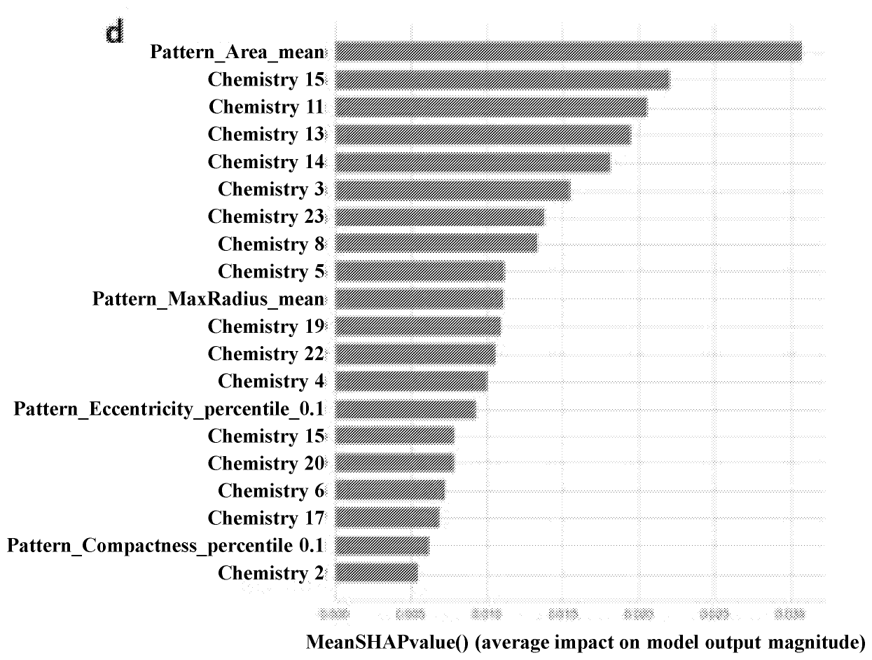

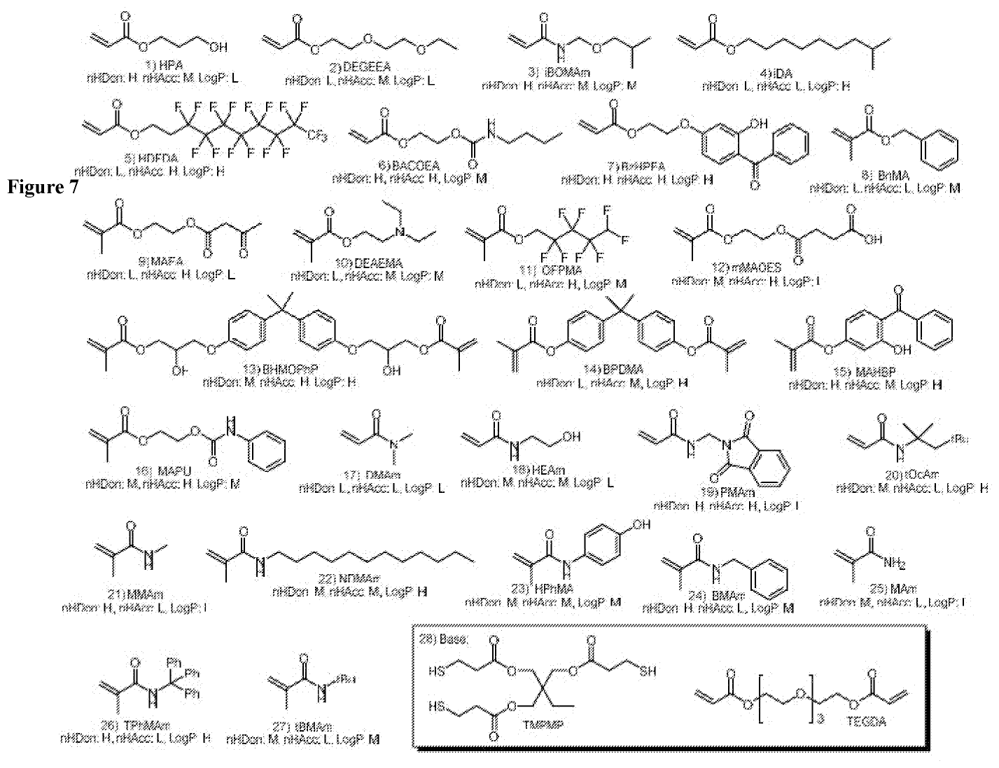

SHapley Additive exPlanation (SHAP) method was used for feature selection to eliminate uninformative and less informative descriptors and less relevant chemistries. SHAP was implemented using the SHAP package in Python 3.7. Regression models were generated using the random forest approach with the scikit-learn package in Python 3.7. The default parameters from version 0.22 were adopted for the random forest models. That is, 100 estimators were considered using gini as the function to measure the quality of the data instances split. And no limit for the maximum depth of the trees was defined. 70% of the data instances were employed for model training and 30% for testing. The performance of the predictive models and the topographical descriptors that contributed most strongly to the attachment and polarisation are shown in FIG. 6. The figure presents the results of the regression models as well as the features selected. The features are ordered from top to bottom based on their average impact on the model output magnitude

Methods Relating to Analysis of Polymers and Mixtures of Polymers (without Topographies Applied)

### Polymer Array Synthesis

Polymer microarrays were synthesized using methods previously described.16, 30 Briefly, polymer microarrays were formed using an XYZ3200 dispensing station (Biodot) and metal pins (946MP6B, Arrayit). The printing conditions were O2<2000 ppm, 25° C., and 35% humidity. To initiate the polymerisation, arrays were irradiated with UV (365 nm) for 1 minute directly after printing and for a further 10 minutes at the end of the print run. Each polymerisation solution was composed of monomer (50%, v/v) in dimethylformamide with photoinitiator 2,2-dimethoxy-2-phenyl acetophenone (1%, w/v). Six replicate spots were printed on each slide. Monomers were purchased from Aldrich, Scientific Polymers and Polysciences and printed onto epoxy-coated slides (Xenopore) dip-coated with poly(2-hydroxyethyl methacrylate) pHEMA (4% w/v, Sigma) in ethanol (95% v/v in water). Arrays were sterilised by exposure to UV light for 15 minutes prior to cell culture. The hits materials were scaled up as polymer coupons formed by pipetting polymerization solution (6 μL) onto a pHEMA coated slide and irradiating for 10 mins at O2<1300 ppm with a UV source (365 nm). Once formed, volatile components were removed from the polymers at <50 mTorr for 7 days. Polymers wettability were characterized by water contact angle measurements and their chemistry were identified by time-of-flight secondary ion mass spectrometry as previously described.31, 32

### Preparation of Scaled-Up Polymers

The polymerisation solution for the selected hits containing the monomer mixed with photo-initiator (1% w/v) was dispensed into 24-well polypropylene plates and polymerised under UV (365 nm) for 1 hour in the presence of, argon. Remaining volatile components were removed at <50 mTorr for 72 hours. The polymer surfaces were UV sterilised for 20 minutes and washed with sterile PBS before use. Tissue culture polystyrene (TCPS) was used as a control surface.

### Monocyte Isolation and Culture for Polymer Only Analysis

Buffy coats were obtained from healthy donors (National Blood Service, Sheffield, UK) after obtaining informed written consent and following ethics committee approval (Research Ethics Committee, Faculty of Medicine and Health Sciences, University of Nottingham). Monocytes were isolated from peripheral blood mononuclear cells (PBMCs). A MACS magnetic cell separation system (CD14 MicroBeads positive selection with LS columns, Miltenyi Biotec) was used for the isolation as previously described.^{33, 34} The purity of monocytes by this method was about 95% as determined by CD14 expression using flow cytometric analysis. Isolated monocytes were prepared to a cell density of 1×10⁶ cells/ml in RPMI-1640 medium (10% foetal bovine serum (FBS), 100 μg/ml streptomycin, 2 mM L-glutamine and 100 U/ml penicillin (Sigma-Aldrich)). For screening, 15 ml of the suspension (15×10⁶ monocytes) were seeded on microarray surfaces and incubated (37° C., 5% CO₂) in a humidified incubator for 6 days.

### Immunostaining of Macrophages on Polymer Arrays

On day 6 all adherent cells on polymer arrays were fixed in paraformaldehyde (4%) (EMS Diasum) in phosphate buffered saline (PBS), then blocked with bovine serum albumin (BSA, 3%, Sigma-Aldrich) and glycine (1%, Fisher Scientific) in PBS. Subsequently, another blocking step was carried out using goat serum (5%, Sigma) in PBS. Adherent cells were stained with anti-human calprotectin mouse IgG1 Ab (2 μg/mL) (Thermo Scientific), and rabbit CD206 (MR) anti-human primary Ab (1 μg/ml) (Abcam) followed by 1 h incubation at room temperature. After washing, cells were stained with Rhodamin-x goat anti-mouse IgG (H+L) secondary antibody (Ab, 8 μg/ml, Invitrogen), and Alexa flour-488 goat anti-rabbit IgG (H+L) secondary antibody (8 μg/ml Invitrogen) for another hour at room temperature. In all samples the nuclei were stained with 4′,6-Diamidino-2-Phenylindole (DAPI, 250 ng/ml, Invitrogen) for 5 minutes at room temperature. Slides were covered with FluorSave™ anti-fade medium (Calbiochem) and mounted with Fluoromount™ (Sigma-Aldrich). Arrays were imaged using an Olympus IX51 fluorescence microscope and a Smart Imaging System (IMSTAR S.A.). Images were analysed using CellProfiler cell image analysis software (http://www.cellprofiler.org/) to identify the number of positively MR and calprotectin-stained cells from four array replicates. To assess polymer induction of macrophage polarisation, we first established reference fluorescence measurements for the expression of these markers in populations of cytokine polarised M1 or M2 macrophages cultured on glass slides. Fluorescence images of a minimum of 100 cells in 9 fields of view were analysed for each cytokine polarisation in two different experiments for the same biological replicate (cell donor) prepared on the same day. The expression levels of calprotectin and MR in cytokine polarised M1 and M2 macrophages (generated as we have previously described) were used for setting the thresholds when analysing macrophage polarisation on the polymer arrays.³⁵ The maximum calprotectin fluorescent pixel intensity for each cell was used to represent its fluorescence expression and the average value was calculated for each cytokine polarised cell to represent the mean cellular expression for M1 polarised cells. The same procedure was followed for the MR fluorescence to obtain a mean cellular fluorescence expression for cytokine polarised M2 cells. Mean threshold fluorescence values for calprotectin and MR expression for cytokine polarised M1 or M2 cells were used to categorise the phenotype of the individual macrophage cells on when they exceeded these levels fluorescence values. The cell populations polarised by cytokines to M1 and M2 were determined to have a M2/M1 cell number ratio of 0.3 and 4.0 respectively, illustrating good categorisation of these reference cell populations. For macrophages on polymer microarrays, cell populations with M2/M1 cell number ratios below or above those found in these reference populations were considered to represent polymers inducing predominantly M1 or M2 differentiation, respectively.

### Cytokine Quantification Assay:

The level of TNF-α, IL-1β, CCL18 and IL-10 secreted into the media by macrophages cultured on scaled up polymers for 6 days was quantified by sandwich ELISA using DuoSet ELISA development kits (R&D Systems).

### Phagocytosis Assay:

Monocytes were cultured in polymer coated tissue culture plates for 6 days to allow differentiation to macrophages without cytokine stimulation. This was followed by addition of Alexa Fluor 488-labelled zymosan A (Saccharomyces cerevisiae) bioparticles (Thermo Fisher Scientific) (≈25 particle/cell). Following an incubation period of for 30 min (at 37° C., 5% CO2) cells were washed with sterile PBS (5 times) to removed un-phagocytosed particles. Tissue culture plastic was used as a control surface. Cells were then imaged with a Zeiss LSM 880 confocal microscope using a 40× oil objective lens (NA=1.30), a 488 nm argon laser, and 500-535 nm emission bandwidth. Images were captured using Zen digital imaging software.

### X-Ray Photoelectron Spectroscopy (XPS) Analysis of Protein Layer on Hit Polymers:

M1 (H24, C170), M2 (C255, C301) and non-polarising (C398, C408) polymers were printed in a microarray format as described earlier. The polymer array was immersed in RPMI (3 mL)—1640 medium (supplemented with 10% foetal bovine serum, 1% L-glutamine, 1% penicillin and streptomycin), in 4-well plates and incubated overnight (˜24 hours at 37° C., 5% CO₂). After incubation, the arrays were gently washed in ultrapure water (10 mL) for 10 minutes. The process was repeated 10 times, after which the samples were vacuum dried for ≥3 days prior to measurement. The protein adsorbate on each polymer spots was assessed at Kratos Analytical (Manchester, UK) with Kratos AXIS Nova X-ray Photoelectron Spectrometer equipped with dual Al/Ag monochromated X-ray source. The protein thickness was calculated from quantification of the nitrogen contribution using a method previously outlined.³⁶

### In-Vivo Murine Model:

Sections of medical grade silicone urinary catheter tube (2.7×5 mm Smith medical 8 Foley catheter) were cut longitudinally in half and served as a model implant. M1 (H24, C170), M2 (C301, C255) and non-polarising (C398, C408) polymers were manually dip-coated onto the silicone tube segments using NuSil MED-163 silicone primer and allowed to dry under ambient conditions for 30 mins. They were then manually dip-coated 3 times in a solution of each of the polymers (1 wt %) in toluene, leaving 30 mins drying time between dips. Coated segments were placed under vacuum (<0.3 mbar) for 1 week prior to use. Catheter sections without a polymer coating served as controls. Sterilisation consisted of exposure to ultraviolet light for a period of 20 min. All in-vivo studies were approved by the University of Nottingham Animal Welfare and Ethical Review Board and were carried out in accordance with home office authorisation under project licence number 30/3238. Age-matched adult female BALB/C mice, Charles River, were housed in IVC under 12 h light cycle with food and water ad libitum. An hour before catheter implantation, analgesia (carprofen) was administered subcutaneously (2.5 mg/kg), animals where anesthetised and hair removed by shaving, the area was sterilised with Hydrex (Ecoblab). A small incision was made in the flank and individual catheter segments were loaded into a trocar needle (9 g) and injected subcutaneously on one side of the mouse, the other side serving as a sham. The wound was sealed using Gluture skin glue. All mice were monitored until they recovered from the anaesthesia and inflammation at the site of implantation, behavioural changes and other adverse reactions were monitored throughout the duration of the experiment. At the end of the experiment, on day 28, mice were humanely sacrificed by CO₂ euthanasia.

### Histological Analysis:

The catheter segment and surrounding skin was excised and placed in zinc fixative for 24 hours. Following fixation, the tissue was loaded into cassettes and placed onto a Leica TP1020 tissue processor for dehydration through a series of ethanol solutions followed by incubation in xylene. Tissue was then embedding in paraffin wax and sliced into sections (7 μm) using a Leica RM2245 microtome before mounting onto poly-lysine coated slides (ThermoFisher Scientific). The foreign body response to the polymer coatings was assessed by staining with haematoxylin and eosin (H&E) and Masson's trichrome (MTC). Samples were observed using a Ventana DP200 (Roche) slide scanner with a ×40 objective. The histological interpretation of the tissue sections was performed by four of the authors including two specialised histopathologists.

### Phenotype Analysis:

Antigen retrieval was carried out by heating tissue sections to 100° C. for 20 min in citrate buffer (pH 6). Following washing in deionized water, cells were permeabilized using triton ×100 (0.1%) for 10 min and rinsed 3×5 min in PBS Tween 20 (0.2%). Non-specific binding was blocked by incubating tissue sections in BSA (5%) with donkey serum (5%) for 1 h at room temperature. Sequential antibody staining was undertaken using goat anti-mouse Arg-1 (1:50; PA5-18392 ThermoFisher Scientific) and rabbit anti-mouse iNOS (1:50; ab15323 Abcam) antibodies at 4° C. overnight. Secondary antibodies, donkey anti-goat IgG (H+L) and donkey anti-rabbit IgG (H+L) labelled with Alexa Fluor 594 and 488 (1:200; A11058 and A21206 ThermoFisher Scientific) respectively were applied for 1 h at room temperature to visualize the macrophage cells. Isotype controls and no primary antibody served as controls and showed little background autofluorescence. Images were acquired on a Zeiss LSM880C confocal microscope and any background fluorescence was subtracted using Image J. The mean raw intensity density of the region of interest around the foreign body site was used to measure the sum of all pixels in the given area. All in-vivo studies were carried out in duplicate on two separate occasions and at least five different fields of view were randomly examined in each tissue section. Polymer coatings were blinded to the researchers and revealed at the end.

### Statistical Analysis:

Statistical significance was calculated using a one-way ANOVA and Tukey's post-hoc analysis, where p≤0.05 was considered as being statistically significant for cytokine, protein thickness and morphological/phenotypical characteristics of macrophages in-vitro and in-vivo. To account for intra experimental variations between polymer replicates on each array, a signal to noise ratio (SNR) of 2 was used as a threshold for detection when evaluating fluorescent intensity, cell adherence and changes in cell morphology. The SNR was calculated using the ratio of the mean value of the signal and the standard deviation of the noise.

### EXAMPLES

### Example 1.1 Induction of Human Mesenchymal Stem Cell (hiMSC) Differentiation Towards an Osteoblastic Lineage

hiMSCs were seeded on 3 replicate chips in 3 independent experiments. After 5 days, samples were fixed and stained with both an α-tubulin (cytoskeletal marker) and for alkaline phosphatase (ALP, an early osteogenic marker), and analysed using an automated high-throughput fluorescence microscope. Images were processed using CellProfiler software to quantify cell number and ALP staining intensity on each individual chemistry-topography combination. The ALP staining intensity was normalised to that of the flat TMPMP-co-TEGDA Topo unit within each ChemoTopoChip sample.

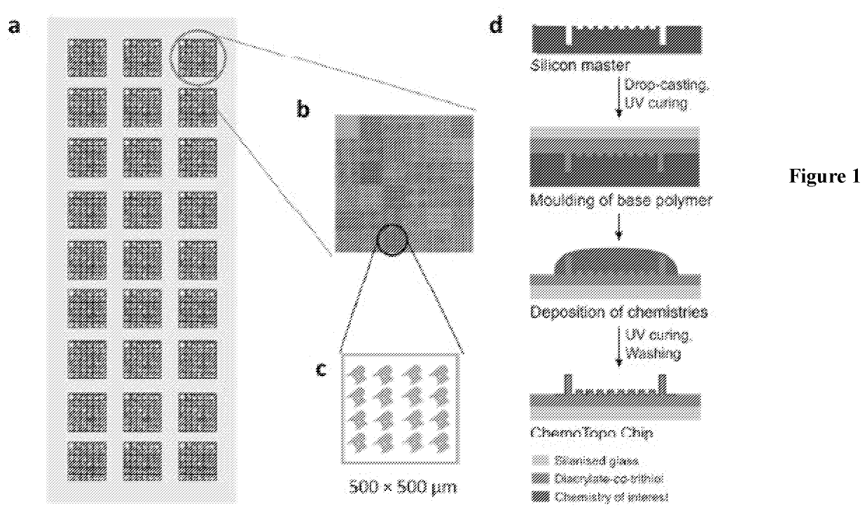

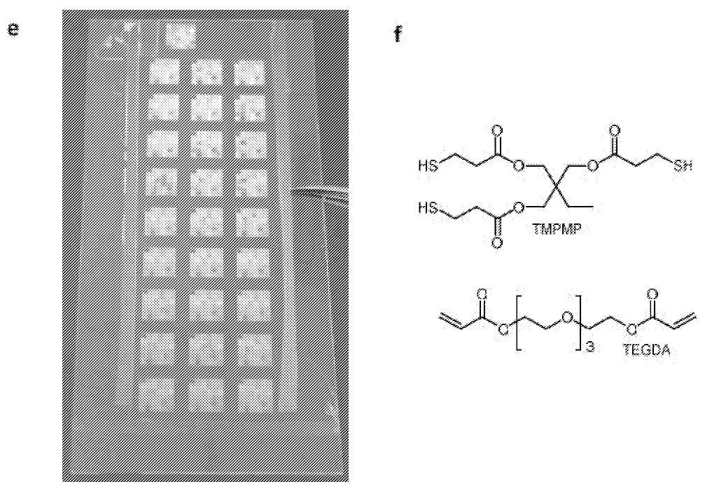

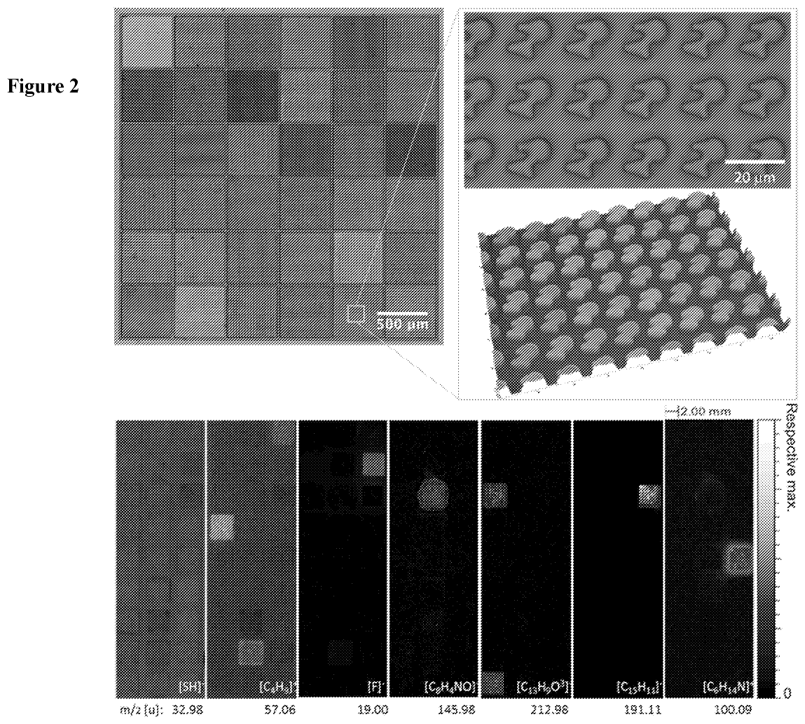

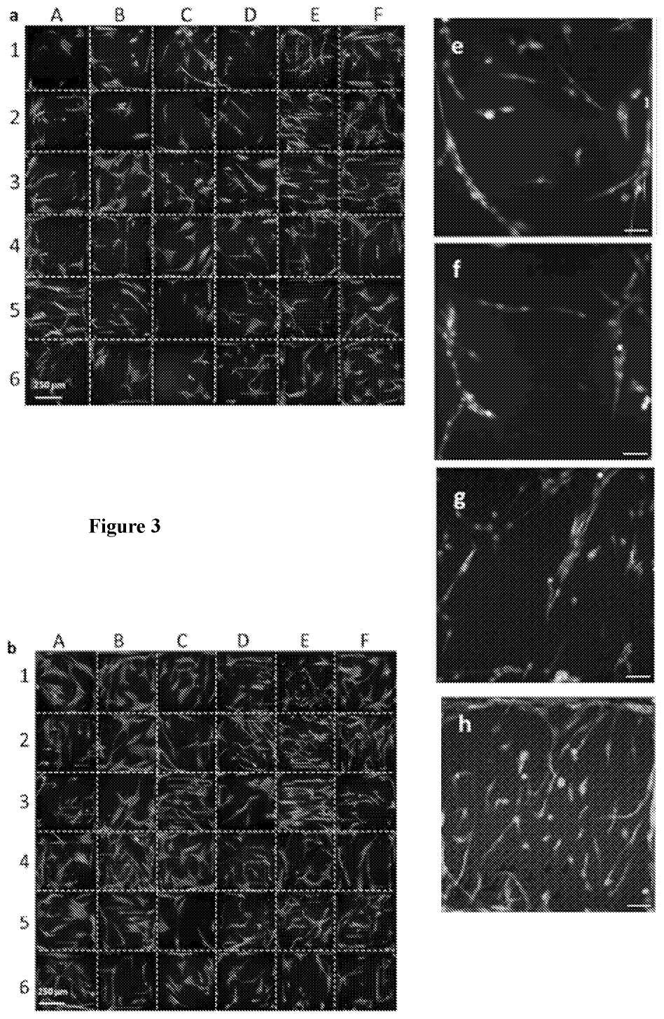

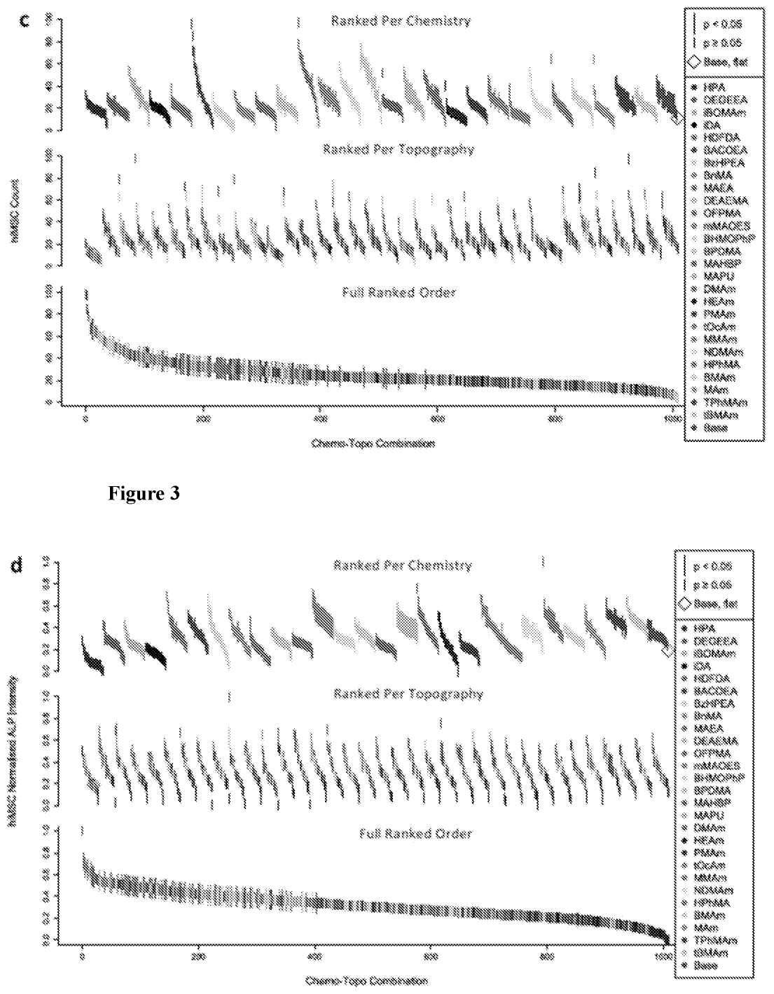

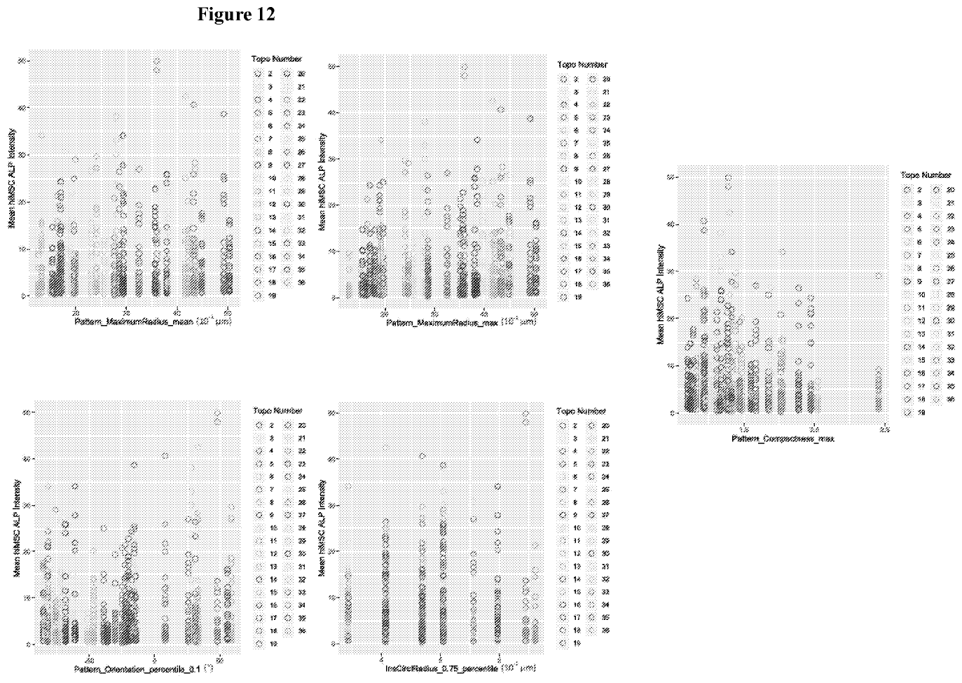

A diverse range of cell morphologies and cell numbers could be seen across the ChemoTopoChip (FIG. 3.a and 3.b, FIG. 3.e-h), with cells displaying an elongated shape and alignment to the topographies on some ChemoTopo units (eg. FIG. 3 a-b E2, E3) in contrast to more uniform cell spreading on others (eg. FIG. 3 a-b B3, C4). ALP expression is a widely used osteogenesis marker as it is known to be involved in bone formation, plays an essential role in matrix mineralisation and is induced by a range of osteogenic molecules. The mean integrated cell ALP intensity for each ChemoTopo unit was plotted as a heatmap to identify trends in chemistry and topography (see FIG. 12). Visual inspection reveals trends in the vertical axis, e.g. monomers 12 (mono-2-(methacryloyloxy)ethyl succinate, mMAOES) and 20 (N-tert-octylacrylamide, tOcAm), suggesting that a selection of chemistries appeared to be increasing ALP intensity relative to the mean; equivalent horizontal trends were not evident indicating no topographical dominance across the range of chemistries used.

It was useful to rank order all the results to see the range for all ChemoTopo units, then by topography and chemistry as shown in FIG. 3.c (see Table 2 for full list). For an exploratory method the combinations which have p-value <0.05 from a two independent sample equal variance t-test are indicated. The values were normalised to the base polymer region on each slide. Analysis of the mean ALP expression per cell for each ChemoTopo combination showed that 113 exhibited the most different amounts (p<0.05) compared to the flat base TMPMP-co-TEGDA polymer Topo unit (FIG. 3.d, see Table 2 for full list), with all of these displaying a higher ALP intensity than this ChemoTopo unit used as a control comparator. A similar situation was observed for cell numbers, with 103 combinations having higher cell count than the flat TMPMP-co-TEGDA control, but none lower (FIG. 3.c, see Table 2 for full list). Cross-referencing the lists of ALP and cell number hits highlighted 24 ChemoTopo combinations, comprised of 6 chemistries and 19 topographies, that showed higher ALP intensity and cell count compared to the base flat material. On the ChemoTopoChip, surface modulus is similar across all chemistry-topography combinations (see Table 1); it is therefore possible to assign the range of ALP expression that is seen to the materials chemistry and topography. The range of values observed for normalised hiMSC ALP intensity was 0.0020-0.28 AU (arbitrary units) for the lowest performing chemistry, and 0.00-1.0 AU for all other chemistries. Considering all chemistries combined with microtopographies, the range of normalised ALP intensity spanned the whole measured range (0.00-1.0 AU), whereas considering all chemistries on just flat areas the range of ALP intensity was only from 0.053-0.50 AU (FIG. 3.d).

The mean ALP fluorescence intensity/cell of the hiMSC positive control which was encouraged to differentiate by culturing in osteogenic media, in contrast to the basal media used in the ChemoTopo chip experiments, was 0.058 AU, across the same hiMSC biological repeats. The mean per ALP fluorescence intensity/cell for the ChemoTopo combinations showing ALP upregulation compared to the flat TMPMP-co-TEGDA area ranged from 0.068-0.043 AU. No difference in ALP upregulation (p<0.05) was observed between the ChemoTopoChip ALP hits and the positive control cultured in osteogenic media (p<0.05). The best materials therefore achieve similar ALP upregulation osteogenic state of the cells as osteo inductive media normally used to differentiate hiMSCs to bone.

Synergistic Combinations of Chemistry and Topography Identified for hiMSCs

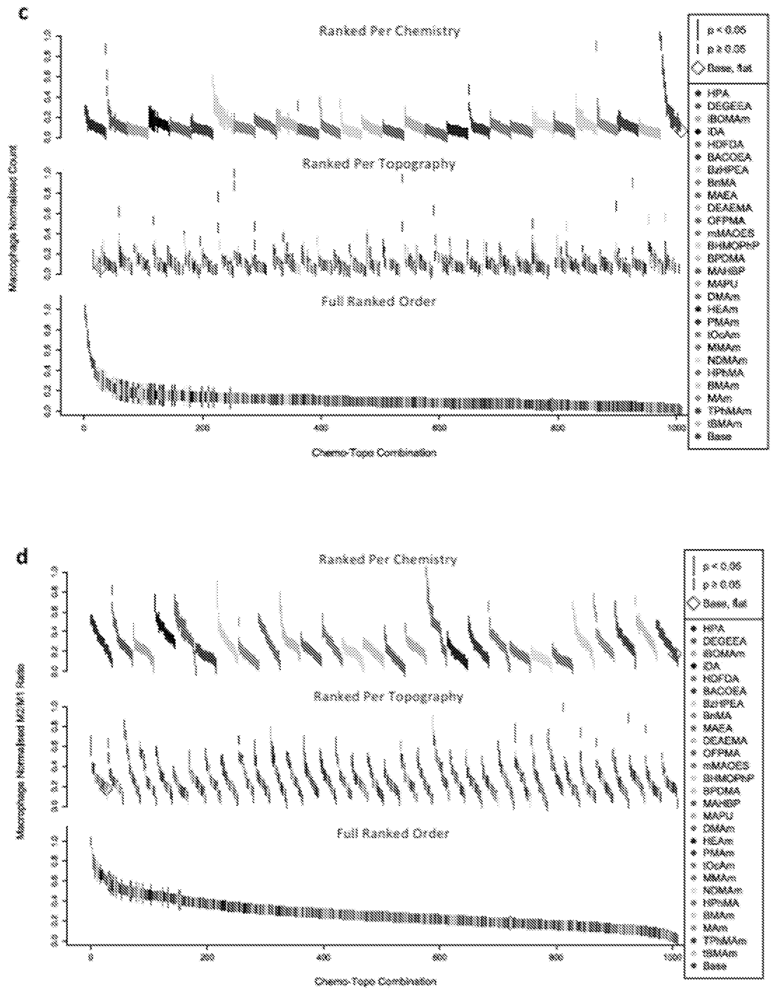

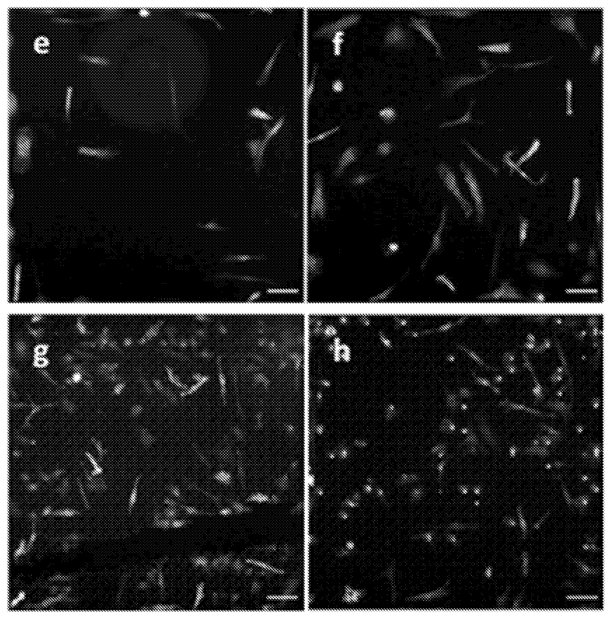

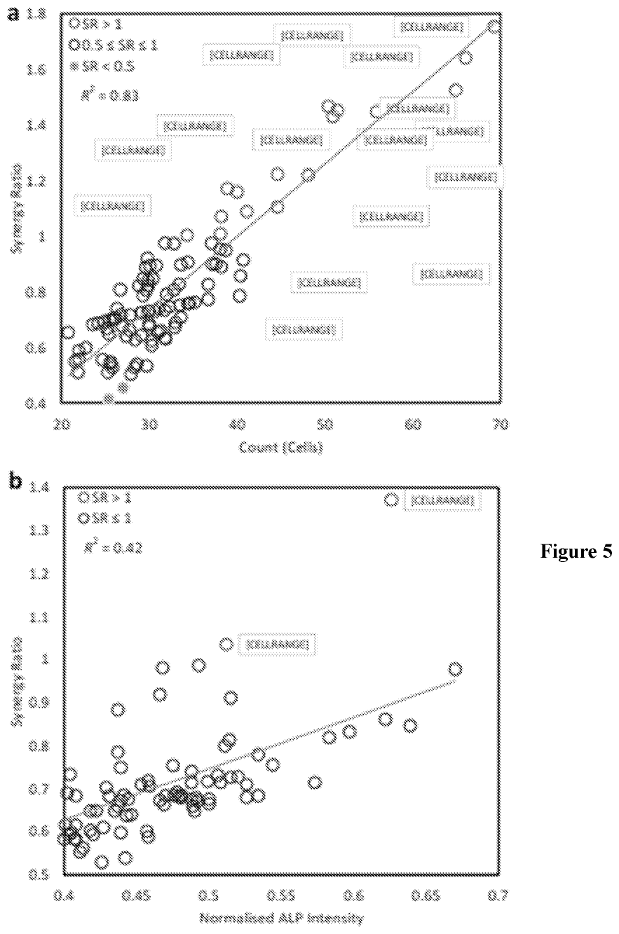

Assessment of the interactions between binary factors (chemistry and topography) is readily performed using a synergy ratio (SR). Taking the response of factor x1 alone (y1), the response of factor x2 alone (y2) and the response of the factors combined x12 (y12), SR is given by SR=y12/(y1+y2). For a synergistic combination, SR >1. In analysis of the ChemoTopoChip data, unfunctionalised TMPMP-co-TEGDA moulded topographies and flat area chemistries were used as the individual factors x1 and x2 to compare with the hit ChemoTopo combinations x12. For the hiMSC data set, of the 103 hit combinations providing statistically greater cell attachment and proliferation than the flat TPMP-co-TEGDA area, 15 were determined to be synergistic with SR >1 (FIG. 5.a, see Table 2 for full analysis of all synergistic combinations); additionally, 2 of the 113 hit combinations directing osteogenic differentiation were determined to be synergistic in nature (FIG. 5.b, see Table 2 for full analysis of all synergistic combinations). Many combinations had SR ≤1; the majority of these combinations can be taken to be additive in nature, but those with SR<0.5 can be interpreted as counteracting each other. For the hiMSC cell number, 2 combinations appeared to be antagonistic (FIG. 5.a) whereas none of the ALP intensity combinations had SR<0.5.

### Example 1.2—Direction of Macrophage Polarisation to Pro- and Anti-Inflammatory Phenotypes

As a second test of this methodology, the response of immune cells important in determining the bodies response to implanted medical devices was investigated. Primary human monocytes were seeded onto ChemoTopoChips and differentiated to macrophages over 6 days to investigate the ability of material chemistry-topography combinations to instruct human immune cell polarisation. Monocytes were isolated from peripheral blood of two independent donors, with 3 replicates carried out for each. To determine the polarisation status of the cells, samples were fixed and stained for intracellular expression of the pro and anti-inflammatory cytokines; tumour necrosis factor α (TNFα, M1 polarisation indicator) and interleukin-10 (IL-10, M2 polarisation indicator) respectively, and analysed using high-throughput fluorescence microscopy. Images were processed using CellProfiler software29 with an image analysis pipeline designed to quantify cell attachment using DAPI nuclear staining and mean fluorescence intensity (MFI) across each Topo unit for the IL-10 and TNFα channels. The IL-10 and TNFα MFI and cell number were normalised to the values from the flat TMPMP-co-TEGDA Topo unit, to correct for variation between biological samples observed for human macrophages. The ratio of M2/M1 cells was taken to be the ratio of the IL-10/TNFα MFIs.

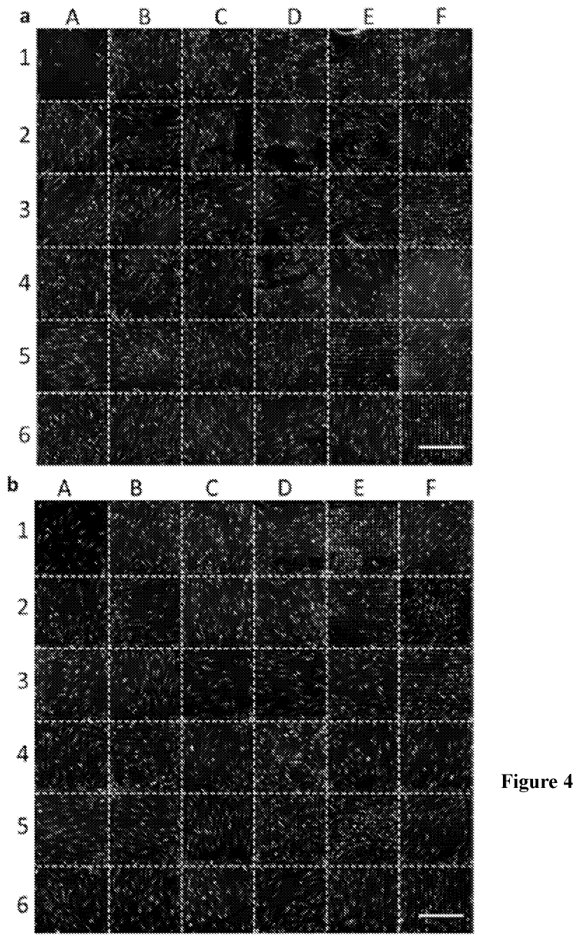

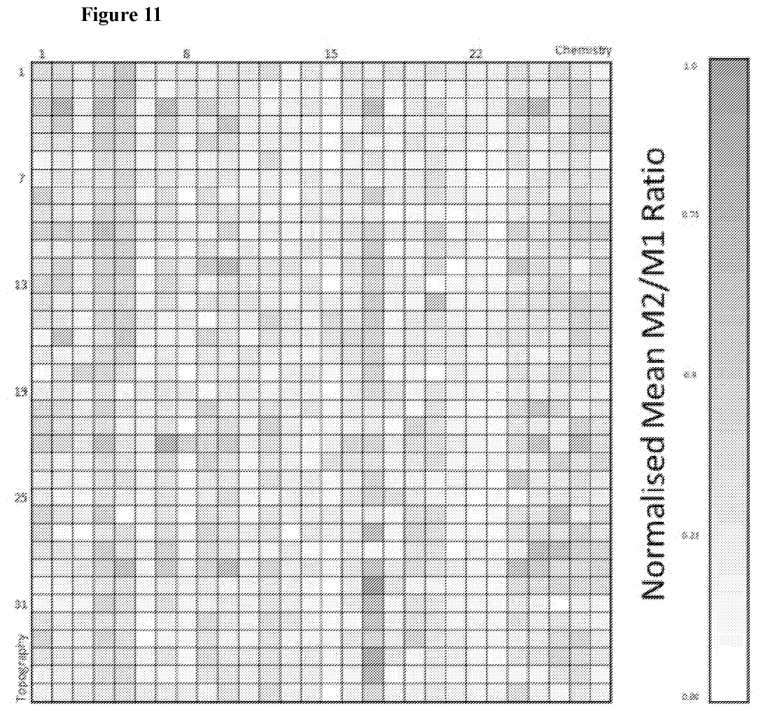

Cell morphologies ranging from elongated (eg. B3, F1) to rounded (eg. D2, D4) were observed across the ChemoTopoChip topographies, as can be seen in FIG. 4. Clear trends for macrophage polarisation could be seen across a selection of chemistries when M2/M1 ratio was plotted as a heatmap (see FIG. 11). Some Topo units appeared to influence polarisation across a range of chemistries, with topographies 10, 22 and 27 having generally higher M2/M1 ratios. Plotting the macrophage M2/M1 ratio and cell count as ranked scatter plots indicated the response to topography appeared as a continuum; conversely, the response to chemistries exhibited jumps showing larger differences (FIG. 4.c-d). Chemistries with lowest M2/M1 ratio across all Topo units ranged from 0.02 to 0.24, with the rest of the chemistries spanning the entire normalised range of 0.00-1.0. A range of 0.11-0.60 for normalised M2/M1 ratio was observed across all flat areas, yet a range of 0.00-1.0 was seen for all areas containing microtopographies. The normalised human macrophage cell number for all flat areas ranged from 0.087-0.14, and for the chemistry with the lowest cell number from 0.00-0.085. For areas containing microtopographies, the normalised cell number ranged from 0.00-1.0, and for the rest of the chemistries from 0.0087-1.0. This suggests that topography and chemistry are both important for driving macrophage attachment. Using the human macrophage cell number, 2 ChemoTopo units exhibited a synergistic effect (SR >1) (FIG. 5.c, and Table 3); whereas of the top 22 human macrophage polarisation ChemoTopo units, 4 were determined to be synergistic (FIG. 5.d, Table 3).

These results demonstrate an unexpected synergy by certain chemistries and topographies in the modulation of hiMSC attachment and osteogenic differentiation. For example, 2-(4-benzoyl-3-hydroxyphenoxy)ethyl acrylate (BzHPEA), in combination with topographies 10 and 22 (FIG. 5.e-f), were synergistic combinations for ALP expression. These topographies have moderate spacing of 5-10 μm. This may be attributed to confinement of cells between the structures, leading to reduced cell spreading compared to cells on flat surfaces.

There are no reports on the combinatorial role of these factors on immune cell fate. Materials chemistry/topography combinations tested in this study demonstrated the effect of topography on macrophage cell attachment. The attachment of macrophages increases in the presence of topographically patterned surface microstructures compared to flat chemistries in all cases. However, synergistic effect is also observed for 4 combinations showing an SR >1 directing macrophages towards an anti-inflammatory M2 phenotype, an integral aspect of increasing medical device biocompatibility (FIG. 5.d). The most synergistic combination was DMAm in combination with topography 27, exhibiting an SR of 1.5. Chemistry BzHPEA, in combination with topography 22, also had the highest normalised M2/M1 ratio of the synergistic hits, identifying this as a biologically important combination. Additionally, this topography includes cylindrical pillars. Promoting macrophage M2 polarisation in vivo plays a significant role in reducing a number of key cell processes including formation of foreign body giant cells, fibrotic encapsulation, and reduced, localised immune response to a biomaterial surface.

### Modelling the Effect of Chemistry and Topography on Cell Response

To investigate the feasibility of extracting rules that could inform future materials development from ChemoTopo Chip screening data, machine learning methods were applied to the data to determine whether structure-activity relationships could be generated. A combination of chemistry descriptors (“1-hot” or “indicator” binary variables indicating the presence or absence of a chemistry in any given combination) and topographical shape descriptors generated from CellProfiler29 was used to model both data sets using the random forest approach (FIG. 6). This method produces a vector with length equal to the number of categories in the data set. If a particular category is present then in the 1-hot vector, that category's position is set to 1 and all other positions in the vector are zero. Thus, 1-hot encoding is a process for converting categorical variables into a form that machine learning algorithms can use to generate predictive models.

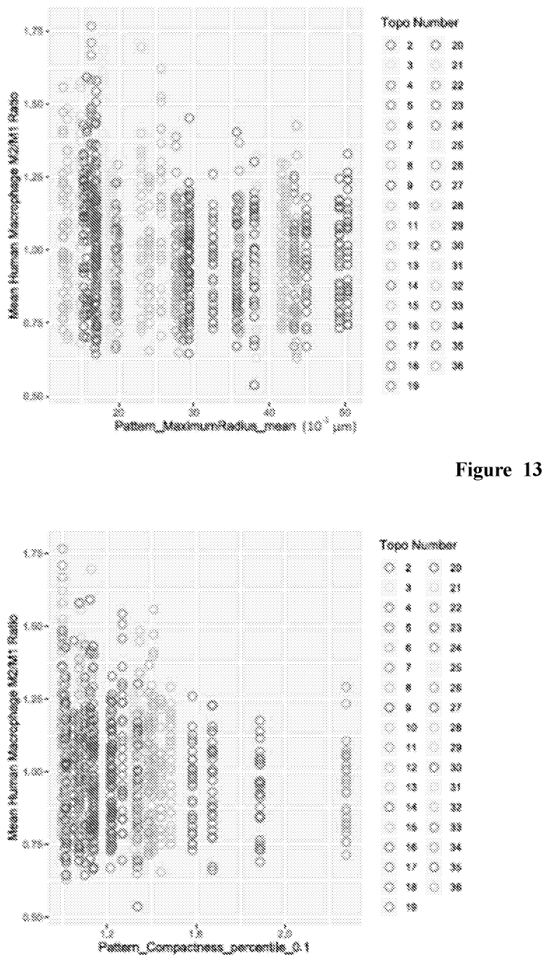

The human macrophage M2/M1 ratio model had a strong correlation between predicted and observed values, with R2=0.73. The size of the topographical features was highlighted as being important for macrophage polarisation, with features having a mean area below 50 μm2 and maximum radii of 1-3 μm providing the greatest M2/M1 ratio (see FIG. 13 for polarisation vs. descriptor correlations). The circularity of the topographical features was also shown to strongly influence the model, with lower eccentricity producing the greatest increase in macrophage M2 polarisation. The topographical descriptors for the human macrophage dataset had a greater average impact on the model M2/M1 magnitude than those of the hiMSC ALP intensity, suggesting a greater impact of topography on macrophage polarisation compared to hiMSC osteoinduction. This correlates with the phagocytic nature of the macrophage cells, which are designed to engulf bacterial cells and small particles. This illustrates the potential for uncovering previously unknown relationships between topography, chemistry, and cell response that offer opportunities in cell phenotype control.

The hiMSC ALP intensity random forest model had a lower correlation between predicted and observed behaviour, with R2=0.46. Difficulties in modelling stem cell response to polymeric biomaterials has been previously noted; 10 in that case dominated by the disparate nature of the relatively small number of polymers with desirable cell response. Of the topographical descriptors highlighted by the hiMSC ALP model, the size of the features was shown to contribute to the model (see Table 4 for list of feature descriptions); features with sizes of around 3.5 μm radius were noted as increasing ALP expression, although the trend was not as strong as that observed for macrophage polarisation. Relative alignment of the features was also noted as contributing to the model, with those Topo units containing a small number (<10%) of features with an increased rotation of >250 (relative to the x-axis Topo unit walls) showing an increase in ALP expression.

### SUMMARY

The utility of the ChemoTopoChip has been demonstrated as a unique and powerful tool for biomaterials discovery. Analysis of the hiMSC and human macrophage datasets has highlighted a range of novel chemistry-topography combinations that surpass the material-instructive cues provided by either alone, by over 30% in 10 cases and up to 80% for the most synergistic combination. This highlights the power of finding unexpected synergistic combinations of surface chemistry and topography to achieve bioinstructive responses for these cell types. The response of both cell types to chemistry and topography exhibited a similar range, suggesting that these two drivers are equally important consideration when designing biomaterials. Modelling of the human macrophage polarisation data showed that small, cylindrical pillars of <10 nm radius directed macrophage polarisation towards an anti-inflammatory phenotype. The size of the features was also shown to be important for hiMSC ALP expression, with features around 3.5 μm radius providing a positive influence on ALP upregulation. Data generated by the ChemoTopoChip are also suitable for analysis by powerful machine learning methods to enable models to be built to aid in design and discovery of the next generating of medical devices.

TABLE 1
AFM Modulus data taken from 4 ROIs:

| | | | | | Mean | |
|---|---|---|---|---|---|---|
| | Modulus | Modulus | Modulus | Modulus | Modulus | |
| Chemistry | A (Mpa) | B (Mpa) | C (Mpa) | D (Mpa) | (Mpa) | p-value |
| HPA | 110 | 120 | 110 | 110 | 112 | 0.31 |
| DEGEEA | 112 | 117 | 125 | 105 | 115 | 0.63 |
| iBOMAm | 111 | 122 | 118 | 108 | 115 | 0.59 |
| iDA | 126 | 110 | 107 | 127 | 117 | 0.96 |
| HDFDA | 105 | 117 | 106 | 115 | 111 | 0.24 |
| BACOEA | 117 | 109 | 118 | 108 | 113 | 0.36 |
| BzHPEA | 109 | 127 | 108 | 109 | 113 | 0.50 |
| BnMA | 70.7 | 119 | 119 | 67.5 | 94.1 | 0.17 |
| MAEA | 139 | 84.4 | 136 | 135 | 124 | 0.68 |
| DEAEMA | 129 | 107 | 121 | 108 | 116 | 0.84 |
| OFPMA | 116 | 106 | 111 | 115 | 112 | 0.29 |
| mMAOES | 99.0 | 109 | 98.1 | 118 | 106 | 0.12 |
| BHMOPhP | 101 | 98.1 | 118 | 98.2 | 104 | 0.06 |
| BPDMA | 122 | 120 | 137 | 104 | 121 | 0.72 |
| MAHBP | 106 | 118 | 104 | 108 | 109 | 0.13 |
| MAPU | 131 | 131 | 101 | 130 | 123 | 0.53 |
| DMAm | 128 | 101 | 128 | 111 | 117 | 0.96 |
| HEAm | 105 | 114 | 103 | 104 | 106 | 0.06 |
| PMAm | 114 | 111 | 112 | 112 | 112 | 0.23 |
| tOcAm | 133 | 138 | 96.4 | 103 | 117 | 0.98 |
| MNAm | 107 | 112 | 105 | 107 | 108 | 0.07 |
| NDMAm | 102 | 119 | 116 | 112 | 113 | 0.38 |
| HPhMA | 120 | 76.7 | 106 | 75.7 | 94.5 | 0.09 |
| BMAm | 104 | 113 | 108 | 102 | 107 | 0.06 |
| Mam | 134 | 107 | 91.6 | 132 | 116 | 0.90 |
| TPhMAm | 113 | 113 | 113 | 112 | 113 | 0.27 |
| tBMAm | 127 | 118 | 103 | 125 | 118 | 0.96 |
| TMPMP-co- | 126 | 108 | 123 | 114 | 118 | 1.00 |
| TEGDA | | | | | | |

TABLE 2
Statistically Signification hiMSC Combinations:

| | | | | Chemo | Topo | |
|---|---|---|---|---|---|---|
| | | | Normal- | Compar- | Compar- | |
| | | | ised | ator | ator | |
| Entry | Chemo | Topo | ALP | ALP | ALP | SR |
| 1 | mMAOES | 3 | 0.670 | 0.388 | 0.297 | 0.977 |
| 2 | mMAOES | 2 | 0.639 | 0.388 | 0.367 | 0.846 |
| 3 | BzHPEA | 10 | 0.626 | 0.173 | 0.283 | 1.372 |
| 4 | mMAOES | 13 | 0.622 | 0.388 | 0.334 | 0.861 |
| 5 | mMAOES | 14 | 0.597 | 0.388 | 0.330 | 0.832 |
| 6 | TPhMAm | 14 | 0.583 | 0.383 | 0.330 | 0.819 |
| 7 | tOcAm | 36 | 0.573 | 0.426 | 0.376 | 0.714 |
| 8 | MAPU | 6 | 0.544 | 0.375 | 0.347 | 0.755 |
| 9 | mMAOES | 19 | 0.534 | 0.388 | 0.298 | 0.779 |
| 10 | mMAOES | 27 | 0.534 | 0.388 | 0.395 | 0.683 |
| 11 | mMAOES | 33 | 0.526 | 0.388 | 0.386 | 0.679 |
| 12 | MAPU | 2 | 0.526 | 0.375 | 0.367 | 0.709 |
| 13 | mMAOES | 15 | 0.520 | 0.388 | 0.327 | 0.727 |
| 14 | mMAOES | 22 | 0.515 | 0.388 | 0.321 | 0.726 |
| 15 | BzHPEA | 26 | 0.515 | 0.173 | 0.392 | 0.910 |
| 16 | MAm | 35 | 0.514 | 0.286 | 0.346 | 0.813 |
| 17 | BzHPEA | 22 | 0.512 | 0.173 | 0.321 | 1.035 |
| 18 | HPhMA | 21 | 0.511 | 0.317 | 0.323 | 0.799 |
| 19 | mMAOES | 21 | 0.508 | 0.388 | 0.323 | 0.714 |
| 20 | mMAOES | 9 | 0.506 | 0.388 | 0.304 | 0.731 |
| 21 | tOcAm | 15 | 0.500 | 0.426 | 0.327 | 0.664 |
| 22 | tOcAm | 11 | 0.500 | 0.426 | 0.314 | 0.675 |
| 23 | mMAOES | 23 | 0.499 | 0.388 | 0.309 | 0.716 |
| 24 | BzHPEA | 15 | 0.493 | 0.173 | 0.327 | 0.986 |
| 25 | mMAOES | 6 | 0.492 | 0.388 | 0.347 | 0.670 |
| 26 | tOcAm | 3 | 0.491 | 0.426 | 0.297 | 0.678 |
| 27 | mMAOES | 25 | 0.490 | 0.388 | 0.369 | 0.647 |
| 28 | mMAOES | 16 | 0.489 | 0.388 | 0.353 | 0.659 |
| 29 | mMAOES | 8 | 0.488 | 0.388 | 0.336 | 0.674 |
| 30 | MAPU | 23 | 0.488 | 0.375 | 0.309 | 0.713 |
| 31 | mMAOES | 7 | 0.488 | 0.388 | 0.271 | 0.740 |
| 32 | mMAOES | 17 | 0.481 | 0.388 | 0.321 | 0.678 |
| 33 | HPhMA | 33 | 0.481 | 0.317 | 0.386 | 0.684 |
| 34 | MAPU | 14 | 0.479 | 0.375 | 0.330 | 0.680 |
| 35 | TPhMAm | 23 | 0.478 | 0.383 | 0.309 | 0.691 |
| 36 | MAPU | 24 | 0.476 | 0.375 | 0.321 | 0.684 |
| 37 | HPhMA | 30 | 0.475 | 0.317 | 0.313 | 0.754 |
| 38 | TPhMAm | 34 | 0.470 | 0.383 | 0.305 | 0.683 |
| 39 | mMAOES | 24 | 0.469 | 0.388 | 0.321 | 0.662 |
| 40 | BzHPEA | 9 | 0.468 | 0.173 | 0.304 | 0.980 |
| 41 | mMAOES | 34 | 0.466 | 0.388 | 0.305 | 0.672 |
| 42 | BzHPEA | 13 | 0.466 | 0.173 | 0.334 | 0.918 |
| 43 | mMAOES | 4 | 0.459 | 0.388 | 0.264 | 0.705 |
| 44 | HPhMA | 24 | 0.458 | 0.317 | 0.321 | 0.718 |
| 45 | mMAOES | 26 | 0.458 | 0.388 | 0.392 | 0.587 |
| 46 | MAPU | 33 | 0.457 | 0.375 | 0.386 | 0.600 |
| 47 | MAPU | 4 | 0.453 | 0.375 | 0.264 | 0.709 |
| 48 | mMAOES | 28 | 0.446 | 0.388 | 0.311 | 0.639 |
| 49 | MAPU | 10 | 0.444 | 0.375 | 0.283 | 0.675 |
| 50 | MAPU | 17 | 0.443 | 0.375 | 0.321 | 0.637 |
| 51 | mMAOES | 32 | 0.442 | 0.388 | 0.432 | 0.538 |
| 52 | MAPU | 7 | 0.441 | 0.375 | 0.271 | 0.684 |
| 53 | MAm | 2 | 0.439 | 0.286 | 0.367 | 0.672 |
| 54 | HPhMA | 29 | 0.439 | 0.317 | 0.270 | 0.749 |
| 55 | mMAOES | 35 | 0.439 | 0.388 | 0.346 | 0.597 |
| 56 | NDMAm | 20 | 0.437 | 0.313 | 0.244 | 0.785 |
| 57 | BzHPEA | 24 | 0.437 | 0.173 | 0.321 | 0.883 |
| 58 | HPhMA | 35 | 0.437 | 0.317 | 0.346 | 0.658 |
| 59 | MAPU | 3 | 0.435 | 0.375 | 0.297 | 0.647 |
| 60 | mMAOES | 20 | 0.431 | 0.388 | 0.244 | 0.682 |
| 61 | NDMAm | 19 | 0.429 | 0.313 | 0.298 | 0.702 |
| 62 | mMAOES | 11 | 0.427 | 0.388 | 0.314 | 0.609 |
| 63 | MAPU | 32 | 0.426 | 0.375 | 0.432 | 0.528 |
| 64 | TPhMAm | 29 | 0.422 | 0.383 | 0.270 | 0.648 |
| 65 | MAPU | 13 | 0.420 | 0.375 | 0.334 | 0.593 |
| 66 | NDMAm | 13 | 0.418 | 0.313 | 0.334 | 0.647 |
| 67 | MAPU | 18 | 0.418 | 0.375 | 0.319 | 0.602 |
| 68 | MAPU | 31 | 0.413 | 0.375 | 0.362 | 0.561 |
| 69 | MAPU | 25 | 0.411 | 0.375 | 0.369 | 0.552 |
| 70 | mMAOES | 5 | 0.408 | 0.388 | 0.277 | 0.614 |
| 71 | mMAOES | 30 | 0.408 | 0.388 | 0.313 | 0.582 |
| 72 | MAEA | 21 | 0.408 | 0.273 | 0.323 | 0.683 |
| 73 | NDMAm | 33 | 0.407 | 0.313 | 0.386 | 0.582 |
| 74 | MAPU | 9 | 0.405 | 0.375 | 0.304 | 0.597 |
| 75 | HDFDA | 13 | 0.404 | 0.218 | 0.334 | 0.733 |
| 76 | NDMAm | 2 | 0.403 | 0.313 | 0.367 | 0.592 |
| 77 | NDMAm | 7 | 0.402 | 0.313 | 0.271 | 0.689 |
| 78 | MAPU | 5 | 0.401 | 0.375 | 0.277 | 0.615 |
| 79 | MAPU | 30 | 0.400 | 0.375 | 0.313 | 0.582 |
| 80 | NDMAm | 25 | 0.398 | 0.313 | 0.369 | 0.584 |
| 81 | MAPU | 26 | 0.398 | 0.375 | 0.392 | 0.519 |
| 82 | MAPU | 21 | 0.398 | 0.375 | 0.323 | 0.570 |
| 83 | MAPU | 15 | 0.398 | 0.375 | 0.327 | 0.567 |
| 84 | MAPU | 34 | 0.396 | 0.375 | 0.305 | 0.583 |
| 85 | MAPU | 11 | 0.396 | 0.375 | 0.314 | 0.575 |
| 86 | BACOEA | 33 | 0.396 | 0.199 | 0.386 | 0.677 |
| 87 | mMAOES | 18 | 0.396 | 0.388 | 0.319 | 0.559 |
| 88 | MAm | 5 | 0.390 | 0.286 | 0.277 | 0.693 |
| 89 | MAPU | 35 | 0.390 | 0.375 | 0.346 | 0.541 |
| 90 | MAPU | 19 | 0.389 | 0.375 | 0.298 | 0.579 |
| 91 | TPhMAm | 12 | 0.389 | 0.383 | 0.243 | 0.621 |
| 92 | NDMAm | 22 | 0.389 | 0.313 | 0.321 | 0.613 |
| 93 | #N/A | 33 | 0.386 | 0.189 | 0.386 | 0.672 |
| 94 | MAPU | 36 | 0.385 | 0.375 | 0.376 | 0.513 |
| 95 | MAPU | 22 | 0.385 | 0.375 | 0.321 | 0.553 |
| 96 | BHMOPhP | 32 | 0.383 | 0.214 | 0.432 | 0.593 |
| 97 | HDFDA | 21 | 0.380 | 0.218 | 0.323 | 0.703 |
| 98 | MAPU | 28 | 0.380 | 0.375 | 0.311 | 0.554 |
| 99 | MAPU | 29 | 0.378 | 0.375 | 0.270 | 0.587 |
| 100 | MAPU | 1 | 0.375 | 0.375 | 0.189 | 0.665 |
| 101 | BPDMA | 33 | 0.370 | 0.373 | 0.386 | 0.487 |
| 102 | #N/A | 25 | 0.369 | 0.189 | 0.369 | 0.662 |
| 103 | #N/A | 2 | 0.367 | 0.189 | 0.367 | 0.660 |
| 104 | BnMA | 5 | 0.363 | 0.202 | 0.277 | 0.758 |
| 105 | DEGEEA | 33 | 0.362 | 0.159 | 0.386 | 0.665 |
| 106 | HDFDA | 30 | 0.361 | 0.218 | 0.313 | 0.680 |
| 107 | NDMAm | 6 | 0.357 | 0.313 | 0.347 | 0.542 |
| 108 | NDMAm | 26 | 0.355 | 0.313 | 0.392 | 0.504 |
| 109 | BnMA | 22 | 0.354 | 0.202 | 0.321 | 0.677 |
| 110 | MAPU | 20 | 0.347 | 0.375 | 0.244 | 0.561 |
| 111 | MMAm | 22 | 0.345 | 0.136 | 0.321 | 0.756 |
| 112 | NDMAm | 9 | 0.342 | 0.313 | 0.304 | 0.555 |
| 113 | DEGEEA | 2 | 0.339 | 0.159 | 0.367 | 0.646 |

| | | | | Chemo | Topo | |
|---|---|---|---|---|---|---|
| | | | Cell | Comparator | Comparator | |
| Entry | Chemo | Topo | Count | Count | Count | SR |
| 1 | BPDMA | 33 | 48.1 | 9.6 | 29.9 | 1.220 |
| 2 | TPhMAm | 18 | 40.4 | 21.2 | 26.0 | 0.856 |
| 3 | MAPU | 3 | 38.7 | 14.4 | 26.3 | 0.948 |
| 4 | BPDMA | 14 | 34.3 | 9.6 | 24.7 | 1.002 |
| 5 | BPDMA | 8 | 64.9 | 9.6 | 33.0 | 1.525 |
| 6 | BPDMA | 18 | 50.9 | 9.6 | 26.0 | 1.429 |
| 7 | TPhMAm | 30 | 31.8 | 21.2 | 29.1 | 0.632 |
| 8 | DMAm | 2 | 30.8 | 9.0 | 25.5 | 0.893 |
| 9 | OFPMA | 33 | 41.1 | 7.9 | 29.9 | 1.088 |
| 10 | DMAm | 36 | 31.8 | 9.0 | 34.4 | 0.733 |
| 11 | mMAOES | 8 | 31.9 | 17.6 | 33.0 | 0.631 |
| 12 | tOcAm | 2 | 38.1 | 14.6 | 25.5 | 0.952 |
| 13 | TPhMAm | 3 | 31.3 | 21.2 | 26.3 | 0.659 |
| 14 | MAPU | 27 | 26.9 | 14.4 | 25.4 | 0.675 |
| 15 | TPhMAm | 12 | 26.7 | 21.2 | 11.8 | 0.807 |
| 16 | tBMAm | 6 | 30.4 | 11.8 | 24.2 | 0.846 |
| 17 | Base | 33 | 29.9 | 11.0 | 29.9 | 0.730 |
| 18 | BPDMA | 21 | 50.4 | 9.6 | 24.9 | 1.466 |
| 19 | Base | 35 | 29.1 | 11.0 | 29.1 | 0.725 |
| 20 | tOcAm | 15 | 37.2 | 14.6 | 26.9 | 0.898 |
| 21 | DMAm | 18 | 28.8 | 9.0 | 26.0 | 0.821 |
| 22 | TPhMAm | 32 | 27.0 | 21.2 | 38.3 | 0.453 |
| 23 | MAPU | 31 | 33.0 | 14.4 | 33.5 | 0.688 |
| 24 | tOcAm | 7 | 25.3 | 14.6 | 35.2 | 0.509 |
| 25 | DEAEMA | 6 | 29.9 | 11.4 | 24.2 | 0.839 |
| 26 | MAPU | 5 | 32.8 | 14.4 | 26.2 | 0.806 |
| 27 | TPhMAm | 11 | 29.1 | 21.2 | 24.1 | 0.642 |
| 28 | HDFDA | 16 | 33.6 | 7.4 | 30.0 | 0.896 |
| 29 | DMAm | 14 | 32.8 | 9.0 | 24.7 | 0.972 |
| 30 | Base | 16 | 30.0 | 11.0 | 30.0 | 0.731 |
| 31 | TPhMAm | 26 | 28.3 | 21.2 | 32.5 | 0.527 |
| 32 | DEAEMA | 8 | 24.7 | 11.4 | 33.0 | 0.555 |
| 33 | iBOMAm | 30 | 29.3 | 8.2 | 29.1 | 0.786 |
| 34 | Base | 36 | 34.4 | 11.0 | 34.4 | 0.757 |
| 35 | BPDMA | 25 | 26.8 | 9.6 | 27.8 | 0.717 |
| 36 | MAPU | 16 | 36.7 | 14.4 | 30.0 | 0.825 |
| 37 | MAPU | 24 | 27.7 | 14.4 | 27.4 | 0.662 |
| 38 | Base | 30 | 29.1 | 11.0 | 29.1 | 0.725 |
| 39 | HDFDA | 17 | 29.6 | 7.4 | 25.9 | 0.887 |
| 40 | BPDMA | 30 | 55.9 | 9.6 | 29.1 | 1.447 |
| 41 | mMAOES | 2 | 38.2 | 17.6 | 25.5 | 0.888 |
| 42 | DEAEMA | 11 | 26.3 | 11.4 | 24.1 | 0.740 |
| 43 | BPDMA | 10 | 38.9 | 9.6 | 23.6 | 1.172 |
| 44 | Base | 6 | 24.2 | 11.0 | 24.2 | 0.687 |
| 45 | MMAm | 31 | 31.1 | 8.4 | 33.5 | 0.741 |
| 46 | MAPU | 13 | 32.9 | 14.4 | 34.6 | 0.671 |
| 47 | DEGEEA | 4 | 22.8 | 13.9 | 24.2 | 0.598 |
| 48 | HPhMA | 23 | 31.8 | 7.0 | 25.6 | 0.974 |
| 49 | mMAOES | 3 | 30.0 | 17.6 | 26.3 | 0.684 |
| 50 | Base | 13 | 34.6 | 11.0 | 34.6 | 0.758 |
| 51 | TPhMAm | 36 | 27.9 | 21.2 | 34.4 | 0.502 |
| 52 | MAPU | 17 | 44.6 | 14.4 | 25.9 | 1.105 |
| 53 | tOcAm | 16 | 40.7 | 14.6 | 30.0 | 0.913 |
| 54 | BMAm | 30 | 38.1 | 8.8 | 29.1 | 1.007 |
| 55 | Base | 31 | 33.5 | 11.0 | 33.5 | 0.752 |
| 56 | tOcAm | 8 | 31.0 | 14.6 | 33.0 | 0.652 |
| 57 | Base | 18 | 26.0 | 11.0 | 26.0 | 0.702 |
| 58 | Base | 17 | 25.9 | 11.0 | 25.9 | 0.701 |
| 59 | Base | 5 | 26.2 | 11.0 | 26.2 | 0.704 |
| 60 | DMAm | 25 | 29.7 | 9.0 | 27.8 | 0.806 |
| 61 | mMAOES | 27 | 27.4 | 17.6 | 25.4 | 0.639 |
| 62 | MAPU | 18 | 33.4 | 14.4 | 26.0 | 0.826 |
| 63 | TPhMAm | 4 | 28.4 | 21.2 | 24.2 | 0.626 |
| 64 | TPhMAm | 22 | 22.0 | 21.2 | 18.4 | 0.556 |
| 65 | MAPU | 21 | 25.4 | 14.4 | 24.9 | 0.648 |
| 66 | TPhMAm | 13 | 29.7 | 21.2 | 34.6 | 0.532 |
| 67 | Base | 25 | 27.8 | 11.0 | 27.8 | 0.716 |
| 68 | DMAm | 8 | 30.7 | 9.0 | 33.0 | 0.730 |
| 69 | TPhMAm | 5 | 25.6 | 21.2 | 26.2 | 0.539 |
| 70 | BHMOPhP | 3 | 29.3 | 8.3 | 26.3 | 0.846 |
| 71 | iBOMAm | 11 | 29.8 | 8.2 | 24.1 | 0.920 |
| 72 | BPDMA | 11 | 30.1 | 9.6 | 24.1 | 0.893 |
| 73 | DMAm | 24 | 29.9 | 9.0 | 27.4 | 0.822 |
| 74 | iBOMAm | 31 | 37.6 | 8.2 | 33.5 | 0.899 |
| 75 | MAPU | 8 | 36.7 | 14.4 | 33.0 | 0.773 |
| 76 | BPDMA | 5 | 38.2 | 9.6 | 26.2 | 1.069 |
| 77 | Base | 10 | 23.6 | 11.0 | 23.6 | 0.682 |
| 78 | Base | 34 | 40.3 | 11.0 | 40.3 | 0.785 |
| 79 | DEAEMA | 30 | 32.0 | 11.4 | 29.1 | 0.790 |
| 80 | MAPU | 10 | 34.4 | 14.4 | 23.6 | 0.905 |
| 81 | Base | 21 | 24.9 | 11.0 | 24.9 | 0.692 |
| 82 | Base | 2 | 25.5 | 11.0 | 25.5 | 0.698 |
| 83 | tBMAm | 18 | 25.2 | 11.8 | 26.0 | 0.667 |
| 84 | DEGEEA | 27 | 21.6 | 13.9 | 25.4 | 0.549 |
| 85 | DEAEMA | 19 | 22.0 | 11.4 | 26.1 | 0.587 |
| 86 | BPDMA | 17 | 51.4 | 9.6 | 25.9 | 1.452 |
| 87 | tOcAm | 32 | 28.6 | 14.6 | 38.3 | 0.540 |
| 88 | BPDMA | 15 | 44.6 | 9.6 | 26.9 | 1.223 |
| 89 | Base | 9 | 20.8 | 11.0 | 20.8 | 0.653 |
| 90 | mMAOES | 33 | 33.6 | 17.6 | 29.9 | 0.707 |
| 91 | mMAOES | 26 | 30.3 | 17.6 | 32.5 | 0.606 |
| 92 | DMAm | 35 | 37.1 | 9.0 | 29.1 | 0.973 |
| 93 | BACOEA | 31 | 66.0 | 6.7 | 33.5 | 1.642 |
| 94 | mMAOES | 35 | 25.6 | 17.6 | 29.1 | 0.547 |
| 95 | TPhMAm | 34 | 25.4 | 21.2 | 40.3 | 0.413 |
| 96 | DEGEEA | 35 | 21.9 | 13.9 | 29.1 | 0.509 |
| 97 | Base | 7 | 35.2 | 11.0 | 35.2 | 0.762 |
| 98 | BACOEA | 25 | 40.0 | 6.7 | 27.8 | 1.160 |
| 99 | TPhMAm | 24 | 30.3 | 21.2 | 27.4 | 0.624 |
| 100 | BPDMA | 16 | 69.3 | 9.6 | 30.0 | 1.753 |
| 101 | mMAOES | 15 | 30.0 | 17.6 | 26.9 | 0.675 |
| 102 | Base | 28 | 32.3 | 11.0 | 32.3 | 0.746 |
| 103 | TPhMAm | 25 | 25.8 | 21.2 | 27.8 | 0.526 |

TABLE 3
Statistically Significant Macrophage Combinations:

| | | | | Chemo | Topo | |
|---|---|---|---|---|---|---|
| | | | Normalised | Comparator | Comparator | |
| Entry | Chemo | Topo | M2/M1 | M2/M1 | M2/M1 | SR |
| 1 | 7 | 22 | 0.822 | 0.240 | 0.392 | 1.299 |
| 2 | 17 | 27 | 0.730 | 0.214 | 0.288 | 1.454 |
| 3 | 10 | 12 | 0.698 | 0.272 | 0.391 | 1.054 |
| 4 | 5 | 29 | 0.668 | 0.599 | 0.446 | 0.639 |
| 5 | 5 | 1 | 0.599 | 0.599 | 0.175 | 0.774 |
| 6 | 5 | 16 | 0.595 | 0.599 | 0.289 | 0.670 |
| 7 | 17 | 11 | 0.565 | 0.214 | 0.293 | 1.112 |
| 8 | 10 | 22 | 0.560 | 0.272 | 0.392 | 0.843 |
| 9 | 17 | 12 | 0.549 | 0.214 | 0.391 | 0.907 |
| 10 | 5 | 12 | 0.540 | 0.599 | 0.391 | 0.546 |
| 11 | 5 | 11 | 0.511 | 0.599 | 0.293 | 0.573 |
| 12 | 7 | 28 | 0.498 | 0.240 | 0.462 | 0.710 |
| 13 | 4 | 12 | 0.495 | 0.377 | 0.391 | 0.646 |
| 14 | 12 | 21 | 0.485 | 0.385 | 0.333 | 0.675 |
| 15 | 27 | 33 | 0.478 | 0.284 | 0.237 | 0.918 |
| 16 | 12 | 29 | 0.468 | 0.385 | 0.446 | 0.564 |
| 17 | 5 | 7 | 0.456 | 0.599 | 0.281 | 0.518 |
| 18 | 12 | 15 | 0.441 | 0.385 | 0.370 | 0.585 |
| 19 | 5 | 17 | 0.435 | 0.599 | 0.287 | 0.491 |
| 20 | 5 | 14 | 0.428 | 0.599 | 0.343 | 0.455 |
| 21 | 5 | 9 | 0.413 | 0.599 | 0.301 | 0.459 |
| 22 | 5 | 35 | 0.409 | 0.599 | 0.148 | 0.548 |
| | | | | Chemo | Topo | |
| | | | Normalised | Comparator | Comparator | |
| Entry | Chemo | Topo | Count | Count | Count | SR |
| 1 | 23 | 1 | 0.145 | 0.145 | 0.061 | 0.703 |
| 2 | 24 | 10 | 0.176 | 0.082 | 1.000 | 0.162 |
| 3 | 0 | 12 | 0.191 | 0.061 | 0.191 | 0.757 |
| 4 | 4 | 12 | 0.152 | 0.075 | 0.191 | 0.574 |
| 5 | 22 | 13 | 0.201 | 0.041 | 0.358 | 0.504 |
| 6 | 4 | 13 | 0.200 | 0.075 | 0.358 | 0.463 |
| 7 | 7 | 13 | 0.319 | 0.140 | 0.358 | 0.640 |
| 8 | 10 | 14 | 0.136 | 0.063 | 0.156 | 0.623 |
| 9 | 7 | 14 | 0.214 | 0.140 | 0.156 | 0.724 |
| 10 | 22 | 15 | 0.145 | 0.041 | 0.157 | 0.733 |
| 11 | 0 | 16 | 0.139 | 0.061 | 0.139 | 0.694 |
| 12 | 0 | 18 | 0.302 | 0.061 | 0.302 | 0.832 |
| 13 | 2 | 18 | 0.166 | 0.080 | 0.302 | 0.435 |
| 14 | 24 | 18 | 0.161 | 0.082 | 0.302 | 0.419 |
| 15 | 25 | 18 | 0.198 | 0.070 | 0.302 | 0.532 |
| 16 | 26 | 18 | 0.154 | 0.066 | 0.302 | 0.418 |
| 17 | 7 | 18 | 0.343 | 0.140 | 0.302 | 0.774 |
| 18 | 10 | 2 | 0.171 | 0.063 | 0.120 | 0.937 |
| 19 | 24 | 2 | 0.228 | 0.082 | 0.120 | 1.128 |
| 20 | 16 | 20 | 0.197 | 0.050 | 0.949 | 0.198 |
| 21 | 24 | 20 | 0.172 | 0.082 | 0.949 | 0.167 |
| 22 | 10 | 21 | 0.175 | 0.063 | 0.152 | 0.813 |
| 23 | 7 | 21 | 0.187 | 0.140 | 0.152 | 0.641 |
| 24 | 7 | 22 | 0.306 | 0.140 | 0.632 | 0.397 |
| 25 | 7 | 23 | 0.168 | 0.140 | 0.143 | 0.591 |
| 26 | 0 | 25 | 0.206 | 0.061 | 0.206 | 0.771 |
| 27 | 7 | 25 | 0.232 | 0.140 | 0.206 | 0.671 |
| 28 | 0 | 26 | 0.208 | 0.061 | 0.208 | 0.772 |
| 29 | 20 | 26 | 0.165 | 0.055 | 0.208 | 0.628 |
| 30 | 7 | 26 | 0.220 | 0.140 | 0.208 | 0.631 |
| 31 | 7 | 27 | 0.141 | 0.140 | 0.104 | 0.577 |
| 32 | 24 | 28 | 0.161 | 0.082 | 0.496 | 0.279 |
| 33 | 4 | 28 | 0.203 | 0.075 | 0.496 | 0.356 |
| 34 | 7 | 28 | 0.209 | 0.140 | 0.496 | 0.328 |
| 35 | 24 | 29 | 0.167 | 0.082 | 0.122 | 0.817 |
| 36 | 4 | 29 | 0.164 | 0.075 | 0.122 | 0.833 |
| 37 | 7 | 29 | 0.281 | 0.140 | 0.122 | 1.072 |
| 38 | 22 | 3 | 0.145 | 0.041 | 0.186 | 0.640 |
| 39 | 25 | 3 | 0.218 | 0.070 | 0.186 | 0.850 |
| 40 | 4 | 3 | 0.160 | 0.075 | 0.186 | 0.612 |
| 41 | 7 | 3 | 0.289 | 0.140 | 0.186 | 0.883 |
| 42 | 4 | 31 | 0.173 | 0.075 | 0.237 | 0.555 |
| 43 | 2 | 32 | 0.141 | 0.080 | 0.116 | 0.719 |
| 44 | 1 | 33 | 0.157 | 0.030 | 0.670 | 0.225 |
| 45 | 26 | 35 | 0.166 | 0.066 | 0.275 | 0.486 |
| 46 | 8 | 35 | 0.136 | 0.110 | 0.275 | 0.353 |
| 47 | 0 | 36 | 0.196 | 0.061 | 0.196 | 0.762 |
| 48 | 2 | 36 | 0.188 | 0.080 | 0.196 | 0.683 |
| 49 | 22 | 36 | 0.148 | 0.041 | 0.196 | 0.627 |
| 50 | 24 | 4 | 0.171 | 0.082 | 0.184 | 0.643 |
| 51 | 7 | 4 | 0.180 | 0.140 | 0.184 | 0.553 |
| 52 | 22 | 5 | 0.144 | 0.041 | 0.533 | 0.251 |
| 53 | 24 | 5 | 0.187 | 0.082 | 0.533 | 0.304 |
| 54 | 26 | 5 | 0.157 | 0.066 | 0.533 | 0.261 |
| 55 | 7 | 5 | 0.230 | 0.140 | 0.533 | 0.341 |
| 56 | 0 | 6 | 0.183 | 0.061 | 0.183 | 0.750 |
| 57 | 10 | 6 | 0.164 | 0.063 | 0.183 | 0.666 |
| 58 | 24 | 7 | 0.170 | 0.082 | 0.384 | 0.364 |
| 59 | 6 | 7 | 0.130 | 0.069 | 0.384 | 0.287 |
| 60 | 10 | 8 | 0.158 | 0.063 | 0.136 | 0.791 |

TABLE 4
Topographical Descriptors

| | | Features | |
|---|---|---|---|
| Basic Shape | | Highlighted | |
| Feature | Description | From Model | Description |
| Eccentricity | The eccentricity of the ellipse | Pattern_ | Eccentricity of the most |
| | that has the same second- | Eccentricity_ | circular structures (lowest |
| | moments as the region. The | percentile_0.1 | 10% eccentricity) on the |
| | eccentricity is the ratio of the | | surface |
| | distance between the foci of | | |
| | the ellipse and its major axis | | |
| | length. The value is between 0 | | |
| | and 1. (0 and 1 are | | |
| | degenerate cases; an ellipse | | |
| | whose eccentricity is 0 is | | |
| | actually a circle, while an | | |
| | ellipse whose eccentricity is 1 | | |
| | is a line segment.) | | |
| Area | The actual number of pixels in | Pattern_area_ | Mean area of pillars on the |
| | the region | mean | surfaces |
| Compactness | The mean squared distance of | Pattern_ | Lowest 10% measured |
| | the object′s pixels from the | Compactness_ | compactness |
| | centroid divided by the area. | percentile_0.1 | Maximum measured |
| | A filled circle will have a | Pattern_ | compactness |
| | compactness of 1, with | Compactness_ | |
| | irregular objects or objects | max | |
| | with holes having a value | | |
| | greater than 1. | | |
| Radius | The length (in pixels) of the | Pattern_ | Mean of the maximum radii |
| | minor axis of the ellipse that | Maximum | on the surface |
| | has the same normalized | Radius_mean | Maximum radii on the |
| | second central moments as | Pattern_ | surface |
| | the region | Maximum | |
| | | Radius_max | |
| Orientation | The angle (in degrees ranging | Pattern_ | Lowest 10% measured |
| | from −90 to 90 degrees) | Orientation_ | relative orientation of |
| | between the x-axis and the | percentile_0.1 | features |
| | major axis of the ellipse that | | |
| | has the same second- | | |
| | moments as the region. | | |
| Inscribed | A number of circles that can | Inscr_Circl_ | The radius of the top 25% |
| Circle Radius | be fitted in gap between | Radius_0.75_ | of inscribed circles |
| | pillars. The algorithm is | percentile | |
| | looped until a circle diameter | | |
| | smaller than 6 μm is found. | | |

¹ Murphy W. L., McDevitt T. C., Engler A. J. Materials as stem cell regulators, Nat. Mater. 13, 547-557 (2014)
¹ Dalby M. J., Gadegaard N., Tare R., Andar A., Riehle M. O., Herzyk P., Wilkinson C. D., Oreffo R. O. The control of human mesenchymal cell differentiation using nanoscale symmetry and disorder. Nat. Mater. 6, 997-1003 (2007)
¹ Celiz A. D., Smith J. G. W., Langer R., Anderson D. G., Winkler D. A., Barrett D. A., Davies M. C., Young L. E., Denning C., Alexander M. R. Materials for stem cell factories of the future. Nat. Mater. 13, 570-580 (2014)
¹ Ball S. G., Shuttleworth C. A., Kielty C. M., Mesenchymal stem cells and neovascularization: Role of platelet-derived growth factor receptors. J. Cell. Mol. Med. 11, 1012-1030 (2007)
¹ Zhao K., Lou R., Huang F., Peng Y., Jiang Z., Huang K., Wu X., Zhang Y., Fan Z., Zhou H. Immunomodulation effects of mesenchymal stromal cells on acute graft-versus-host disease after hematopoietic stem cell transplantation. Biol. Blood Marrow Transplant. 21, 97-104 (2015)
¹ Li A. W., Sobral M. C., Badrinath S., Choi Y., Graveline A., Stafford A. G., Weaver J. C., Dellacherie M. O., Shih T. Y., Ali O. A., Kim J., Wucherpfennig K. W., Mooney D. J. A facile approach to enhance antigen response for personalized cancer vaccination. Nat. Mater. 17, 528-534 (2018)
¹ Veiseh O., Doloff J. C., Ma M., Vegas A. J., Tam H. H., Bader A. R., Li J., Langan E., Wyckoff J., Loo W. S., Jhunjhunwala S., Chiu A., Siebert S., Tang K., Hollister-Lock J., Aresta-Dasilva S., Bochenek M., Mendoza-Elias J., Wang Y., Qi M., Lavin D. M., Chen M., Dholakia N., Thakrar R., Lacik I., Weir G. C., Oberholzer J., Greiner D. L., Langer R., Anderson D. G. Size- and shape-dependent foreign body immune response to materials implanted in rodents and non-human primates. Nat. Mater. 14, 643-651 (2015)
¹ Zhang, L., Cao, Z., Bai, T., Carr, L., Ella-Menye, J.-R., Irvin, C., Ratner, B. D. & Jiang, S. Zwitterionic hydrogels implanted in mice resist the foreign-body reaction. Nat Biotechnol. 31, 553-556 (2013)
¹ Kohn J. New approaches to biomaterials design. Nat. Mater. 3, 745-747 (2004)
¹ Celiz A. D., Smith J. G. W., Patel A. K., Hook A. L., Rajamohan D., George V. T., Patel M. J., Epa V. C., Singh T., Langer R., Anderson D. G., Allen N. D., Hay D. C., Winkler D. A., Barrett D. A., Davies M. C., Young L. E., Denning C., Alexander M. R. Discovery of a Novel Polymer for Human Pluripotent Stem Cell Expansion and Multi-Lineage Differentiation. Adv. Mater. 27, 4006-4012 (2015)
¹ Zhang R., Mjoseng H. K., Hoeve M. A., Bauer N. G., Pells S., Besseling R., Velugotla S., Tourniaire G., Kishen R. E. B., Tsenkina Y., Armit C., Duffy C. R. E., Helfen M., Edenhofer F., de Sousa P. A., Bradley M. A thermoresponsive and chemically defined hydrogel for long-term culture of human embryonic stem cells. Nat. Commun. 4, 1335 (2013)
¹ Patel A. K., Celiz A. D., Rajamohan D., Anderson D. G., Langer R., Davies M. C., Alexander M. R., Denning C. A defined synthetic substrate for serum-free culture of human stem cell derived cardiomyocytes with improved functional maturity identified using combinatorial materials discovery. Biomaterials 61, 257-265 (2015)
¹ Hay D. C., Pernaglio S., Diaz-Mochon J. J., Medine C. N., Greenhough S., Hannoun Z., Schrader J., Black J. R., Fletcher J., Dalgetty D., Thompson A. I., Newsome P. N., Forbes S. J., Ross J. A., Bradley M., Iredale J. P. Unbiased screening of polymer libraries to define novel substrates for functional hepatocytes with inducible drug metabolism, Stem. Cell. Res. 6, 92-102 (2011)
¹ Hook A. L., Chang C. Y., Yang J., Luckett J., Cockayne A., Atkinson S., Mei Y., Bayston R., Irvine D. J., Langer R., Anderson D. G., Williams P., Davies M. C., Alexander M. R. Combinatorial discovery of polymers resistant to bacterial attachment. Nat. Biotechnol. 30, 868-875 (2012)
¹ Jeffery N., Kalenderski K., Dubern J., Lomiteng A., Dragova M., Frost A., Macrae B., Mundy A., Alexander M., Williams P., Andrich D. A new bacterial resistant polymer catheter coating to reduce catheter associated urinary tract infection (CAUTI): A first-in-man pilot study. Eur. Urol. Suppl. 18, e377 (2019)
¹ Curtis A., Wilkinson C. Topographical control of cells. Biomaterials. 18, 1573-1583 (1997)
¹ Amin Y. Y., Runager K., Simoes F., Celiz A., Taresco V., RosR., Enghild J. J., Abildtrup L. A., Kraft D. C., Sutherland D. S., Alexander M. R., Foss M., Ogaki R. Adv. Mater. 28, 1472-1476 (2016)
¹ Ngandu Mpoyi E., Cantini M., Reynolds P. M., Gadegaard N., Dalby M. J., Salmerón-Sánchez M. Protein Adsorption as a Key Mediator in the Nanotopographical Control of Cell Behavior. ACS Nano 10, 6638-6647 (2016).
¹ Roach P., Parker T., Gadegaard N., Alexander M. R. A bio-inspired neural environment to control neurons comprising radial glia, substrate chemistry and topography. Biomater. Sci. 1, 83-93 (2013)
¹ Yang J., Rose F. R. A. J., Gadegaard N., Alexander M. R. A High-Throughput Assay of Cell-Surface Interactions using Topographical and Chemical Gradients. Adv. Mater. 21, 300-304 (2009)
¹ Unadkat, H. V., Hulsman M., Cornelissen K., Papenburg B. J., Truckenmüller R. K., Carpenter A. E., Wessling M., Post G. F., Uetz M., Reinders M. J. T., Stamatialis D., van Blitterswijk C. A., de Boer J. An algorithm-based topographical biomaterials library to instruct cell fate. Proc. Natl. Acad. Sci. USA 108, 16565-16570 (2011)
¹ Hulshof F. F. B., Papenburg B., Vasilevich A., Hulsman M., Zhao Y., Levers M., Fekete N., de Boer M., Yuan H., Shantanu S., Beijer N., Bray M. A., Logan D. J., Reinders M., Carpenter A. E., van Blitterswijk C., Stamatialis D., de Boer J. Mining for osteogenic surface topographies: In silico design to in vivo osseo-integration. Biomaterials 137, 49-60 (2017)
¹ Vasilevich A. S., Mourcin F., Mentink A., Hulshof F., Beijer N., Zhao Y., Levers M., Papenburg B., Singh S., Carpenter A. E., Stamatialis D., van Blitterswijk C., Tarte K., de Boer J. Designed Surface Topographies Control ICAM-1 Expression in Tonsil-Derived Human Stromal Cells. Front. Bioeng. Biotechnol. 6, 87 (2018)
¹ Vasilevich A., Vermeulen S., Kamphuis M., Roumans N., Eroume S., Hebels D. G. A. J., Reihs R., Beijer N., Carlier A., Carpenter A. E., Singh S., de Boer J., in preparation
¹ Nair D. P., Cramer N. B., Scott T. F., Bowman C. N., Shandas R. Photopolymerized Thiol-Ene Systems as Shape Memory Polymers. Polymer (Guildf) 51, 4383-4389 (2010)
¹ Carpenter A. E., Jones T. R., Lamprecht M. R., Clarke C., Kang I. H., Friman O., Guertin D. A., Chang J. H., Lindquist R. A., Moffat J., Golland P., Sabatini D. M. CellProfiler: image analysis software for identifying and quantifying cell phenotypes. Genome Biology 7, 100-111 (2006)
¹ Rutkovskiy A., Stensløkken K. O., Vaage I. J. Osteoblast Differentiation at a Glance. Med. Sci. Monit. Basic Res. 22, 95-106 (2016)
¹ Prins H. J., Schulten E. A. J. M., Ten Bruggenkate C. M., Klein-Nulend J., Helder M. N. Bone regeneration using the freshly isolated autologous stromal vascular fraction of adipose tissue in combination with calcium phosphate ceramics. Stem Cells Transl. Med. 5, 1362-1374 (2016)
¹ Davis H. E., Miller S. L., Case E. M., Leach J. K. Supplementation of fibrin gels with sodium chloride enhances physical properties and ensuing osteogenic response. Acta Biomater. 7, 691-9 (2011)
¹ Li D., Zheng Q., Wang Y., Chen H., Combining surface topography with polymer chemistry: exploring new interfacial biological phenomena. Polym. Chem. 5, 14-24 (2014)
¹ Ling Q., Zhang B., Kasoju N., Ma J., Yang A., Cui Z., Wang H., Ye H. Differential and Interactive Effects of Substrate Topography and Chemistry on Human Mesenchymal Stem Cell Gene Expression. Int. J. Mol. Sci. 19, 2344 (2018)
¹ Yu Q., Li X., Zhang Y., Yuan L., Zhao T., Chen H. The synergistic effects of stimuli-responsive polymers with nano-structured surfaces: wettability and protein adsorption. RSC Adv. 1, 262-269 (2011)
¹ Calvo A., Yameen B., Williams F. J., Soler-Illia G. J. A. A., Azzaroni O. Mesoporous Films and Polymer Brushes Helping Each Other To Modulate Ionic Transport in Nanoconfined Environments. An Interesting Example of Synergism in Functional Hybrid Assemblies. J. Am. Chem. Soc. 131, 10866-10868 (2009)
¹ Li J., Kwiatkowska B., Lu H., Voglstatter M., Ueda E., Grunze M., Sleeman J., Levkin P. A., Nazarenko I. Collaborative Action of Surface Chemistry and Topography in the Regulation of Mesenchymal and Epithelial Markers and the Shape of Cancer Cells. ACS Appl. Mater. Interfaces 8, 28554-28565 (2016)
¹ Li Q., Zhang B., Kasoju N., Ma J., Yang A., Cui Z., Wang H., Ye H. Differential and Interactive Effects of Substrate Topography and Chemistry on Human Mesenchymal Stem Cell Gene Expression. Int. J. Mol. Sci. 19, 2344 (2018)
¹ Bartneck. M, Schulte. V. A, Paul. N. E, Diez. M, Lensen. M. C., Zwadlo-Klarwasser. G. Induction of specific macrophage subtypes by defined micro-patterned structures. Acta Biomater. 6, 3864-3872 (2010)
¹ Vishwakarma A., Bhise N. S., Evangelista M. B., Rouwkema J., Dokmeci M. R., Ghaemmaghami A., Vrana N. E., Khademhosseini A. Engineering Immunomodulatory Biomaterials To Tune the Inflammatory Response. Trends Biotechnol. 34, 470-482 (2016)
¹ Breiman L., Random Forests. Mach. Learn. 45, 5-32 (2001)
¹ Mikulskis P., Alexander M. R., Winkler D. A. Toward Interpretable Machine Learning Models for Materials Discovery. Adv. Intel. Syst. 190045 (2019)

### Example 2—Identification of Polymers which Modulate Cellular Processes on Monocytes and APCs

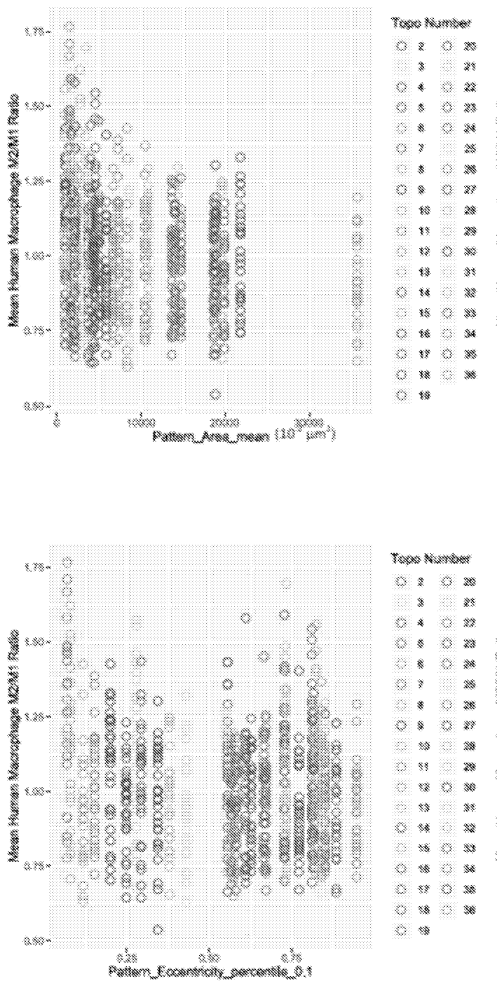

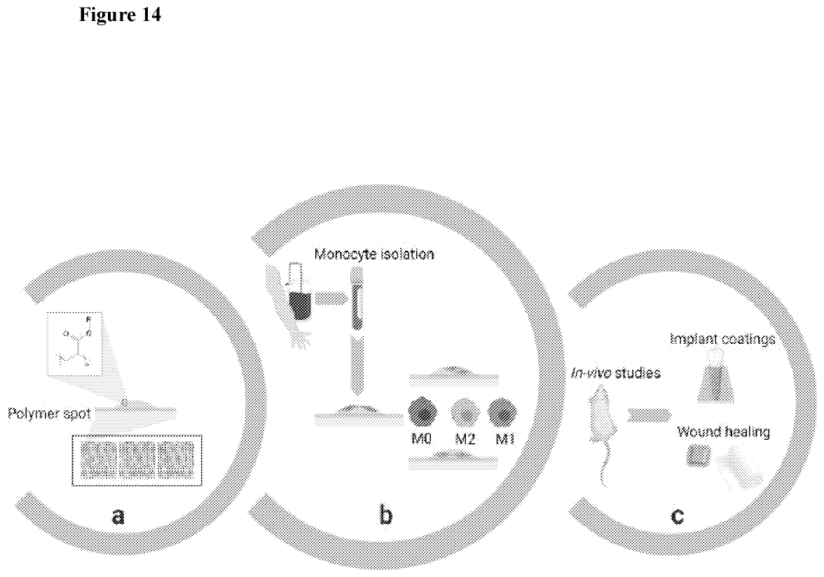

A library of homopolymers consisting of 141 (meth)acrylates and (meth)acrylamides monomers were screened for their ability to induce the differentiation of human monocytes to distinct macrophage phenotypes using fluorescent labels of surface markers to categorise cells to M1-like or M2-like phenotypes. Homopolymers of interest were selected from this screen to produce a second-generation polymer library by co-polymerising the monomers. A 400-member co-polymer array was produced, which was screened for ‘hit’ materials selected based on their ability to induce M1- and M2-like phenotypes in macrophages. These were then scaled up and used in a series of in vitro and in vivo experiments to assess their ability to modulate macrophage phenotype and response to an implanted foreign body (FIG. 14).

### High Throughput Polymer Chemistry Screening Identifies Immunomodulatory Materials

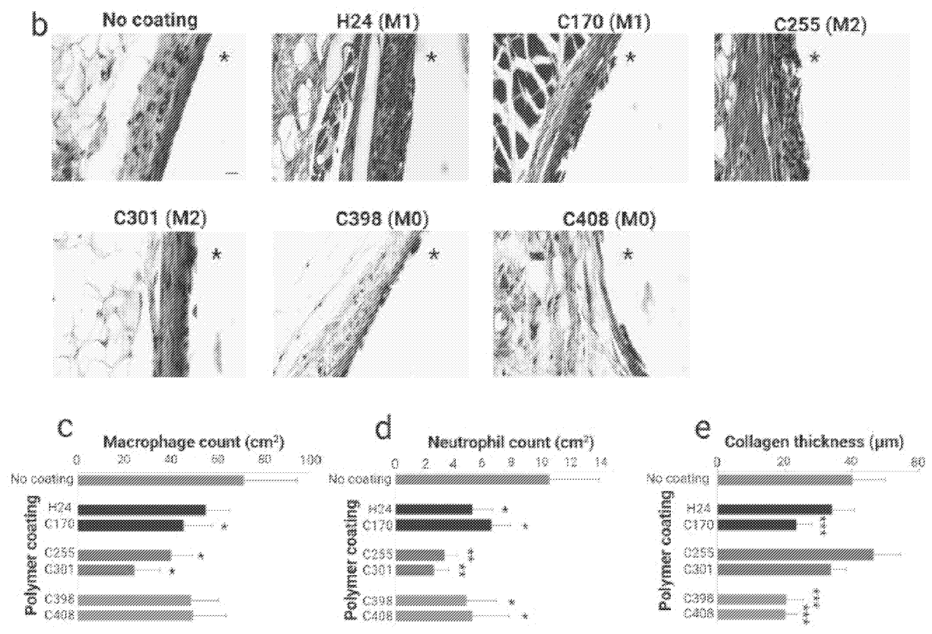

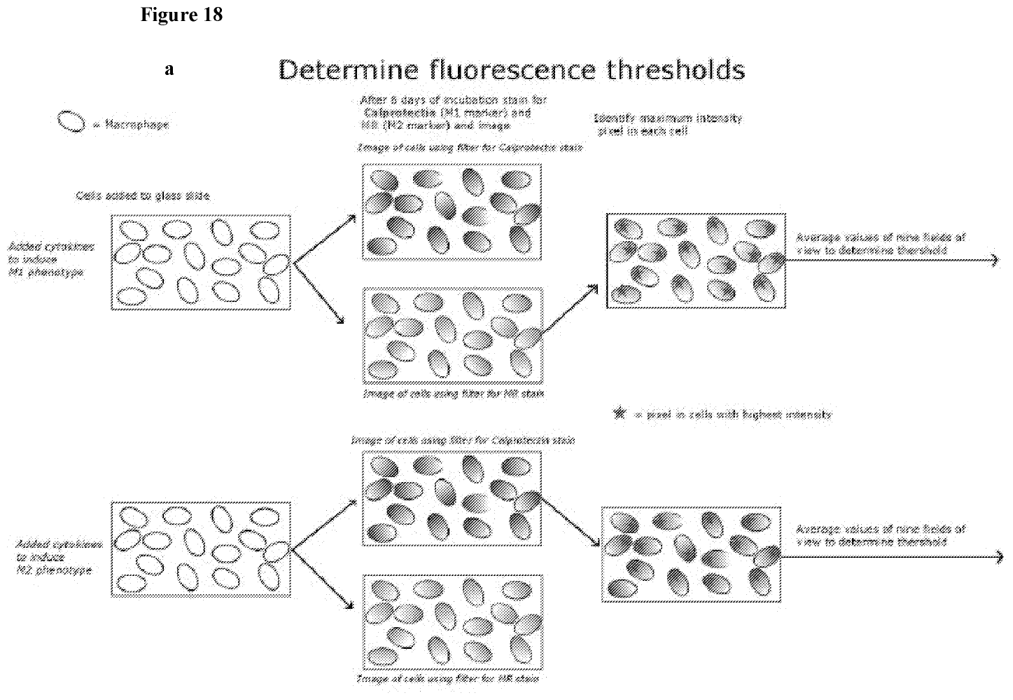

Using a high throughput screening strategy, the effect of a combinatorial library of polymers on macrophage attachment, morphology and phenotype over a 6-day culture was investigated. Monocytes from three different healthy donors were cultured on the first-generation array composed of 3 replicates of 141 unique (meth)acrylate homopolymers intended to screen a broad range of chemistries (Supplementary Table 1). Cell surface marker expression is widely used to assess macrophage phenotype and it can be readily applied to high throughput assessment of cells adhered to polymer microarrays using automated microscopy.17 The proportion of pro-inflammatory M1-like macrophages was quantified using expression of calprotectin and anti-inflammatory M2-like phenotypes using mannose receptor (MR) expression, first establishing reference fluorescence measurements in cytokine polarised M1 or M2 macrophages (on glass) (FIG. 18).

The average M2/M1 cell number ratio (from 3 spots), using cells from 3 different donors, was calculated for each polymer to identify ‘hit’ materials with the ability to induce M1-like or M2-like differentiation (FIGS. 15a and b). The homopolymer H47: poly N-[tris(hydroxymethyl)methyl] acrylamide (FIG. 15c) was most effective in polarising macrophages towards the M2-like phenotype, with an M2/M1 cell number ratio of nearly 7, but the total cell number average was relatively low (FIG. 15b). Two other polymers with high M2/M1 cell number ratios were H37: poly(methacrylamide) (FIG. 15d) and H9: poly(tridecafluorooctyl acrylate) with M2/M1 cell number ratio of 4.5 and 3.5 respectively, again with relatively low total cell numbers average. There were a number of polymers with M2/M1 cell number ratios close to 1, suggesting either evenly split M1 and M2 populations or naïve macrophage domination (cells stained equally with both markers), e.g. H132: poly (benzyl acrylate). However, H126: poly(isobutyl acrylate) (FIG. 15e), H98: poly(hydroxypropyl acrylate) (FIG. 15f), and H135: poly(ethylene glycol phenyl ether methacrylate) were the most effective at polarising cells towards the M1 phenotype with M2/M1 cell number ratios of 0.22, 0.41 and 0.42 respectively. The total cell number on each polymer varied across the library by over an order of magnitude from 9.8±3.9 cells observed on H39: poly(tridecafluorooctyl methacrylate) (FIG. 15i) to 230±65 cells observed on H42: poly(cyclohexyl methacrylate) (FIG. 15h). Since these cells do not proliferate, the observed differences in cell number indicate differential cell attachment. A number of homopolymers showed high levels of cell adhesion (H133, H90, H103, H21, H94, H24, H69, H96, H92 and H33) (FIG. 15g), with average cell attachment numbers of 209±48, 197+69, 171+32, 169+18, 168+33, 151+120, 147+8.2, 137+53, 132±39 and 127+25 cells, respectively.

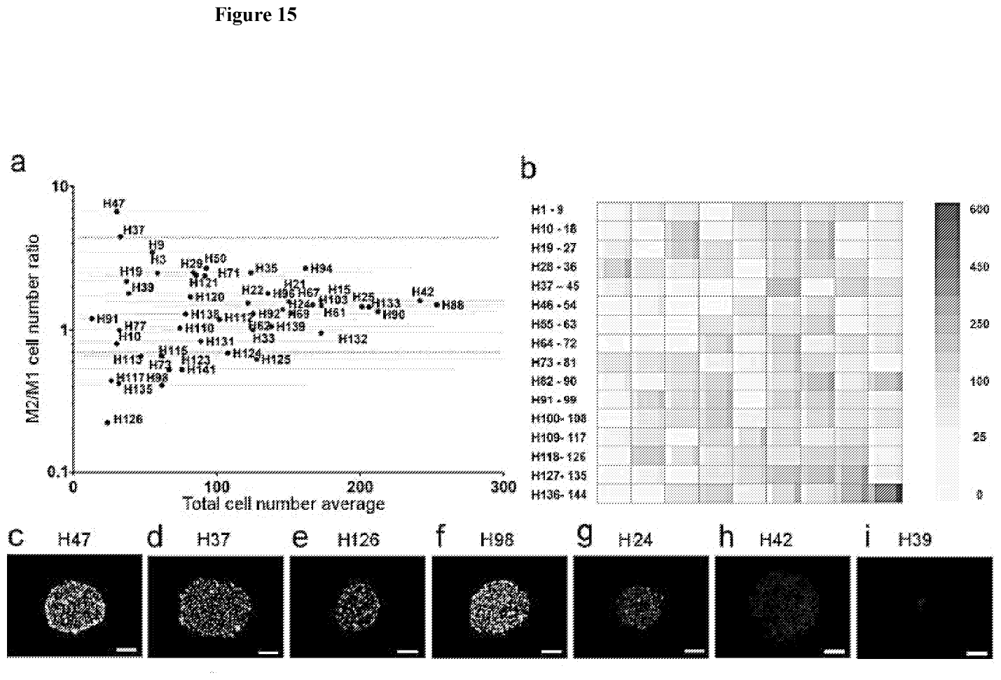

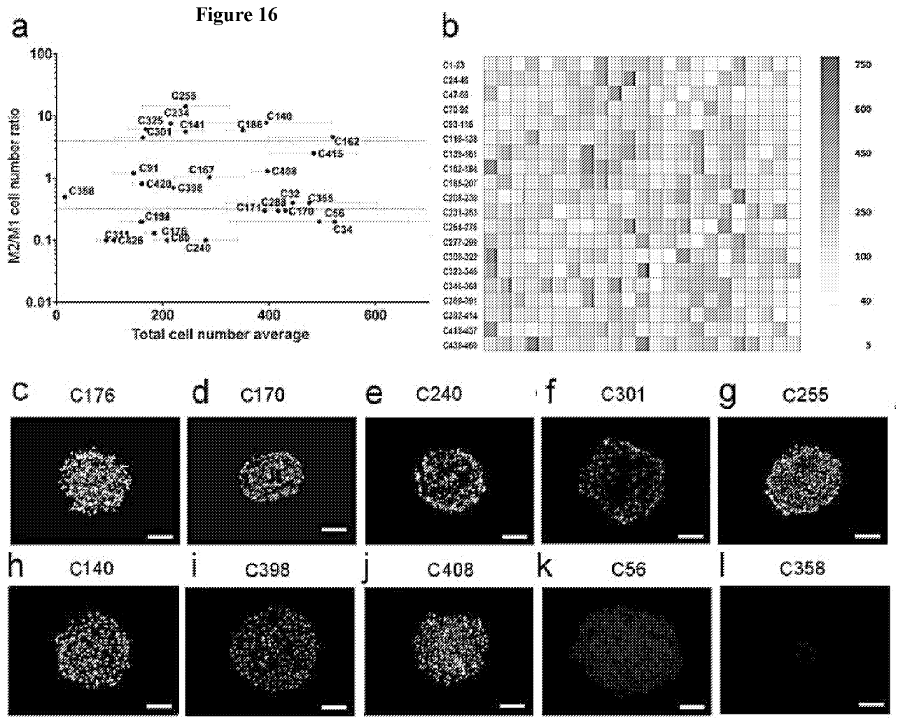

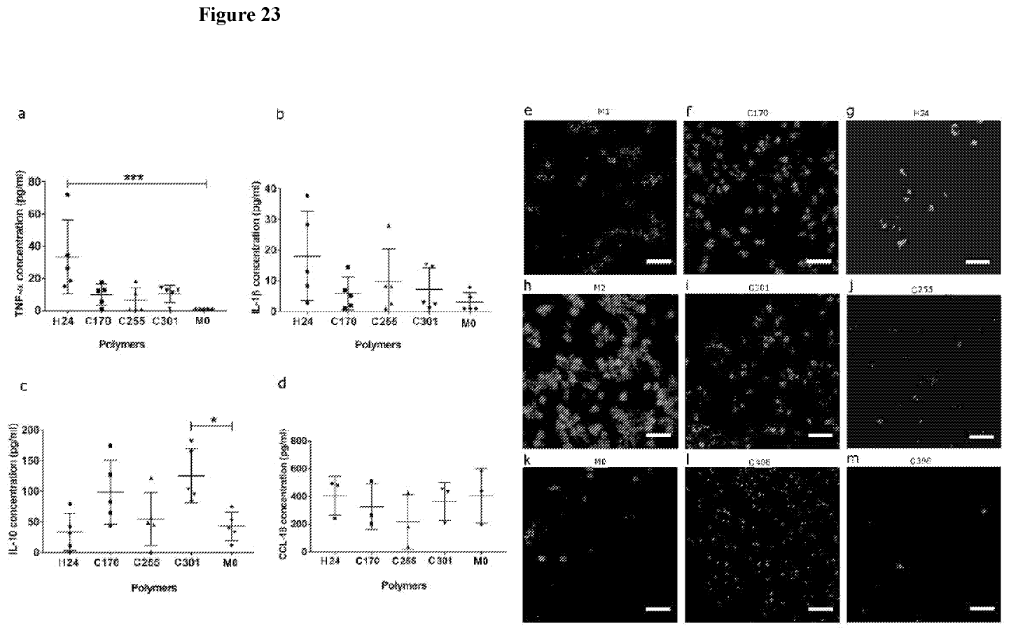

To investigate whether homopolymers inducing macrophage polarisation could be combined with those promoting high cell attachment, co-polymerisation was used to form a second-generation combinatorial polymer library. For this we selected the top ten homopolymers able to induce M2-like (M2/M1 cell number ratios: 2.4) or M1-like phenotypes (M2/M1 cell number ratio ≤0.66) together with ten homopolymers showing the highest cell attachment (Supplementary Table 2) to create a combinatorial library of 400 co-polymers (Supplementary Table 3). Using the same procedure as for the first-generation array, purified monocytes were incubated on co-polymer arrays with each individual adhered cell categorised as M1-like or M2-like after 6 days (FIG. 23); the ratio is plotted against the number of adherent cells in FIG. 16a.

The highest level of mannose receptor (MR) expression, an M2 marker, was observed from monocytes seeded on C255 (H88-co-H25), C140 (H94-co-H126), C186 (H29-co-H126) in FIG. 16f, g, h. Monocyte polarisation towards M2 was evidenced by 5-fold higher number of M2 than M1 cells on these co-polymers. A similar number of M2 and M1 cells were observed on C398 (H15-co-H113) (FIG. 16i) and C408 (H9-co-H117) (FIG. 16j) hence they were considered as M0 co-polymers. A number of M1 polarising co-polymers were identified as evidenced by high levels of calprotectin expression, which resulted in a 10-fold lower M2/M1 ratio: C176 (H125-co-H133) (FIG. 16c), C170 (H41-co-H42) (FIG. 16d) and C240 (H3-co-H29) (FIG. 16e).

The highest number of adhered cells were observed on co-polymer C56 (H50-co-H29), (411±143 cells) whilst C358 (H29-co-H115) had the lowest number of the cells (19±6 cells) (FIGS. 16k and l). Co-polymers C56 (H50-co-H29) (411±143 cells), C386 (H71-co-H126) (363±99 cells), C32 (H25-co-H67), C347 (H3-co-H126) (328±126 cells) and C295 (H94-co-H71) (347±166 cells) from the second generation array had the highest number of attached cells however interestingly non-of their constituent homo-polymers had a significant high cell attachment suggesting a synergistic effect upon combination.

Co-polymers C358 (H29-co-H115) (18.8±6), C209 (H35-co-H126) (47±22), C434 (H35-co-H123) (51±17), C94 (H35-co-H47) (54±9) and C48 (H50-co-H47) (56±19) on the other hand had the lowest number of attached cells, consistent with low cell attachment to their constituent homo-polymers. Monomer H35 poly(hexyl acrylate) was a constituent of the second, third and fourth least adherent co-polymers, indicating that this monomer may be involved in preventing cell attachment. Such different cell attachment did not associate with a particular macrophage polarisation status. Amongst the polymers that induced M2 or M1-like polarisation, C162 (H42-co-H126) (M2) and C170 (H42-co-H141) (M1) (FIG. 16d) were the most cell-attractive polymers with 520±2 cells and 431±54 cells respectively, whilst C164 (H24-co-H98) (M2) and C311 (H24-co-H61) (M1) were the least cell-attractive polymers with 125±9 and 94±24 cells attached, respectively (FIG. 16b).

Correlation of Immune-Instructive Behaviour with Polymer Chemistry Using Machine Learning

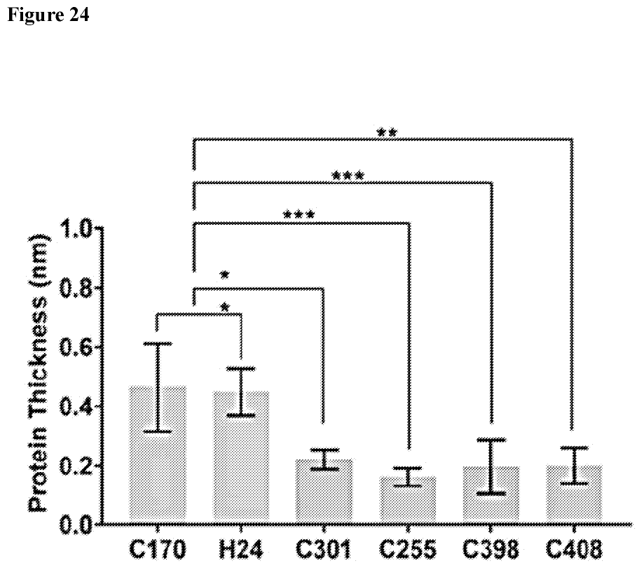

Data generated by high throughput experiments can be used to develop polymer structure-cell response models using machine learning. These models can enable the prediction of the immune-instructive properties of new materials yet to be synthesised by identification of the types of chemical features that promote or prevent macrophage attachment and polarisation.18, 19, 20 To test the applicability of this approach to our data set we undertook a computational study to identify important chemical descriptors in macrophage attachment and polarisation. As cell attachment and polarization were both equally important (Supplementary Table 4 and 5), we trained machine learning models to predict a composite dependent variable, log(M2/M1 ratio) multiplied by the cell attachment. This variable has large positive or negative values for desirable materials with high attachment and polarization (M2 or M1) and low values for those with low attachment and/or low polarization. We generated a two-class predictive model for this parameter by assigning materials with most positive value for the composite variable to the anti-inflammatory phenotype class, and the materials with most negative values for the composite variable into the pro-inflammatory class. The anti- and pro-inflammatory classes were defined after clustering the dataset and selecting those instances from the clusters with the highest and lowest values found for the composite variable (FIG. 24). To provide chemically informative models we encoded the various polymer chemistries using molecular signature descriptors that relate directly to polymer structure.21 A least absolute shrinkage and selection operator (LASSO), to eliminate uninformative and less informative descriptors, and generated two-class models using Random Forest, Support Vector Machines, and Multilayer Perceptron Models was used.22, 23, 24, 25 All three of these non-linear methods generated models of similar accuracy. The model with the greatest accuracy assigned the materials to the correct classes with an accuracy of 81%. The molecular features that generated the highest values of the composite variable (log (M2 polarisation)×total cell attachment), i.e. that induced the most anti-inflammatory macrophage phenotype with a high cell attachment, which also shows the performance of this model. Alkoxy molecular fragments were found to contribute strongly to the model, with propyloxy, 2,3-dimethylpropyloxy and ethylene glycol fragments appearing at the top of the list. A fluorinated tert-butyl and methacrylamide fragment also appeared to have a strong influence on the model, as does a more sterically hindered 2,2,3-trimethylbutyl structure. These observations provide some guidance to the choice of monomer structure for future rational design of monomers, with the selective combination of branched and alkoxy fragments potentially able to enhance this effect. An implication from this model is that consideration of both steric and electronic factors are important for macrophage response, exemplifying the feasibility of combining high throughput experiments and machine learning to identify materials chemistry to be a significant driver of immune cell polarisation.

### Functional Assays Confirm Phenotype Determined by High Throughput Experiments

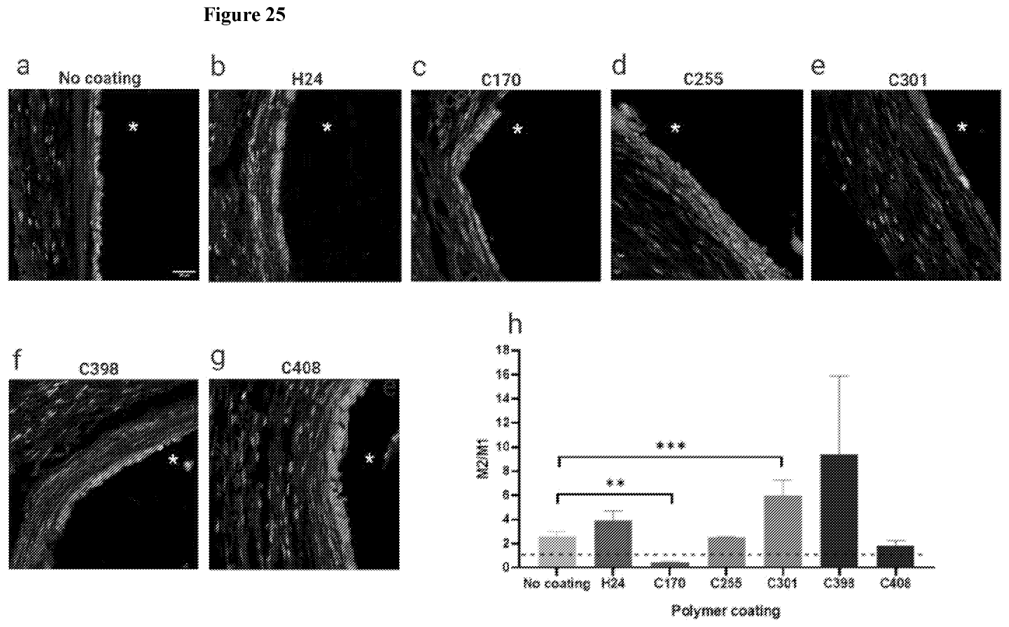

To see whether the changes in surface marker expression towards M1 or M2-like phenotypes on different polymers was also reflected in cell function, we investigated the cytokine profile and phagocytic ability of macrophages differentiated on a selection of polymer (FIG. 25). Based on array data and scalability, 4 polymers were selected that were shown in-vitro to induce either M1-like (H24 and C170) or M2-like (C255 and C301) phenotypes. These polymers were scaled up to a 24-well format followed by culturing monocytes on polymer coated plates for 6 days before cytokine quantification. Cells cultured on tissue culture plastic with no polarising cytokines (naïve macrophages) were used as controls. Cells differentiated on H24 produced significantly higher levels of the pro-inflammatory cytokine TNFα compared to naïve macrophages (FIG. 25a-d). This polymer also induced the production of higher levels of IL-1β, another pro-inflammatory cytokine, but this did not reach statistical significance. Interestingly, C170, the other M1 polarising polymer, induced higher levels of IL-10 than all other polymers in this assay except for the M2 polarising polymer C301, which also induced significantly higher levels of IL-10 production. Therefore, cells polarised on H24 and C301 had a cytokine profile in line with their surface phenotypes and for other M1 and M2 polarising polymers we detected a mixed cytokine profile. The cell viability was comparable in all conditions. Our data highlighted the ability of different polymers to drive macrophage polarisation towards distinct functional phenotypes that are akin with their cytokine polarised counterparts but not identical. The phagocytosis of zymosan particles (a model for recognition of microbes by the innate immune system) by macrophages polarised on C170 (M1 polarising polymer) was significantly higher than naïve macrophages followed by cells polarised on polymer C301 (M2 polarising polymer) (FIG. 25 e-h).

Following overnight incubation in media, the resultant deposited protein thickness on the polymers was measured by X-ray photoelectron spectroscopy (XPS). The protein layer on M1 biased polymers (H24 and C170) was found to be two-fold thicker than the thickness of the protein layer on the naïve (C398 and C408) and M2 biased polymers (C255 and C301) (FIG. 24, this is consistent with previous findings.26 This suggests that it is possible that the total amount of adsorbed protein plays a role in differential polarisation of macrophages.

### M1 and M2 Polymer Hits Induce Differential Tissue Response as Evidenced by Collagen Deposition and Immune Cell Infiltration

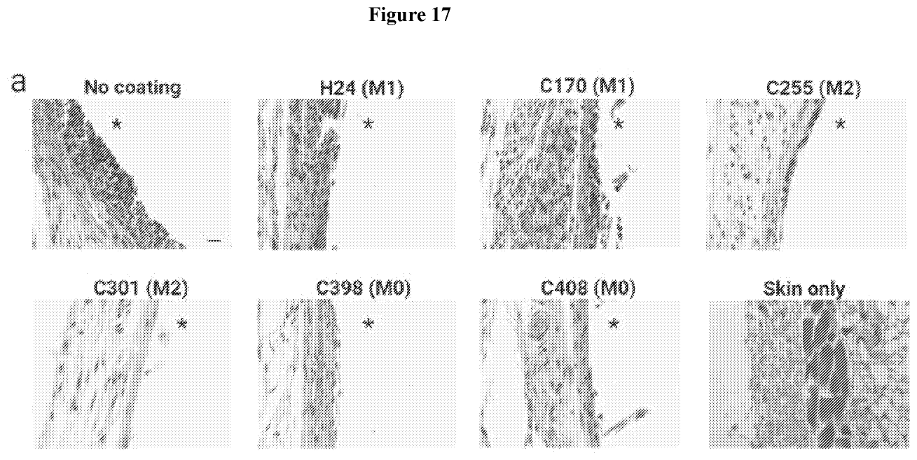

Polymer hits H24, C170, C255, C301, C398 and C408 were coated onto silicone rubber tube segments using a dip-coating process and implanted subcutaneously into mice for a period of 28 days. Haematoxylin and Eosin (H&E) together with Masson's trichrome (MTC) stains were used to assess the tissue inflammatory response in terms of inflammatory cell components, angiogenesis and collagen deposition (FIGS. 17a and b). A typical FBR involves rapid and early infiltration of neutrophils, closely followed by macrophages. The intensity of cell infiltration varied between polymers, from an overt cell infiltration in non-coated silicone followed by H24 and C170 polymers, and mild/sparse immune cell infiltration for C398 and C408, to significantly fewer cells for C255 and C301 coatings (FIG. 17a). This is consistent with the in-vitro observations, where H24 and C170 elicited a more M1-like phenotype response and C255 and C301 a more anti-inflammatory M2-like response whereas C398 and C408 were assigned as M0 inducing polymers. It has been shown that as pro-inflammatory macrophages increase, additional neutrophils are recruited.27 Consistent with this in FIGS. 17c and d, increases in macrophage and neutrophils were detected near the surface of H24 and C170. Macrophages, especially those of an M2-like phenotype, are involved in tissue repair and remodelling through release of growth factors and cytokines.27 These promote the recruitment of fibroblasts, which secrete matrix molecules and collagen to bring about tissue repair or excessive ECM deposition and fibrosis if activated continuously. Collagen deposition at the perilesional tissue was shown using MTC in FIG. 17b. Non-coated silicone and C255 showed the thickest collagen layer, along with C301 and H24 (FIG. 17e) consistent with the induction of M2-like behaviour of the co-polymers C255 and C301 observed in-vitro. Intriguingly, C398 and C408, the M0 inducing polymers, showed the least amount of collagen deposition. These observations suggest that while sustained and selective M1 or M2 like macrophage activation could lead to brisk inflammation or excessive collagen deposition respectively, presence of both M1 and M2 like cell types at similar level (seen in M0 polymers) at foreign body site reduces fibrotic tissue formation. This is in line with evidence from different experimental models of inflammatory tissue injury and fibrosis28. Characterising the macrophage phenotype at the catheter-tissue interface was carried out using the pro-inflammatory marker inducible nitric oxide synthase (iNOS) and the anti-inflammatory marker arginase-1 (Arg-1). A double-stain immunofluorescence method was used to stain the tissue sections with cells expressing iNOS labeled in green and cells expressing Arg-1 labeled in red. Representative images of cells exposed to non-coated catheter segments and catheters coated in polymers H24, C170, C255, C301, C398 and C408 are shown in Supplementary FIG. 25a-g. Macrophages display a spectrum of activation phenotypes, and it is the relative proportion of M1 or M2 markers that can be used as a handle to determine the type of activation status.29 Supplementary FIG. 25h shows the ratio of M2/M1 cells in the tissue near the polymer surface. An M2/M1 ratio close to 1.0 was shown by polymer C408 (M0 polymers), where the presence of equal numbers of iNOS or Arg-1 expressing cells seemed to support tissue homeostasis. A more pronounced M1-like phenotype was shown by polymer C170 (M1 polymer) and a more pronounced M2-like phenotype was shown by polymer C301 (M2 polymer). This data is broadly in line with the pro- and anti-inflammatory phenotypes observed in in-vitro high throughput screening and in vivo histological data.

### Machine Learning Models to Predict the Immune-Instructive Properties of New Materials

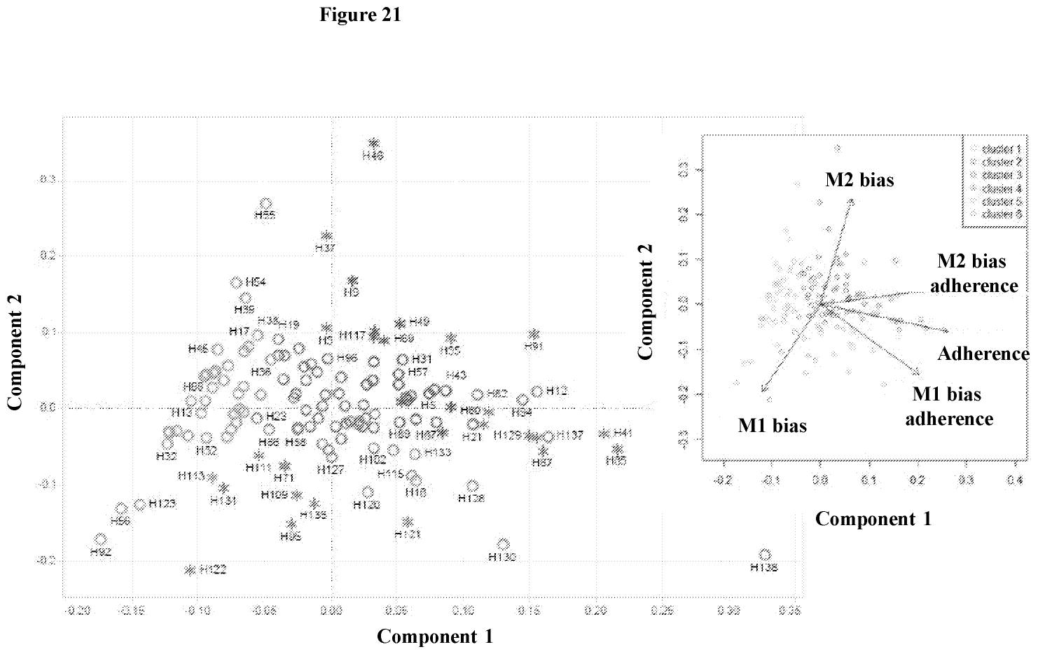

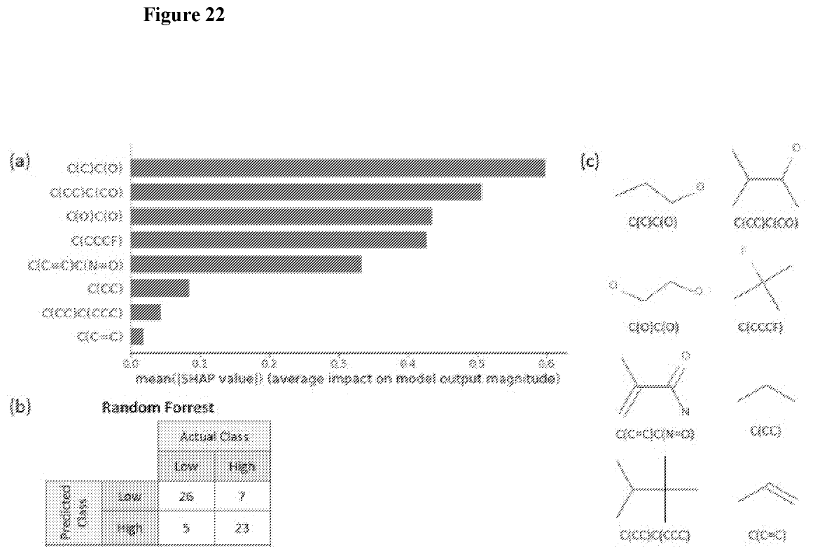

An initial proof-of-concept computational study was conducted using the data generated in our experiments for co-polymers. As attachment and polarisation were both important, we trained three types of machine learning models to predict a composite dependent variable, the M2/M1 ratio multiplied by the attachment value. Initially, we conducted a consensus clustering analysis of the new composite dependent variable to separate the co-polymers by their function. We initially obtained three clusters, with high, medium and low values for M2/M1*attachment. We excluded the instances with medium values, as they were not of interest for generating immune instructive materials. Subsequently, we created two class predictive models by assigning materials with highest value for the composite variable to the ‘active’ class, and the materials with smallest values for the composite variable into the ‘inactive’ class. We encoded the various polymer chemistries using molecular signature descriptors that relate directly to particular functional groups in the polymers. We used an L1 sparse feature selection method, LASSO, to eliminate uninformative and less informative descriptors, and generated two-class models using random forest, support vector machines, and multilayer perceptron models. The performance of this model and the structures of the molecular entities that contributed most strongly to the attachment and polarisation are shown in FIG. 22a. The figure presents the results of the SHapley Additive exPlanation (SHAP) method employed to the dataset features. The features are ordered from top to bottom based on their average impact on the model output magnitude. These molecular features are constituents of the best immune instructive material described above. Supplementary FIG. 22b shows the confusion matrix obtained by the best classifier model. The classes considered were high and low values for the composite variable Log(M2 polarization)×total cell attachment.

### SUMMARY

It is clearly shown that unbiased in-vitro screening of a large array of polymer chemistries with monocytes successfully identified novel materials with potent immune-modulatory properties, validated in a murine in-vivo model where pro- or anti-inflammatory responses were shown by histological examination. The polymer structure-cell response relationships could be modelled using machine learning using descriptors of the monomer chemistry, highlighting the potential to undertake ‘immune-instructive’ rational design. Macrophage polarisation towards pro- and anti-inflammatory phenotypes was closely linked to the extent of protein deposition on the polymers. Identifying new polymers with immune-modulatory properties and elucidating the molecular mechanisms involved offers exciting possibilities for the rational design of novel bio-instructive materials with numerous clinical applications from implants and vaccine adjuvants to tissue regeneration and drug delivery.

TABLE 5
The first generation homo-polymer microarray libraries with their code, smiles and chemical structures

| Codes | Monomers | Chemical Structures |
|---|---|---|
| H1 | N-(4-Hydroxyphenyl)methacrylamide | 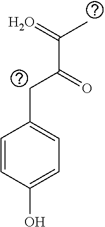 |
| H2 | N,N′-Methylenebismethacrylamide | 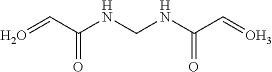 [SMILES: O=CC(=O)NCNC(=O)C=O] |
| H3 | Octafluoro-2-hydroxy-6-(trifluoromethyl) heptyl methacrylate | 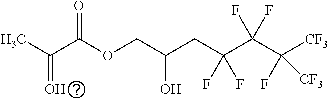 [SMILES: CC(=O)C(=O)OCC(O)CC(F)(F)C(F)(F)C(F)(C(F)(F)F)C(F)(F)F] |
| H4 | Ethyl methacrylate | 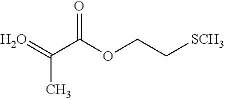 |
| H5 | Octafluoropentyl acrylate | 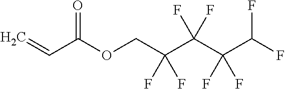 [SMILES: C=CC(=O)OCC(F)(F)C(F)(F)C(F)(F)C(F)F] |
| H6 | Benzyl methacrylate | 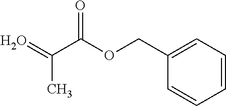 |
| H7 | Octafluoropentyl methacrylate | 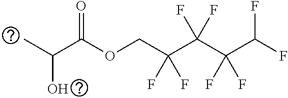 [SMILES: CC(O)C(=O)OCC(F)(F)C(F)(F)C(F)(F)C(F)F] |
| H8 | Neopentyl glycol propoxylate diacrylate | 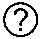 |
| H9 | Tridecafluorooctyl acrylate | 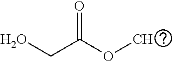 |
| H10 | Acrylamide | 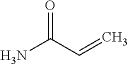 |
| H11 | Diacetone acrylamide | 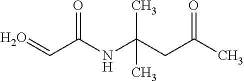 |
| H12 | Trimethylhexyl acrylate | 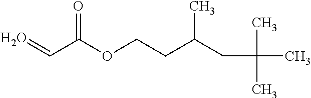 |
| H13 | Ter-butyl methacrylate | 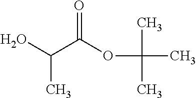 |
| H14 | Poly(propylene glycol) acrylate | 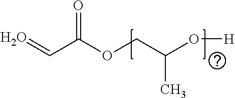 |
| H15 | Ethylene glycol dimethacrylate | 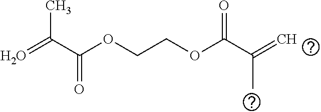 |
| H16 | Tetrahydrofurfuryl acrylate | 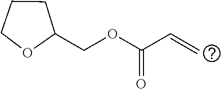 |
| H17 | Lauryl acrylate | 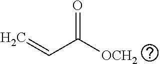 |
| H18 | Tetra(ethylene glycol) diacrylate | 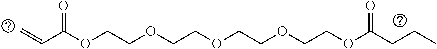 [SMILES: C=CC(=O)OCCOCCOCCOCCOC(=O)CCC] |
| H19 | N-Methylmethacrylamide | 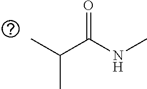 |
| H20 | N-Isopropylacrylamide | 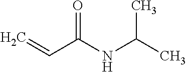 |
| H21 | Tetraethylene glycol dimethacrylate | 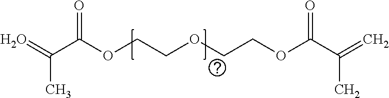 |
| H22 | Ethoxyethyl methacrylate | 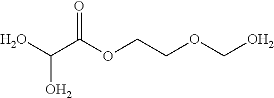 [SMILES: O=C(OCCOCO)C(O)O] |
| H23 | Di(ethylene glycol) 2-ethylhexyl ether acrylate | 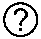 |
| H24 | t-Butylcyclohexyl methacrylate | 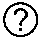 |
| H25 | sodecyl methacrylate | 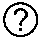 |
| H26 | Butyl acrylate | 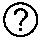 |
| H27 | Trimethylolpropane ethoxylate triacrylate | 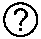 |
| H28 | N-[3-(Dimethylamino)propyl]methacrylamide | 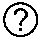 |
| H29 | N-(1, 1,3,3-tetramethylbutyl) acrylamide | 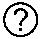 |
| H30 | 3-Sulfopropyl methacrylate potassium salt | 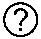 |
| H31 | Bisphenol A ethoxylate diacrylate | 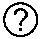 |
| H32 | Butyl methacrylate | 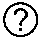 |
| H33 | Hexanediol ethoxylate diacrylate | 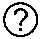 |
| H34 | Tetrahydrofurfuryl methacrylate | 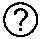 |
| H35 | Hexyl acrylate | 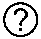 |
| H36 | Trimethylolpropane triacrylate | 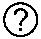 |
| H37 | Methacrylamide | 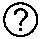 |
| H38 | N,N′-(1,2-Dihydroxyethylene)bisacrylamide | 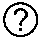 |
| H39 | Tridecafluorooctyl methacrylate | 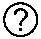 |
| H40 | Di(ethylene glycol) methyl ether methacrylate | 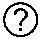 |
| H41 | Hexafluorobutyl acrylate | 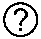 |
| H42 | Cyclohexyl methacrylate | 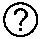 |
| H43 | Heptafluorobutyl methacrylate | 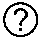 |
| H44 | Neopentyl glycol diacrylate | 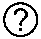 |
| H45 | Dodecafluoroheptyl acrylate | 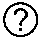 |
| H46 | N-(Isobutoxymethyl) acrylamide | 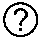 |
| H47 | N-[Tris(hydroxymethyl) methyl]acrylamide | 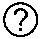 |
| H48 | Ethoxyethyl acrylate | 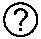 |
| H49 | Hydroxypropyl methacrylate | 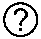 |
| H50 | Decyl methacrylate | 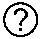 |
| H51 | Hydroxy-3-phenoxypropyl acrylate | 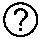 |
| H52 | Methylthioethyl methacrylate | 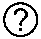 |
| H53 | Ethyl acrylate | 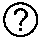 |
| H54 | Trimethylolpropane ethoxylate methyl ether diacrylate | 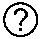 |
| H55 | N-(3-Methoxypropyl) acrylamide | 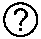 |
| H56 | Acrylamide | 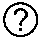 |
| H57 | Tri(ethylene glycol) dimethacrylate | 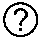 |
| H58 | Hydroxypivalyl hydroxypivalate bis[6-(acryloyloxy)hexanoate] | 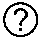 |
| H59 | Ethyl 2-ethylacrylate | 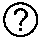 |
| H60 | Tricyclodecane-dimethanol diacrylate | 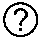 |
| H61 | Ethylhexyl methacrylate | 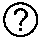 |
| H62 | Propargyl acrylate | 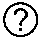 |
| H63 | Hexamethylene diacrylate | 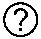 |
| H65 | N-[2-(1H-indol-3-yl) ethyl]acrylamide | 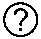 |
| H66 | 2-Methacryloyloxyethyl phosphorylcholine | 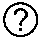 |
| H67 | Tert-butylcyclohexylacrylate | 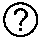 |
| H68 | Vinyl methacrylate | 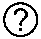 |
| H69 | Butanediol diacrylate | 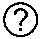 |
| H70 | Furfuryl methacrylate | 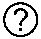 |
| H71 | Ethylhexyl acrylate | 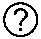 |
| H72 | Butanediol-1,3 diacrylate | 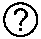 |
| H73 | N-Phenylmethacrylamide | 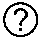 |
| H74 | N, N′-Methylenebisacrylamide | 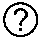 |
| H75 | Hexafluorobutyl methacrylate | 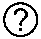 |
| H76 | Allyl methacrylate | 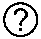 |
| H77 | Hexadecafluoro-9-(trifluoromethyl) decyl acrylate | 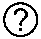 |
| H78 | Methacryloyloxy)ethyl acetoacetate | 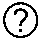 |
| H79 | Hexafluoropent-1,5-diyl diacrylate | 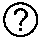 |
| H80 | sodecyl acrylate | 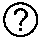 |
| H81 | Hexafluoroisopropyl acrylate | 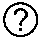 |
| H82 | N-[3-(Dimethylamino)propyl]acrylamide | |
| H83 | N-tert-Butylacrylamide | [symbol] |
| H84 | Poly(ethylene glycol) phenyl ether acrylate | 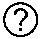 |
| H85 | Acryloyloxy-2-hydroxypropyl methacrylate | 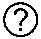 |
| H86 | Methyl 3-hydroxy-2-methylenebutyrate | 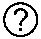 |
| H87 | Poly(ethylene glycol) methyl ether acrylate | 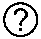 |
| H88 | Phenyl methacrylate | 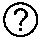 |
| H89 | Glycidyl acrylate | 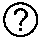 |
| H90 | Di(ethylene glycol) diacrylate | 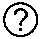 |
| H91 | N-Hydroxyethyl acrylamide | 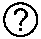 |
| H92 | N-Dodecylacrylamide | 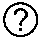 |
| H93 | Sulfopropyl acrylate potassium salt | 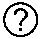 |
| H94 | 3-Hydroxy-2,2-dimethylpropyl 3-hydroxy-2,2- dimethylpropionate diacrylate | 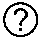 |
| H95 | Hydroxyethyl methacrylate | 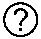 |
| H96 | Carboxyethyl acrylate | 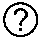 |
| H97 | Isobornyl methacrylate | 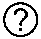 |
| H98 | Hydroxypropyl acrylate | 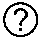 |
| | | 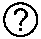 |
| H99 | Methyl-1,2-ethanediyl bis[oxy(methyl-2,1- ethanediyl)]diacrylate | 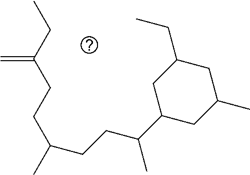 [SMILES: C=C(CC)CCC(C)CCC(C)C1CC(C)CC(CC)C1] |
| H101 | N, N′-Methylenebismethacrylamide | 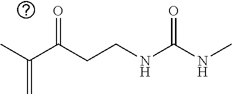 |
| H102 | 1,3,5-Triacryloylhexahydro-1,3,5-triazine | 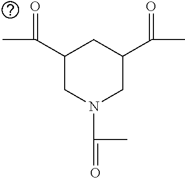 |
| H103 | Hexyl methacrylate | 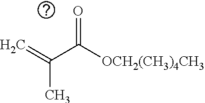 |
| H104 | Hydroxybutyl methacrylate | 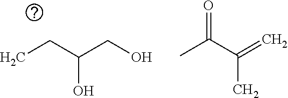 |
| H105 | Poly(propylene glycol) diacrylate | 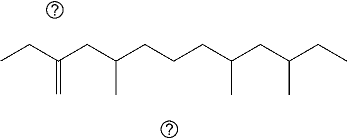 [SMILES: C=C(CC)CC(C)CCCC(C)CC(C)CC] |
| H106 | Lauryl methacrylate | 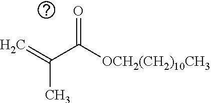 |
| H107 | Ethylene glycol methyl ether methacrylate | 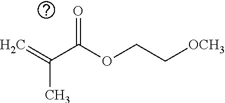 |
| H108 | sobornyl acrylate | 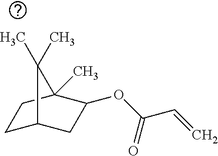 |
| H109 | N-(3-Aminopropyl)methacrylamide hydrochloride | 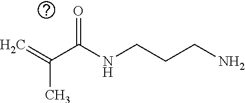 |
| H110 | 1,4-Bis(acryloyl)piperazine | 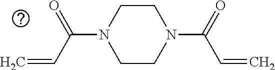 |
| H111 | Hexafluoroisopropyl methacrylate | 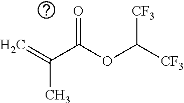 |
| H112 | Acryloyloxy-B, B-dimethyl-Y-butyrolactone | 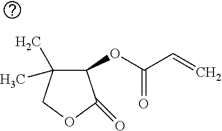 |
| H113 | Heptadecafluorodecyl methacrylate | 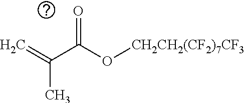 |
| H114 | Stearyl methacrylate | 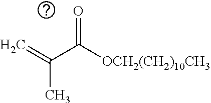 |
| H115 | Dodecafluoro-7-(trifluoromethyl)-octyl acrylate | 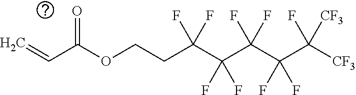 [SMILES: C=CC(=O)OCCC(F)(F)C(F)(F)C(F)(F)C(F)(F)C(F)(C(F)(F)F)C(F)(F)F] |
| H116 | Isooctyl acrylate | 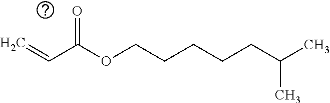 |
| H117 | Heptafluorobutyl acrylate | 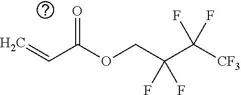 [SMILES: C=CC(=O)OCC(F)(F)C(F)(F)C(F)(F)F] |
| H118 | N,N′-Dimethylacrylamide | 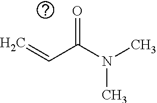 |
| H119 | N-(Butoxymethyl)acrylamide | 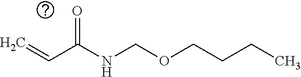 |
| H120 | Glycerol dimethacrylate | 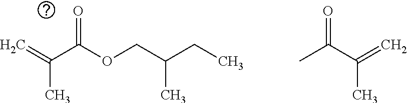 |
| H121 | Butoxyethyl methacrylate | 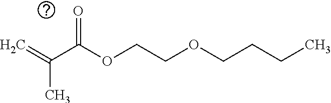 |
| H122 | Ethylene glycol diacrylate | 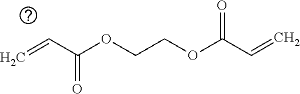 |
| H123 | Chloro-2-hydroxy-propyl methacrylate | 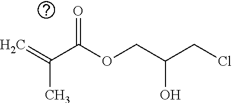 |
| H124 | Caprolactone 2-(methacryloyloxy)ethyl ester | 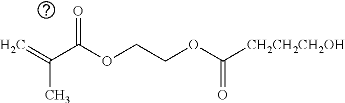 |
| H125 | Di(ethylene glycol) ethyl ether acrylate | 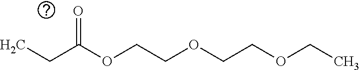 |
| H126 | Isobutyl acrylate | 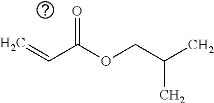 |
| H127 | N, N′-Dimethylmethacrylamide | 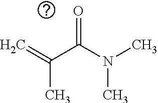 |
| H128 | N-(Hydroxymethyl) acrylamide | 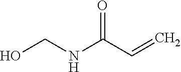 |
| H129 | [2-(Methacryloyloxy)ethyl]dimethyl-(3- sulfopropyl) ammonium hydroxide | 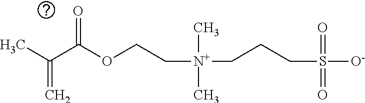 [SMILES: C=C(C)C(=O)OCC[N+](C)(C)CCCS(=O)(=O)[O-]] |
| H130 | Tris[2-(acryloyloxy)ethyl] isocyanurate | 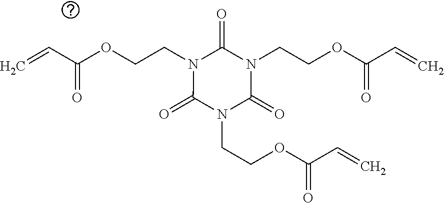 [SMILES: C=CC(=O)OCCn1c(=O)n(CCOC(=O)C=C)c(=O)n(CCOC(=O)C=C)c1=O] |
| H131 | Glycidyl methacrylate | 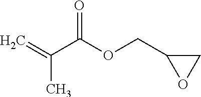 |
| H132 | Benzyl acrylate | 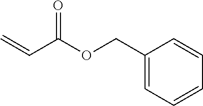 |
| H133 | Norbornyl methacrylate | 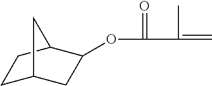 |
| H134 | Ethylene glycol phenyl ether methacrylate | 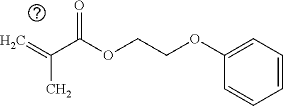 |
| H135 | Glycerol propoxylate triacrylate | 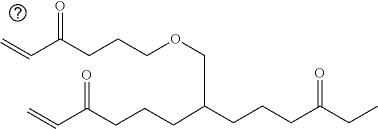 [SMILES: C=CC(=O)CCCOCC(CCCC(=O)C=C)CCCC(=O)CC] |
| H137 | N-tert-Butylmethacrylamide | 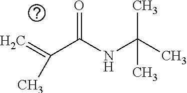 |
| H138 | Dimethylamino-ethyl acrylate | 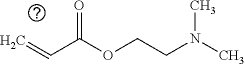 |
| H139 | Cyanoethyl acrylate | 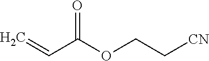 |
| H140 | Tert-butylamino-ethyl methacrylate | 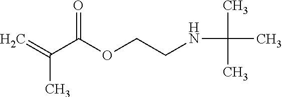 |
| H141 | Dimethylamino-ethyl methacrylate | 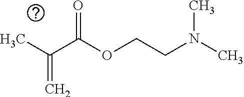 |
| H142 | Diethylaminoethyl methacrylate | 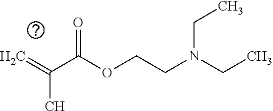 |
| H143 | Dimethylamino-propyl acrylate | 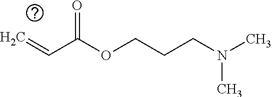 |
| H144 | Diethylamino ethyl acrylate | 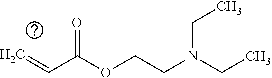 |

[symbol]

indicates data missing or illegible when filed

TABLE 6
Top ten homo-polymers with the highest M2/M1 cell ratio (M2) and the lowest M2/M1 cell ratio
(M1) together with the top ten homo-polymers showing the highest cell attachment
M1(biased) monomers

| Code | Monomer name | Monomer structure |
|---|---|---|
| H73 | N-Phenylmethacrylamide | 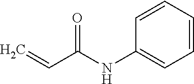 |
| H98 | Hydroxypropyl acrylate | 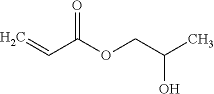 |
| | | 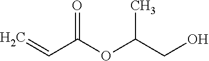 |
| H113 | Heptadecafluorodecyl methacrylate | 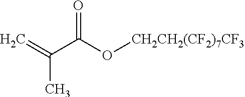 |
| H115 | Dodecafluoro-7-(trifluoromethyl)- octyl acrylate | 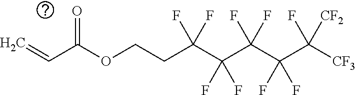 |
| H117 | Heptafluorobutyl acrylate | 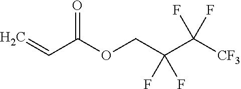 [SMILES: C=CC(=O)OCC(F)(F)C(F)(F)C(F)(F)F] |
| H123 | Chloro-2-hydroxy-propyl methacrylate | 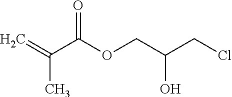 |
| H125 | Di(ethylene glycol) ethyl ether acrylate | 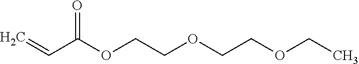 |
| H126 | Isobutyl acrylate | 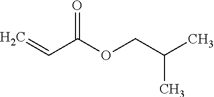 |
| H135 | Glycerol propoxylate triacrylate | 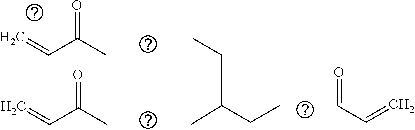 |
| H141 | Dimethylamino-ethyl methacrylate | 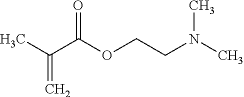 |

M2(biased) monomers

| Code | Monomer name | Monomer structure |
|---|---|---|
| H3 | Octafluoro-2-hydroxy-6- (trifluoromethyl)heptyl methacrylate | 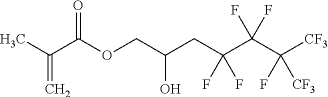 [SMILES: C=C(C)C(=O)OCC(O)CC(F)(F)C(F)(F)C(F)(C(F)(F)F)C(F)(F)F] |
| H9 | Tridecafluorooctyl acrylate | 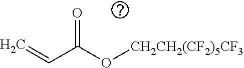 |
| H29 | N-(1,1,3,3-tetramethylbutyl)acrylamide | 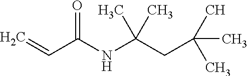 |
| H35 | Hexyl acrylate | 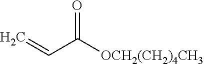 |
| H37 | Methacrylamide | 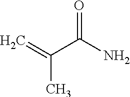 |
| H47 | N-[Tris(hydroxymethyl)methyl]acrylamide | 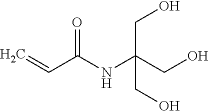 |
| H50 | Decyl methacrylate | 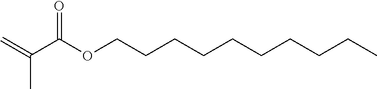 [SMILES: C=C(C)C(=O)OCCCCCCCCCC] |
| H71 | Ethylhexyl acrylate | 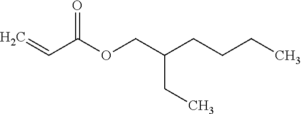 |
| H94 | 3-Hydroxy-2,2-dimethylpropyl 3-hydroxy- 2,2-dimethylpropionate diacrylate | 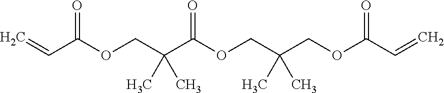 [SMILES: C=CC(=O)OCC(C)(C)COC(=O)C(C)(C)COC(=O)C=C] |
| H121 | Butoxyethyl methacrylate | 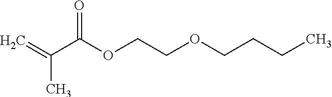 |

Pro-adherence monomers

| Code | Monomer name | Monomer structure |
|---|---|---|
| H15 | Ethylene glycol dimethacrylate | 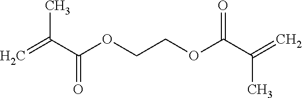 |
| H22 | Ethoxyethyl methacrylate | 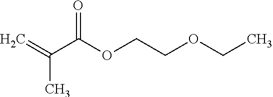 |
| H24 | t-Butylcyclohexyl methacrylate | 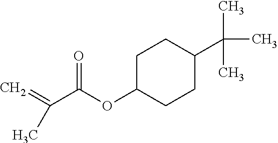 [SMILES: C=C(C)C(=O)OC1CCC(C(C)(C)C)CC1] |
| H25 | Isodecyl methacrylate | [symbol] |
| H42 | Cyclohexyl methacrylate | 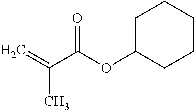 |
| H61 | Ethylhexyl methacrylate | 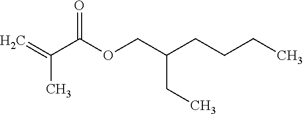 |
| H67 | Tert-butyl cyclohexylacrylate | 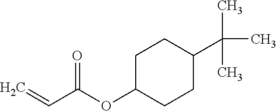 |
| H88 | Phenyl methacrylate | 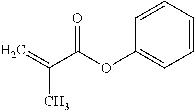 |
| H90 | Di(ethylene glycol) diacrylate | 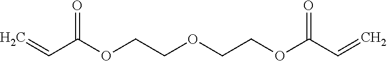 |
| H133 | Norbornyl methacrylate | 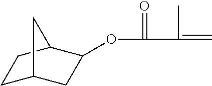 |

[symbol]

indicates data missing or illegible when filed

TABLE 7
layout of the second-generation polymer array showing copolymers codes and their constituent
monomers. There are 18 spots on each array with no polymers printed (blank)

| C1 | C2 | C3 | C4 | C5 | C6 | C7 | C8 | C9 | C10 | C11 | C12 |
|---|---|---|---|---|---|---|---|---|---|---|---|
| 66% | 66% | 66% | 66% | 66% | 66% | 66% | 66% | 66% | 66% | 66% | 66% |
| (H88) | (H9) | (H90) | (H35) | (H47) | (H42) | (H50) | (H24) | (H88) | (H9) | (H90) | (H35) |
| 33% | 33% | 33% | 33% | 33% | 33% | 33% | 33% | 33% | 33% | 33% | 33% |
| (H88) | (H47) | (H25) | (H37) | (H9) | (H15) | (H94) | (H90) | (H67) | (H29) | (H24) | (H35) |
| C24 | C25 | C26 | C27 | C28 | C29 | C30 | C31 | C32 | C33 | C34 | C35 |
| 66% | 66% | 66% | 66% | 66% | 66% | 66% | 66% | 66% | 66% | 66% | 66% |
| (H25) | (H94) | (H67) | (H3) | (H37) | (H15) | (H29) | (H61) | (H25) | (H94) | (H67) | (H3) |
| 33% | 33% | 33% | 33% | 33% | 33% | 33% | 33% | 33% | 33% | 33% | 33% |
| (H88) | (H47) | (H25) | (H37) | (H9) | (H15) | (H94) | (H90) | (H67) | (H29) | (H24) | (H35) |
| C47 | C48 | C49 | C50 | C51 | C52 | C53 | C54 | C55 | C56 | C57 | C58 |
| 66% | 66% | 66% | 66% | 66% | 66% | 66% | 66% | 66% | 66% | 66% | 66% |
| (H42) | (H50) | (H24) | (H88) | (H9) | (H90) | (H35) | (H47) | (H42) | (H50) | (H24) | (H88) |
| 33% | 33% | 33% | 33% | 33% | 33% | 33% | 33% | 33% | 33% | 33% | 33% |
| (H88) | (H47) | (H25) | (H42) | (H9) | (H15) | (H94) | (H50) | (H67) | (H29) | (H24) | (H61) |
| C70 | C71 | C72 | C73 | C74 | C75 | C76 | C77 | C78 | C79 | C80 | C81 |
| 66% | 66% | 66% | 66% | 66% | 66% | 66% | 66% | 66% | 66% | 66% | 66% |
| (H15) | (H29) | (H61) | (H25) | (H94) | (H67) | (H3) | (H37) | (H15) | (H29) | (H61) | (H25) |
| 33% | 33% | 33% | 33% | 33% | 33% | 33% | 33% | 33% | 33% | 33% | 33% |
| (H88) | (H47) | (H25) | (H42) | (H9) | (H15) | (H94) | (H50) | (H67) | (H29) | (H24) | (H61) |
| C93 | C94 | C95 | C96 | C97 | C98 | C99 | C100 | C101 | C102 | C103 | C104 |
| 66% | 66% | 66% | 66% | 66% | 66% | 66% | 66% | 66% | 66% | 66% | 66% |
| (H90) | (H35) | (H47) | (H42) | (H50) | (H24) | (H88) | (H9) | (H90) | (H35) | (H47) | (H42) |
| 33% | 33% | 33% | 33% | 33% | 33% | 33% | 33% | 33% | 33% | 33% | 33% |
| (H88) | (H47) | (H37) | (H42) | (H9) | (H15) | (H90) | (H50) | (H67) | (H29) | (H35) | (H61) |
| C116 | C117 | C118 | C119 | C120 | C121 | C122 | C123 | C124 | C125 | C126 | C127 |
| 66% | 66% | 66% | 66% | 66% | 66% | 66% | 66% | 66% | 66% | 66% | 66% |
| (H88) | (H9) | (H90) | (H35) | (H47) | (H42) | (H50) | (H24) | (H88) | (H9) | (H90) | (H35) |
| 33% | 33% | 33% | 33% | 33% | 33% | 33% | 33% | 33% | 33% | 33% | 33% |
| (H126) | (H126) | (H98) | (H98) | (H135) | (H117) | (H117) | (H113) | (H141) | (H141) | (H125) | (H125) |
| C139 | C140 | C141 | C142 | C143 | C144 | C145 | C146 | C147 | C148 | C149 | C150 |
| 66% | 66% | 66% | 66% | 66% | 66% | 66% | 66% | 66% | 66% | 66% | 66% |
| (H25) | (H94) | (H67) | (H3) | (H37) | (H15) | (H29) | (H61) | (H25) | (H94) | (H67) | (H3) |
| 33% | 33% | 33% | 33% | 33% | 33% | 33% | 33% | 33% | 33% | 33% | 33% |
| (H126) | (H126) | (H98) | (H98) | (H135) | (H117) | (H117) | (H113) | (H141) | (H141) | (H125) | (H125) |
| C162 | C163 | C164 | C165 | C166 | C167 | C168 | C169 | C170 | C171 | C172 | C173 |
| 66% | 66% | 66% | 66% | 66% | 66% | 66% | 66% | 66% | 66% | 66% | 66% |
| (H42) | (H50) | (H24) | (H88) | (H9) | (H90) | (H35) | (H47) | (H42) | (H50) | (H24) | (H88) |
| 33% | 33% | 33% | 33% | 33% | 33% | 33% | 33% | 33% | 33% | 33% | 33% |
| (H126) | (H126) | (H98) | (H135) | (H135) | (H117) | (H117) | (H113) | (H141) | (H141) | (H125) | (H115) |
| C185 | C186 | C187 | C188 | C189 | C190 | C191 | C192 | C193 | C194 | C195 | C196 |
| 66% | 66% | 66% | 66% | 66% | 66% | 66% | 66% | 66% | 66% | 66% | 66% |
| (H15) | (H29) | (H61) | (H25) | (H94) | (H67) | (H3) | (H37) | (H15) | (H29) | (H61) | (H25) |
| 33% | 33% | 33% | 33% | 33% | 33% | 33% | 33% | 33% | 33% | 33% | 33% |
| (H126) | (H126) | (H98) | (H135) | (H135) | (H117) | (H117) | (H113) | (H141) | (H141) | (H125) | (H115) |
| C208 | C209 | C210 | C211 | C212 | C213 | C214 | C215 | C216 | C217 | C218 | C219 |
| 66% | 66% | 66% | 66% | 66% | 66% | 66% | 66% | 66% | 66% | 66% | 66% |
| (H90) | (H35) | (H47) | (H42) | (H50) | (H24) | (H88) | (H9) | (H90) | (H35) | (H47) | (H42) |
| 33% | 33% | 33% | 33% | 33% | 33% | 33% | 33% | 33% | 33% | 33% | 33% |
| (H126) | (H126) | (H98) | (H135) | (H135) | (H117) | (H113) | (H113) | (H141) | (H141) | (H125) | (H115) |
| C231 | C232 | C233 | C234 | C235 | C236 | C237 | C238 | C239 | C240 | C241 | C242 |
| 66% | 66% | 66% | 66% | 66% | 66% | 66% | 66% | 66% | 66% | 66% | 66% |
| (H67) | (H3) | (H37) | (H15) | (H29) | (H61) | (H25) | (H94) | (H67) | (H3) | (H37) | (H15) |
| 33% | 33% | 33% | 33% | 33% | 33% | 33% | 33% | 33% | 33% | 33% | 33% |
| (H88) | (H47) | (H37) | (H42) | (H9) | (H15) | (H90) | (H50) | (H67) | (H29) | (H35) | (H61) |
| C254 | C255 | C256 | C257 | C258 | C259 | C260 | C261 | C262 | C263 | C264 | C265 |
| 66% | 66% | 66% | 66% | 66% | 66% | 66% | 66% | 66% | 66% | 66% | 66% |
| (H24) | (H88) | (H9) | (H90) | (H35) | (H47) | (H42) | (H50) | (H24) | (H88) | (H9) | (H90) |
| 33% | 33% | 33% | 33% | 33% | 33% | 33% | 33% | 33% | 33% | 33% | 33% |
| (H88) | (H25) | (H37) | (H42) | (H9) | (H94) | (H90) | (H50) | (H67) | (H24) | (H35) | (H61) |
| C277 | C278 | C279 | C280 | C281 | C282 | C283 | C284 | C285 | C286 | C287 | C288 |
| 66% | 66% | 66% | 66% | 66% | 66% | 66% | 66% | 66% | 66% | 66% | 66% |
| (H61) | (H25) | (H94) | (H67) | (H3) | (H37) | (H15) | (H29) | (H61) | (H25) | (H94) | (H67) |
| 33% | 33% | 33% | 33% | 33% | 33% | 33% | 33% | 33% | 33% | 33% | 33% |
| (H88) | (H25) | (H37) | (H42) | (H9) | (H94) | (H90) | (H50) | (H67) | (H24) | (H35) | (H61) |
| C300 | C301 | C302 | C303 | C304 | C305 | C306 | C307 | C308 | C309 | C310 | C311 |
| 66% | 66% | 66% | 66% | 66% | 66% | 66% | 66% | 66% | 66% | 66% | 66% |
| (H47) | (H42) | (H50) | (H24) | (H88) | (H9) | (H90) | (H35) | (H47) | (H42) | (H50) | (H24) |
| 33% | 33% | 33% | 33% | 33% | 33% | 33% | 33% | 33% | 33% | 33% | 33% |
| (H47) | (H25) | (H37) | (H42) | (H15) | (H94) | (H90) | (H50) | (H29) | (H24) | (H35) | (H61) |
| C323 | C324 | C325 | C326 | C327 | C328 | C329 | C330 | C331 | C332 | C333 | C334 |
| 66% | 66% | 66% | 66% | 66% | 66% | 66% | 66% | 66% | 66% | 66% | 66% |
| (H37) | (H15) | (H29) | (H61) | (H25) | (H94) | (H67) | (H3) | (H37) | (H15) | (H29) | (H61) |
| 33% | 33% | 33% | 33% | 33% | 33% | 33% | 33% | 33% | 33% | 33% | 33% |
| (H47) | (H25) | (H37) | (H42) | (H15) | (H94) | (H90) | (H50) | (H29) | (H24) | (H35) | (H61) |
| C346 | C347 | C348 | C349 | C350 | C351 | C352 | C353 | C354 | C355 | C356 | C357 |
| 66% | 66% | 66% | 66% | 66% | 66% | 66% | 66% | 66% | 66% | 66% | 66% |
| (H67) | (H3) | (H37) | (H15) | (H29) | (H61) | (H25) | (H94) | (H67) | (H3) | (H37) | (H15) |
| 33% | 33% | 33% | 33% | 33% | 33% | 33% | 33% | 33% | 33% | 33% | 33% |
| (H126) | (H126) | (H98) | (H135) | (H135) | (H117) | (H113) | (H113) | (H141) | (H141) | (H125) | (H115) |
| C369 | C370 | C371 | C372 | C373 | C374 | C375 | C376 | C377 | C378 | C379 | C380 |
| 66% | 66% | 66% | 66% | 66% | 66% | 66% | 66% | 66% | 66% | 66% | 66% |
| (H24) | (H88) | (H9) | (H90) | (H35) | (H47) | (H42) | (H50) | (H24) | (H88) | (H9) | (H90) |
| 33% | 33% | 33% | 33% | 33% | 33% | 33% | 33% | 33% | 33% | 33% | 33% |
| (H126) | (H98) | (H98) | (H135) | (H135) | (H117) | (H113) | (H113) | (H141) | (H125) | (H125) | (H115) |
| C392 | C393 | C394 | C395 | C396 | C397 | C398 | C399 | C400 | C401 | C402 | C403 |
| 66% | 66% | 66% | 66% | 66% | 66% | 66% | 66% | 66% | 66% | 66% | 66% |
| (H61) | (H25) | (H94) | (H67) | (H3) | (H37) | (H15) | (H29) | (H61) | (H25) | (H94) | (H67) |
| 33% | 33% | 33% | 33% | 33% | 33% | 33% | 33% | 33% | 33% | 33% | 33% |
| (H126) | (H98) | (H98) | (H135) | (H135) | (H117) | (H113) | (H113) | (H141) | (H125) | (H125) | (H115) |
| C415 | C416 | C417 | C418 | C419 | C420 | C421 | C422 | C423 | C424 | C425 | C426 |
| 66% | 66% | 66% | 66% | 66% | 66% | 66% | 66% | 66% | 66% | 66% | 66% |
| (H47) | (H42) | (H50) | (H24) | (H88) | (H9) | (H90) | (H35) | (H47) | (H42) | (H50) | (H24) |
| 33% | 33% | 33% | 33% | 33% | 33% | 33% | 33% | 33% | 33% | 33% | 33% |
| (H126) | (H98) | (H98) | (H135) | (H117) | (H117) | (H113) | (H113) | (H141) | (H125) | (H125) | (H115) |
| C438 | C439 | C440 | C441 | C442 | C443 | C444 | C445 | C446 | C447 | C448 | C449 |
| 66% | 66% | 66% | 66% | 66% | 66% | 66% | 66% | 66% | 66% | 66% | 66% |
| (H37) | (H15) | (H29) | (H61) | (H25) | (H94) | (H67) | (H3) | (H37) | (H15) | (H29) | (H61) |
| 33% | 33% | 33% | 33% | 33% | 33% | 33% | 33% | 33% | 33% | 33% | 33% |
| (H126) | (H98) | (H98) | (H135) | (H117) | (H117) | (H113) | (H113) | (H141) | (H125) | (H125) | (H115) |

| C13 | C14 | C15 | C16 | C17 | C18 | C19 | C20 | C21 | C22 | C23 |
|---|---|---|---|---|---|---|---|---|---|---|
| 66% | 66% | 66% | 66% | 66% | 66% | 66% | 66% | 66% | 66% | 66% |
| (H47) | (H133) | (H90) | (H22) | (H121) | (H9) | (H71) | (H35) | (H88) | (H133) | (H121) |
| 33% | 33% | 33% | 33% | 33% | 33% | 33% | 33% | 33% | 33% | 33% |
| (H3) | (H42) | (H133) | (H24) | (H47) | (H121) | (H50) | (H71) | (H73) | (H73) | (H71) |
| C36 | C37 | C38 | C39 | C40 | C41 | C42 | C43 | C44 | C45 | C46 |
| 66% | 66% | 66% | 66% | 66% | 66% | 66% | 66% | 66% | 66% | blank |
| (H37) | (H133) | (H67) | (H22) | (H121) | (H94) | (H71) | (H3) | (H25) | (H121) | |
| 33% | 33% | 33% | 33% | 33% | 33% | 33% | 33% | 33% | 33% | |
| (H3) | (H15) | (H133) | (H61) | (H37) | (H121) | (H29) | (H71) | (H73) | (H73) | |
| C59 | C60 | C61 | C62 | C63 | C64 | C65 | C66 | C67 | C68 | C69 |
| 66% | 66% | 66% | 66% | 66% | 66% | 66% | 66% | 66% | 100% | blank |
| (H9) | (H133) | (H24) | (H88) | (H121) | (H50) | (H71) | (H88) | (H42) | (H126) | |
| 33% | 33% | 33% | 33% | 33% | 33% | 33% | 33% | 33% | | |
| (H3) | (H90) | (H133) | (H22) | (H9) | (H121) | (H35) | (H123) | (H73) | | |
| C82 | C83 | C84 | C85 | C86 | C87 | C88 | C89 | C90 | C91 | C92 |
| 66% | 66% | 66% | 66% | 66% | 66% | 66% | 66% | 66% | 100% | blank |
| (H94) | (H133) | (H61) | (H25) | (H121) | (H29) | (H71) | (H25) | (H15) | (H135) | |
| 33% | 33% | 33% | 33% | 33% | 33% | 33% | 33% | 33% | | |
| (H3) | (H67) | (H133) | (H22) | (H94) | (H121) | (H3) | (H123) | (H73) | | |
| C105 | C106 | C107 | C108 | C109 | C110 | C111 | C112 | C113 | C114 | C115 |
| 66% | 66% | 66% | 66% | 66% | 66% | 66% | 66% | 66% | 100% | blank |
| (H50) | (H133) | (H22) | (H42) | (H121) | (H35) | (H47) | (H42) | (H90) | (H113) | |
| 33% | 33% | 33% | 33% | 33% | 33% | 33% | 33% | 33% | | |
| (H3) | (H24) | (H88) | (H22) | (H50) | (H121) | (H71) | (H123) | (H73) | | |
| C128 | C129 | C130 | C131 | C132 | C133 | C134 | C135 | C136 | C137 | C138 |
| 66% | 66% | 66% | 66% | 66% | 66% | 66% | 66% | 66% | 66% | 66% |
| (H47) | (H133) | (H113) | (H22) | (H121) | (H135) | (H71) | (H125) | (H47) | (H22) | (H71) |
| 33% | 33% | 33% | 33% | 33% | 33% | 33% | 33% | 33% | 33% | 33% |
| (H115) | (H135) | (H133) | (H125) | (H126) | (H121) | (H113) | (H71) | (H73) | (H73) | (H121) |
| C151 | C152 | C153 | C154 | C155 | C156 | C157 | C158 | C159 | C160 | C161 |
| 66% | 66% | 66% | 66% | 66% | 66% | 66% | 66% | 66% | 66% | blank |
| (H37) | (H133) | (H141) | (H22) | (H121) | (H117) | (H71) | (H115) | (H37) | (H71) | |
| 33% | 33% | 33% | 33% | 33% | 33% | 33% | 33% | 33% | 33% | |
| (H115) | (H117) | (H133) | (H115) | (H98) | (H121) | (H141) | (H71) | (H73) | (H73) | |
| C174 | C175 | C176 | C177 | C178 | C179 | C180 | C181 | C182 | C183 | C184 |
| 66% | 66% | 66% | 66% | 66% | 66% | 66% | 66% | 66% | 100% | blank |
| (H9) | (H133) | (H125) | (H126) | (H121) | (H113) | (H71) | (H47) | (H9) | (H98) | |
| 33% | 33% | 33% | 33% | 33% | 33% | 33% | 33% | 33% | | |
| (H115) | (H113) | (H133) | (H22) | (H135) | (H121) | (H125) | (H123) | (H73) | | |
| C197 | C198 | C199 | C200 | C201 | C202 | C203 | C204 | C205 | C206 | C207 |
| 66% | 66% | 66% | 66% | 66% | 66% | 66% | 66% | 66% | 100% | blank |
| (H94) | (H133) | (H115) | (H98) | (H121) | (H141) | (H71) | (H37) | (H94) | (H117) | |
| 33% | 33% | 33% | 33% | 33% | 33% | 33% | 33% | 33% | | |
| (H115) | (H141) | (H133) | (H22) | (H117) | (H121) | (H115) | (H123) | (H73) | | |
| C220 | C221 | C222 | C223 | C224 | C225 | C226 | C227 | C228 | C229 | C230 |
| 66% | 66% | 66% | 66% | 66% | 66% | 66% | 66% | 66% | 100% | blank |
| (H50) | (H133) | (H22) | (H135) | (H121) | (H125) | (H126) | (H9) | (H50) | (H141) | |
| 33% | 33% | 33% | 33% | 33% | 33% | 33% | 33% | 33% | | |
| (H115) | (H125) | (H126) | (H22) | (H113) | (H121) | (H71) | (H123) | (H73) | | |
| C243 | C244 | C245 | C246 | C247 | C248 | C249 | C250 | C251 | C252 | C253 |
| 66% | 66% | 66% | 66% | 66% | 66% | 66% | 66% | 66% | 100% | blank |
| (H29) | (H133) | (H22) | (H15) | (H121) | (H3) | (H37) | (H15) | (H67) | (H125) | |
| 33% | 33% | 33% | 33% | 33% | 33% | 33% | 33% | 33% | | |
| (H3) | (H61) | (H25) | (H22) | (H29) | (H121) | (H71) | (H123) | (H73) | | |
| C266 | C267 | C268 | C269 | C270 | C271 | C272 | C273 | C274 | C275 | C276 |
| 66% | 66% | 66% | 66% | 66% | 66% | 66% | 66% | 66% | 100% | blank |
| (H35) | (H88) | (H22) | (H90) | (H121) | (H71) | (H9) | (H90) | (H24) | (H123) | |
| 33% | 33% | 33% | 33% | 33% | 33% | 33% | 33% | 33% | | |
| (H3) | (H133) | (H42) | (H22) | (H35) | (H47) | (H71) | (H123) | (H73) | | |
| C289 | C290 | C291 | C292 | C293 | C294 | C295 | C296 | C297 | C298 | C299 |
| 66% | 66% | 66% | 66% | 66% | 66% | 66% | 66% | 66% | 100% | blank |
| (H3) | (H25) | (H22) | (H67) | (H121) | (H71) | (H94) | (H67) | (H61) | (H133) | |
| 33% | 33% | 33% | 33% | 33% | 33% | 33% | 33% | 33% | | |
| (H3) | (H133) | (H15) | (H22) | (H3) | (H37) | (H71) | (H123) | (H73) | | |
| C312 | C313 | C314 | C315 | C316 | C317 | C318 | C319 | C320 | C321 | C322 |
| 66% | 66% | 66% | 66% | 66% | 66% | 66% | 66% | 66% | 66% | blank |
| (H133) | (H42) | (H22) | (H24) | (H47) | (H71) | (H50) | (H24) | (H133) | (H133) | |
| 33% | 33% | 33% | 33% | 33% | 33% | 33% | 33% | 33% | 33% | |
| (H88) | (H133) | (H90) | (H22) | (H121) | (H9) | (H71) | (H123) | (H123) | (H22) | |
| C335 | C336 | C337 | C338 | C339 | C340 | C341 | C342 | C343 | C344 | C345 |
| 66% | 66% | 66% | 66% | 66% | 66% | 66% | 66% | 66% | 100% | blank |
| (H133) | (H15) | (H22) | (H61) | (H37) | (H71) | (H29) | (H61) | (H121) | (H121) | |
| 33% | 33% | 33% | 33% | 33% | 33% | 33% | 33% | 33% | | |
| (H25) | (H133) | (H67) | (H22) | (H121) | (H94) | (H71) | (H123) | (H123) | | |
| C358 | C359 | C360 | C361 | C362 | C363 | C364 | C365 | C366 | C367 | C368 |
| 66% | 66% | 66% | 66% | 66% | 66% | 66% | 66% | 66% | 100% | blank |
| (H29) | (H133) | (H22) | (H117) | (H121) | (H115) | (H98) | (H94) | (H29) | (H115) | |
| 33% | 33% | 33% | 33% | 33% | 33% | 33% | 33% | 33% | | |
| (H115) | (H115) | (H98) | (H22) | (H141) | (H121) | (H71) | (H123) | (H73) | | |
| C381 | C382 | C383 | C384 | C385 | C386 | C387 | C388 | C389 | C390 | C391 |
| 66% | 66% | 66% | 66% | 66% | 66% | 66% | 66% | 66% | 100% | blank |
| (H35) | (H126) | (H22) | (H113) | (H121) | (H71) | (H135) | (H50) | (H35) | (H73) | |
| 33% | 33% | 33% | 33% | 33% | 33% | 33% | 33% | 33% | | |
| (H115) | (H133) | (H135) | (H22) | (H125) | (H126) | (H71) | (H123) | (H73) | | |
| C404 | C405 | C406 | C407 | C408 | C409 | C410 | C411 | C412 | C413 | C414 |
| 66% | 66% | 66% | 66% | 66% | 66% | 66% | 66% | 66% | 100% | blank |
| (H3) | (H98) | (H22) | (H141) | (H121) | (H71) | (H117) | (H29) | (H3) | (H22) | |
| 33% | 33% | 33% | 33% | 33% | 33% | 33% | 33% | 33% | | |
| (H115) | (H133) | (H117) | (H22) | (H115) | (H98) | (H71) | (H123) | (H73) | | |
| C427 | C428 | C429 | C430 | C431 | C432 | C433 | C434 | C435 | C436 | C437 |
| 66% | 66% | 66% | 66% | 66% | 66% | 66% | 66% | 66% | 66% | blank |
| (H133) | (H135) | (H22) | (H125) | (H126) | (H71) | (H113) | (H35) | (H22) | (H22) | |
| 33% | 33% | 33% | 33% | 33% | 33% | 33% | 33% | 33% | 33% | |
| (H126) | (H133) | (H113) | (H22) | (H121) | (H135) | (H71) | (H123) | (H123) | (H133) | |
| C450 | C451 | C452 | C453 | C454 | C455 | C456 | C457 | C458 | C459 | C460 |
| 66% | 66% | 66% | 66% | 66% | 66% | 66% | 66% | 66% | 100% | blank |
| (H133) | (H117) | (H22) | (H115) | (H98) | (H71) | (H141) | (H3) | (H71) | (H71) | |
| 33% | 33% | 33% | 33% | 33% | 33% | 33% | 33% | 33% | | |
| (H98) | (H133) | (H141) | (H22) | (H121) | (H117) | (H71) | (H123) | (H123) | | |

TABLE 8
Numerical polymer microarray data format

| | Donor 1 | | | | | | | | | | Donor 2 | | | | | | | | | |
|---|---|---|---|---|---|---|---|---|---|---|---|---|---|---|---|---|---|---|---|---|
| | | | | | | | | | | | | | | | | | | Repeat | | Don |
| Polymer | Repeat 1 | | | Repeat 2 | | | | Repeat n | | | Repeat 1 | | | Repeat 2 | | | | n | | or k |
| ID | tin | MR | Cells | tin | MR | Total | . . . | tin | MR | Cells | tin | MR | Cells | tin | MR | Cells | . . . | . . . | . . . | . . . |
| 1 | 15 | 7 | 15 | 2 | 6 | 8 | 0 | 0 | 0 | 15 | 7 | 15 | 21 | 60 | 6 | 67 | | . . . | | . . . |
| 2 | 12 | 6 | 18 | 2 | 1 | 8 | 0 | 2 | 3 | 12 | 6 | 18 | 2 | 11 | 18 | 29 | | . . . | | . . . |
| . . . | . . . | | | | | | | | | | | | | | | | | | | . . . |
| m | 11 | 8 | 19 | 1 | 2 | 3 | 4 | 16 | 20 | 11 | 7 | 18 | 13 | 28 | 41 | 69 | | . . . | | |

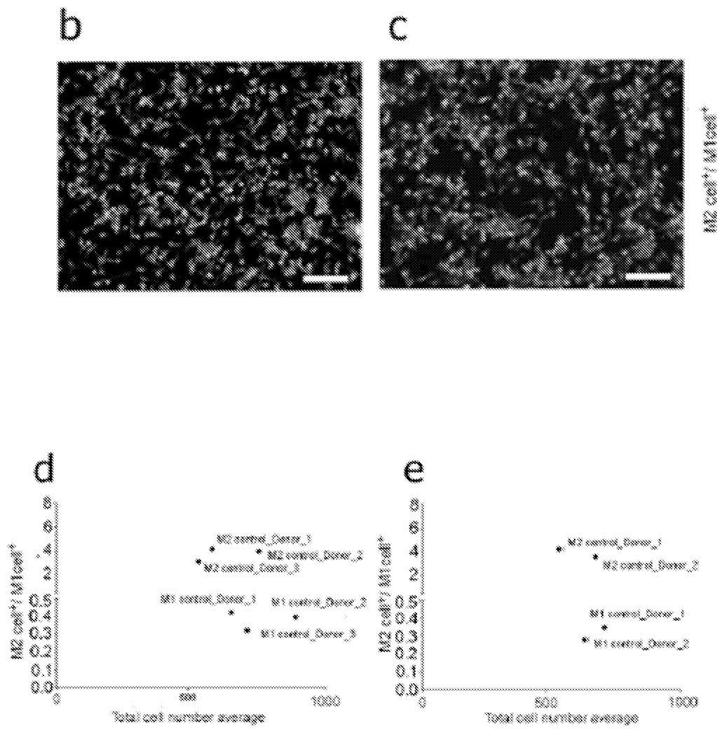

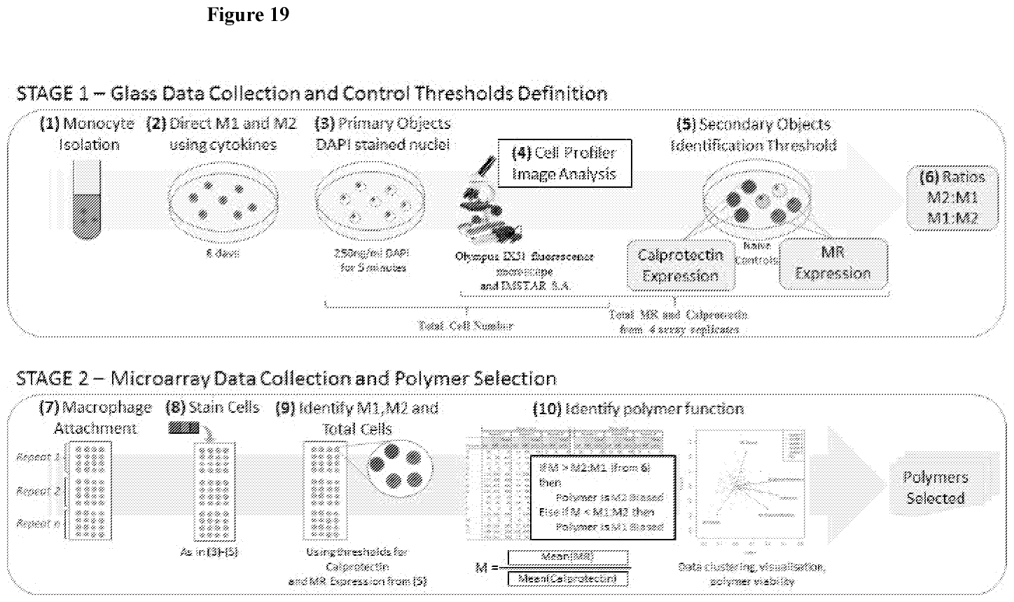

For FIG. 19. The first stage of the data collection occurs on glass. The purpose is to obtain the fluorescence threshold values for Calprotectin and MR expression (step 5 in FIG. 19) as well as M2:M1 and M1:M2 ratios for the experiments using polymers (step 6). Calprotectin and MR thresholds allow for the automatic image identification of M1 and M2 macrophage types (macrophage polarisation), respectively. To obtain these values, six main steps need to be followed (top part of FIG. 19), as described in the Methods section of the manuscript. After the macrophages are isolated (step 1), directed by cytokines (step 2) and stained (steps 3 and 5), the average fluorescence values for M1 biased and M2 biased macrophages is determined. These values are obtained with image analysis (step 4). Fluorescence images of a minimum of 100 cells in 9 fields of view are therefore selected, considering each cytokine polarisation in two different experiments for the same biological donor prepared on the same day. The maximum calprotectin fluorescent pixel intensity for each cell is selected to represent the cell florescence with regards to M1 (FIG. 18a). Similarly, the maximum MR fluorescent pixel is selected to represent M2 florescence. The average value for all cells (pixels with maximum fluorescence) is calculated for calprotectin and MR to establish the M1 and M2 expression thresholds (obtained in step 5). These thresholds are subsequently employed on stage 2 (bottom part of FIG. 19) to identify cell polarisation on images taken from polymer microarrays.

Arrays are imaged using an Olympus IX51 fluorescence microscope and a Smart Imaging System (IMSTAR S.A.). The software used for the image analysis is cell profiler. The program is configured to identify the DAPI stained nucleus (for total cell count) as well as macrophages with either M1 or M2 expression.

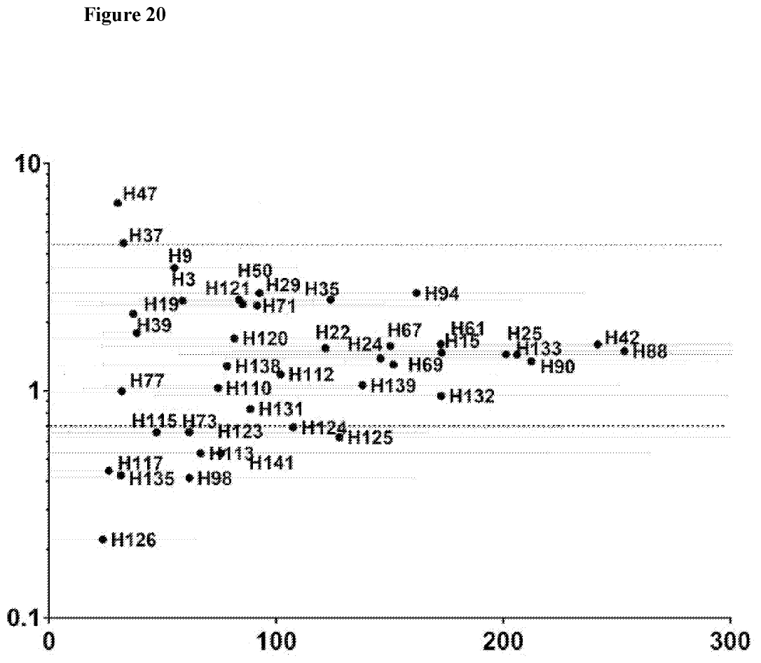

After M1 and M2 biased macrophages are quantified, the next step is to determine the ratios M1:M2 and M2:M1 cells for cytokine directed macrophages (step 6 in FIG. 19). M1:M2 and M2:M1 thresholds in glass determine the lower bound values to establish cell polarisation. For instance, with regards to the homo-polymer study, FIG. 20 shows that the thresholds established considering M2/M1 are 0.3 and 4.0. This means that polymers with polarisation values above 4.0 are considered M2 biased, while those with values below 0.3 are M1 biased. These thresholds are subsequently employed on the determination of macrophages polarisation within the polymer microarrays on the second stage (step 10).

After the thresholds are established, a similar process is repeated for the polymer microarray (steps 7, 8 and 9). The objective is to identify the polymers' properties with regards to macrophage polarisation (M1 biased, M2 biased) as well as cell attachment.

For the second stage of the data collection, monocytes from k different donors are cultured on microarrays composed of n repeats of m polymers (step 7). After 6 days of cell culture the arrays are washed and stained using the calprotectin and MR (M1 and M2 markers) (step 8).

Using the MR and calprotectin fluorescent intensities from cytokine polarised cells as a classification threshold for each cell (step 5), the number of individual M2 cells and M1 cells on each polymer spot is quantified (step 9). For each polymer the average M2/M1 ratio (from n spots) using cells from n different donors is calculated to identify polymers with the ability to induce M1 or M2 differentiation. The numerical dataset produced at this step has the format introduced in Supplementary Table 4. The first column contains the polymer unique identifier, followed by the donors' microarray data. For each donor, within each experiment repeat, the cell counts for M1 (calprotectin), M2 (MR) and Total Cells (M1+M2+M0) are stored.

Step 10 in FIG. 19 regards the analysis of the data from Supplementary Table 4 objective is to identify those polymers with the desirable cell attachment and polarisation properties. For the homo-polymer's data, the average across donors for calprotectin, MR and Total Cells is calculated as shown in equations 1, 2 and 3, respectively:

$$Average\_Calprotectin = \frac{{\sum }_{i = 1}^{k} {\sum }_{j = 1}^{n} {Calprotectin}_{{donor}_{i}}^{{repeat}_{j}}}{k} \qquad ( 1 )$$

$$Average\_MR = \frac{{\sum }_{i = 1}^{k} {\sum }_{j = 1}^{n} {MR}_{{donor}_{i}}^{{repeat}_{j}}}{k} \qquad ( 2 )$$

$$Average\_Total \_Cells = \frac{{\sum }_{i = 1}^{k} {\sum }_{j = 1}^{n} {Total\_Cells}_{{donor}_{i}}^{{repeat}_{j}}}{k} \qquad ( 3 )$$

The values for M2/M1 (M2 bias metric) and M1/M2 (M1 bias metric) are calculated as shown in equations 4 and 5, respectively:

$$\frac{M 2}{M 1} = \frac{Average\_MR}{Average\_Calprotectin} \qquad ( 4 )$$

$$\frac{M 1}{M 2} = \frac{Average\_Calprotectin}{Average\_MR} \qquad ( 5 )$$

The selection of the homo-polymers is based on the top 10 performers (highest numerical values) regarding M1 polarisation, M2 polarisation, overall attachment and viability. In addition, a consensus clustering approach is conducted to elucidate the set of core groups within the homo-polymers based on their function, in order to further validate the selection process. The variables clustered are M2/M1 (M2 biased), M1/M2 (M1 biased), total cell number (adherence), M2/M1×total cell number (M2 biased adherence), M1/M2×total cell number (M1 biased adherence). Six core clusters were identified, as shown in FIG. 21. In the figure, each point locates a polymer and its function within a two-dimensional representation using two main principal components. The cluster represented by the orange data points comprises the low attachment polymers. The grey points are polymers mostly M1 biased; cyan data points are M1 biased with higher adherence. The green points have medium adherence, and some are M2 biased; red points indicate M2 biased polymers with high adherence. Purple data points are high adherence. The polymers represented by a star in the graph are those chosen for the second generation (Table 7).

A similar analysis has been conducted for the co-polymers. In this second data set, the ratios of M2:M1 for cytokine-directed cells after stage 2 is applied are 0.8 for the lower bound and 2.3 for the upper bound. The average across donors for calprotectin, MR and Total Cells is calculated, as shown in equations 6, 7 and 8, respectively:

$$Average\_Calprotectin = \frac{{\sum }_{i = 1}^{k} ( \frac{{\sum }_{j = 1}^{n} {Calprotectin}_{{donor}_{i}}^{{repeat}_{j}}}{n} )}{k} \qquad ( 6 )$$

$$Average\_MR = \frac{{\sum }_{i = 1}^{k} ( \frac{{\sum }_{j = 1}^{n} {Calprotectin}_{{donor}_{i}}^{{repeat}_{j}}}{n} )}{k} \qquad ( 7 )$$

$$Average\_Total \_Cells = \frac{{\sum }_{i = 1}^{k} ( \frac{{\sum }_{j = 1}^{n} {Total\_Cells}_{{donor}_{i}}^{{repeat}_{j}}}{n} )}{k} \qquad ( 8 )$$

Similarly, the standard deviation for Calprotectin, MR and Total cell is calculated. The signal to noise ratio (SNR) (SNR=average/standard deviation) is determined, and the co-polymers selected are those with SNR value above two. Supplementary Table 5 shows a summary of parameters and values employed in the two generations of polymers.

TABLE 9
Parameters used for the polymer generations

| | First Generation | Second Generation |
|---|---|---|
| | (Homo-Polymers) | (Co-Polymers) |
| Number of Polymers | 141 | 460 |
| Investigated | | |
| M2: M1 Lower | 0.3 | 0.8 |
| Threshold | | |
| M2: M1 Upper | 4 | 2.3 |
| Threshold | | |
| Number of Donors | 3 | 2 |
| Number of Repeats | Donor 1: 4 repeats | 3 |
| per Donor | Donor 2: 3 repeats | |
| | Donor 3: 2 repeats | |
| Average (M2/M1) | Average | Average |
| | (All Donors) | (Average(Donor_i)) |
| Calculation | | i: 1 . . . k |
| Number of polymers | 30 | 5 |
| selected for next | | |
| stage | | |
| Number of polymers | 1 | 5 |
| selected for in vivo | | |
| experiments | | |

### REFERENCES

1. Anderson J M, Rodriguez A, Chang D T. Foreign body reaction to biomaterials. Seminars in immunology 20, 86-100 (2008).
2. Grainger D W. All charged up about implanted biomaterials. Nature biotechnology 31, 507-509 (2013).
3. Vegas A J, et al. Combinatorial hydrogel library enables identification of materials that mitigate the foreign body response in primates. Nature biotechnology 34, 345-352 (2016).
4. Higgins D M, Basaraba R J, Hohnbaum A C, Lee E J, Grainger D W, Gonzalez-Juarrero M. Localized Immunosuppressive Environment in the Foreign Body Response to Implanted Biomaterials. American Journal of Pathology 175, 161-170 (2009).
5. Veiseh O, et al. Size- and shape-dependent foreign body immune response to materials implanted in rodents and non-human primates. Nature materials 14, 643-651 (2015).
6. Biswas S K, Mantovani A. Macrophage plasticity and interaction with lymphocyte subsets: cancer as a paradigm. Nat Immunol 11, 889-896 (2010).
7. Price J V, Vance R E. The Macrophage Paradox. Immunity 41, 685-693 (2014).
8. Xue J, et al. Transcriptome-Based Network Analysis Reveals a Spectrum Model of Human Macrophage Activation. Immunity 40, 274-288 (2014).
9. Visan I. Macrophage core program. Nat Immunol 17, 1141-1141 (2016).
10. Vishwakarma A, et al. Engineering Immunomodulatory Biomaterials To Tune the Inflammatory Response. Trends Biotechnol 34, 470-482 (2016).
11. Rostam H M, Singh S, Vrana N E, Alexander M R, Ghaemmaghami A M. Impact of surface chemistry and topography on the function of antigen presenting cells. Biomaterials Science 3, 424-441 (2015).
12. Celiz A D, et al. Discovery of a novel polymer for human pluripotent stem cell expansion and multilineage differentiation. Adv Mater 27, 4006-4012 (2015).
13. Celiz A D, et al. Materials for stem cell factories of the future. Nature Materials 13, 570-579 (2014).
14. Patel A K, et al. A defined synthetic substrate for serum-free culture of human stem cell derived cardiomyocytes with improved functional maturity identified using combinatorial materials microarrays. Biomaterials 61, 257-265 (2015).
15. Venkateswaran S, et al. Bacteria repelling poly(methylmethacrylate-co-dimethylacrylamide) coatings for biomedical devices. Journal of Materials Chemistry B 2, 6723-6729 (2014).
16. Hook A L, et al. Combinatorial discovery of polymers resistant to bacterial attachment. Nature biotechnology 30, 868 (2012).
17. Murray P J, et al. Macrophage activation and polarization: nomenclature and experimental guidelines. Immunity 41, 14-20 (2014).
18. Le T, Epa V C, Burden F R, Winkler D A. Quantitative Structure-Property Relationship Modeling of Diverse Materials Properties. Chem Rev 112, 2889-2919 (2012).
19. Le T C, Penna M, Winkler D A, Yarovsky I. Quantitative design rules for protein-resistant surface coatings using machine learning. Sci Rep 9, (2019).
20. Mikulskis P, et al. Prediction of Broad-spectrum Pathogen Attachment to Coating Materials for Biomedical Devices. ACS Applied Materials & Interfaces 10, 139-149 (2018).
21. Churchwell C J, et al. The signature molecular descriptor-3. Inverse-quantitative structure-activity relationship of ICAM-1 inhibitory peptides. Journal of Molecular Graphics & Modelling 22, 263-273 (2004).
22. Breiman L. Random forests. Machine Learning 45, 5-32 (2001).
23. Cortes C, Vapnik V. Support-Vector Networks. Machine Learning 20, 273-297 (1995).
24. Rosenblatt F. Principles of Neurodynamics: Perceptrons and the Theory of Brain Mechanisms. Spartan Books (1961).
25. Rumelhart D E, Hinton G E, Williams R J. Learning Internal Representations by Error Propagation. In: Parallel distributed processing: Explorations in the microstructure of cognition (ed\hat{ }(eds David E. Rumelhart, James L. McClelland, group Pr). MIT Press (1986).
26. Rostam H M, et al. The impact of surface chemistry modification on macrophage polarisation. Immunobiology 221, 1237-1246 (2016).
27. Butterfield T A, Best T M, Merrick M A. The dual roles of neutrophils and macrophages in inflammation: a critical balance between tissue damage and repair. J Athl Train 41, 457-465 (2006).
28. Wynn T A, Vannella K M. Macrophages in Tissue Repair, Regeneration, and Fibrosis. Immunity 44, 450-462 (2016).
29. Thomas A C, Mattila J T. “Of mice and men”: arginine metabolism in macrophages. Front Immunol 5, 479-479 (2014).
30. Anderson D G, Levenberg S, Langer R. Nanoliter-scale synthesis of arrayed biomaterials and application to human embryonic stem cells. Nature biotechnology 22, 863-866 (2004).
31. Taylor M, Urquhart A J, Zelzer M, Davies M C, Alexander M R. Picoliter Water Contact Angle Measurement on Polymers. Langmuir 23, 6875-6878 (2007).
32. Hook A L, Scurr D J. ToF-SIMS analysis of a polymer microarray composed of poly(meth)acrylates with C6 derivative pendant groups. Surface and interface analysis: SIA 48, 226-236 (2016).
33. Garcia-Nieto S, et al. Laminin and fibronectin treatment leads to generation of dendritic cells with superior endocytic capacity. PloS one 5, e10123 (2010).
34. Salazar F, et al. The mannose receptor negatively modulates the Toll-like receptor 4-aryl hydrocarbon receptor-indoleamine 2,3-dioxygenase axis in dendritic cells affecting T helper cell polarization. The Journal of allergy and clinical immunology 137, 1841-1851.e1842 (2016).
35. Rostam H M, Reynolds P M, Alexander M R, Gadegaard N, Ghaemmaghami A M. Image based Machine Learning for identification of macrophage subsets. Sci Rep 7, 3521 (2017).
36. Ray S, Shard A G. Quantitative analysis of adsorbed proteins by X-ray photoelectron spectroscopy. Analytical chemistry 83, 8659-8666 (2011).

### Example 3—Fibroblast Behaviour can be Influenced by Polymers at a Surface

The polymer microarray platform described above was used for high throughput screening of polymers for fibroblast behaviour. The array has approximately 300 homo-polymers printed on it belonging to the acrylate, methacrylate and acrylamide library. Fibroblasts were assessed for cell adhesion, size, proliferation and for myofibroblast marker; alpha-smooth muscle actin. The images acquired were analysed by image analysis routines developed in FIJI. Homo-polymers were shortlisted based on A) cell attachment vs size: homo-polymers with cells greater than 20 cells and size greater than 60% relative to TCP B) A robust criteria of 3×standard deviation was applied to ensure polymers with low variation were selected. C) Homo-polymers listed from A) and B) were plotted with fold change in alpha-SMA and and proliferative index (+/−TGF-B1) to better understand the modulatory effect of polymer on the behaviour of fibroblasts.

### Human Lung Fibroblasts (MRC-5)

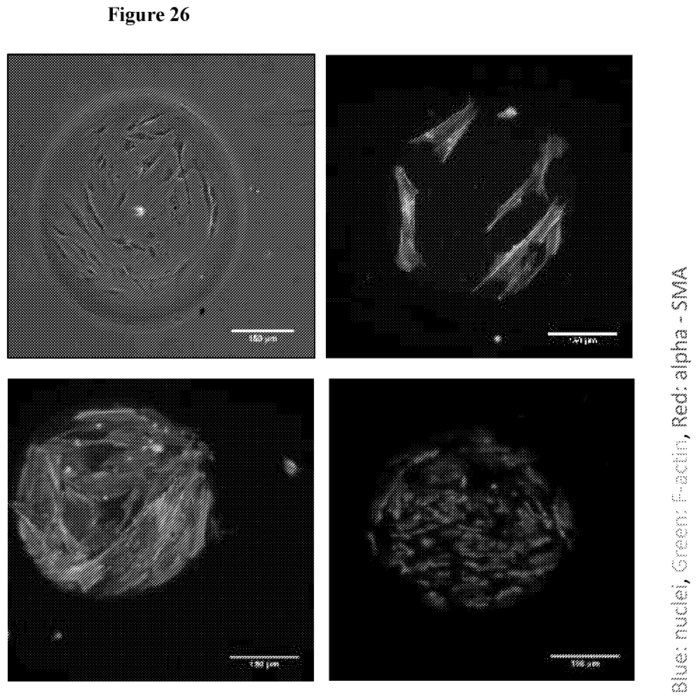

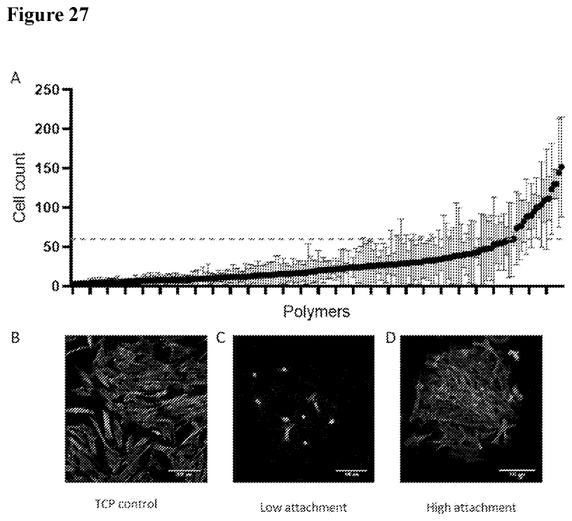

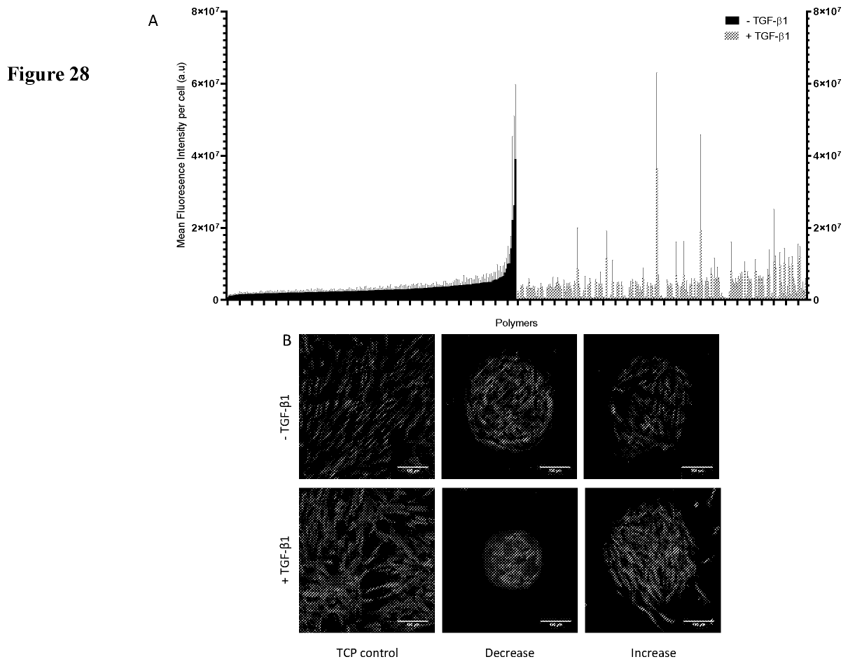

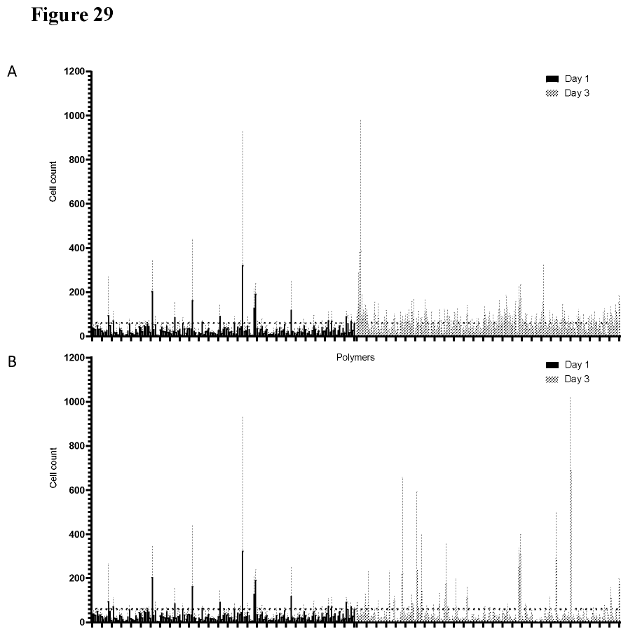

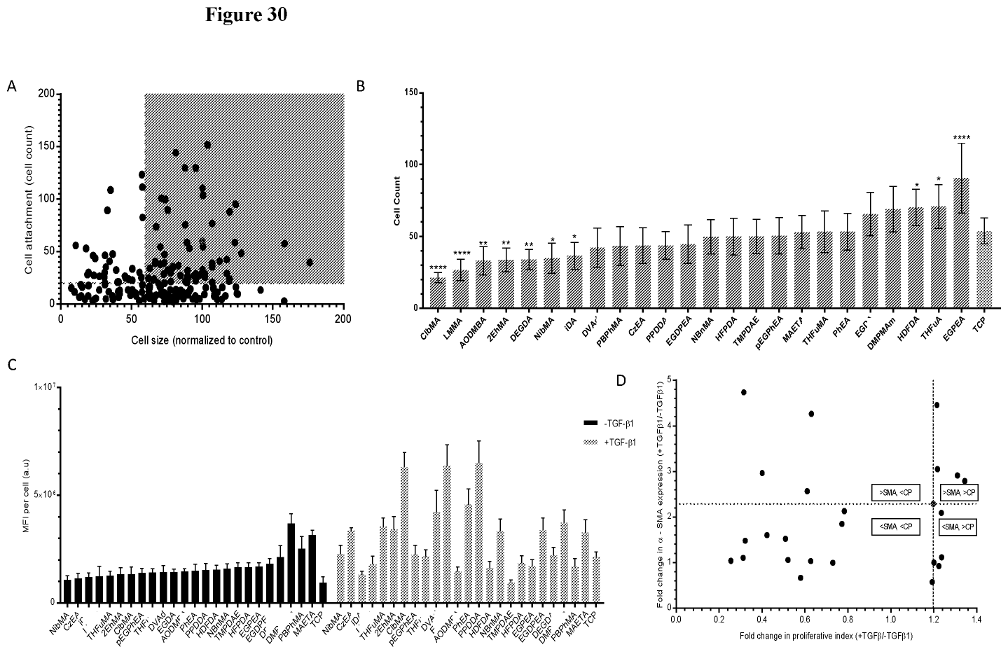

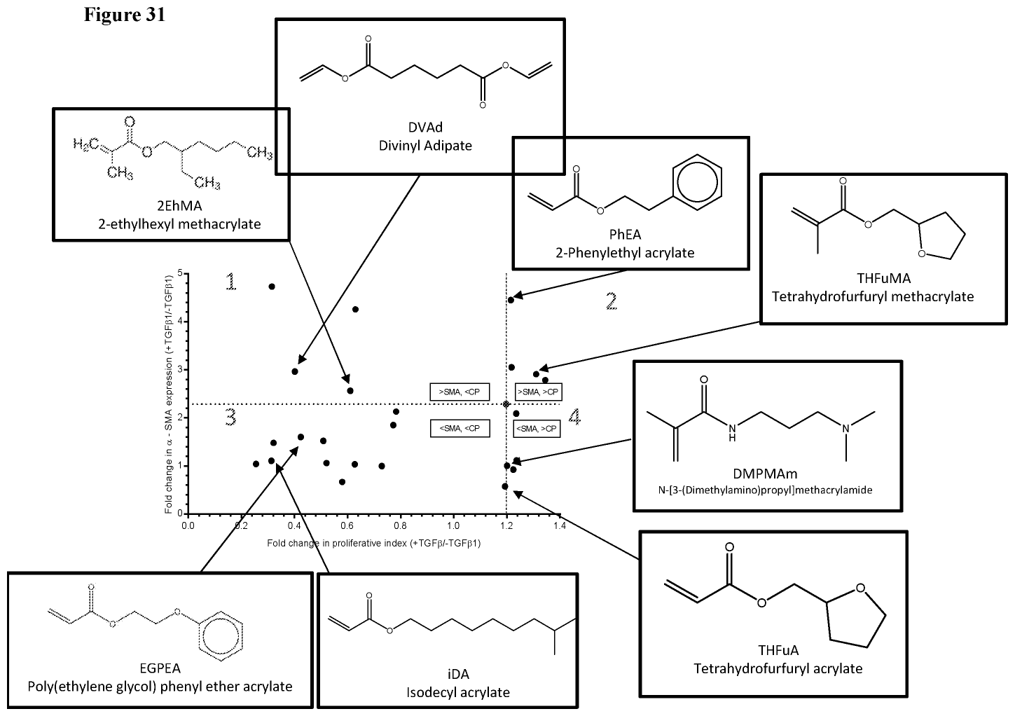

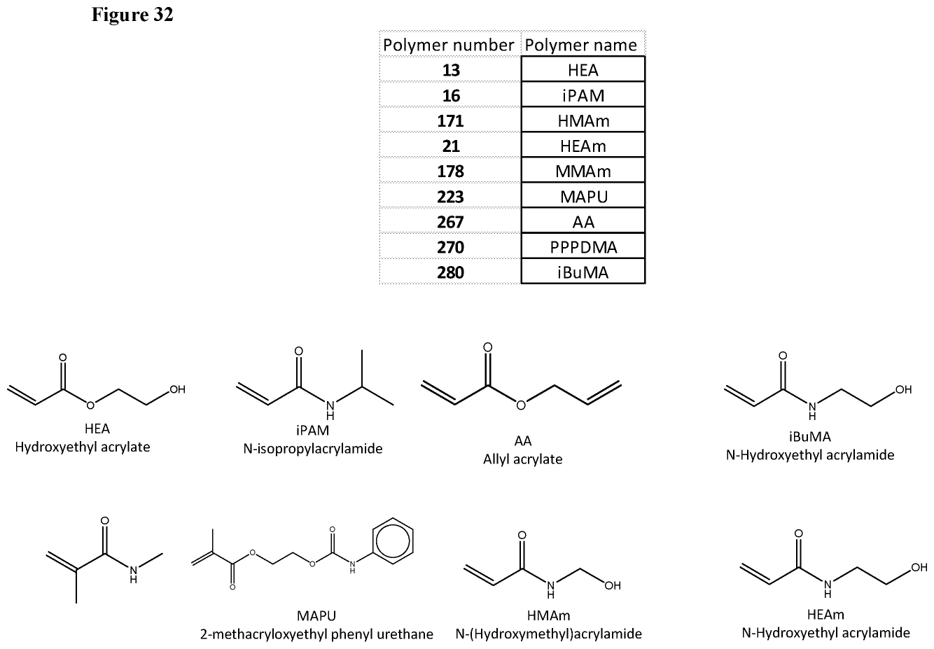

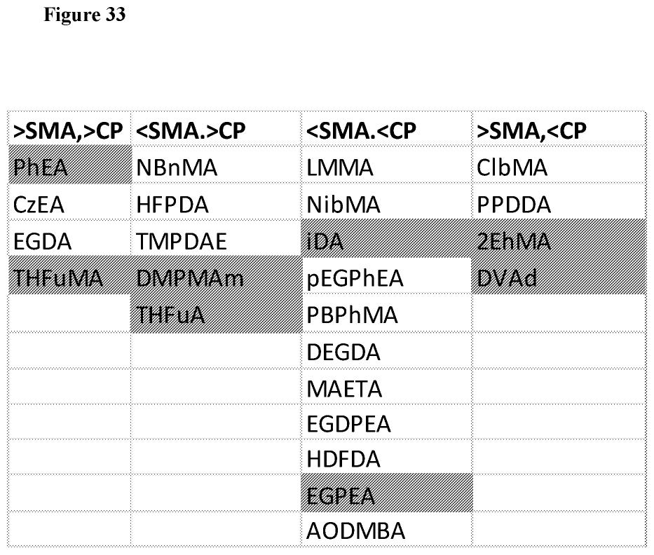

Markers—Cells were immunostained for nuclei, F-actin and alpha-smooth muscle actin
Cell attachment—Counted number of cell nuclei per cm2.
Cell size—Measured area of cell (in um2).
Cell proliferation—Cell count at 24 hours and 72 hours after cell seeding.
α smooth muscle actin—Measured relative fluorescence intensity of alpha—smooth muscle actin (normalized to cell number). See FIGS. 26-33 for details.

### Example 4 Discovery of (Meth)Acrylate Polymers that Resist Colonization by Fungi Associated with Pathogenesis and Biodeterioration

The capacity for fungi to cause disease, spoilage and biodeterioration is a major scourge for society. Fungal infections of humans are associated with high mortality rates (˜50% in hospitalized patients) killing over 1.6 million people annually, more than malaria or breast cancer (1, 2). Fungi also destroy crops and post-harvest foods sufficient to feed 600 million people annually (3, 4). This has spawned the development of sizable antifungals and fungicides industries with a combined worth $30Bn globally, even without accounting for fungicides used to tackle fungal biodeterioration of valuable products and materials. Antifungal drugs and fungicides provide our first line of defence against fungi. However, efficacy of the current arsenal of approved agents is being eroded by drug resistance (2). The issues of resistance, tightening antifungal/fungicide regulations, and mounting concerns for human and environmental health issues resulting from excessive chemical use, have combined to underscore the urgent need for alternative, sustainable strategies for fungal control. A pre-requisite for many of the problems that fungi cause is their capacity to attach to surfaces, both biological (e.g., epithelia, leaf surfaces) and inert (e.g., medical devices, household surfaces). Furthermore, in some scenarios (e.g., human infection) attached fungi can form biofilms—communities bounded by a biomaterial matrix. This property plays a crucial role in fungal virulence (5, 6). Therefore, limiting attachment of fungal cells or spores to surfaces is a primary target for combatting fungal colonization. Most strategies for tackling fungi in this context rely on antifungals and fungicides, either incorporated into or applied to surfaces. In the case of surface colonization by human pathogens, ‘lock’ therapy is designed to eradicate biofilm formation on catheters prior to their contact with patients, by pre-treating the devices with high concentrations of antifungal drug (7, 8). Medical devices can also be coated or impregnated with inhibitory agents (9). To control fungal phytopathogens in agriculture, fungicides are commonly sprayed onto crops. Fungicide resistance is a major concern here. Another chemical-based crop protection strategy is the use of actives that perturb attachment, cell-to-cell communication or dispersion, without necessarily killing the fungi. The plant-derived bioactive zosteric acid, which alters oxidative balance of cells by targeting the NADH:quinone reductase (10, 11), has been shown at sub-lethal concentrations to reduce adhesion of the phytopathogens Magnaprothe grisea and Colletotrichum lindemuthianum (12), and food-spoilage fungi Aspergillus niger and Penicillium citrinum (13). These strategies all rely on the use of bioactive agents which, as outlined earlier, hold diminishing appeal for long-term fungal control into the future. However, since fungal attachment is essentially a passive process, it is reasoned that passive approaches could hold promise for effective control of fungi at the crucial surface-attachment step. A passive intervention like an attachment-resistant material could be expected to exert less selective pressure for resistance than bioactive drugs, for example. This is because non-resistance should be less commonly fatal in the case of an anti-attachment surface, while development of resistance would typically require a gain of new function (i.e., ability to attach). Despite these advantages, such materials would be difficult to design rationally with our limited current knowledge of the mechanistic bases for fungal interactions with different surfaces.

Human pathogens like Candida albicans avidly form biofilms, including on biomedical devices. Fungal biofilms necessitate replacement of expensive indwelling devices like voice prostheses every few months (22). Moreover, a passive, anti-attachment technology for fungi could have far wider applications, considering the range of problems caused by fungi including those impacting food security and longevity of commercial materials. There is no expectation that anti-attachment materials developed against bacteria, should also be effective against fungi. Indeed, bacteria and fungi have very different cell-surface characteristics, e.g., their cell walls comprise distinct major components, in peptidoglycan and chitin respectively.

Here, a group of polymeric materials are characterised that significantly reduce the attachment and biofilm formation of key fungi onto diverse surfaces.

### Methods

### Polymer Array Synthesis

Polymer microarrays were prepared using a modified version of the previously described procedure (23). Polymer microarrays were printed using a XYZ3200 dispensing station (BioDot) using quilled steel pins (Arrayit, 946MP6B). Printing was carried out under an argon atmosphere maintaining O₂<2000 ppm, 25° C. and 30-35% relative humidity. Diluted polymerisation solutions were composed of monomer (50% v/v for oils, 50% w/v for solids) in N,N′-dimethylformamide, 1:1 N,N′-dimethylformamide:water or 1:1 N,N′-dimethylformamide:toluene depending on solubility. The photoinitiator 2,2-dimethoxy-2-phenyl acetophenone (1% w/v) was added to all solutions. A total of three replicates were printed on each slide. Monomers were purchased from Sigma, Scientific Polymers, Acros or Polysciences and were used as received. Spacing between the printed spots in each row was 1500 μm in the x axis, with an alternating +750 μm/−750 μm offset in the x axis between each row and a 750 μm spacing between each row in the y axis. After printing was completed, arrays were dried in a Heraeus Vacuum Oven (35° C., 0.3 mbar) for 7 days.

### High-Throughput Surface Characterization

Time-of-flight secondary-ion mass spectrometry (ToF-SIMS) measurements were conducted using a ToF-SIMS IV (IONTOF GmbH) instrument operated using a 25 kV Bi₃⁺ primary ion source exhibiting a pulsed target current of ˜1 pA. Samples were scanned at a pixel density of 100 pixels per mm, with 8 shots per pixel over a given area. The analysis area was 20000×20000 μm. An ion dose of 2.45×10¹¹ ions per cm² was applied to each sample area ensuring static conditions were maintained throughout. Both positive and negative secondary ion spectra were collected (mass resolution of >7000 at m/z=29). Owing to the non-conductive nature of the samples, a low energy (20 eV) electron flood gun was applied to provide charge compensation.

### Computational Modelling

The replicate fungal fluorescence values for each of the polymers screened (3 replicates for B. cinerea and 6 for C. albicans) were averaged and the standard deviations calculated. As the fluorescence values spanned a wide range, the log of the fluorescence values was used as the dependent variable in the computational models, as is common practice for quantitative structure-activity relationship modelling. Polymers with low signal to noise ratio (<1.5) were excluded from the B. cinerea (173 polymers) and C. albicans (197 polymers) attachment datasets. For modelling, least absolute shrinkage and selection operator (LASSO) was employed to select sparse subsets of features from larger pools of possibilities in a context-dependent manner.

Partial least square (PLS) regression was conducted using Matlab R2018a 9.4.0.813654. ToF-SIMS positive and negative data were concatenated into a single data matrix to be used as the X-variables for the model. X-variables were mean centred and variance scaled prior to analysis. Data were randomly split into training and test sets (3:1) in order to validate the model produced. The number of latent variables used in the model was selected based upon a minimum in the root mean square error of cross validation (RMSECV). Three latent variables were selected for models for each fungal species.

Extreme gradient boosting (XGBoost) regression machine learning, a robust nonlinear machine learning (ML) method (24), was used to generate models relating chemical features to fluorescence (and therefore attachment). The chemical features used to train the models were of two types: signature molecular descriptors (25, 26) generated by computationally fragmenting molecules, and ToF-SIMS ion peaks derived from actual molecular fragmentation by probe ions. Although independent test sets are the best way of assessing the predacity of ML models, due to the high variability and noise present in the datasets, leave-one-out (LOO) cross validation was used for this purpose.

The XGBoost algorithm (24) (version 0.22) with default parameters was used to generate the models and LOO cross validation was implemented using the package leaveOneOut from sklearn.model_selection (both codes implemented in Python 3.7). LASSO feature selection was implemented in Matlab R2017a using the lassoglm function selecting the features that provide the minimum value for the squared error for the lambda parameter. Their rank in importance is given by the XGBoost descriptor importance parameter, which provides a score indicating how useful each descriptor was in constructing the boosted decision trees within the model, using Gini as performance measure. This importance was calculated for each descriptor and averaged across the multiple trees, allowing attributes to be ranked and compared to each other.

### Free Radical Polymerisation Scale-Up for Performance Validation, Inkjet 3D Printing and Leaf Coating

Polymerisation method for biological performance validation: The synthesis of selected compounds was up scaled to allow the validation of the biological performance observed in the pin printing assays. This was achieved by coating the 6.4 mm diameter wells of 96-well plates. Plates were prepared by adding 50 μl of monomer solution into each well. Polymerization was initiated by addition of 2,2-dimethoxy-2-phenylacetophenone (Sigma) to a final concentration of 1% (w/v). Samples were irradiated with UV (Blak-Ray XX-15L UV Bench Lamp, 230V ˜50 Hz, 15 Watt, 365 nm) for 1 h with O₂<2,000 ppm. The samples were dried at <50 mTorr for 7 days. Wells were then washed briefly with isopropanol and left for 2 days at 37° C. in distilled water. Plates were then washed briefly with isopropanol and distilled water, and air dried before irradiation with UV for 20 min to sterilize the samples.

Polymerisation method for validation of inkjet 3D printing performance: Exploring the potential printability of a monomer for inkjet based 3D printing requires consideration of several key factors including viscosity, surface tension, printing conditions etc. Following existing methods for the efficient formulation development of inkjet based 3D printing inks (27-30), candidate monomers that were suitable for the inkjet 3D printing process were identified and then associated ink formulations were prepared by dissolving 1% (w/v) 2,2-dimethoxy-2-phenylacetophenone (Sigma) into 5 ml of the candidate monomer. The mixture was stirred at 800 rpm at room temperature until the initiator was fully dissolved. The ink was then purged with nitrogen gas for 15 min and filtered through a 5 μm nylon syringe filter. The final ink formulation was left at 4° C. overnight to degas. A Dimatix DMP-2830 material printer was used for printing, equipped with a 10 pl cartridge containing 16 nozzles, each with a square cross-section with a side length of 21 μm. The jetting voltage and waveform were adjusted until stable droplet formation was achieved. A 365 nm UV LED unit (800 mW/cm²) was used for in-line swath-by-swath ink curing after deposition. The whole printing process was carried out in a nitrogen environment, where the oxygen level was 0.2±0.05%.

Polymerisation method for leaf coating: For investigating the fungal infection of polymer-coated plant leaves, polymerisation of the materials identified as candidates for resistance to fungal infection was performed by free radical polymerisation using a thiol chain transfer agent (CTA) to limit the molecular weight of the final material and ensure that it was processable. Candidate monomers were dissolved in cyclohexanone (Acros Organics) (1:3, v/v) and the CTA (1-Dodecanethiol (Acros organics), 5% mol/mol with respect to the monomer) and initiator (2′-Azobis(2-methylpropionitrile) (AIBN; Sigma-Aldrich), 0.5% w/w) were added. The reaction mixture was then held at 75° C. for 24 h. Isolation of the polymer was achieved by precipitation into an excess of either; (a) heptane (Fisher Scientific; DEGEEA, DEGMA, EGMMA, TEGMA), or (b) chloroform (Fisher Scientific; mMAOES). The non-solvent to reaction mixture ratio used for the precipitations was 5:1 (v/v). Precipitated materials were collected in vials and incubated in a vacuum oven for at least 24 h before use. NMR spectroscopic analysis was performed with the crude polymerization solution to determine polymer conversion and on the final precipitate to assess purity. To evaluate the molecular weight of the materials, purified samples were dissolved in HPLC grade tetrahydrofuran (THF) for analysis by Gel Permeation Chromotography (GPC).

¹H-Nuclear Magnetic Resonance Spectroscopy (¹H NMR)

¹H NMR spectra were recorded at 25° C. using a Bruker DPX-300 spectrometer (400 MHz). Chemical shifts were recorded in 5H (ppm). Samples were dissolved in deuterated chloroform (CDCl₃), to which chemical shifts were referenced (residual chloroform at 7.26 ppm).

### Gel Permeation Chromatography (GPC)

GPC analysis was performed using an Agilent 1260 Infinity instrument, equipped with a double detector in the light scattering configuration. Two mixed columns at 25° C. were employed, using THE as the mobile phase at a flow rate of 1 ml min⁻¹. GPC samples were prepared in HPLC grade THE and filtered before injection to the GPC system. Analysis was carried out using Astra software. The molecular weight (number average, Mn) and polydispersity (D) were calculated, with reference to a calibration curve created using commercially purchased poly(methyl methacrylate) standards.

### Fungal Growth Conditions

Fungal strains used in this study were the yeast Candida albicans CAF2-yCherry (kindly provided by R. Wheeler, University of Maine, US; (31)), and the filamentous fungi Botrytis cinerea SAR109940, Zymoseptoria tritici K4418 and Aspergillus brasiliensis CBS 246.65. C. albicans was maintained and grown in YPD medium [2% peptone (Oxoid, Basingstoke, United Kingdom), 1% yeast extract (Oxoid), 2% D-glucose] (32). Where necessary, medium was solidified with 2% (w/v) agar (Sigma, UK). The filamentous fungi were routinely maintained and grown on Potato Dextrose Agar or Broth [PDA (Oxoid) or PDB (Sigma, UK)].

### Polymer Microarray Screening for Fungal Attachment

Prior to testing against fungi, the microarray slides were washed by immersion in distilled water for 10 min, air-dried and UV sterilized. For screening with C. albicans (yCherry-tagged), single colonies were used to inoculate YPD broth cultures in Erlenmeyer flasks and incubated at 37° C. with orbital shaking at 150 rev·min⁻¹. Overnight cultures were washed twice in RPMI-1640 (Sigma) and adjusted to OD₆₀₀ □10. Microarray slides were incubated statically at 37° C. for 2 h with 15 ml of the cell suspension. For tests with B. cinerea, spores were harvested from 7 day old PDA plates, washed twice in PDB medium, and resuspended in PDB at a concentration of 2×10⁷ spores ml⁻¹. As with C. albicans, microarray slides were incubated statically with 15 ml of the cell suspension, but at room temperature for 6 h and stained for 10 min with 0.5% Congo red. As controls, slides were also incubated with non-inoculated medium. After the period of attachment, the slides were removed and washed three times with 15 ml PBS at room temperature. After rinsing with distilled water to remove salts then air drying, fluorescence images from the slides were captured using either a GenePix Autoloader 4200AL (C. albicans; Molecular Devices, US) or 4000B (B. cinerea; Molecular Devices, US) Scanner, with a 635 nm red laser and red emission filter. The total fluorescence signal from each polymer spot was determined using GenePix Pro 6 software (Molecular Devices, US). The fluorescence signal attributable to fungal attachment to each polymer was determined by subtracting the fluorescence signal in the medium-only control incubation from that in the incubation with fungus. For polymers where the fluorescence was below the limit of detection, fluorescence was recorded as zero, as discussed in (15). Fungal-attachment to each polymer is expressed as a percentage relative to the median value (=100%) across all polymers for each fungus.

### Fungal Biofilm Assessment

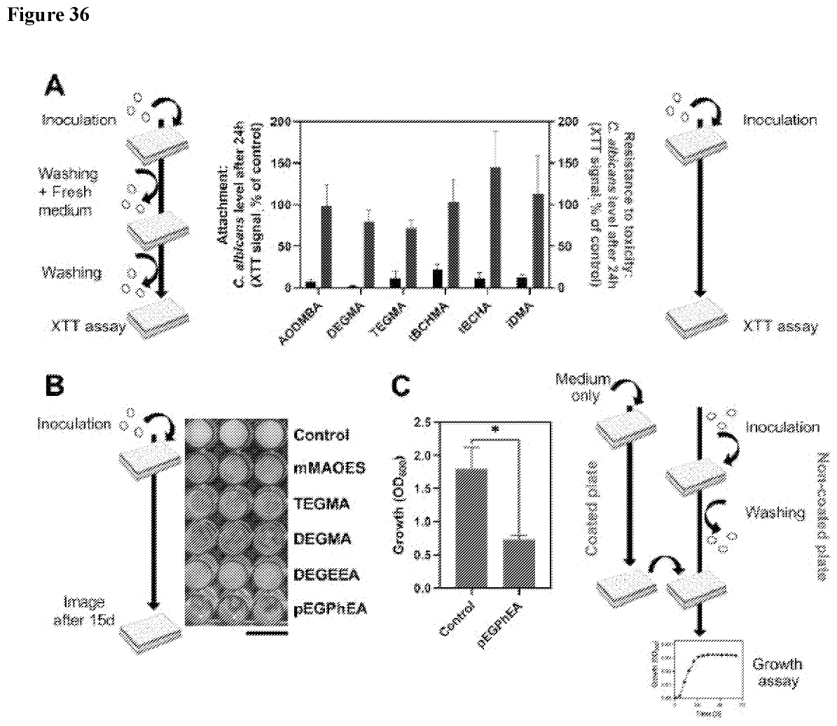

Biofilm metabolic activity was measured by the XTT (tetrazolium salt, 2,3-bis[2-methyloxy-4-nitro-5-sulfophenyl]-2H-tetrazolium-5-carboxanilide) (Sigma) reduction assay. For C. albicans, single colonies were used to inoculate YPD broth cultures in Erlenmeyer flasks and incubated overnight at 37° C. with orbital shaking at 150 rev·min⁻¹. Cultures were washed twice in RPMI-1640 and diluted to 125,000 cells ml-1. Aliquots (100 μl) of the cell suspension were transferred to 96-well microtitre plates (Greiner Bio-One; Stonehouse, UK), either coated with the polymers of interest or containing coupons 3D-printed with polymer as described above, then incubated statically for 2 h. Similarly, 100 μl of fungal spores (2.5×10⁶ spores ml⁻¹ in PDB) from 7-d old PDA plates were transferred to coated 96-well plates for 6 h at room temperature. In all cases, coupons were subsequently transferred to fresh 96-well plates. Non-adherent cells or spores were removed by three gentle washes with PBS, then 100 μl of fresh medium were added to each well and plates were incubated at 37° C. up to 24 h post inoculation. Coupons were again transferred to fresh plates. The wells were washed three times with PBS and the XTT reaction was initiated by adding XTT and menadione to RPMI (for C. albicans) to final concentrations of 210 μg ml⁻¹ and 4.0 μM respectively, or to PBS (for B. cinerea) to final concentrations of 400 μg·ml⁻¹ and 25 μM (final volume per well, 200 μl) (PBS was used instead of PDB as the XTT reaction does not work in PDB medium). After 2 h and 6 h respectively, 100 μl of the reaction solutions were transferred to fresh 96-well plates and the absorbance at 490 nm was measured using a BioTek EL800 microplate spectrophotometer. To assess the impact of the polymers on fungal growth, washing steps were omitted as presented in FIG. 36. Contrary to RPMI and as mentioned above, the XTT reaction cannot be performed in PDB medium and fungi are not able to grow in PBS. Therefore B. cinerea was cultivated for 15 days on the polymers in the presence of PDB and growth effects were assessed visually.

Biofilm formation was also assessed on prosthesis valve flaps, either printed (above) or commercial manufactures from silicone (kindly provided by Atos Medical; raw material is Silastic® Q7-4735 Dow Corning). The latter was used as the control material. The materials were immersed in the presence of 1×10⁶ cells in RPMI-1640 (1 ml final volume) in 12-well plates (Greiner Bio-One). After 2 h of static incubation at 37° C., valve flaps were transferred to new plates and washed 3 times with PBS to remove non-adherent cells. Fresh RPMI-1640 was added. After 46 h at 37° C. with orbital shaking at 100 rev·min⁻¹, RPMI-1640 was removed and biofilm stained with 0.5% (w/v) crystal violet for 1 min. The valve flaps were washed three times with PBS to remove non-adherent biofilm and excess stain, before image capture. For quantification, the crystal violet was dissolved with 1 ml ethanol and 100 μl of the reaction was transferred to 96-well plates. Absorbances at 600 nm were measured using a BioTek EL800 microplate spectrophotometer.

### Fungal Infection of Plant Leaves

Polymer solutions [20% (w/v), prepared using 20% (v/v) isopropanol as solvent] were sprayed onto 1.5 cm diameter leaf discs prepared from fresh lettuce. Discs were placed onto water agar [sterile distilled water, 2% (w/v) agar] in square Petri plates (Greiner), then incubated at room temperature for up to 3 days. To measure resilience of coated polymer to rinses with water, some lettuce-leaf discs were washed by submersion in water. Spores of B. cinerea were harvested from 7-day old PDA plates, washed twice with PDB, and adjusted in PDB to a concentration of 5×105 spores ml. Once dried, leaf discs were infected with B. cinerea by aliquoting 5 μl of spore suspension to the middle of the discs (2,500 spores per leaf disc). Images were captured every day up to 3 days post-infection to assess lesions. To assess potential toxicity of polymers to the plant material, leaf discs were sprayed with the polymers but not infected with B. cinerea.

### Results

### Identification of Candidate Fungal Anti-Attachment Polymers by Microarray Screening

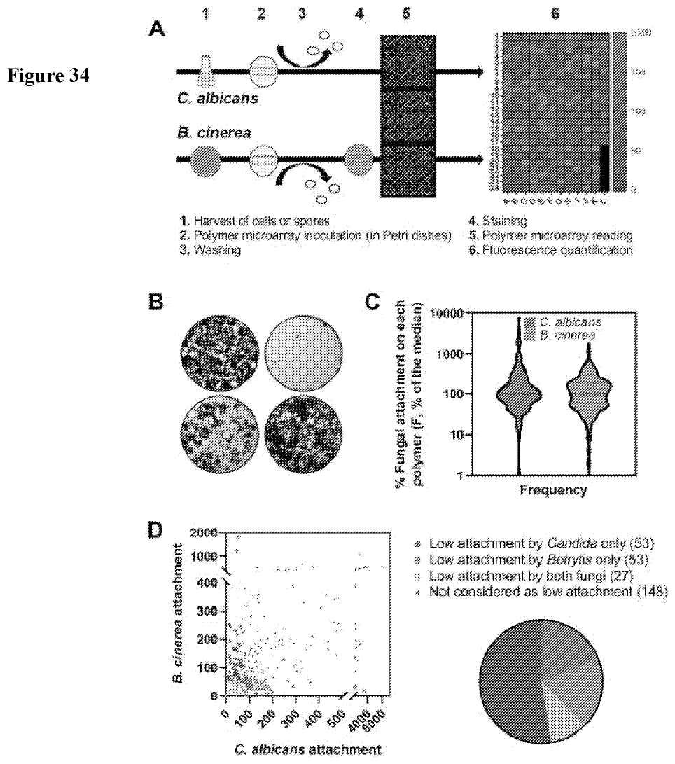

To identify materials that may resist fungal attachment, we screened 281 acrylate and methacrylate homopolymers printed in a microarray format. These encompassed bacterial anti-attachment candidates described previously (15) and other commercially available monomers which exhibited a wide chemical diversity within the groups pendant to the backbone chain. Fungal attachment was determined after incubating suspensions of cells (C. albicans, yCherry-tagged) or spores (B. cinerea, Congo red stained) with the polymer microarrays for 2 h or 6 h, respectively (FIG. 34A). These incubation times were sufficiently short to allow attachment while precluding subsequent overgrowth of hyphae and mycelium onto neighbouring polymer spots (FIG. 34B). Fluorescence signals from the labelled fungi were used to quantify relative attachment to the 281 homopolymers. The distribution of C. albicans and B. cinerea attachment levels across the array was broad: respectively, 03.9% and 1.1% of the polymers gave very high attachment (>1000% of the median attachment value), while 2.5% and 3.9% were strongly resistant to attachment (<10% of the median) (FIG. 34C). Levels of attachment of the two fungi to the same polymer surfaces varied, showing only a weak positive correlation, albeit significant (Pearson correlation, R²=0.097; p<0.0001) (FIG. 34D). Differences in the responses of the two fungi were not unexpected given that the representative forms initiating their attachment (cells versus spores) have quite different surface properties (33), and the organisms themselves are phylogenetically distant among the ascomycete fungi (34).

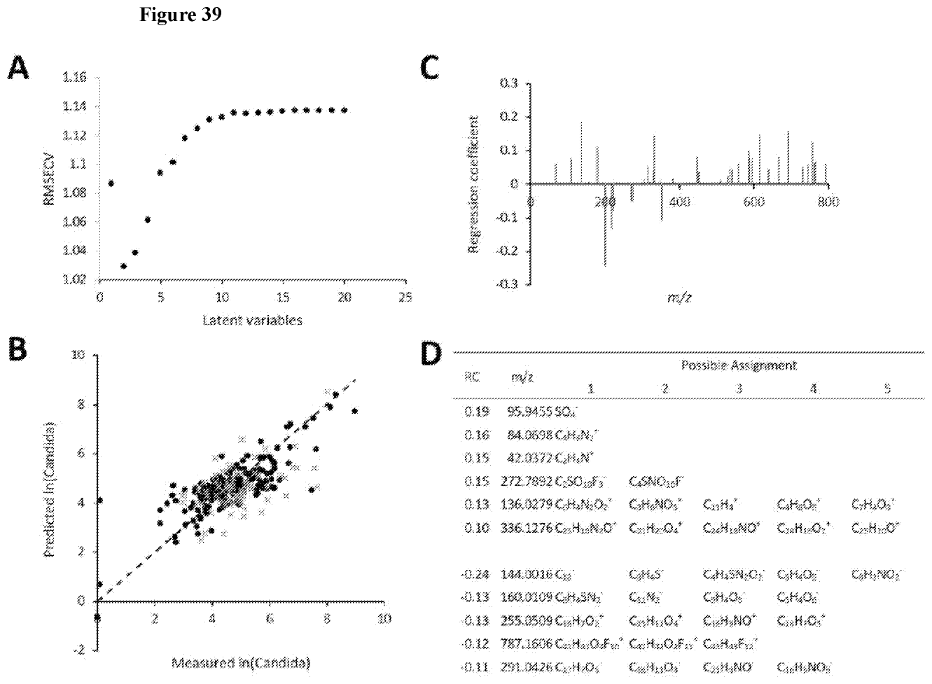

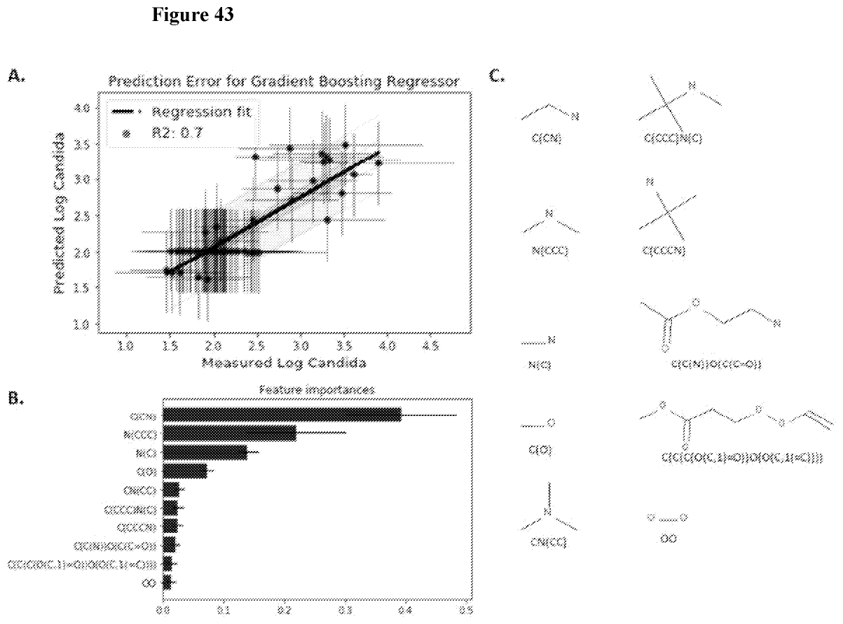

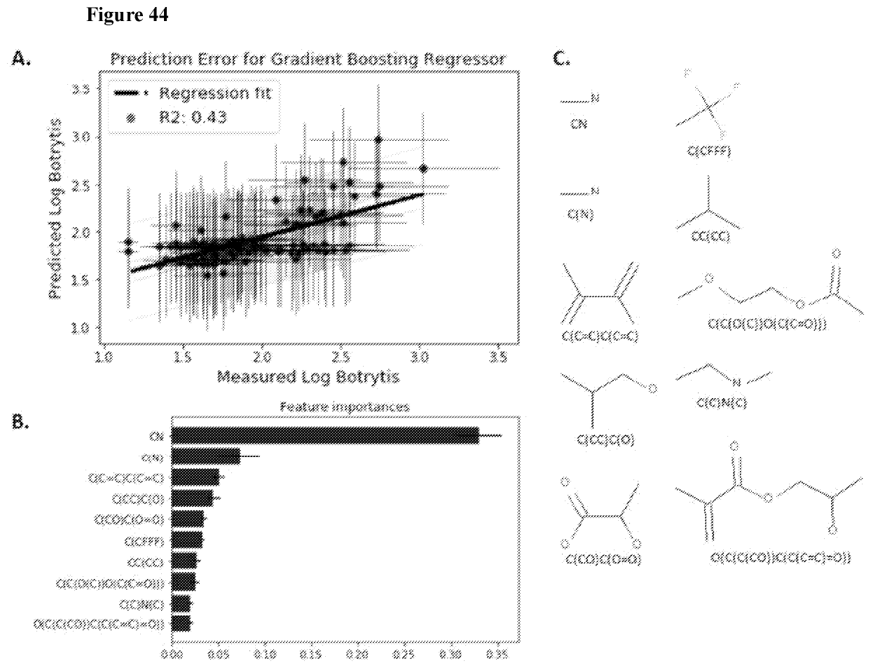

Machine learning (ML) methods were employed to generate predictive models for C. albicans and B. cinerea attachment, in order to assess the relationship between surface chemistry and the attachment of each fungus. Signature molecular descriptors and Time-of-Flight Secondary Ion Mass Spectrometry (ToF-SIMS) descriptors were generated for the polymers investigated. Prior to modelling, sparse feature selection was used to eliminate less informative descriptors. Leave-one-out cross validation was used to determine the predictive power of the fungal attachment models. For both fungal species, signature descriptor (computed molecular fragments) produced the best models. Non-linear ML models produced a small performance improvement over linear PLS regression (FIG. 39-42). The predictive performance of the XGBoost and PLS models for C. albicans and B. cinerea attachment using these descriptors is presented in FIGS. 43 and 44, respectively. There was only a moderate numerical relationship between signature molecular descriptors and observed log attachment values for B. cinerea, with R²=0.43 and root mean square error (RMSE) 0.29 log fluorescence (FIG. 44). The fragment descriptor most strongly associated with low B. cinerea attachment was the keto ether O(C(C(CO))C(C(C═C)=O)), although the weak predictive power of the model limits the importance of this observation. For C. albicans, the model was stronger, with R²=0.70 and RMSE 0.29 log fluorescence (FIG. 43). The molecular features that were most strongly associated with high and low C. albicans attachment, respectively, were methylene nitrile C(CN) and carbonyl C(O).

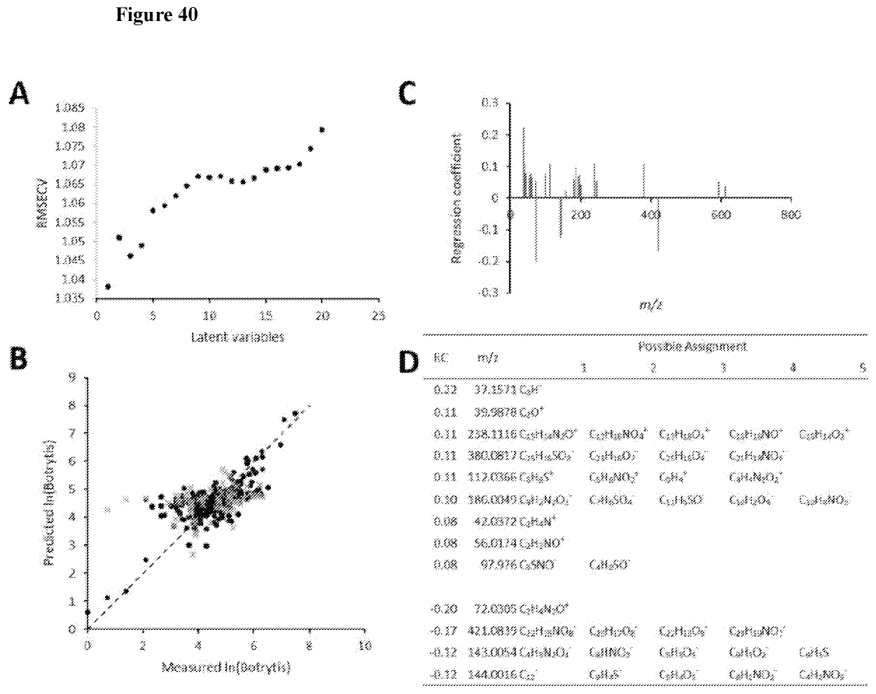

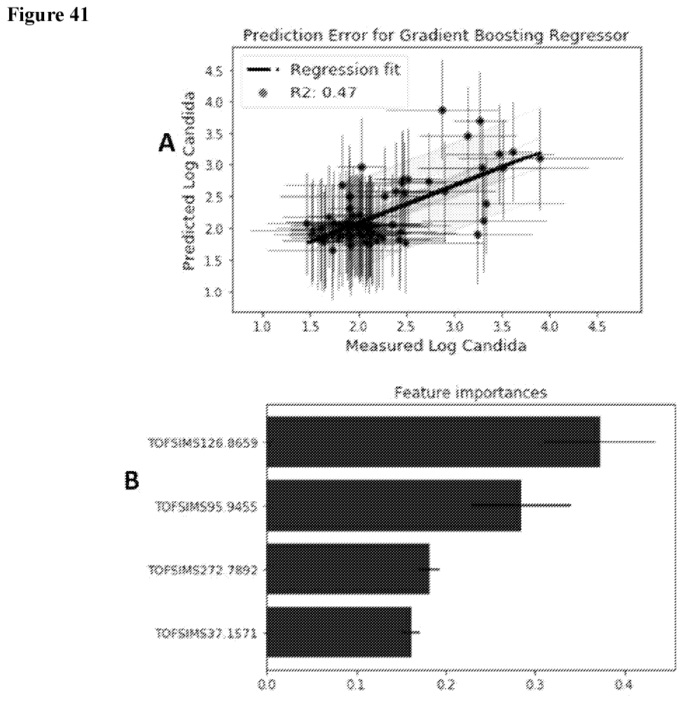

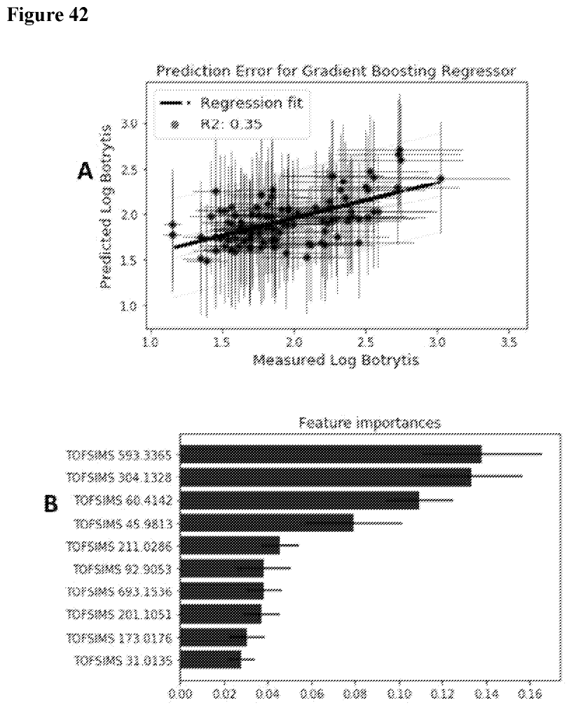

Note for FIG. 39, ¹ A weak correlation was observed by PLS models between surface chemistry as represented by ToF-SIMS spectra and the attachment of C. albicans or B. cinerea (R²=0.43 and 0.20, respectively) (FIG. 39-40). Thus, although surface chemistry contributes to the attachment of the fungi to the different polymers, other variables such as cell or spore morphology and the polymers' physical form also likely play a significant role in determining the extent of attachment. Ions likely associated with nitrate and sulphate groups were assigned positive regression coefficients for models generated for both fungal species, suggesting these molecular groups are associated with high fungal attachment, possibly through an electrostatic interaction. Conversely, aliphatic carbon and high oxygen containing fragments were assigned low regression coefficients in the PLS models for both fungal species. Using non-linear ML improved the predictive power of the models compared with PLS regression for both fungal species (FIG. 41-42). For C. albicans the R² value increased from 0.43 to 0.47 and for B. cinerea the R² value increased from 0.20 to 0.35. The small increase suggests that the relationship between fungal attachment and surface chemistry was largely linear in nature.

Due to the noise associated with fluorescence data (FIG. 41-44) and the relatively weak predictive power of the computational models, we adopted a reasonably liberal approach in selecting candidate materials from the screen for further interrogation, reasoning that autofluorescence, differences in polymer-spot geometry and/or instability evident with certain polymers during the assays introduced elements of error. Therefore, for each fungus, we selected for further study 80 polymers that gave the lowest attachment; 27 of these were common to both fungi (FIG. 34D).

Assay Scale-Up Indicates Acrylate and Methacrylate Polymers that are Most Resistant to Fungal Colonization

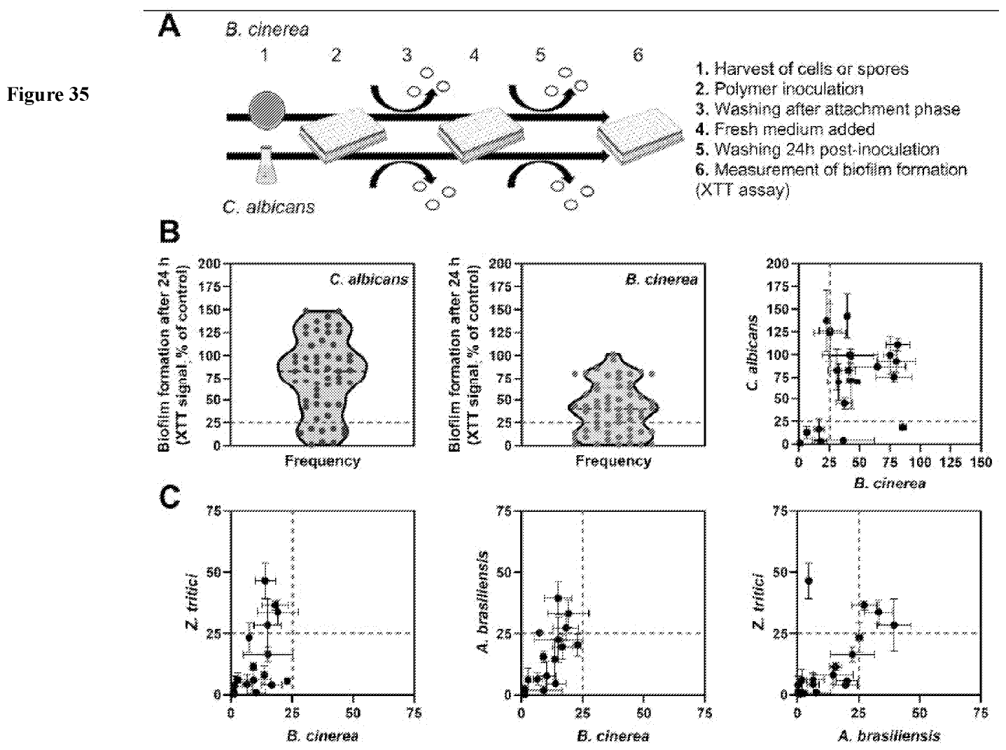

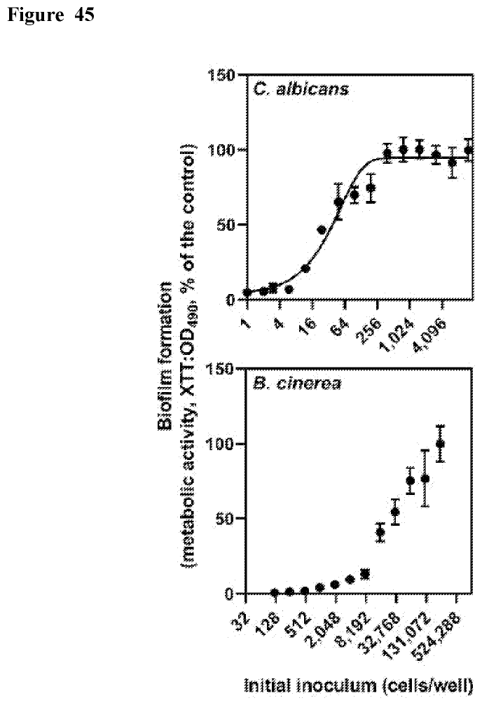

To investigate the scalability of the polymers' physiochemical properties and biological performance, the 80 polymers supporting the least attachment of each fungus from the microarray screen were deposited as a coating covering the 6.4 mm diameter wells of 96-well microplates. Several of the polymers proved to exhibit surface cracking post incubation under vacuum and so were excluded from the analysis due to the presence of these additional topological features. Incubations of fungus with polymers for 24 h were longer than in the screen, to allow some outgrowth and biofilm formation for a more sensitive measure of preceding attachment events; non-adherent cells or spores were removed by washing at the end of an initial attachment phase (FIG. 35A). Biofilm was detected with a metabolic XTT reduction assay, which eliminated the issue with autofluorescence of certain polymers. Materials of interest were designated as those supporting <25% biofilm formation compared to the control (non-coated well): <25% is equivalent to a biofilm that would result from a >95% reduction in attachment by the test fungi (FIG. 45). For C. albicans, nine of the scaled-up polymers supported <25% biofilm formation (FIG. 35B and Table 10), whereas 19 of the test polymers had such efficacy against B. cinerea (FIG. 35B, Table 11). Of the 27 materials that were common to the two organisms in the screen, and which did not exhibit surface cracking (see above), only four yielded <25% biofilm formation for both organisms in the scale-up assay. In the scale-up assay there was a similarly weak positive correlation between results for C. albicans cells and B. cinerea spores as in the preceding screen, but this was not significant in the case of the scale-up where only 23 polymers were assayed (Pearson correlation, R²=0.077, p=0.20) (FIG. 35B). We extended the scale-up assay to test the 19 polymers giving <25% B. cinerea biofilm also against another major plant pathogen, Zymoseptoria tritici, and an environmental filamentous fungus that colonizes diverse materials, Aspergillus brasiliensis. There were closer correlations between results for spores of these three fungi than with C. albicans, with the strongest correlation between B. cinerea and A. brasiliensis (R²=0.558, p=0.0002) (FIG. 35C). Interestingly, 15 of the 19 polymers tested were resistant to the attachment of at least two of the three filamentous fungi (Table 11).

Based on available information for the polymers' costs, chemistries and toxicities, we selected nine leads deemed suitable by these criteria for further investigation: six from the C. albicans assay and five from B. cinerea (two were common to both fungi). As the focus was on materials that passively resisted fungal attachment, we tested for potential toxicity effects to exclude polymers that might be actively inhibiting the fungi. Here, the main technical difference with the above “anti-attachment” assays was the omission of the washing steps; all cells including any dead cells were therefore retained in the wells (FIG. 36A,B). As there were no washing steps, the PDB medium could not be replaced with PBS to perform the XTT assay for toxicity in B. cinerea; therefore, B. cinerea was cultivated for 15 days on the materials in the presence of PDB and growth effects assessed visually (FIG. 36B). C. albicans growth was not inhibited by any of the polymers; there were no significant differences between the polymer and control (uncoated-well) incubations (FIG. 36A). However, pEGPhEA strongly inhibited the growth of B. cinerea (FIG. 36B). This may have been caused by residual or leached toxic material in the medium rather than a direct action of the coated polymer. To test this hypothesis, we added PDB medium to a well containing UV-polymerized pEGPhEA and, after 24 h, transferred this medium to wells containing B. cinerea spores. After 24 h subsequent growth, the OD₆₀₀ was 2.5-fold lower than in a control incubation (i.e., spores incubated with polymer-free medium) (FIG. 36C). This result corroborated that materials present in the medium (e.g. monomers or short oligomers leached from pEGPhEA) were toxic to B. cinerea growth. Given this complication, pEGPhEA was excluded from further study. In summary, the attachment data obtained for several polymers indicated that scaling up materials could alter their biological performance. However, several lead materials maintained a strong fungal anti-attachment effect, which was not attributable to active growth inhibition.

3D-Printed Polymer-Forms Resist Colonization by Candida albicans

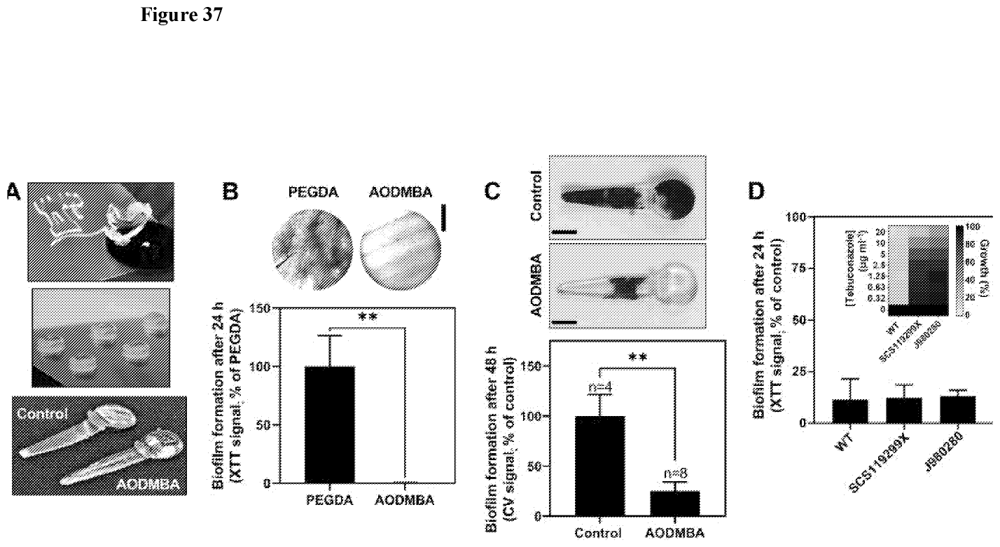

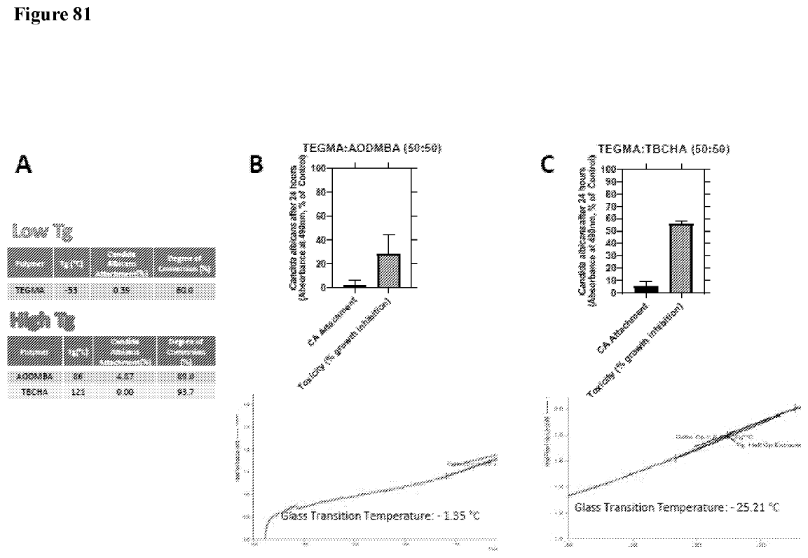

To evaluate the potential of the polymers-of-interest (above) as biofilm-resistant coatings (e.g., for medical devices), we attempted to dip coat silicone coupons using polymer solutions prepared with the candidate materials. The resultant surfaces proved either to be cracked [(R)-α-acryloyloxy-β,β-dimethyl-γ-butyrolactone (AODMBA) and tertbutylcyclohexyl methacrylate (tBCHMA)], or to produce coatings that were both poorly-adhered and prone to exhibit high levels of creep [di(ethylene glycol) methyl ether methacrylate (DEGMA) and tri(ethylene glycol) methyl ether methacrylate (TEGMA)]. Consequently, instead we attempted to 3D-print the target geometry directly. All the candidate monomers showed stable droplet formation during initial assessment of printability. However, during the actual printing process for 3D structures, only an AODMBA based formulation solidified and formed stable geometries. The other candidates either remained as a tacky solid phase, which was attributed to a low glass transition temperature (T_{g}), or they collapsed during the printing. Coupons (3-mm diameter) manufactured with AODMBA were used initially to test the anti-attachment properties of the printed polymer (FIG. 37A). Polyethylene glycol diacrylate (PEG₅₇₅DA) gave good C. albicans attachment in the previous tests (comparable to the attachment on a non-coated well) (FIG. 35B), so coupons 3D-printed with PEG₅₇₅DA were used as attachment positive-control. A □100% reduction in C. albicans attachment was apparent with the AODMBA-printed coupons compared to those that were PEG₅₇₅DA-printed (FIG. 37B). As a more commercially relevant example, next we used AODMBA for printing valve-flap forms for voice prostheses. This example was chosen because commercial silicone-manufactured valve flaps are highly susceptible in vivo to develop C. albicans biofilms (22). Our results showed that AODMBA-printed valve flaps were more resistant to fungal attachment than a standard silicone-manufactured product, with a mean 84% reduction in biofilm biomass and up to 100% reduction in some replicates (n=8) (FIG. 37C). In conclusion, AODMBA was demonstrated to; (a) be 3D-printable and (b) exhibit strong anti-attachment properties that were retained in AODMBA-printed forms. As drug-resistant C. albicans poses particular problems for therapy, we also tested the lead material against strains resistant to azole drugs (FIG. 37D). These tests showed that anti-attachment by AODMBA is just as effective against the drug-resistant isolates as against a standard C. albicans strain, further supporting the potential for clinical application (FIG. 81). This co-polymer retained strong anti-attachment properties against C. albicans and low toxicity, while exhibiting an intermediate glass transition temperature. Similar results were obtained for a TEGMA:TBCHA co-polymer.

Lead Polymers can Protect Plant Leaves from Fungal Infection

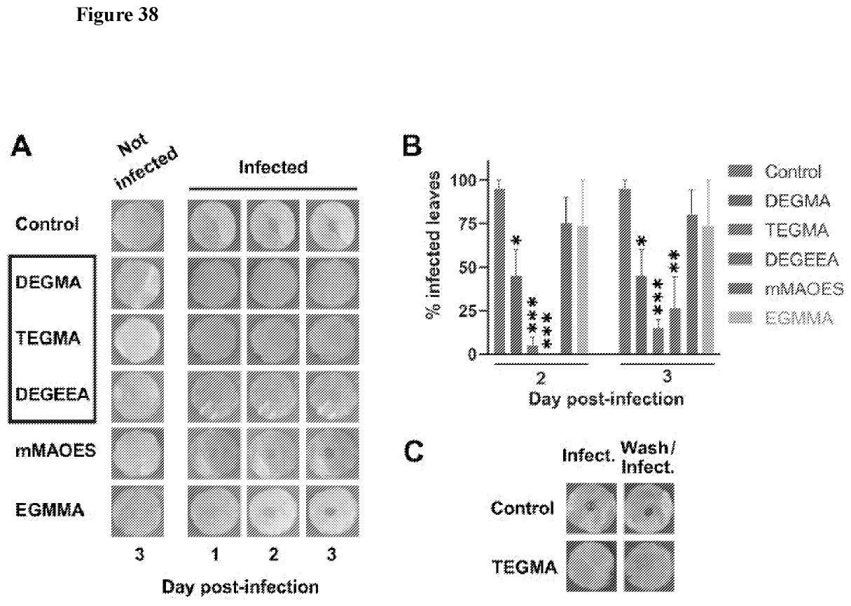

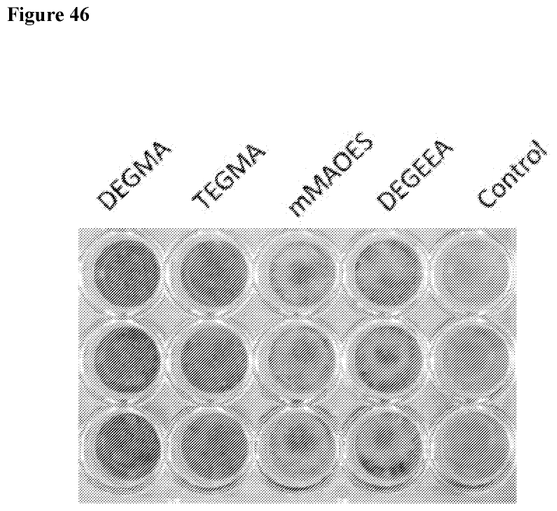

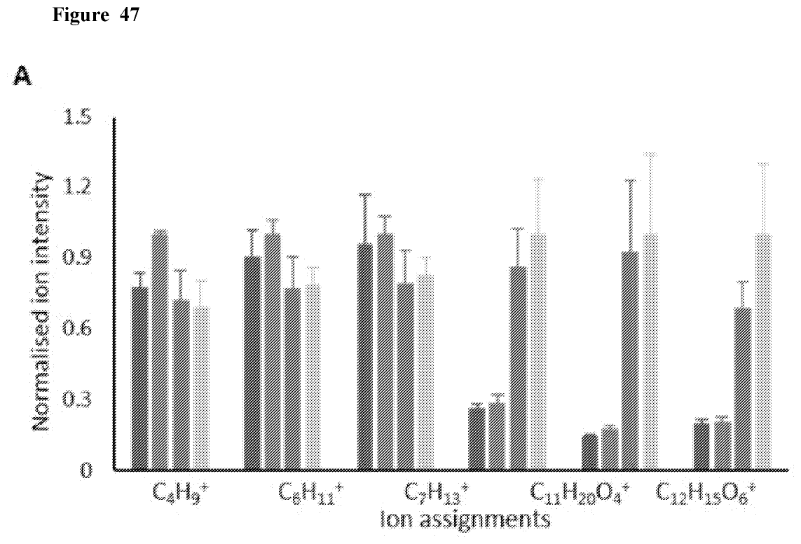

We then hypothesised that the lead polymers could find novel applications for protecting plant (crop) surfaces from fungal infection. To explore this possibility, first we tested for potential plant toxicity with polymers that had given good anti-attachment against B. cinerea in vitro (FIG. 35B). Polymer solutions (≥85% monomer conversion; Table 12) were sprayed on to lettuce-leaf discs. No leaf lesions were observed up to 3 days after spraying with polymer, suggesting an absence of toxicity (leaf samples deteriorated after 3 days regardless of polymer application) (FIG. 38A, left panel). To test for effects on fungal infection, leaf-discs treated either with polymer or with solvent alone (control) were inoculated with B. cinerea spores. Whereas fungal lesions were evident after 2 days in all of the controls, leaves treated with either di(ethylene glycol) ethyl ether acrylate (DEGEEA), DEGMA, or TEGMA were significantly resistant to B. cinerea infection (FIGS. 38A, middle panel, and 38B). Fewer than 15% of TEGMA treated leaf samples showed any sign of infection up to 3 days. In contrast, mono-2-(methacryloyloxy)ethyl succinate (mMAOES) did not confer any apparent protection as lesions appeared after 2 days: the outcome for mMAOES was similar to the untreated leaves or leaves treated with ethylene glycol methyl ether methacrylate (EGMMA), which had been selected as an attachment positive-control. As B. cinerea could grow in the presence of these synthesised polymer batches in vitro (FIG. 46), the effects on infection could not be ascribed to toxicity to the fungus. Finally, we tested the resilience of the best performing polymer (TEGMA) to rinsing with water. TEGMA was sprayed onto the leaf-discs and air dried as above, before the leaves were rinsed 3 times with water and subsequently infected. After 3 days, no lesions were observed (FIG. 38C). This indicated that the anti-attachment property of the polymer conferred to the leaf surface was resilient to rinsing with water, such as may occur in the natural environment during rainfall. The presence of TEGMA after washing was confirmed by ToF-SIMS; no significant change was observed between the washed or unwashed leaf sections (FIG. 47). The data were consistent with a potential for application of these materials in agriculture.

### Table S3

TABLE 53
Identities and structures of polymers resistant to colonization by filamentous fungi

| | | Biofilm formation - 24 h (%) [symbol] | | | |
|---|---|---|---|---|---|
| Acronym | Full name | B. cinerea | S. tritici | A. brasiliensis | WCA ( [symbol] )^{b} |
| ZrCEA | Zirconium carboxyethyl acrylate | 1.0 ± 0.3 | 0.6 ± 0.1 | 2.7 ± 1.4 | 20.8 ± 3.6 |
| HfCEA | Hafnium carboxyethyl acrylate | 1.1 ± 0.8 | 0.9 ± 0.5 | 1.4 ± 0.7 | 23.4 ± 6.5 |
| PDA | 1,4-Phenylene diacrylate | 1.2 ± 0.3 | 0.2 ± 0.1 | 0.6 ± 0.1 | 62.4 ± 2.9 |
| DEAEA | Diethylamino ethyl acrylate | 1.3 ± 1.1 | 3.9 ± 3.8 | 0.2 ± 0.2 | 47.8 ± 13.0 |
| mMAOES | mono-2-(Methacryloyloxy)ethyl succinate | 1.4 ± 0.6 | 0.9 ± 0.8 | 0.2 ± 0.2 | 50.5 ± 4.8 |
| MAA | Methyl 2-acetamidoacrylate | 2.8 ± 1.1 | 6.2 ± 2.8 | 6.3 ± 4.7 | 52.9 ± 15.7 |
| TEGMA | Tri(ethylene glycol) methyl ether methacrylate | 6.6 ± 1.9 | 4.3 ± 4.1 | 6.4 ± 2.4 | 23.7 ± 3.5 |
| DiPEMA | 2-Diisopropylaminoethyl methacrylate | 7.4 ± 0.4 | 23.3 ± 6.1 | 25.4 ± 1.3 | 53.4 ± 6.7 |
| pEGPhEA | Poly(ethylene glycol) phenyl ether acrylate | 9.1 ± 1.0 | 11.5 ± 1.5 | 15.6 ± 2.4 | 33.2 ± 5.8 |
| MAAH | Methacrylic anhydride | 9.2 ± 7.4 | 6.1 ± 4.3 | 1.8 ± 1.8 | 49.2 ± 9.2 |
| DEGEEA | Di(ethylene glycol) ethyl ether acrylate | 10.3 ± 3.8 | 0.9 ± 0.8 | 7.7 ± 5.8 | 46.4 ± 12.2 |
| EOEA | Ethoxyethyl acrylate | 13.7 ± 1.3 | 8.1 ± 3.8 | 14.6 ± 8.2 | 41.7 ± 1.7 |
| PMAm | N-(Phthalimidomethyl)acrylamide | 14.0 ± 4.2 | 46.6 ± 7.3 | 4.6 ± 0.5 | 67.1 ± 13.3 |
| tBAm | N-tert-Butylacrylamide | 15.0 ± 5.7 | 28.5 ± 10.7 | 39.5 ± 6.9 | 53.0 ± 5.6 |
| LaA | Lauryl acrylate | 15.1 ± 10 | 16.5 ± 3.0 | 22.5 ± 9.1 | 43.4 ± 8.3 |
| DEGMA | Di(ethylene glycol) methyl ether methacrylate | 16.7 ± 4.2 | 3.9 ± 0.1 | 19.6 ± 5.0 | 47.9 ± 15.6 |
| CNEA | Cyanoethyl acrylate | 18.2 ± 5.3 | 36.5 ± 1.9 | 27.3 ± 5.2 | 71.8 ± 10.1 |
| CMAOE | Caprolactone 2-(methacryloyloxylethyl ester | 19.2 ± 8.4 | 33.7 ± 5.0 | 33.2 ± 5.7 | 34.8 ± 4.0 |
| EGMEA | Ethylene glycol methyl ether acrylate | 23.0 ± 1.2 | 5.7 ± 0.5 | 20.4 ± 4.5 | 55.2 ± 11.8 |

^{a}Mean value from at least three independent experiments ±SEM; according to XTT signal as a percentage of the signal obtained in non-coated wells. Polymers shown are those giving <25% attachment with B. cinerea.
^{b}Water contact angle; mean value from three independent experiments ± 5D. [symbol]
indicates data missing or illegible when filed

### Discussion

This study reveals polymer materials with the potential to stop fungal colonization by passively blocking attachment and demonstrates potential applications for tackling at least some of the diverse problems that fungi cause. Currently, antifungal and fungicidal agents are widely used to combat fungal pathogens, fungi causing biodeterioration and spoilage fungi. However, with an increased incidence of fungal isolates resistant to current treatments and tightening antifungal and fungicide regulations, novel methods for fungal management are needed. Controlling fungal attachment to surfaces in a passive manner (i.e., without active killing of organisms) presents an alternative, attractive intervention at the initial step of fungal colonization. Attachment via adhesion is a pre-requisite for most adverse effects of fungi, including formation of biofilms that are an important virulence factor in microbial pathogenesis. Therefore, inhibition of attachment should be an effective target for controlling most fungi. The passive control described here could reduce the potential development of resistant organisms, as selection pressure for resistance (to anti-attachment polymers) should be considerably lower where non-resistance is not fatal and, in some cases, may have negligible disadvantage. Furthermore, resistance in this case could require organisms to gain a new function, in order to achieve attachment, which raises greater evolutionary hurdles (35).

For the prevention of C. albicans attachment on acrylic resins, chemical or physical surface alterations such as modification of surface charge (36, 37), increasing surface wettability or decreasing surface energy (38-41) previously gave lower C. albicans adhesion. Hydrophilic polymers have also been used in surface modification (39, 40) as hydrophobic fungi preferentially adhere to hydrophobic surfaces (42). The present study utilised high throughput screening to identify new polymers that are able to reduce fungal attachment from a library of over 250 unique materials. Thus, this study is the largest assessment of material-fungi interactions to date. The recent development of a methodology for discovery of polymers resistant to bacterial adhesion, using high-throughput surface characterization and chemometrics, helped characterise chemical moieties that reduced bacterial attachment to coated medical devices in vivo (15). This class of materials could not have been predicted from the current understanding of bacteria-material interactions. Adopting this strategy for fungi, we identified both acrylate and methacrylate polymers that resisted fungal attachment, to either biological or inert surfaces; in the latter case, the novel anti-fungal polymer outperforms the current market leading silicone-manufactured material.

In this study, we were able to 3D-print using AODMBA, a material not previously reported to be printable. Fungal anti-attachment properties of AODMBA were retained after printing, including in a printed medical device part (valve flap for a voice prosthesis). One of the advantages of manufacturing such parts with polymer rather than coating the polymer onto target devices, is the fact that the polymer will not delaminate and therefore expose regions of (potentially attachment-prone) native surface. The AODMBA-printed valve flaps showed >80% reduction of biofilm formation compared with a standard silicone-manufactured product. However, AODMBA forms a hard glassy polymer that would therefore be, in its homopolymer form, too inflexible for valve-flap applications. This is analogous to the anti-bacterial polymer EGDPEA development, in which the homopolymer was also not suitable to produce a viable catheter coating (16), rather an optimised copolymer had to be developed. Similarly, to develop a commercially viable coating, the mechanical properties of that material were improved by copolymerization with a co-monomer (DEGMA) that has a lower glass transition temperature (T_{g}). In practice, the T_{g} values that were exhibited by poly(EGDPEA-co-DEGMA) polymers, synthesised in various different co-monomer ratios, were used as a high throughput screening guide to predict the copolymer compositions that should match the flexibility of the commercial catheter material. Pin printing assays using those copolymers with acceptable T_{g}'s confirmed that they still retained the bacterial attachment-resistance (16). Similar optimization could be an aim of future work to improve the mechanical properties of AODMBA-based forms. The present work is a key step toward deriving a combined set of molecular and material descriptors for building a set of design rules to define even better molecular structures, which could be synthesised to improve performance further. Our analyses showed that surface chemistry is not a very strong differentiator for fungal attachment, suggesting that material properties will have a more significant part to play in the definition of performance compared with the bacterial work.

In agriculture, polymer materials have found applications for improving physical properties of soil and as adjuvants in polymeric biocide and herbicide formulations. These latter are controlled release formulations designed to reduce the possible side effects accompanying the overuse of biologically active agents. However, the passive application proposed in the current study is novel, as a potential replacement for active agents in formulations. Anti-attachment was effective against B. cinerea and Z. tritici, two major crop pathogens. Furthermore, three of four selected polymers conferred plant protection against B. cinerea infection. TEGMA, the best performing polymer, showed resistance to the attachment of all four fungi used in this study suggesting a broad spectrum of action of this methacrylate material. Broad spectrum agents are particularly valued in common antimicrobial applications, including for crop protection.

In conclusion, this work unveils a panel of novel polymers which are resistant to fungal attachment and therefore reduce fungal biofilm formation and infection. Besides the therapeutic and crop protection potential, such acrylate and methacrylate polymers could have wider applications as demonstrated by their effect on the attachment of A. brasiliensis, known to colonize synthetic products and materials. This study comprised the first step toward the targeted design of efficacious materials, tailored for different anti-fungal applications.

TABLE 10
Identities and structures of polymers resistant to colonization
by C. albicans

| | | Biofilm | |
|---|---|---|---|
| | | formation | |
| | | after 24 h | |
| Acronym | Full name | (%) | |
| AODMBA | (R)-α-Acryloyloxy-β,β-dimethyl-γ- | 4.1ª | ± 3.0 |
| | butyrolactone | | |
| CNEA | Cyanoethyl acrylate | 3.1 | ± 1.6 |
| tBCHMA | Tertbutylcyclohexyl methacrylate | 13.6 | ± 9.6 |
| PDA | 1,4-Phenylene diacrylate | 1.0 | ± 0.2 |
| DEGMA | Di(ethylene glycol) methyl ether | 16.3 | ± 11.3 |
| | methacrylate | | |
| TEGMA | Tri(ethylene glycol) methyl ether | 13.0 | ± 6.7 |
| | methacrylate | | |
| PHPMA | 3-Phenoxy 2 hydroxy propyl | 18.4 | ± 3.2 |
| | methacrylate | | |
| tBCHA | Tert-butylcyclohexylacrylate | 18.5 | ± 4.5 |
| iDMA | Isodecyl methacrylate | 19.1 | ± 2.0 |

^{a}Mean value from at least three independent experiments ±SEM; according to XTT signal as a percentage of the signal obtained in non-coated wells. Polymers shown are those giving <25% attachment.

TABLE 11
Identities and structures of polymers resistant to colonization by filamentous fungi

| | | Biofilm formation after 24 h (%) | | |
|---|---|---|---|---|
| Acronym | Full name | B. cinerea | S. tritici | A. brasiliensis |
| ZrCEA | Zirconium carboxyethyl acrylate | 1.0 ± 0.3 | 0.6 ± 0.1 | 2.7 ± 1.4 |
| HfCEA | Hafnium carboxyethyl acrylate | 1.1 ± 0.8 | 0.9 ± 0.5 | 1.4 ± 0.7 |
| PDA | 1,4-Phenylene diacrylate | 1.2 ± 0.3 | 0.2 ± 0.1 | 0.6 ± 0.1 |
| DEAEA | Diethylamino ethyl acrylate | 1.3 ± 1.1 | 3.9 ± 3.8 | 0.2 ± 0.2 |
| mMAOES | mono-2-(Methacryloyloxy)ethyl succinate | 1.4 ± 0.6 | 0.9 ± 0.8 | 0.2 ± 0.2 |
| MAA | Methyl 2-acetamidoacrylate | 2.8 ± 1.1 | 6.2 ± 2.8 | 6.3 ± 4.7 |
| TEGMA | Tri(ethylene glycol) methyl ether | 6.6 ± 1.9 | 4.3 ± 4.1 | 6.4 ± 2.4 |
| | methacrylate | | | |
| DiPEMA | 2-Diisopropylaminoethyl methacrylate | 7.4 ± 0.4 | 23.3 ± 6.1 | 25.4 ± 1.3 |
| pEGPhEA | Poly(ethylene glycol) phenyl ether acrylate | 9.1 ± 1.0 | 11.5 ± 1.5 | 15.6 ± 2.4 |
| MAAH | Methacrylic anhydride | 9.2 ± 7.4 | 6.1 ± 4.3 | 1.8 ± 1.8 |
| DEGEEA | Di(ethylene glycol) ethyl ether acrylate | 10.3 ± 3.8 | 0.9 ± 0.8 | 7.7 ± 5.8 |
| EOEA | Ethoxyethyl acrylate | 13.7 ± 1.3 | 8.1 ± 3.8 | 14.6 ± 8.2 |
| PMAm | N-(Phthalimidomethyl)acrylamide | 14.0 ± 4.2 | 46.6 ± 7.3 | 4.6 ± 0.5 |
| tBAm | N-tert-Butylacrylamide | 15.0 ± 5.7 | 28.5 ± 10.7 | 39.5 ± 6.9 |
| LaA | Lauryl acrylate | 15.1 ± 10 | 16.5 ± 3.0 | 22.5 ± 9.1 |
| DEGMA | Di(ethylene glycol) methyl ether methacrylate | 16.7 ± 4.2 | 3.9 ± 0.1 | 19.6 ± 5.0 |
| CNEA | Cyanoethyl acrylate | 18.2 ± 5.3 | 36.5 ± 1.9 | 27.3 ± 5.2 |
| CMAOE | Caprolactone 2-(methacryloyloxy)ethyl ester | 19.2 ± 8.4 | 33.7 ± 5.0 | 33.2 ± 5.7 |
| EGMEA | Ethylene glycol methyl ether acrylate | 23.0 ± 1.2 | 5.7 ± 0.5 | 20.4 ± 4.5 |

^{a}Mean value from at least three independent experiments ±SEM; according to XTT signal as a percentage of the signal obtained in non-coated wells. Polymers shown are those giving <25% attachment with B. cinerea.

TABLE 12
Synthesis of the fungal anti-attachment materials by free radical
polymerisation using a thiol chain transfer agent. Percentage of
conversion of (meth)acrylate monomers to cured polymers, molecular
weight (number average, Mₙ) and polydispersity (Ð) for each material
were determined by ¹H-NMR and GPC analysis.

| Acronym | % conversion | Mₙ (Da, GPC) | Ð | Mₙ (Da, ¹H-NMR) |
|---|---|---|---|---|
| DEGEEA | >90 | 4202 | 1.27 | 5096 |
| DEGMA | >90 | 5778 | 1.40 | 6154 |
| EGMMA | 90 | 4197 | 1.46 | 2951 |
| TEGMA | >90 | 7246 | 1.41 | 4231 |
| mMAOES | 80-85% | — | — | 8464 |

### REFERENCES

1. Brown G D, et al. (2012) Hidden killers: human fungal infections. Sci Transl Med 4:165rv113.
2. Fisher M C, Hawkins N J, Sanglard D, & Gurr S J (2018) Worldwide emergence of resistance to antifungal drugs challenges human health and food security. Science 360:739-742.
3. Anonymous (2017) Stop neglecting fungi. Nat Microbiol 2:17120.
4. Avery S V, Singleton I, Magan N, & Goldman G H (2019) The fungal threat to global food security. Fungal Biol 123:555-557.
5. Cavalheiro M & Teixeira M C (2018) Candida biofilms: Threats, challenges, and promising strategies. Front Med 5:28.
6. Villa F, Cappitelli F, Cortesi P, & Kunova A (2017) Fungal biofilms: Targets for the development of novel strategies in plant disease management. Front Microbiol 8:654.
7. Carratala J (2002) The antibiotic-lock technique for therapy of ‘highly needed’ infected catheters. Clin Microbiol Infect 8:282-289.
8. Cateau E, Berjeaud J M, & Imbert C (2011) Possible role of azole and echinocandin lock solutions in the control of Candida biofilms associated with silicone. Int J Antimicrob Agents 37:380-384.
9. Bonne S, et al. (2015) Effectiveness of minocycline and rifampin vs chlorhexidine and silver sulfadiazine-impregnated central venous catheters in preventing central line-associated bloodstream infection in a high-volume academic intensive care unit: a before and after trial. J Am Coll Surg 221:739-747.
10. Villa F, Remelli W, Forlani F, Vitali A, & Cappitelli F (2012) Altered expression level of Escherichia coli proteins in response to treatment with the antifouling agent zosteric acid sodium salt. Environ Microbiol 14:1753-1761.
11. Catto C, et al. (2015) Unravelling the structural and molecular basis responsible for the anti-biofilm activity of zosteric acid. PLoS One 10:e0131519.
12. Stanley M S, et al. (2002) Inhibition of fungal spore adhesion by zosteric acid as the basis for a novel, nontoxic crop protection technology. Phytopathology 92:378-383.
13. Villa F, et al. (2010) Hindering biofilm formation with zosteric acid. Biofouling 26:739-752.
14. Anderson D G, Levenberg S, & Langer R (2004) Nanoliter-scale synthesis of arrayed biomaterials and application to human embryonic stem cells. Nat Biotechnol 22:863-866.
15. Hook A L, et al. (2012) Combinatorial discovery of polymers resistant to bacterial attachment. Nat Biotechnol 30:868-875.
16. Adlington K, et al. (2016) Application of targeted molecular and material property optimization to bacterial attachment-resistant (meth)acrylate polymers. Biomacromolecules 17:2830-2838.
17. Tyler B J, et al. (2017) Development and characterization of a stable adhesive bond between a poly(dimethylsiloxane) catheter material and a bacterial biofilm resistant acrylate polymer coating. Biointerphases 12:02C412.
18. Sanni O, et al. (2015) Bacterial attachment to polymeric materials correlates with molecular flexibility and hydrophilicity. Adv Healthc Mater 4:695-701.
19. Dundas A A, et al. (2019) Validating a predictive structure-property relationship by discovery of novel polymers which reduce bacterial biofilm formation. Adv Mater 31:1903513.
20. Hook A L, Alexander M R, & Winkler D A (2014) Chapter 8—Materiomics: a toolkit for developing new biomaterials. Tissue Engineering, eds Blitterswijk A V & Boer J D (Academic Press, Oxford), 2nd Ed, pp 253-281.
21. Dundas A A, et al. (2019) Methodology for the synthesis of methacrylate monomers using designed single mode microwave applicators. React. Chem. Eng. 4:1472-1476.
22. Talpaert M J, et al. (2015) Candida biofilm formation on voice prostheses. J Med Microbiol 64:199-208.
23. Hook A L, et al. (2012) Polymer microarrays for high throughput discovery of biomaterials. J Vis Exp 59:e3636.
24. Chen T Q, Guestrin C, & Assoc Comp M (2016) XGBoost: a scalable tree boosting system. Kdd'16: Proceedings of the 22nd Acm Sigkdd International Conference on Knowledge Discovery and Data Mining, (Assoc Computing Machinery, New York), pp 785-794.
25. Carbonell P, Carlsson L, & Faulon J L (2013) Stereo signature molecular descriptor. J Chem Inf Model 53:887-897.
26. Faulon J L, Visco D P, & Pophale R S (2003) The signature molecular descriptor. 1. Using extended valence sequences in QSAR and QSPR studies. J Chem Inf Comput Sci 43:707-720.
27. Begines B, Hook A L, Alexander M R, Tuck C J, & Wildman R D (2016) Development, printability and post-curing studies of formulations of materials resistant to microbial attachment for use in inkjet based 3D printing. Rapid Prototyp J 22:835-841.
28. He Y F, et al. (2017) A new photocrosslinkable polycaprolactone-based ink for three-dimensional inkjet printing. J Biomed Mater Res B—Appl Biomater 105:1645-1657.
29. Zhang F, et al. (2016) Inkjet printing of polyimide insulators for the 3D printing of dielectric materials for microelectronic applications. J Appl Polym Sci 133:43361.
30. Zhou Z X, et al. (2019) High-throughput characterization of fluid properties to predict droplet ejection for three-dimensional inkjet printing formulations. Addit Manufact 29:UNSP 100792.
31. Brothers K M, Newman Z R, & Wheeler R T (2011) Live imaging of disseminated candidiasis in zebrafish reveals role of phagocyte oxidase in limiting filamentous growth. Eukaryot Cell 10:932-944.
32. O'Brien D M, et al. (2019) Epoxy-amine oligomers from terpenes with applications in synergistic antifungal treatments. J Mat Chem B 7:5222-5229.
33. Beauvais A & Latge J P (2018) Special issue: Fungal cell wall. J Fungi 4:91.
34. Hibbett D S, et al. (2007) A higher-level phylogenetic classification of the Fungi. Mycol Res 111:509-547.
35. Cohen-Gihon I, Sharan R, & Nussinov R (2011) Processes of fungal proteome evolution and gain of function: gene duplication and domain rearrangement. Phys Biol 8:035009.
36. Park S E, Periathamby A R, & Loza J C (2003) Effect of surface-charged poly(methyl methacrylate) on the adhesion of Candida albicans. J Prosthodont 12:249-254.
37. Park S E, Blissett R, Susarla S M, & Weber H P (2008) Candida albicans adherence to surface-modified denture resin surfaces. J Prosthodont 17:365-369.
38. Lazarin A A, et al. (2014) Candida albicans adherence to an acrylic resin modified by experimental photopolymerised coatings: an in vitro study. Gerodontology 31:25-33.
39. Lazarin A A, et al. (2013) Effect of experimental photopolymerized coatings on the hydrophobicity of a denture base acrylic resin and on Candida albicans adhesion. Arch Oral Biol 58:1-9.
40. Izumida F E, et al. (2014) In vitro evaluation of adherence of Candida albicans, Candida glabrata, and Streptococcus mutans to an acrylic resin modified by experimental coatings. Biofouling 30:525-533.
41. Yodmongkol S, et al. (2014) The effects of silane-SiO₂ nanocomposite films on Candida albicans adhesion and the surface and physical properties of acrylic resin denture base material. J Prosthet Dent 112:1530-1538.
42. Krasowska A & Sigler K (2014) How microorganisms use hydrophobicity and what does this mean for human needs? Front Cell Infect Microbiol 4:112.

### Example 5—Identification of Neutrophil Instructive Polymers

Methodology; Neutrophils were Purified from Fresh Human Blood Using Magnetic Separation/Isolation Kit (MACS Express)

Neutrophil attachment was screened using the polymer microarray as previously described. Isolated cells were incubated with the arrays for 1 hour.

Microarrays were washed, fixed and stained with DAPI (nucleus counterstain). Images were acquired using the Zeiss widefield system and nuclei quantified using custom CellProfiler pipelines. Cluster analysis performed per donor across the monomers tested. Behaviour of Monomer classified as:

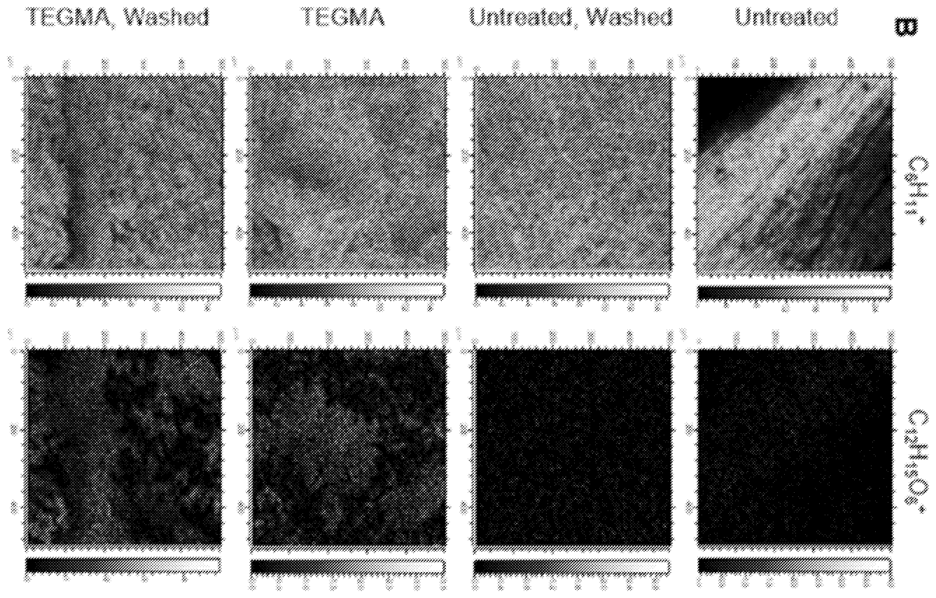

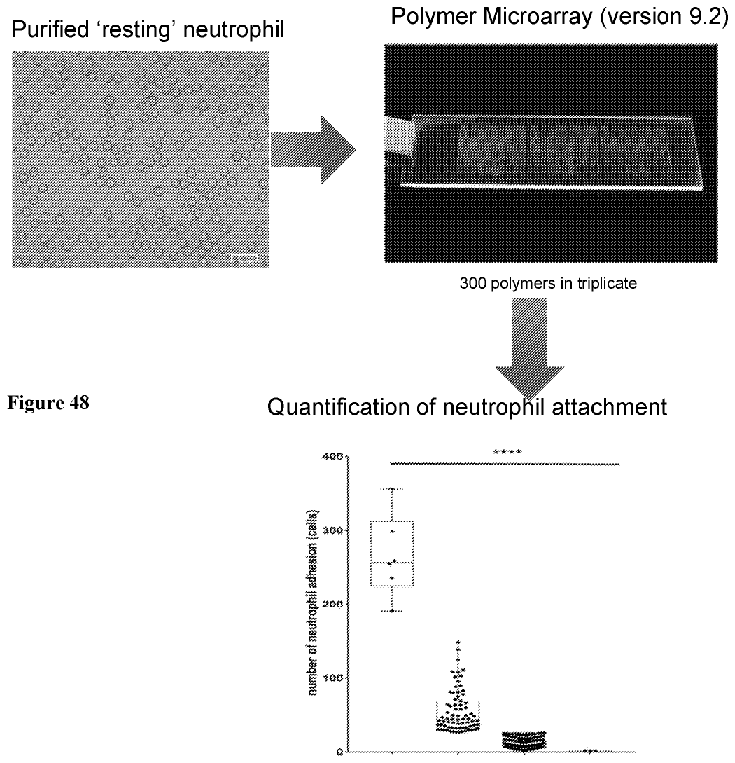

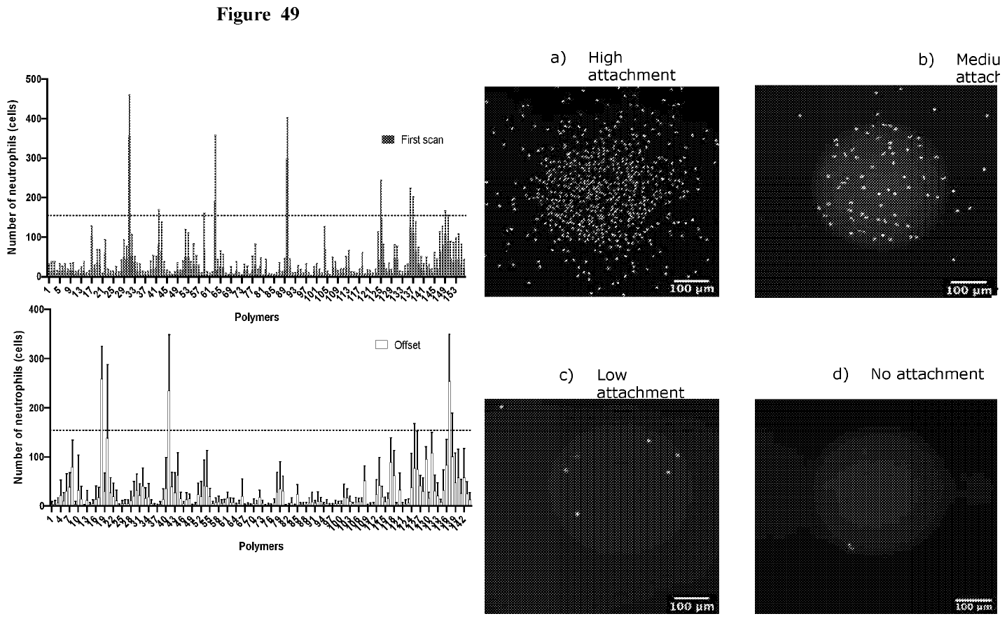

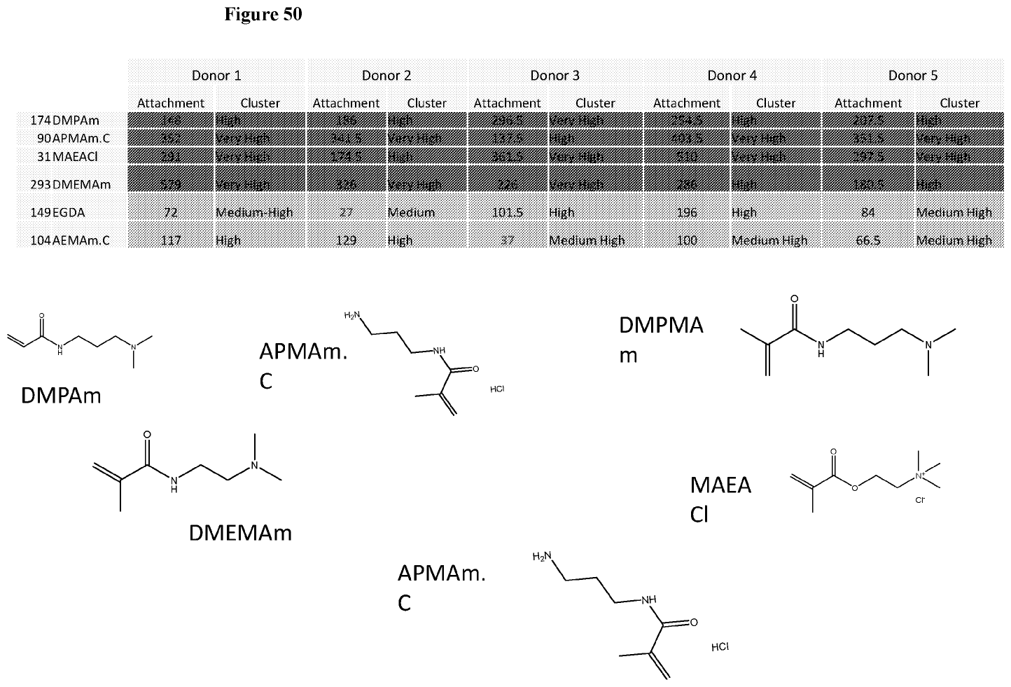

Very High/High/Medium High/Medium/Low attachment (Correlations >=0.8 (80%)). Consistent performance across donors for very high/high attachment See FIGS. 48-50.

### Example 6—Discovery of Novel Polymer Substrates for Xeno-Free, Long-Term Culture of Human Pluripotent Stem Cell Expansion

Human pluripotent stem cells (hPSCs) are capable of rapid self-renewal and multi-lineage differentiation into the three germ layers to form any adult tissue type. To realise their potential use for regenerative medicine applications, fully-defined hPSC culture systems need to be identified. Current synthetic surfaces incorporate biological substrates too expensive for large-scale use or require the use of serum or albumin containing culture medium for maintaining hPSC expansion. Here, rapid assessment of hPSC cell-polymer interactions in the xeno-free defined Essential 8™ medium using a multi-generational polymer microarray platform (284 monomers and 486 pairwise monomer combinations tested in individual assays) identifies a polymer substrate for long-term hPSC expansion. This study presents the scale-up of a novel polymer substrate consisting of a nanoscale blend of polymers tricyclodecane-dimethanol diacrylate and Butanediol diacrylate (70:30% w/v respectively) coated onto standard plastic cultureware, capable of supporting pluripotent hPSCs expansion (at least 8 serial passages) and subsequent directed differentiation to the three germ layers, including cardiomyocytes, neural progenitors and definitive endodermal cells. Follow-up mechanistic studies subsequently provide the first characterisation of hPSC cell-polymer interactions without the use of xenogenic components, thereby providing a useful cost-effective model for producing clinically relevant cells for stem cell research applications.

In recent years, in vitro hPSC culture has moved away from the use of animal-derived feeder layers to fully-defined xeno-free culture systems to improve their use for regenerative medicine applications.¹⁻² To be complaint with good manufacturing practice (GMP) regulations, clinically relevant hPSCs can be produced providing both the candidate growth substrate and culture medium are free from xenogenic contaminants. The use of high-throughput polymeric screening platforms, have led to significant advancements for assessing biomaterial-cell interactions and have led to the identification of the peptide based surfaces including Synthemax.³ However these leading commercially available surfaces incorporate biological substrates which considerably increases costs, prohibiting their scalability for large-scale production (costing approximately $10,000-$15,000 to produce 1 billion hPSCs for a single patient intervention).¹ Therefore, despite being animal-derived, Matrigel™, still remains the current most cost-effective and widely used growth substrate for hPSC research.

We have previously used the polymer microarray platform as a rapid strategy to assess polymer-hPSC interactions, demonstrating how proteins adsorbed from growth matrices (eg. fibronectin) and serum-containing medium are influenced by chemical structure and combinatorial mixing of polymers which subsequently affects cellular response.⁴⁻⁵ Follow-up studies acknowledged the relationship between protein adsorption and hPSC expansion to identify N-(4-hydroxyphenyl) methacrylamide and poly(2-hydroxyethylmethacrylate) poly(HPhMA-co-HEMA) co-polymers for hPSC culture in albumin containing medias mTESR1 and STEMPRO, which were the predominant commercially used culture medias at the time of the study.⁶ The current commercially available fully defined xeno-free E8™ medium for hPSC culture, only contains 8 components (fibroblast growth factor 2 (bFGF2), transforming growth factor beta (TGF-β), insulin, selenium, transferrin, L-ascorbic acid in DMEM/F12 basal medium with pH adjusted with NaHCO₃).⁷ The purpose of this study, is to identify a polymeric substrate that can support hPSC culture using Essential 8 medium, without a dependence on xenogenic components.

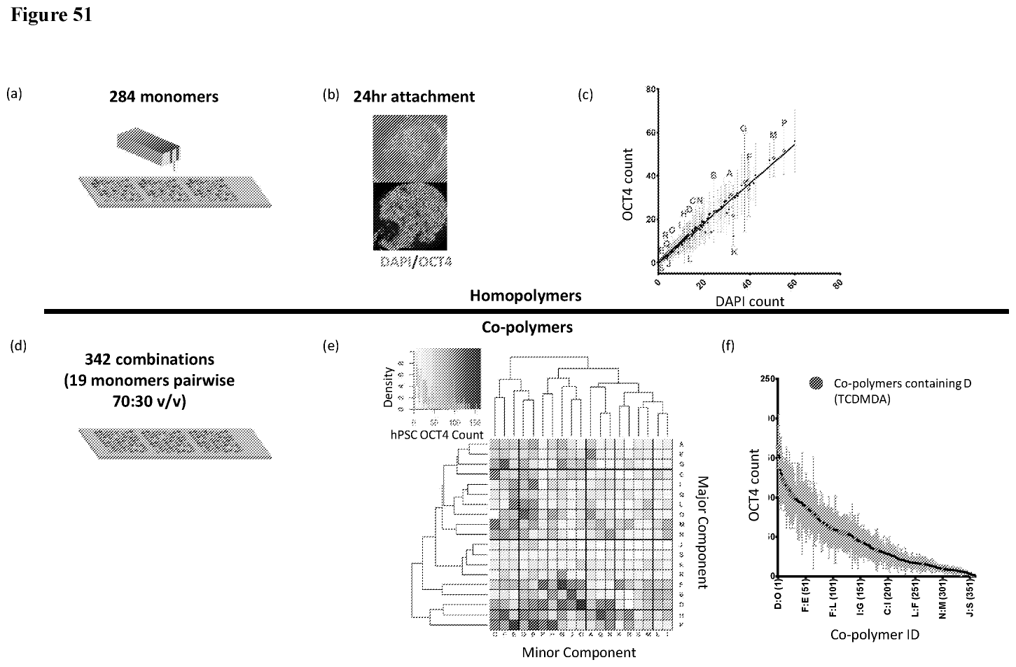

The multigenerational high-throughput polymer microarray approach was used to identify materials for supporting attachment and pluripotency of hiPSC line ReBI-PAT in Essential 8 medium. A first generation array consisting of a chemically diverse library of 284 monomers (photo-curable and readily commercially available) were pin-printed and UV polymerised (as previously described) as spots anchored to poly(2-hydroxyethyl methacrylate) (pHEMA) coated slides (FIG. 51a).⁸⁻⁹ ReBI-PATs were seeded on arrays and cultured in Essential 8 medium supplemented with pro-survival ROCK inhibitor (ROCKi, Y-27632) for the initial 24 hrs of culture following standard hPSC culture procedures and without the presence of ROCKi for a further 48 hrs. A 24 hrs screen, where samples were fixed and stained for the pluripotency marker, OCT4, provided quantitative data for ranking initial ReBI-PAT attachment to all materials screened (FIG. 51b-c).

Co-polymerisation has been shown to improve hPSC attachment.^{4, 6} Therefore, materials ranked by OCT4+ REBI-PAT attachment from the 24 hrs monomer screen were used to select a library of 23 monomers for combinatorial mixing. Monomers selected for a second-generation co-polymer array screen were selected with a range of OCT4+ attachment and were structurally diverse. The OCT4+ cell number is plotted against the cell number in FIG. 51c, with the monomers selected for the second generation screen denoted as red spots. In this plot, when the data points lie on the y=x line, that represented a population made up of OCT4+ cells, the deviation below the line represented a lower proportion of pluripotent cells in the cells residing on the polymer spot.

To produce 486 co-polymer combinations monomers were mixed pairwise prior to printing: 70/30% v/v mixtures were used where each monomer was combined as a major and minor component. Homopolymers were also included for comparison of the cell response, giving a total 509 chemistries printed in triplicate. The OCT4+ ReBI-PAT attachment is presented in FIG. 51e. Synergistic monomer combinations were assessed from the OCT4+ cell attachment after 24 hrs of culture (FIG. 51f). Co-polymers were synergistic if greater hPSC attachment was observed for a co-polymer (scaled to the proportion of each monomer) compared to their homopolymer counterparts. In total, 164 co-polymer combinations improved OCT4+ attachment and were deemed synergistic (synergy ratio ≥1) and therefore no clear co-polymer candidate could be identified. hPSC attachment was therefore reassessed qualitatively (data not shown) at 48 hrs to identify the best performing materials.

The co-polymers that supported high OCT4+ attachment for at least 48 hrs in the micro-array screens (1 monomer, P and 8 co-polymers mixed 70/30% w/v: D:Q, B:L, E:M, H:N, D:F, B:P, B:O and D:O) were scaled-up to tissue culture plastic (TCP) 96 well plates using UV polymerisation methods used for the array screens and compared with the current most widely-used ECM substrate, Matrigel™.

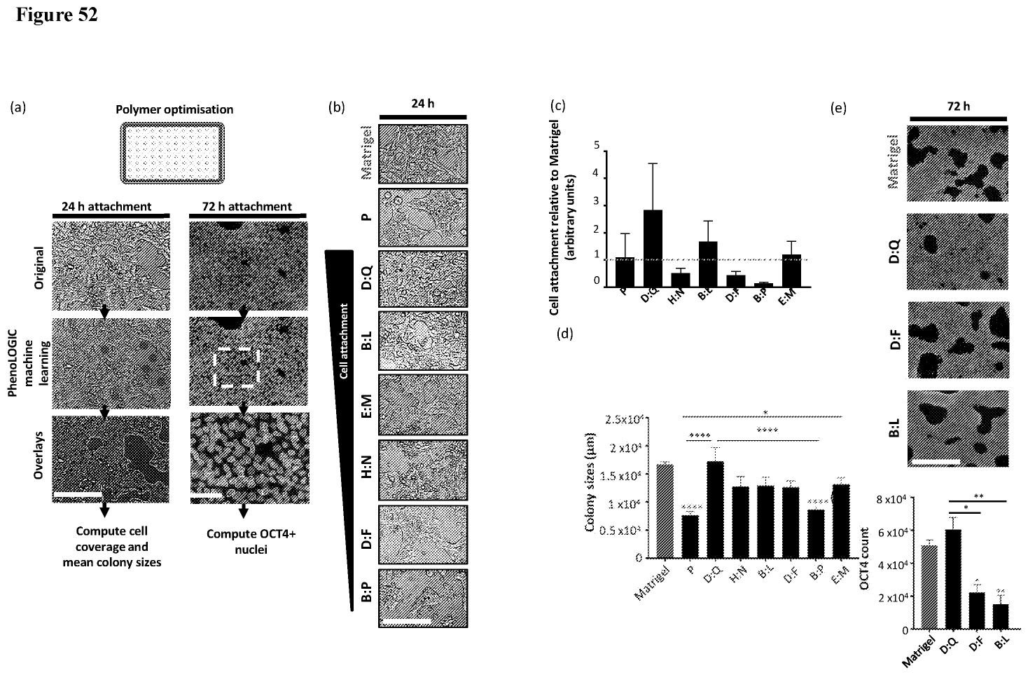

Initial 24 hr cell attachment was quantified as percentage hPSC cell coverage (relative to total area imaged/field of view) and mean sizes of colonies from live-cell brightfield images (FIG. 52a). Cells that remained attached after 72 hrs were fixed and stained for OCT4 expression. The mean cell coverage of REbl-PAT to polymerised Tetrahydrofurfuryl acrylate (THFuA, denoted P) after 24 hrs of culture was comparable to Matrigel (FIG. 52b-c), whilst the mean size of colonies formed were significantly smaller than all co-polymers tested (FIG. 52d).

Of the eight co-polymers tested, two containing 4-methacryloxyethyl trimellitic anhydride (O) failed to support attachment (data not shown). Three co-polymers: tricyclodecane-dimethanol diacrylate: butyl acrylate (D:Q), Neopentyl glycol diacrylate: hydroxylethyl methacrylate (B:L) and Tetraethylene glycol dimethacrylate: Ethylene glycol dicyclopentenyl ether acrylate (E:M) performed better than Matrigel™ in terms of mean percentage cell coverage, albeit variable.

Whilst the remaining three co-polymers:—Glycerol dimethacrylate: Furfuryl methacrylate (H:N), tricyclodecane-dimethanol diacrylate: Butanediol diacrylate (D:F) and Neopentyl glycol diacrylate: Tetrahydrofurfuryl acrylate (B:P) performed worse than Matrigel™ by demonstrating lower initial mean percentage cell coverage.

Based on the microarray screening results, hPSC attachment observed at 72 hrs signify robust materials. REbl-PATs were therefore cultured on co-polymers up to 72 hrs where they were fixed and stained for OCT4 expression (FIG. 52e-f). Only 3 co-polymers:—D:Q, D;F and B:L which had also formed similar sized colonies after 24 hrs (FIG. 52d) and were >90% positive for OCT4 expression were still maintained at 72 hrs. REbl-PAT attachment (quantified by DAPI nuclei) was significantly higher on D:Q compared to co-polymers D:F and B:L and equivalent to matrigel after 72 hrs of culture.

Overall, D:Q homopolymer components showed moderate attachment in the first generation array and moderate synergy (1.1) at co-polymerization. The ability to maintain attachment can in part be explained by structural and chemical surface analysis. However, in this study it is important to investigate the mechanisms of attachment coupled with the minimal E8 medium used for culturing hPSC cells since to our knowledge this is the first study to investigate hPSC attachment on a synthetic surface without the use of xenogenic components. In order to investigate this, we first explored whether serial passaging of HPSCs could be achieved at a larger sized plasticware (6 well plates). Herein, D:Q will be referenced to their homopolymer components tricyclodecane-dimethanol diacrylate (TCDMDA, denoted as D) and butyl acrylate (BA, denoted as Q) as poly(TCDMDA-blend-BA) for clarity.

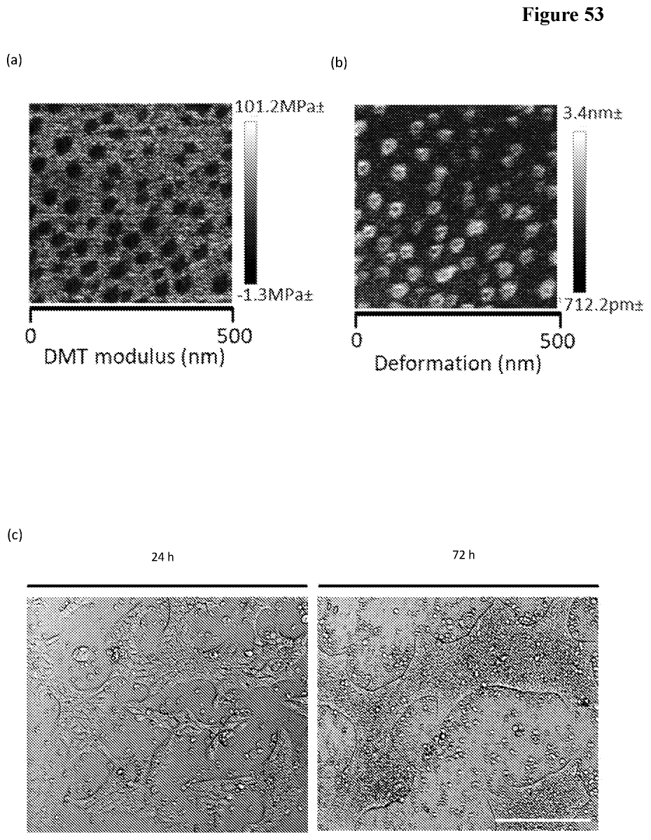

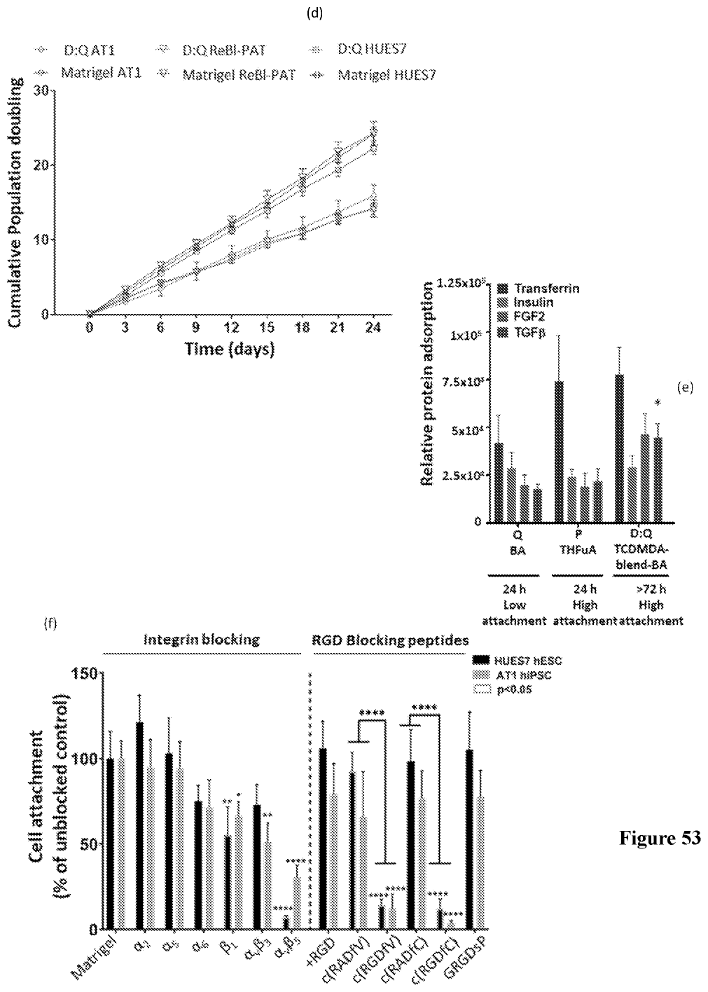

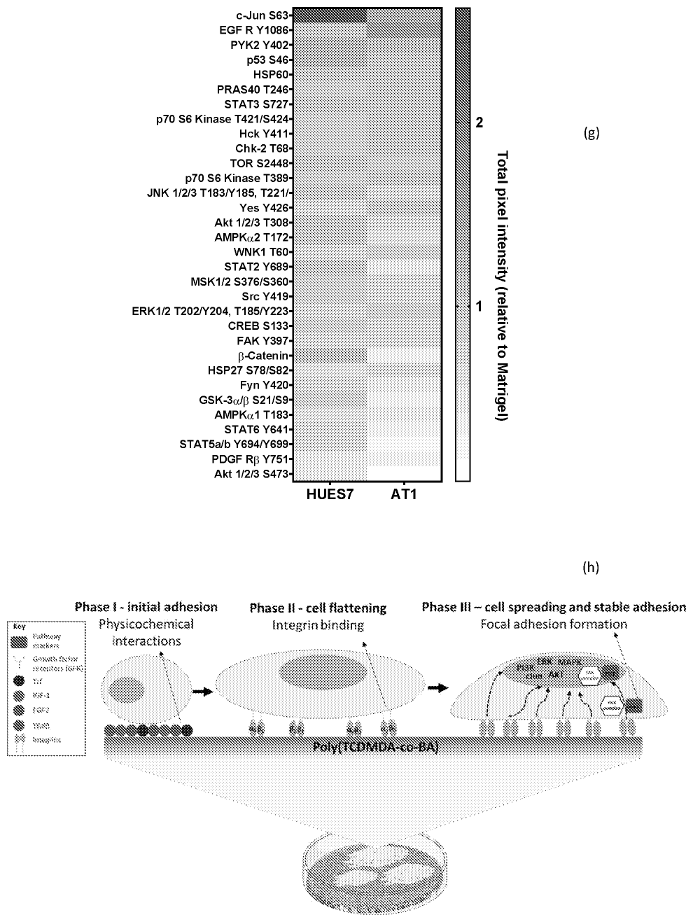

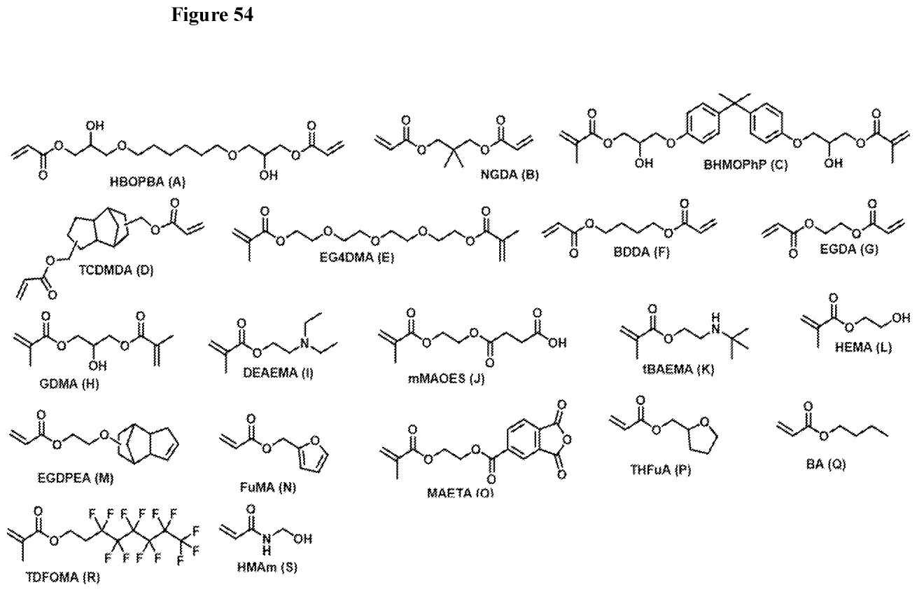

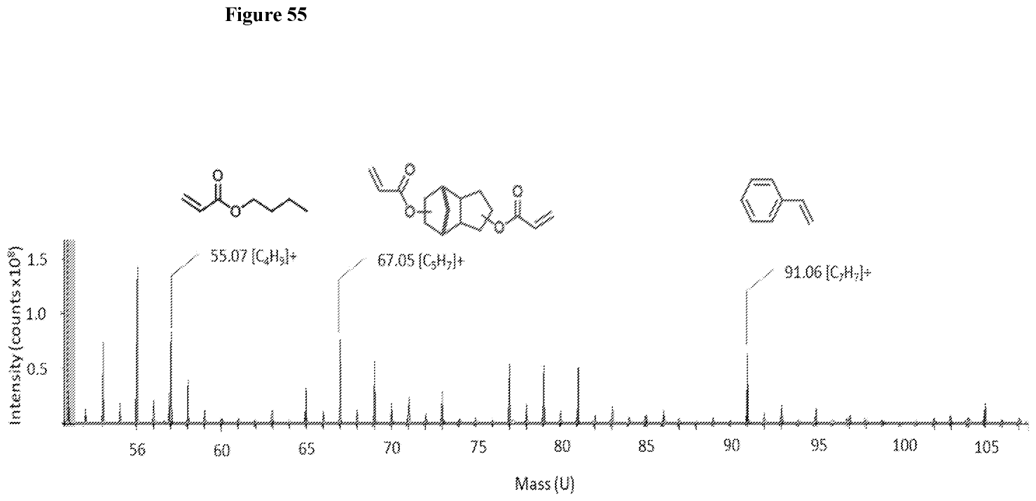

Scaling up of poly(TCDMDA-blend-BA) coated onto poly(styrene) six well-plates were transparent and surface chemistry analysed by TOF-SIMS after washing and sterilisation procedures showed the presence of peaks characteristic of each of the components TCDMDA (C₅H₇⁺ m/z=67.05) and BA(C₄H₉⁺ m/z=57.07) (FIG. 55). Atomic force microscopy revealed a nanoscale blend of poly-BA (minor component, 30% w/v as ˜50 nm islands) in poly-TCD (major component, 70% w/v), identifying that the poly(TCDMDA-blend-BA) coating exists as a mixture rather than a co-polymer (FIG. 53).

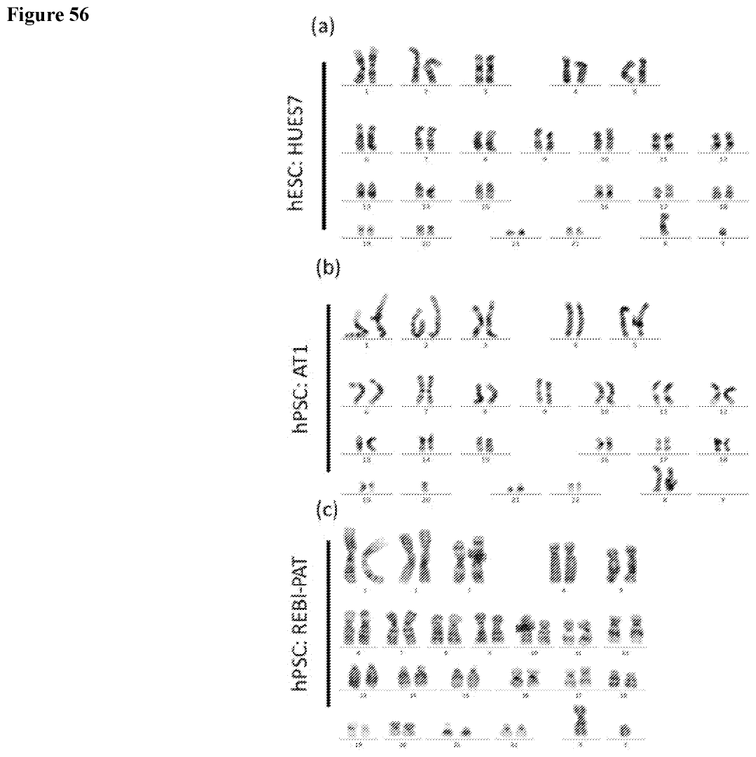

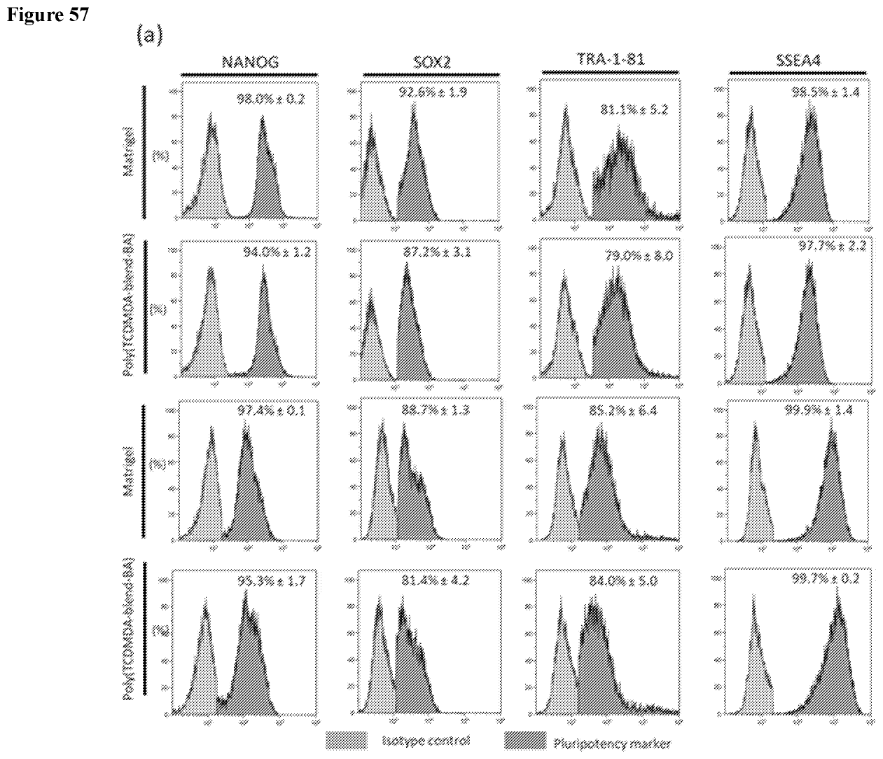

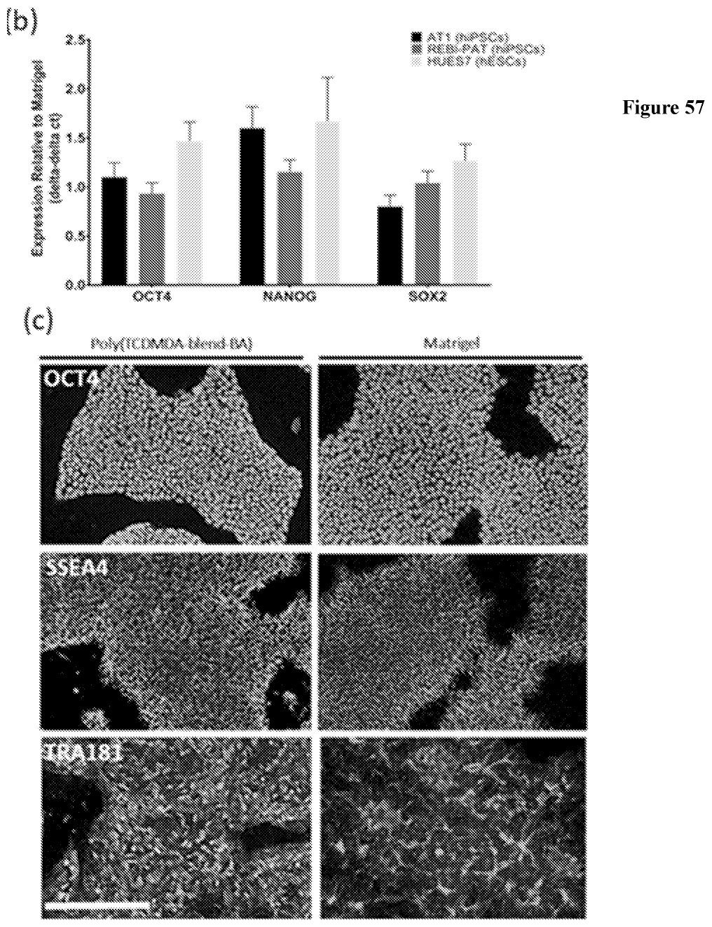

Antibiotic-free hPSC expansion with three independent hPSCs (hESC HUES7 and hiPSCs AT1 and REBI-PAT) for 8 serial passages, with each passage maintaining hPSCs to confluency at 72 hrs was successfully achieved on poly(TCDMDA-blend-BA) surfaces (FIG. 53c-d). HPSC growth, assessed by cumulative population doubling on poly(TCDMDA-blend-BA) was up to 2 fold slower over the eight serial passages than Matrigel™ (FIG. 53d) which contains an number of xenogenic factors known to stimulate growth.¹⁰ HPSCs cultured on poly(TCDMDA-blend-BA) for at least 5 serial passages retained stable karyotype (46 XY HUES7, REBI-PAT 46 XY and AT1 46 XX; karyograms presented in FIG. 56) and maintained pluripotent marker expression of OCT4, NANOG, SOX2, TRA181 and SSEA4 confirmed by immunostaining (>80%), flow cytometry (>85%) and quantitative real-time PCR (FIG. 57).

Since hPSCs were demonstrated to retain normal phenotype on poly(TCDMDA-blend-BA); mechanistic studies were performed to investigate hPSC attachment and expansion in this fully defined culture system. Integrins important for initial hPSC attachment were identified with antibodies blocking key integrins for the initial 24 hrs post-seeding (FIG. 53d). hPSC attachment was significantly reduced by blocking of β₁ (HUES7: p<0.01; AT1: p<0.05), αᵥβ₃ (AT1, p<0.01) and αᵥβ₅ (p<0.0001). hPSC attachment was more dramatically reduced with αᵥβ₃− (p<0.0001) and αᵥβ₅− (p<0.0001) RGD blocking peptides (c(RGDfV), and (c(RGDfC), compared to matrigel and their controls c(RADfV) and c(RADfC). Overall, β1 and αv containing integrins which are known to bind to laminin and vitronectin sites individually and are likely to interact in a complex manner to mediate hPSC attachment to sites present on D:Q surfaces; consistent with previously reported hPSC poly (HPhMA-co-HEMA) chemistry.⁶

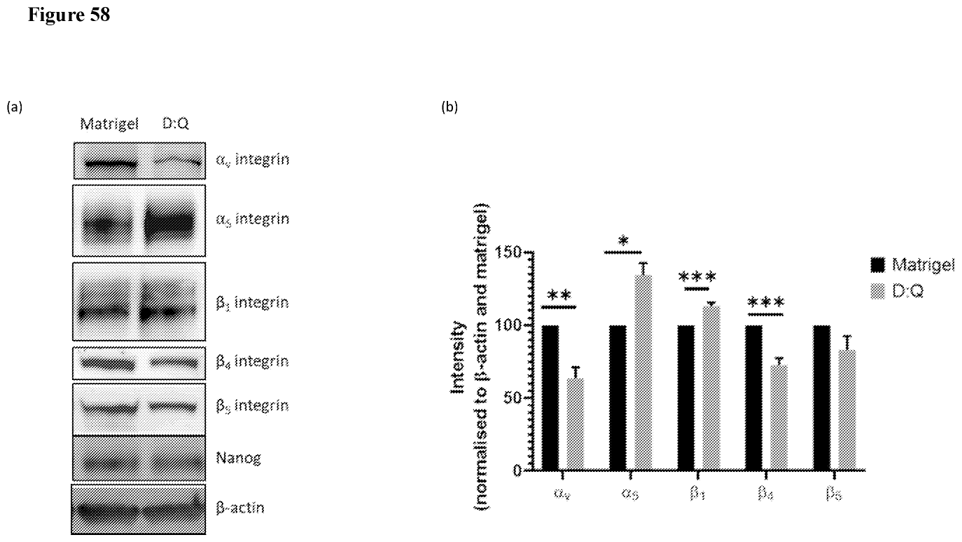

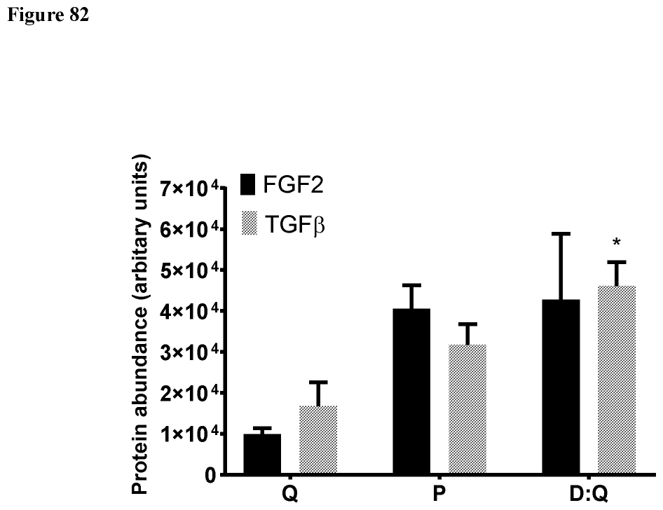

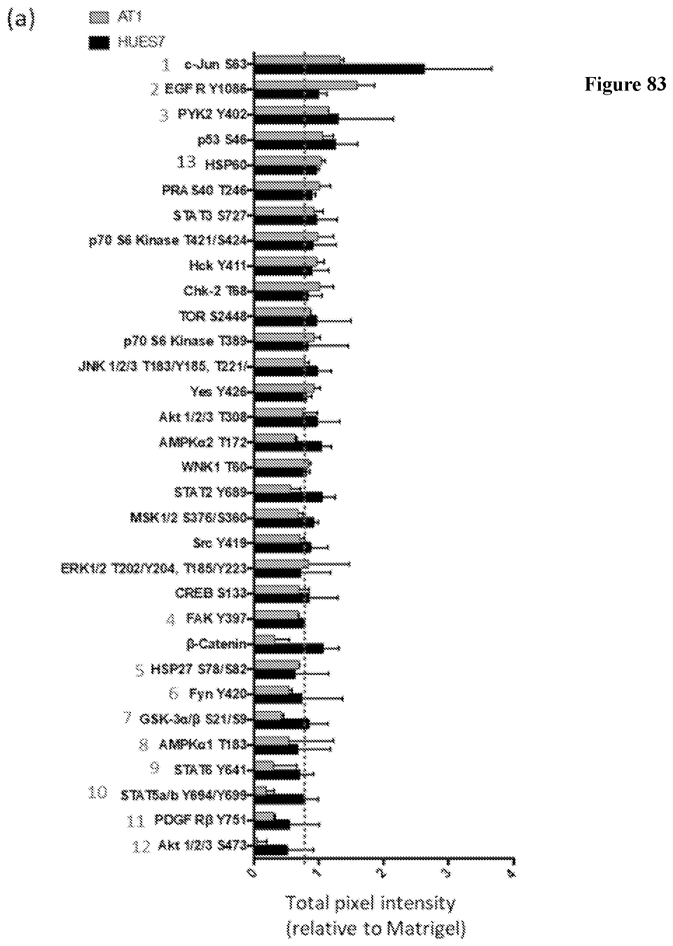

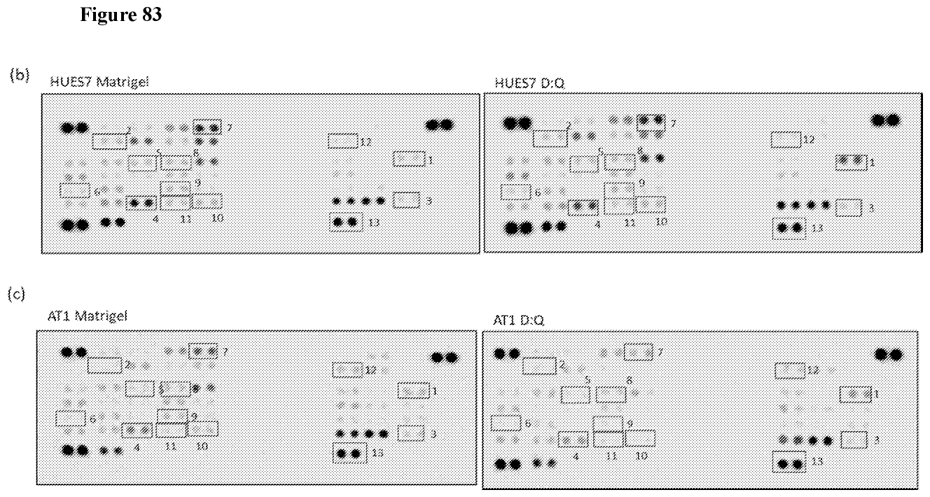

Integrins can also mediate attachment by binding to sites present from proteins adsorbed from culture medium.¹⁻¹² Proteins adsorbed from E8 medium FGF2 and TGF-B (factors required for maintained hPSC pluripotency)⁷ were assessed by liquid extraction surface analysis-tandem mass spectrometry (LESA-MS/MS) on low attachment polyBA (minor component of poly(TCDMDA-blend-BA), THFuA (P) which maintained attachment from 1^{st} generation array and scaled-up poly(TCDMDA-blend-BA). FGF2 and TGFβ (p<0.05) levels were higher than polyBA and equivalent to polyTHFuA (FIG. 82). These proteins have been reported to play a significant role in regulating expression of α5 and β1 integrins both of which were elevated on poly (TCDMDA-blend-BA) compared to matrigel when assessed by western blot (FIG. 58).¹³⁻¹⁵ These results corroborate with activated signalling of MAPK/ERK and JNK pathways responsible for hPSC proliferation, identified from phosphokinase array assay (R&D) where observed elevated levels of downstream markers c-Jun (S63), EGFR (Y1086) and PYK2 (Y402) were identified (FIG. 83).¹⁶⁻¹⁸ These signalling profiles are representative of hPSCs grown on established ECM substrates but also offers the opportunity to identify key signalling mechanisms without contamination of xenogenic factors.

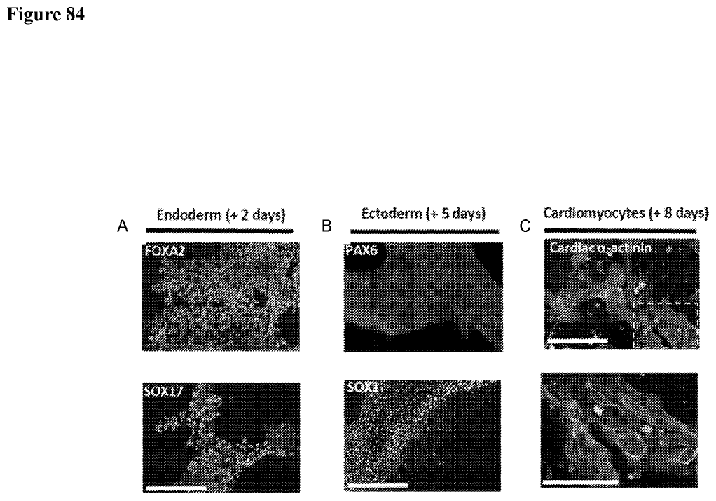

Differentiation capacity holds great importance for hPSCs research and has opened up huge possibilities for disease modelling. To ensure that hPSCs maintained their differentiation capacity after serial passaging on poly(TCDMDA-blend-BA), directed differentiation protocols towards the three germ layers were performed. Definitive endoderm SOX17 and FOXA2 positive cells were achieved after two days of WNT pathway activation with the GSK-3 inhibitor CH199021 (FIG. 84E). hPSC differentiation to SOX1 and PAX6 positive neural progenitors of the ectoderm lineage was achieved with modulators of the transforming growth factor beta (TGF-β) superfamily (dual SMAD inhibitors dorsomorphin and SB431542) and WNT (XAV393) pathways (FIG. 84f). Functional contractile cardiomyoytes (mesoderm) cells were formed using modulators of the TGF-β (activing A and BMP4) and WNT (KY02111 and XAV393) pathways for 8-12 days (FIG. 84g).

In summary, a high throughput combinatorial approach was used to identify a synthetic polymer substrate for xeno-free expansion of hPSCs. To our knowledge, this is the first defined synthetic culture system for long-term hPSC culture in the fully-defined E8 medium without the addition of biological substrates. Stability of hPSCs cells was confirmed by maintenance of key integrins for attachment and modulators of important signalling pathways after serial passaging. Directed differentiation to each of the three germ layers including functional cardiomyocytes confirmed the potential for utilising these hPSCs for disease modelling applications. Poly(TCDMDA-blend-BA) is amenable for scaling up to tissue culture plasticware. Overall, this system has great potential to provide an attractive, more cost-effective alternative to current commercially available synthetic substrates (eg. Synthemax) for industrial scale production of current good manufacturing practices (cGMP) complaint hPSCs that could be used for regenerative medicine applications and therapies.

### Methods

Polymer microarray synthesis and preparation: Polymer microarrays were fabricated using methods previously described.¹² Briefly, polymer microarrays were printed onto polyHEMA (4% w/v Sigma, in ethanol (95% v/v in water)) dip coated glass slides using a XYZ3200 dispensing station (Biodot) and quilled metal pins (946MP6B, Arrayit) under an argon atmosphere maintaining O₂<2000 ppm, 25° C. and 35% humidity. Polymerization solutions consisted of polymer (50% v/v) in dimethylformamide with photoinitiator 2,2-dimethoxy-2-phenyl acetophenone (1% w/v). Three replicates of 284 monomers were printed on each slide for the first generation array. For the second generation array, the polymer portion of the polymerisation solution consisted of major and minor monomers in a 70/30% v/v ratio. Three replicates of 23 monomers and subsequent 486 co-polymers combinations were printed. Monomers were purchased from Sigma, Scientific Polymers and Polysciences. Top and bottom array surfaces were sterilised with UV light for 15 minutes and washed with sterile Ca²⁺/Mg²⁺-free Phosphate Buffer Saline (PBS, Gibco) before culturing with hPSCs.

Cell culture: Three hPSC lines used in this study, including the hESC line, HUES7 and the hiPSC cell lines: ReBI-PAT derived from a skin punch biopsy from a male subject and AT1 derived from dental pulp of a female subject, as previously described³ were routinely maintained on 1:100 Matrigel (BD Biosciences, UK) in Essential 8 medium (LifeTechnologies). Cells were passaged at 70-80% confluency by washing once PBS, followed by incubation with TrypLE Select (LifeTechnologies) for 3 minutes at 37° C., with tapping of flasks to dissociate cells.

Microarray screening: 0.75×10⁶ REBI-Pat cells were seeded in E8 medium supplemented with 10 μM Y-27632 (ROCKi, Tocris Bioscience) on each array and incubated at 37° C. with 5% CO₂ for up to 48 hrs. Array samples used for quantification were fixed with 4% paraformaldehyde at 24 hrs, immunostained for OCT4 expression (described below) and mounted with Vectashield Antifade mounting medium (Vector Laboratories, imaged using automated fluorescence microscopy (IMSTAR) and analysed using CellProfiler ver. 2.2.0 (Broad Institute) image analysis software.

Time-of-flight secondary-ion mass spectrometry surface analysis: Measurements were taken using a TOF-SIMS 4 (IONTOF GmbH) instrument using a 25 kV Bi3⁺⁺ primary ion source with a pulsed target current of ˜1 pA as previously described.²

Atomic Force Microscopy (AFM): Hydrated AFM measurements were acquired using a Bruker Dimension FastScan in PeakForce™ mode using SCANASYST-FLUID+ probes. Samples assessed for surface analysis were incubated in ultrapure MilliQ water (18.2 Ohm) and the probes were calibrated using a 2.6 GPa Bruker polystyrene film sample.

hPSC culture on scaled up surfaces: Polymerisation solutions (consisting of monomers alone or two monomers mixed at 70/30% w/v in dimethylformamide with photoinitiator 2,2-dimethoxy-2-phenyl acetophenone (1% w/v) prepared in isopropanol) were coated onto oxygen plasma treated (pᵢ=0.7 mbar, 100 W for 10 minutes) tissue culture plastic well-plates and UV polymerised (365 nm) for 1 hr. Polymerization solutions for coatings containing the monomer 4-Methacryloxyethyl trimellitic anhydride (MAETA) were re-optimised at scale-up and were subsequently prepared in methanol (25% w/v) with applied heat for protein adsorption experiments. Well-plates were washed three times with isopropanol to remove unreacted polymer, washed in dH₂O for 48 hrs at 37° C. Well-plates were subsequently sterilized with 70% IMS and washed three times with sterile PBS. hPSCs were seeded at 7×10⁴ cells/cm² in E8 medium supplemented with 10 μM Y-27632 dihydrochloride for the initial 24 hrs of culture. Medium was exchanged every 24 hrs until cells reached 70-80% confluency at 72 hrs when cells were fixed or passaged by dissociating with TryPLE select (as described above). After 5 serial passages HPSC were karyotyped as previously described.³

Protein adsorption analysis of polymers coated in well plates: Sterilized and washed polymer coated plates were incubated in E8 medium supplemented with 10 μM Y-27632 dihydrochloride for 1 hr at 37° C. Plates were washed with dH₂O (18.2 MΩ, ElgaPure LabWater). Proteins were digested in-situ using microwave-assisted techniques using 0.05 μg/ml trypsin (sequencing grade; Promega, UK) in acetic acid with 100 mM ammonium bicarbonate (BioUltra, ≥99.5%, Sigma-Aldrich) adapted from previously described methods.⁴ Standard methods were used to extract proteins using an extraction solution consisting of acetonitrile (CHROMASOLV®, Riedel-de Haen) and 200 mM ammonium acetate (≥99.0%; Sigma-Aldrich, Gillingham, UK) (1:9 v/v) in LC-MS grade water (CHROMASOLV®, Riedel-de Haen). Samples were analysed by liquid extraction surface analysis-mass spectrometry (LESA-MS) and introduced to a TriVersa Nanomate (Advion Biosciences, Ithaca, NY) coupled to a Q Exactive plus mass spectrometer (Thermo, San Jose, CA) via nanoelectrospray ionisation (ESI Chip™, Advion Biosciences) using 1.6 kV voltage and 0.6 psi gas pressure (N₂).

Growth-rate assessment: hPSCs growth was assessed using an automated cell-viability counter (CEDEX Hi Res Analyser) at each passage (every 72 hrs). Doubling time (www.aloc.org; [duration of culture×log₂]/[log₁₀ (final cell concentration/seeding concentration)] was calculated for hPSCs and was plotted cumulatively.

Immunostaining: Adherent cells were fixed in 4% paraformaldehyde (Sigma-Aldrich, UK) at room temperature (RT) for 20 minutes and permeabilized with 0.1% Triton-×100 (Sigma-Aldrich, UK) in PBS at RT for 15 minutes. Non-specific binding was blocked with 4% serum (Sigma-Aldrich, UK) in PBS at RT for 1 hour. Samples were incubated overnight at 4° C. with primary antibodies OCT4 (1:200, Santa Cruz Biotechnology, SC-5279), TRA181 (1:200, Millipore, MAB4381), SSEA4 (1:100, Millipore), FOXA2 (1:500, Sigma-Aldrich 07-633), SOX17 (1:100, R&D AF1924), SOX1 (1:100, R&D AF3369), PAX6 (1:100, R&D AF8150) and cardiac α-actinin (1:800, Sigma-Aldrich A7811) diluted in blocking solution with the addition of 0.1% Triton X-100 for nuclear stains. Samples were washed with 0.1% Tween-20 (Sigma-Aldrich, UK) and incubated with Alexa Fluor secondary antibodies (Life Technologies) 1:400 in blocking solution for 1 hr at RT in the dark. Cells were washed with 0.1% Tween-20 and nuclei were counterstained with 0.5 μg/ml DAPI (4′,6-diamidino-2-phenylindole, Sigma-Aldrich D9542).

Polymer optimisation in 96-well plates: ReBI-PAT hPSCs were seeded at 4.5×10⁴ cells/cm² on co-polymers selected for scale-up in E8 medium supplemented with Y-27632 where each co-polymer was tested in triplicate wells. Matrigel controls were also included for comparison. Images of five separate fields were obtained per well (n=3 independent repeat) using the Operetta high-content imaging system (Perkin Elmer). Images were analysed using Harmony high-content image analysis software (Perkin Elmer) developed with PhenoLOGIC machine learning algorithms to quantify percentage cell coverage (relative to total areas imaged per well) and mean area of colonies (total cell coverage/no. of colonies). Adhered cells at 72 hrs were fixed in 4% paraformaldehyde and immunostained for OCT4 and fluorescence microscopy using the Operetta and Harmony was used to quantify total and OCT4+ nuclei (5 fields/well).

Flow cytometry: hPSCs serially passaged on polymer substrate (≥3 passages) were dissociated into single-cell suspension and fixed with 4% paraformaldehyde. Samples were permeabilized with 0.1% Tween-20 in PBS for intracellular markers and incubated with primary antibodies NANOG (1:100, APCH7 conjugated, BD Biosciences, 560109), SOX2 (1:20, Alexa Fluor 647-conjugated, R&D Systems, IC2018R), TRA181 (1:100, PE-conjugated, Invitrogen, 12-8883-82) and SSEA4 (1:20, fluorescein-conjugated, R&D Systems, FAB1435F) diluted in PBS for 1 hr at RT. The FC500 flow cytometer (Beckman Coulter) was used to acquire measurements and expression was quantified with Kaluza analysis software (Beckman Coulter).

Attachment blocking: hPSCs were harvested and re-seeded in E8 medium with the addition of integrin blocking antibodies (10 μg/ml for each antibody) or RGD-blocking peptides (15 μg/ml) for 24 hrs. Cells were washed three times with PBS, fixed with 4% paraformaldehyde and counterstained with DAPI. Fluorescence images acquired using the Operetta (Perkin Elmer) were quantified for total nuclei count per condition in Harmony image analysis software (Perkin Elmer).

Integrin expression by Western Blot: hPSCs serially passaged on polymer (≥3 passages) were lysed using RIPA buffer (Cell Signalling Technologies #9806) supplemented with PMSF (Phenylmethylsulfonyl fluoride, Sigma 10837091001). Total lysate protein was determined using Pierce BCA Protein Assay Kit (Thermo Fisher Scientific #23225) following manufacturer's instructions. LDS NuPAGE Sample Buffer (4×) with 2.5% 2-mercaptoethanol was added to 30 μg of protein lysate and run on NuPAGE NOVEX Bis-Tris Gels with MOPS SDS Running Buffer (Thermo Fisher Scientific #NP0008, #NP0001). Samples were transferred to an Amersham Protran 0.45m nitrocellulose blotting membrane (GE Healthcare Life Science #10600124). Membranes were then stained with primary and secondary antibodies listed within supplementary materials*. Membranes were developed using West Pico PLUS Chemiluminescent Substrate (Thermo Fisher Scientific #34577) on an LAS-400 Imaging system.

Proteome Profiler Array: Human Phospho-Kinase Array (R&D systems, ARY003B) was performed according to manufacturer's instructions (www.rndsystems.com) on hPSCs serially passaged on polymer and Matrigel™ in parallel (3 passages). Array blots were imaged using ImageQuant LAS-4000 (Fujitsu Life Sciences) and analysed using Image Studio Software (LI-COR, version 5.2.5) where individual total signal intensity was measured by manual gating. All intensity values were normalized to background intensity and HSP60 internal control. Changes were quantified by comparison between Matrigel™ and polymer conditions.

Tri-lineage differentiation: hPSCs serially passaged (3 passages) were harvested and seeded at 2×10⁴-1×10⁵ cell/cm² and expanded in E8 medium for 2 days with daily media exchanges. All directed differentiation protocols were performed on hPSCs at day 2. For definitive endoderm differentiation, media was replaced by RPMI supplemented with B27 without insulin (LifeTechnologies 0080085-SA) and CHIR99021 (2 μM; STEMCELL Technologies, 72052) for a further 2 days with daily media exchanges. To produce neural progenitors of the ectoderm lineage, media was replaced by Advanced DMEM/F-12 (LifeTechnologies) supplemented with 1% L-glutamine (Life Technologies), 1% CD Lipid Concentrate (Life Technologies) 7.5 μg/ml Transferrin (Sigma-Aldrich), 14 μg/ml Insulin (Sigma Aldrich), 0.1 mM β-mercapto-ethanol, 10 μM SB431542 (Tocris) and 1 μM Dorsomorphin-1 (Tocris) and 2 μM XAV939 (STEMCELL Technologies) for 5 days with daily media exchanges. Differentiation to cardiomyocytes was achieved using methods previously described.³

Statistical tests: Experiments were performed in at least three independent experiments unless otherwise stated. Statistical tests (as stated in text) were performed using GraphPad Prism (version 8.1.2, San Diego CA).

### REFERENCES

1. Celiz, A. D.; Smith, J. G.; Langer, R.; Anderson, D. G.; Winkler, D. A.; Barrett, D. A.; Davies, M. C.; Young, L. E.; Denning, C.; Alexander, M. R., Materials for stem cell factories of the future. Nat Mater 2014, 13 (6), 570-9.
2. Smith, J. G.; Celiz, A. D.; Patel, A. K.; Short, R. D.; Alexander, M. R.; Denning, C., Scaling human pluripotent stem cell expansion and differentiation: are cell factories becoming a reality? Regen Med 2015, 10 (8), 925-30.
3. Jin, S.; Yao, H.; Weber, J. L.; Melkoumian, Z. K.; Ye, K., A synthetic, xeno-free peptide surface for expansion and directed differentiation of human induced pluripotent stem cells. Plos One 2012, 7 (11), e50880.
4. Mei, Y.; Gerecht, S.; Taylor, M.; Urquhart, A. J.; Bogatyrev, S. R.; Cho, S. W.; Davies, M. C.; Alexander, M. R.; Langer, R. S.; Anderson, D. G., Mapping the Interactions among Biomaterials, Adsorbed Proteins, and Human Embryonic Stem Cells. Adv Mater 2009, 21 (27), 2781-2786.
5. Hammad, M.; Rao, W.; Smith, J. G.; Anderson, D. G.; Langer, R.; Young, L. E.; Barrett, D. A.; Davies, M. C.; Denning, C.; Alexander, M. R., Identification of polymer surface adsorbed proteins implicated in pluripotent human embryonic stem cell expansion. Biomater Sci 2016, 4 (9), 1381-91.
6. Celiz, A. D.; Smith, J. G.; Patel, A. K.; Hook, A. L.; Rajamohan, D.; George, V. T.; Flatt, L.; Patel, M. J.; Epa, V. C.; Singh, T.; Langer, R.; Anderson, D. G.; Allen, N. D.; Hay, D. C.; Winkler, D. A.; Barrett, D. A.; Davies, M. C.; Young, L. E.; Denning, C.; Alexander, M. R., Discovery of a Novel Polymer for Human Pluripotent Stem Cell Expansion and Multilineage Differentiation. Adv Mater 2015, 27 (27), 4006-12.
7. Chen, G.; Gulbranson, D. R.; Hou, Z.; Bolin, J. M.; Ruotti, V.; Probasco, M. D.; Smuga-Otto, K.; Howden, S. E.; Diol, N. R.; Propson, N. E.; Wagner, R.; Lee, G. O.; Antosiewicz-Bourget, J.; Teng, J. M. C.; Thomson, J. A., Chemically defined conditions for human iPS cell derivation and culture. Nat Methods 2011, 8 (5), 424-9.
8. Hook, A. L.; Anderson, D. G.; Langer, R.; Williams, P.; Davies, M. C.; Alexander, M. R., High throughput methods applied in biomaterial development and discovery. Biomaterials 2010, 31 (2), 187-98.
9. Celiz, A. D.; Smith, J. G. W.; Patel, A. K.; Langer, R.; Anderson, D. G.; Barrett, D. A.; Young, L. E.; Davies, M. C.; Denning, C.; Alexander, M. R., Chemically diverse polymer microarrays and high throughput surface characterisation: a method for discovery of materials for stem cell culture. 2014.
10. Hughes, C. S.; Postovit, L. M.; Lajoie, G. A., Matrigel: a complex protein mixture required for optimal growth of cell culture. Proteomics 2010, 10 (9), 1886-90.
11. Braam, S. R.; Zeinstra, L.; Litjens, S.; Ward-van Oostwaard, D.; van den Brink, S.; van Laake, L.; Lebrin, F.; Kats, P.; Hochstenbach, R.; Passier, R.; Sonnenberg, A.; Mummery, C. L., Recombinant vitronectin is a functionally defined substrate that supports human embryonic stem cell self-renewal via alphavbeta5 integrin. Stem Cells 2008, 26 (9), 2257-65.
12. Irwin, E. F.; Gupta, R.; Dashti, D. C.; Healy, K. E., Engineered Polymer-Media Interfaces for the Long-term Self-renewal of Human Embryonic Stem Cells. Biomaterials 2011, 32 (29), 6912-9.
13. Zambruno, G.; Marchisio, P. C.; Marconi, A.; Vaschieri, C.; Melchiori, A.; Giannetti, A.; De Luca, M., Transforming growth factor-beta 1 modulates beta 1 and beta 5 integrin receptors and induces the de novo expression of the alpha v beta 6 heterodimer in normal human keratinocytes: implications for wound healing. J Cell Biol 1995, 129 (3), 853-65.
14. Yamasaki, S.; Taguchi, Y.; Shimamoto, A.; Mukasa, H.; Tahara, H.; Okamoto, T., Generation of Human Induced Pluripotent Stem (iPS) Cells in Serum- and Feeder-Free Defined Culture and TGF-β1 Regulation of Pluripotency. In Plos One, 2014; Vol. 9.
15. Collo, G.; Pepper, M. S., Endothelial cell integrin alpha5beta1 expression is modulated by cytokines and during migration in vitro. J Cell Sci 1999, 112 (Pt 4), 569-78.
16. Haghighi, F.; Dahlmann, J.; Nakhaei-Rad, S.; Lang, A.; Kutschka, I.; Zenker, M.; Kensah, G.; Piekorz, R. P.; Ahmadian, M. R., bFGF-mediated pluripotency maintenance in human induced pluripotent stem cells is associated with NRAS-MAPK signaling. In Cell Commun Signal, 2018; Vol. 16.
17. Hortala, M.; Estival, A.; Pradayrol, L.; Susini, C.; Clemente, F., Identification of c-Jun as a critical mediator for the intracrine 24 kDa FGF-2 isoform-induced cell proliferation. Int J Cancer 2005, 114 (6), 863-9.
18. Lin, A. H.; Eliceiri, B. P.; Levin, E. G., FAK Mediates the Inhibition of Glioma Cell Migration by Truncated 24 kDa FGF-2. Biochem Biophys Res Commun 2009, 382 (3), 503-7.

### Example 7—Identification of Immune-Instructive Polymers in a Methacrylate and Acrylate Library: Bioengineering of Dendritic Cell Phenotype and Function

Biomaterial-based immunotherapies have recently emerged as new efficient methods to treat illnesses and modulate human immune responses in situ, without the need for ex vivo cell manipulation, while also providing the opportunity to not add external stimulants such as cytokines. Usually these biomaterial-based immunotherapies incorporate loading or co-delivery with a cytokine or other immune modulatory agents [1], [2]. The central role of dendritic cells (DCs) in orchestrating adaptive immune responses has made them the target of choice for many immunotherapy interventions [3]-[9]. DCs act as the bridge between the innate and the adaptive arms of the immune system with an integral role in the regulation of responses to foreign material while maintaining peripheral tolerance [10]-[12]. In the process of DC-based immune responses, immature DCs move into the infection or injury site where they assess the nature of tissue damage or infection. Due to their vast repertoire of pattern recognition receptors (PRRs), DCs are capable of recognizing pathogens and cellular changes associated with cellular stress and tissue damage, generally referred to as “pathogen/damage-associated molecular patterns” (PAMPs or DAMPs) [13]. These pathogens are internalised, processed and their antigens are presented in the context of major histocompatibility complexes class I or II (MHC I and II) depending on the source of antigens [14]. Once activated in peripheral tissues, DCs migrate along a chemokine gradient of chemokine ligands (CCL) 19/21 via the lymph stream to the lymph nodes, where they prime naïve T cells leading to clonal expansion and differentiation of specific T cells [13]. Depending on the nature of antigen they present, and the co-stimulatory signals and cytokines they provide, DCs are able to polarize naïve T cells into Th1, Th2, Th17, Treg T helper cells or cytotoxic T lymphocytes (CTLs) [15]. During steady-state conditions, DCs maintain immune tolerance against self-antigens; this important function makes them crucial for maintaining peripheral tolerance which is often compromised in autoimmune diseases [16], [17].

In DC-based immunotherapies against cancer, DCs are usually isolated from the patient and are treated with tumour antigen and other activating agents ex vivo to be later transferred back into the patient, with the goal of inducing strong antigen-specific anti-tumour immune responses. In the past, this has proven to be successful, albeit the clinical efficacy is low due to decreased DC persistence and their poor functionality [4], [6], [8]. Recent development around in vivo DC modulation has been made by Mooney et al., using PLG scaffolds that are loaded with GM-CSF and tumour antigens to recruit DCs [18]. In another study they prepared crosslinked methacrylated PEG and methacrylated alginate into macroporous scaffolds, which were then loaded with GM-CSF, CpG-ODN (a toll-like receptor (TLR) ligand), and tumour antigens [19]. This approach of in vivo DC modulation is thought to improve poor functionality and the low persistence of DCs.

The type and magnitude of immune responses is influenced in part by the level of DC activation, where a “mature” DC phenotype typically supports a pro-inflammatory reaction and, conversely, an “immature” phenotype induces anergy or a ‘regulatory’ immune response [20], [21]. Modulating DC phenotype ex vivo, using adjuvants, cytokines or antibodies, followed by adoptive transfer of cells to patients, has been tried with various degree of success. Some of the major disadvantages include 1) cost, 2) complexity, 3) and induced low clinical efficacy due to low number of cells [4], [6], [8]. While cost and complexity are issues that also apply for many other therapeutic interventions, the main disadvantage (on the clinical side) is the low functionality of cells. DCs that have been modulated ex vivo encounter a different microenvironment (such as different immune cells and the cytokines, chemokines they are producing in response to the situation on hand) after transfer into the patient, which can ultimately undo any desired phenotype modulations [22], [23]. An alternative for these ex vivo cell manipulations are biomaterials; they can be used to control cell behaviour directly in vivo, without the need for costly and complex ex vivo modulation, which represents a significant advantage over cell therapies. DCs instruct immune responses via sensing of their environment; therefore, to modulate the immune response in situ, ‘immune-instructive’ biomaterials can be a powerful tool to locally direct DC function, and therefore, the immune response.

Modulation of the immune system is challenging, even with extensive immune-bioengineering research done in the last decades. Materials, that have been found to modulate macrophage phenotype and function have been reported intensively [24]-[29], however modulations of DCs and their effects into the adaptive immune response using biomaterials has been less thoroughly investigated. Some routinely used biomaterials such as chitosan, agarose, titanium have been observed to affect DC phenotype activation levels [30]-[33]. Recent in-vitro studies have shown that some polymers are able to induce upregulation of surface molecules and the secretion of pro-inflammatory cytokines in murine and human DCs [34], [35]. In addition, poly(lactic-co-glycolic acid) has been shown to support DC maturation, while agarose has been shown to supress it [24], [32]-[34], [36], [37]. A study by Shokouhi et al., proposed that DC function altered by biomaterials, can directly affect the immune response at the implantation site [38]. Therefore, it is hypothesized that the effect of biomaterials on DC phenotype may influence the adaptive immunity against a co-delivered antigen.

The knowledge of DC-biomaterial interactions, mechanisms and their consequences is still in its infancy, making rationale design of materials with the ability to influence different aspects of DC function rather challenging. Nevertheless, the concept of developing immune-instructive materials with the ability to promote DCs to distinct pro- or anti-inflammatory responses, is very attractive with potential applications as vaccine adjuvants to enhance or maximize a protective immunity or suppress deleterious immune response in inflammatory diseases. There is evidence suggesting that DCs are able to detect and interact with biomaterials specifically via toll-like-receptors 2, 4 and 6, β-integrins or “biomaterial-associated molecular patterns” (BAMPs) [36], [39], [40], however the exact mechanisms and pathways underpinning DCs interaction with different biomaterials are yet to be elucidated.

Polymers have been found to be tuneable in their physical, chemical and biological properties making them attractive candidates for developing bio-instructive materials [41]-[43]. In this study a library of 222 commercially available meth(acrylamides) and meth(acrylates) was used to identify polymers that have modulatory effects on DCs. High throughput screening has previously shown to be effective in the identification of new polymers [29], [44]-[46]. These homo-polymers were assessed in a series of in vitro experiments to establish their ability to modulate DCs viability, phenotype and cytokine profile and subsequent modulation of the adaptive immune response (as well as effect on anti-tumour activity). lysate.

### Methods

### Monocyte-Derived Dendritic Cell Differentiation

DCs were generated as described previously[47]. Buffy coats were obtained from healthy donors (National Blood Service, Sheffield, UK) after obtaining informed written consent and following ethics committee approval (Research Ethics Committee, Faculty of Medicine and Health Sciences, University of Nottingham). Peripheral blood mononuclear cells (PBMCs) were obtained from buffy coats by Histopaque-1077 (Sigma-Aldrich) density gradient centrifugation. Monocytes were isolated from PBMCs using the MACS magnetic cell separation system (positive selection with CD14 MicroBeads and LS columns, Miltenyi Biotec, Bergisch-Gladbach, Germany) as described before [48], [49]. Purified monocytes were suspended in RPMI-1640 medium supplemented with 10% FBS, 2 mM L-glutamine, 100 U/mL penicillin, 100 μg/mL streptomycin (“complete media”) and for differentiation into DCs further supplemented with 50 ng/mL of GM-CSF and 250 U/mL of IL-4 (R&D Systems, Oxford, UK) in 24-well plates for 6 days.

### Polymerisation of Monomer Library

Polymer coatings were generated as described previously [29]. Briefly, polymerisation solution containing monomers mixed with 1% (w/v) photo-initiator was dispensed into 24- or 96-well propylene plates. Plates were then put under UV light at a wavelength of 265 nm for 1 hour in the presence of Argon (02 concentration below 2%/2000 ppm). Remaining volatile components and residual monomers were removed at <50 mTorr for 72 h, followed by 3× isopropanol rinsing and then dH2O was added and plates were incubated for 72 hours (37° C. and 5% CO2). The polymer surfaces were dried, UV sterilized for 20 min (265 nm) and incubated with complete media overnight.

### Viability Assay

DCs were seeded onto the polymers after being fully differentiated. DCs were seeded in a concentration of 5×105 cells/well in duplicate and incubated at 37° C. and 5% CO2 for 6 hours. Cell viability was measured using CytoToxGlo (Promega) following the manufacturer's instructions. All cytotoxicity data is normalised to respective cell number per well. In order to present viability data for each cytotoxicity data point, the corresponding viability was calculated.

### Culture of Immature DC on Polymer Coated Surfaces

After preparing the polymer plates for cell culture as previously described, immature DC were harvested, washed once, re-suspended in fresh complete media and transferred to the coated wells. For investigating the effect of the polymers on immature DC, cells were cultured on polymers for 24 hours, while for polymer effect on DC maturation, DCs were conditioned for 6 hours on the polymers and then stimulated with 0.1 μg/mL lipopolysaccharide (LPS from E. coli, Sigma-Aldrich) and cultured for a further 18 hours. Cells were then used for the assays described below or analysed for expression of surface markers.

### Staining for Cell Surface Markers

Staining and acquisition were performed as described earlier [50]. Polymer-treated and control DCs were harvested, washed twice with PBS (supplemented with 0.5% BSA and 0.1% NaN3, later mentioned as PBA) and stained for CD83 (FITC, clone REA714, IgG1, Miltenyi Biotec) and CD86 (PE, clone REA968, IgG1, Miltenyi Biotec) for 20 minutes at 4° C. The cells were then washed with PBA and fixed in 1% paraformaldehyde in PBS. Cells were usually analysed within 24 hours. Flow cytometry acquisition was performed using a FACS Canto and 10000 events were collected for each sample. Dead cells were excluded by forward and side scatter using the Kaluza software.

### Cytokine Analysis

Culture supernatants from 3 independent experiments were collected after 24 hours culture. IL-6, IL-10, IL-12 and IFN-γ were measured by DuoSet ELISA kit (R&D Systems) as per the manufacturer's instructions. Modifications of the protocol allowed analysis of cytokines in a 384-well plate format, giving the opportunity to read duplicates from each donor.

### Endocytosis Assay

This assay was performed as described earlier [48]. DCs were cultured on scaled up polymers in 24-well plates and appropriate controls for 24 hours. DC were washed once with PBS, re-suspended in complete media with human AB serum and transferred into FACS tubes (4×105 cells in 400 μl). Dextran-FITC (40,000 kDa, Sigma) was added to a final concentration of 1 mg/mL and the tubes were incubated for 90 minutes at 37° C. or 4° C. Cells were harvested, washed twice with ice-cold PBS, then fixed in 1% paraformaldehyde in PBS to be immediately analysed by flow cytometry.

### DC-T Cell Co-Cultures

Autologous Pan T cells were separated by negative selection from the whole T cell fraction of PBMCs using a Pan T cell kit and CD45RO beads (Miltenyi Biotec) [50]. They were then co-cultured with Polymer-conditioned DCs in a 10:1 ratio and kept for 8 days in culture. Half of the media was exchanged on day 3 with fresh media containing IL-2. T cells stimulated with anti-CD3 and anti-CD28 on day 7 were used as positive control. BrdU colorimetric assay (Roche) was used to determine the proliferation rate of Pan T cell, and the supernatants were analysed in ELISAs to determine IFN-γ production/T cell activation.

### Tumour Killing Assay

For the tumour killing assays a adapted version of a previously described method was performed [51] Autologous naïve CD8 T cells and monocytes were isolated from bought PBMC vials. For this, DCs were cultured on flat polymer coatings for 24 hours, followed by addition of MCF7 (a breast cancer cell line) cells that have been rendered non-proliferative by mitomycin c (MMC) for another 24 hours. These DCs were then co-cultured with autologous naïve CD8 T lymphocytes for 8 days in a 1:10 ratios (DC: T cells). Every 3 days, 100 ul of cell culture media was removed and substituted with fresh media containing 200 IU/mL IL-2. Proliferated tumour specific CTLs were then seeded on MCF7 monolayers for 6 hours in differing ratios (1:5 to 1:20). Cells were then live/dead stained and imaged to calculate % of specific lysis.

### Statistical Analysis

All graphs and statistical analyses were carried out using Prism 8 (GraphPad software, Inc., USA). For statistical testing, student T-test or one-way-ANOVA with multiple comparisons with a Bonferroni post-tests were performed. P values. Supplementary

Cells were cultured on the polymers and subsequently subjected to a viability assay (CytoToxGlo, Promega). All data points were normalised against the tissue culture plastic control (TCP) which was set as 100%, and the overall ranked order of viability portrays a form of a slope, which plateaus shortly before the TCP control. The top 55% of viable polymers (120 out of 222) were chosen to take forward to the next screening steps. The rationale of choice here was to discard polymers that did not fall into the bracket of TCP viability—5×SD of TCP. The viability range of selected polymers is 93% to 116%—compared to TCP.

TABLE 13
Monomer concentration in DC polymer- well plate culture.
Analysis via mass spectrometry.

| Monomer | Actual concentration | | Actual Amount |
|---|---|---|---|
| DEAEA | 8.26E−02 | mM | 2.41313 × 10⁻¹⁴ g |
| pEGMEMA | 4.91E−02 | uM | 1.70514 × 10⁻¹⁴ g |
| ZnA | 9.26E−02 | uM | 2.24765 × 10⁻¹¹ g |

### Results

In order to examine DC viability on polymers, immature DCs were seeded in polymer coated 96 well plates for 6 hrs. This approach was taken in comparison to a polymer microarray screening format used to assess adherent cell responses because DCs are a non-adherend cell type and the array format could not be used. One disadvantage of this well-plate based approach, compared to micro array screening is that the polymers are far larger, and this was found to introduce cytotoxicity into a significant proportion of the samples fabricated (100 from 222) which were discarded from subsequent assessment.

### DCs are Activated by Polymer Coatings

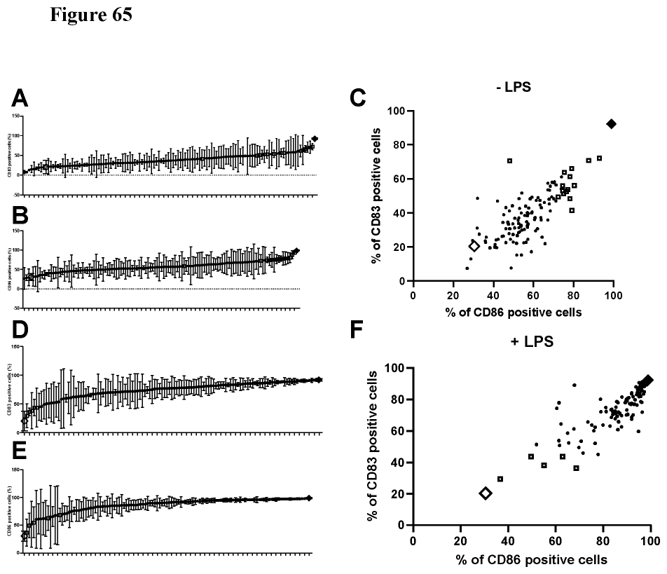

A total of 120 polymers were shown to maintain good cell viability, which were evaluated for their ability to either stimulate or suppress DC maturation. Immature DCs were seeded on polymer coated wells and cultured in a stationary dish within an incubator for 24 hours followed by assessment of DC surface phenotype using flow cytometry. To evaluate the activation level of polymer conditioned DCs, a number of cell surface markers associated with DC maturation were examined (FIG. 65). CD83 and CD86 are well documented DC activation markers: CD86 binds to CD28 and CTLA4 on T cells in order to provide a costimulatory signal to the T lymphocyte [52] and is essential in evoking an effector T cell response. CD83 is broadly used as a maturation marker however it is thought to act as a co-stimulatory receptor too. Although its exact function is not clear yet, it has been shown that downregulation of CD83 leads to less potent induction of allogeneic T cell proliferation and less priming potential of DCs in general [53].

In FIG. 65 a ranked order of the polymer-treated DCs for CD83 (65-A) and CD86 (65-B) expression, shows that most of the polymers had a stimulatory effect on both markers. Overall, the polymers induced higher expression of CD86 than CD83 (FIG. 65-C), but both markers correlate well with the other (R2=0.931). Seventeen polymers (later referred to as stimulatory polymers) consistently inducing upregulation of both CD83 and CD86 similar to levels achieved by LPS (FIG. 65-C) in all donors (more than 3×SD difference from the unstimulated TCP control) were chosen for further investigation.

To investigate whether DCs remain responsive to further stimulation after being conditioned on polymers, we also studied the activation level of these cells after first culturing them on the polymers followed by LPS stimulation. DCs conditioned with the majority of different polymers were still able to fully mature in response to LPS stimulation. A small number of polymers were able to prevent DC maturation even in the presence of a potent stimulus such as LPS as evidenced by low expression of both CD83 and CD86 (FIG. 65-D-F, later referred to as inhibitory polymers). From those also those with limited biological variance were chosen.

### Impact of Polymers on DC Cytokine Profile and Endocytic Ability

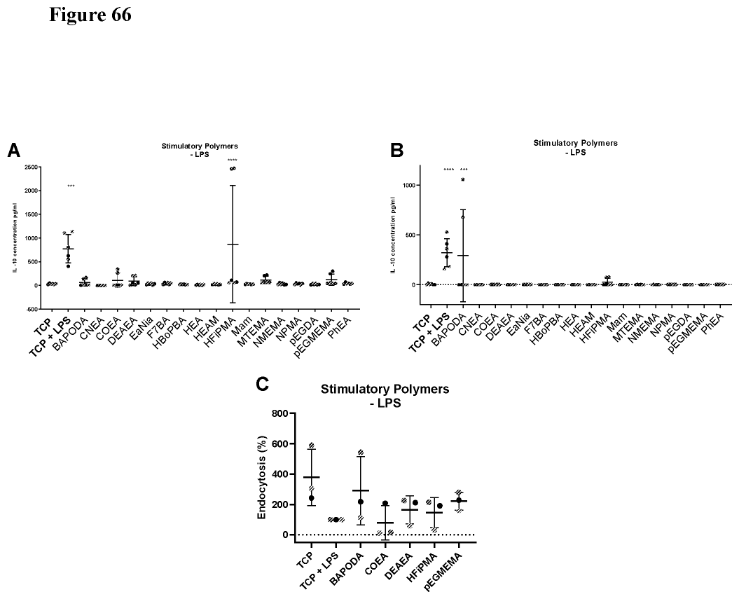

A selection of stimulatory and inhibitory polymers with more than 3×SD above/below the mean of TCP condition (without or with LPS stimulation) were selected for these functional assays. To understand the effect of polymers on DC functional phenotype we investigated how different polymers could change DC cytokine secretion, focusing on IL-12 and IL-10 as signature pro and anti-inflammatory cytokines, and their endocytic ability as two important determinants of DC induced T cell activation. IL-10 secretion was slightly elevated compared to the TCP control when DCs were cultured on several stimulatory polymers (BAPODA, DEAEA, COEA, EaNia, F7BA, HFiPMA, MTEMA, NMEMA, NPMA, pEGMEMA), whereas only two polymers (BAPODA, HFiPMA) increased IL-12 secretion (FIG. 66 A-B). One of the characteristic features of immature DCs is their high endocytic ability. After maturation, this capacity decreases, while simultaneously expressing antigen-presenting complexes (MHCs) on the cell surface, allowing DCs to present the antigens they have captured in the periphery to T cells. To simulate uptake of soluble antigens, we analysed dextran-FITC uptake by DCs after treatment with selected polymers. Due to limited ability to perform multiple endocytosis in one run, 5 stimulatory polymers were chosen from the stimulatory polymers (the ones that showed higher cytokines secretion than TCP), while all 5 inhibitory polymers were taken into the endocytosis assay. From the stimulatory polymers BAPODA, COEA, DEAEA, HFiPMA and DEAEA were chosen. Selection criteria were modulation of cytokine secretion, while also keeping the phenotype modulation data in mind to choose the strongest stimulators of DC activity.

COEA decreased uptake ability the most of all polymers, with DEAEA and HFiPMA intermediately decreased and pEGMEMA showing similar uptake ability to TCP cultured DCs. BAPODA showed a high variability between donors and their uptake ability was between stimulated and non-stimulated DCs (FIG. 66-C).

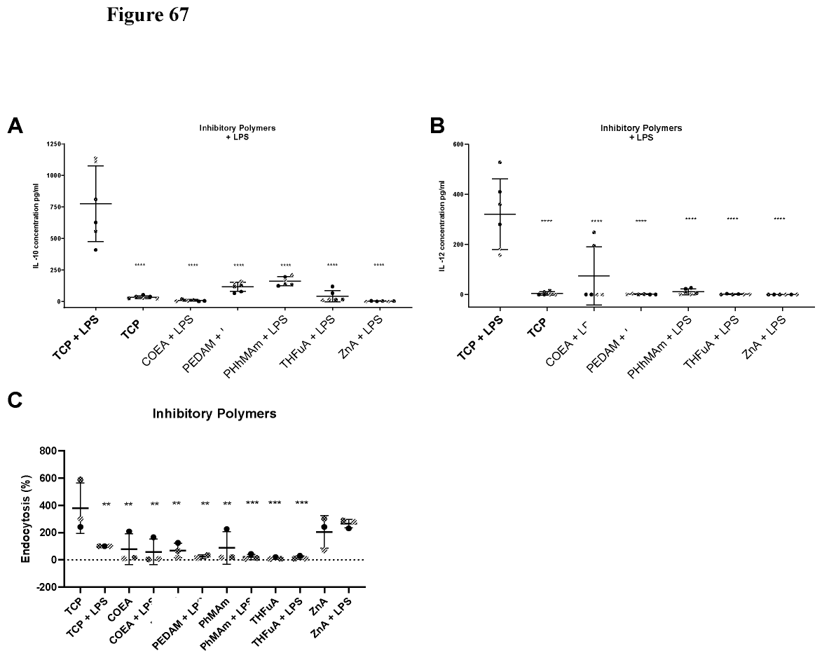

Characterisation of the inhibitory polymers show, that IL-12p70 and IL-10 secretion levels were significantly decreased compared to TCP+LPS when DCs where initially conditioned on polymers coatings, and then stimulated with LPS (see FIG. 67-A-B). Interestingly, when investigating the endocytic ability for the hit polymers that suggested a ‘more immature’ profile, DCs cultured on three of the five ‘hits’ had less endocytic ability than the TCP DCs (FIG. 67). The only polymer that maintained a high endocytic ability, even in presence of LPS stimulation, was ZnA- whose endocytic ability level are around the ones from TCP cultured DCs (P<0.0332).

### DCs Conditioned on Stimulatory or Inhibitory Polymers can Induce or Suppress IFN-γ Production and T Cell Proliferation Respectively

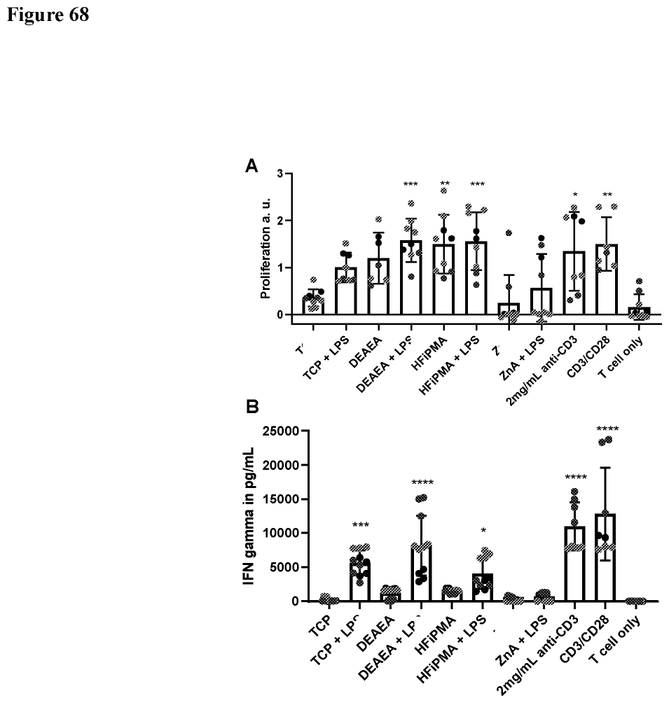

To investigate whether DCs conditioned on stimulatory and inhibitory polymers could differentially stimulate T cell activation, we set up co-cultures between polymer treated DCs and autologous pan T cells followed by quantifying T cells proliferation and IFN-γ production after 8 days of co-culture. The polymers taken forward from the previous experiment were the ones that had the most effect on phenotype modulation, with limited variations between donors. For this reason, DEAEA, HFiPMA and ZnA were chosen for the T cell studies T cells that were co-cultured with DEAEA and HFiPMA conditioned DCs showed higher proliferation levels than TCP (and in case of some donors higher than TCP+LPS), while ZnA conditioned DCs induced lower T cell proliferation levels (FIG. 68-A). Similar results were observed with naïve T cells and memory T cells (data not shown).

DEAEA and HFiPMA conditioning of DCs induced elevated IFN-γ production of T cells, although not as high as TCP+LPS conditioning. T cells that were co-cultured with ZnA polymer-treated DCs produced non detectable amounts of IFN-γ compared to T cells cultured with TCP control DCs (FIG. 68-B). Interestingly ZnA nearly completely suppressed IFN-γ production in all cases.

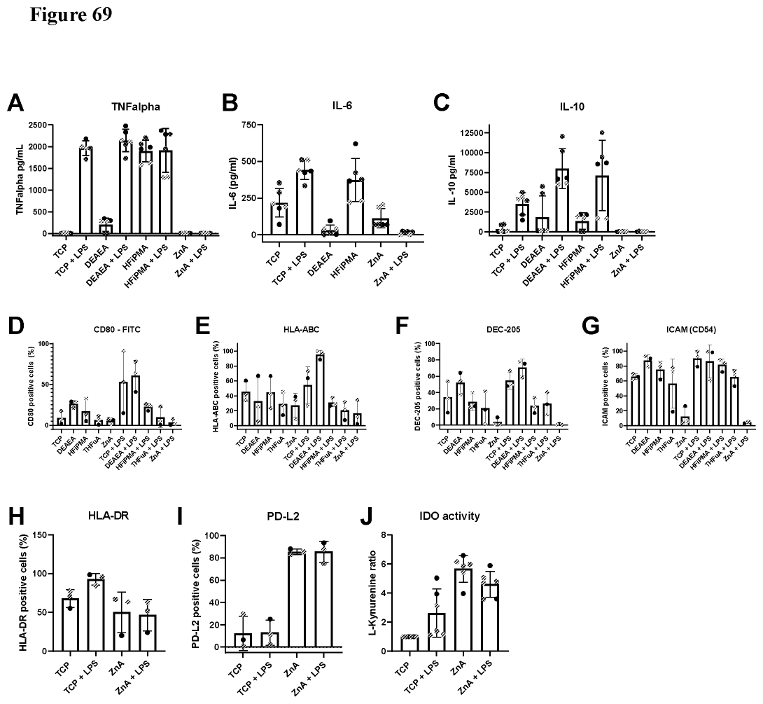

To better understand the basis of differential T cell activation profiles induced by stimulatory and inhibitory polymer conditioned DCs we undertook a more detailed analysis of conditioned DCs phenotype and cytokine secretion (FIG. 69). Conditioning DCs on the stimulatory polymer HFiPMA induced higher secretion of pro-inflammatory cytokines (TNFα, IL-6) than on the TCP control—with similar levels to the TCP+LPS control). IL-6 is connected to B cell differentiation into plasma cells, T cell proliferation, differentiation of CTLs and IL-2 production. Specifically IL-6 has been described to participate in the differentiation of Th2 and Th17 cells, which display pro-inflammatory functions [11].

The stimulatory polymer DEAEA equally increased secretion of TNFα but not IL-6—compared to TCP control. A detailed profile of the DC phenotype presented following polymer culture showed (when compared to TCP control condition), that stimulatory polymers DEAEA and HFiPMA increased or maintained the expression levels of CD80 (activation marker), HLA-ABC (part of the MHC I complex), DEC-205 (uptake receptor) and ICAM-1 (CD54, an adhesion molecule, upregulated on activated DCs) (FIG. 69-D-G). The addition of LPS usually induced a combinatorial effect with the polymer DEAEA (adding up to expression levels above TCP+LPS). ZnA culture lead to the presentation of low secretion of TNFα, IL-6 and IL-10, and expression of CD80, HLA-ABC, DEC-205 and ICAM-1 below the levels presented as TCP—even with the addition of LPS to ZnA. ZnA culture additionally was found to decrease the expression of HLA-DR (part of MHC II), with the simultaneous increase of PD-L2 on DCs (FIG. 69-H-I). Indications of ZnA's ability to induce a regulatory immune response was also found in the increase of IDO activity in DCs cultured on DCs (FIG. 69-J).

### DCs Conditioned by Stimulatory Polymers Induce Higher Instruction of Tumour Specific Cytotoxic T Lymphocytes

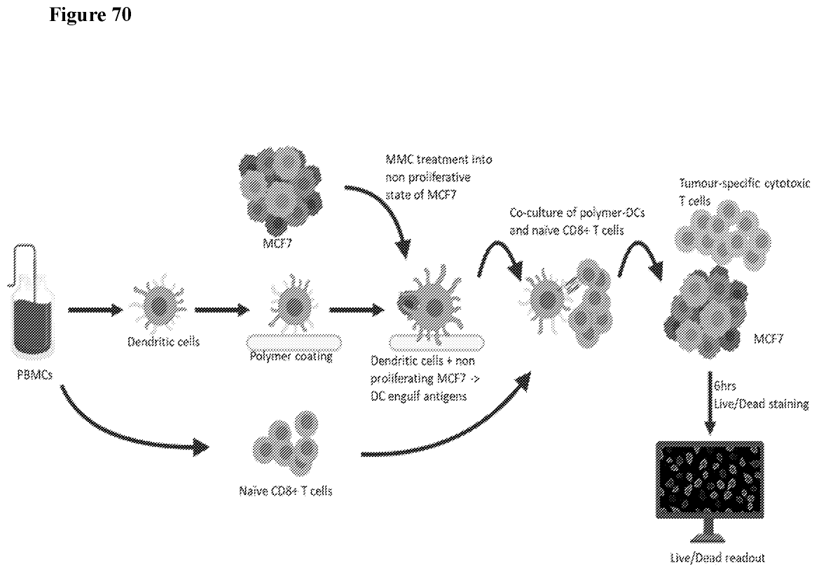

Polymers that induce DC activation are hypothesised to be able to instruct antigen-specific T cell responses, which could be useful for immune-oncology treatment. To test this hypothesis, we examined whether DEAEA or HFiPMA-treated DCs loaded with tumour antigen can gain the ability to prime naïve CD8+ T cells into tumour-specific CTLs in a class I MHC-restricted manner by cross-presentation. Indications of increased cross-presenting ability were observed in the increased expression of HLA-ABC of DCs cultured with DEAEA and stimulated with LPS. A schematic of the experimental approach can be seen in FIG. 70.

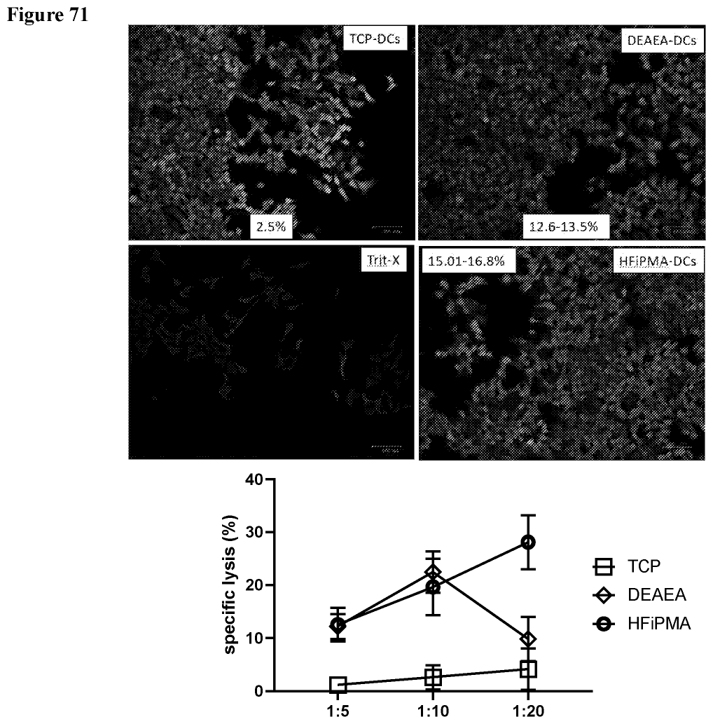

MCF7 breast cancer cells were strongly killed by the CD8+ T cells induced by MHC-matched DCs pre-treated with either DEAEA or HFiPMA (FIG. 71). These findings indicate that DEAEA and HFiPMA-treated DCs can gain the ability to cross-present captured tumour antigens via class I MHCs and can prime and activate neighbouring HLA-matched naïve CD8+ T cells into tumour-specific CD8+ CTLs.

### Assessment of Biomaterial-Induced Cytotoxicity

After 6 hours of polymer treatment, DCs were subjected to assessment of viability with the CytoToxGlo assay (Promega). CytoToxGlo uses a luminogenic peptide substrate, the AAF-Glo™ substrate to measure dead-cell protease activity, which is released from cells that have lost membrane integrity. The AAF-Glo™ substrate cannot cross the intact membrane of live cells and does not generate any appreciable signal from the live-cell population. The assay relies on the properties of Ultra-Glo™ Recombinant Luciferase, which uses aminoluciferin as a substrate to generate a stable “glow-type” luminescent signal and is formulated to improve performance across a wide range of assay conditions. After reading the initial dead-cell protease activity, each well population is lysed with Digitonin to read the total cell protease activity in order to amount for varying cell numbers per well.

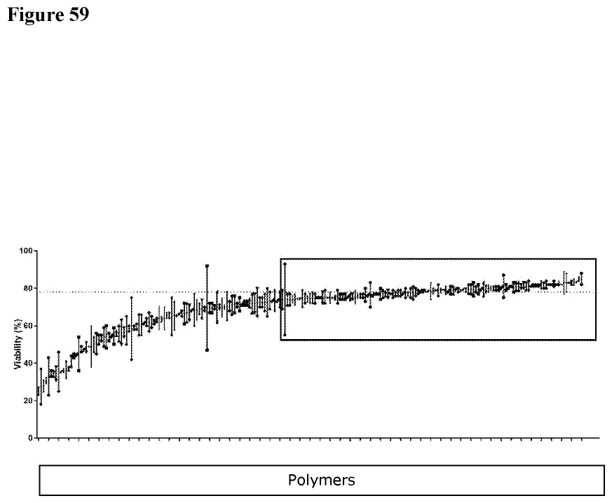

The tissue culture plastic control (TCP) viability was measured as 78% (see dotted line in FIG. 59), and the overall ranked order of viability portrays a form of a slope, that flattens out shortly before the TCP control. Therefore, a threshold for polymers to be seen as ‘viable’ was put on the start of the flattening slope, setting it to 75%. Interestingly, 33% of the investigated polymers supported DC survival more efficiently than the tissue culture plastic, which could be an interesting aspect to keep in mind for (ex vivo) DC cultures. The first screening led to 120 Polymers, that made the threshold of over 75% viability and those polymers were chosen to take into the next screening step.

### Assessment of Biomaterial-Induced Modulation of DC Phenotype

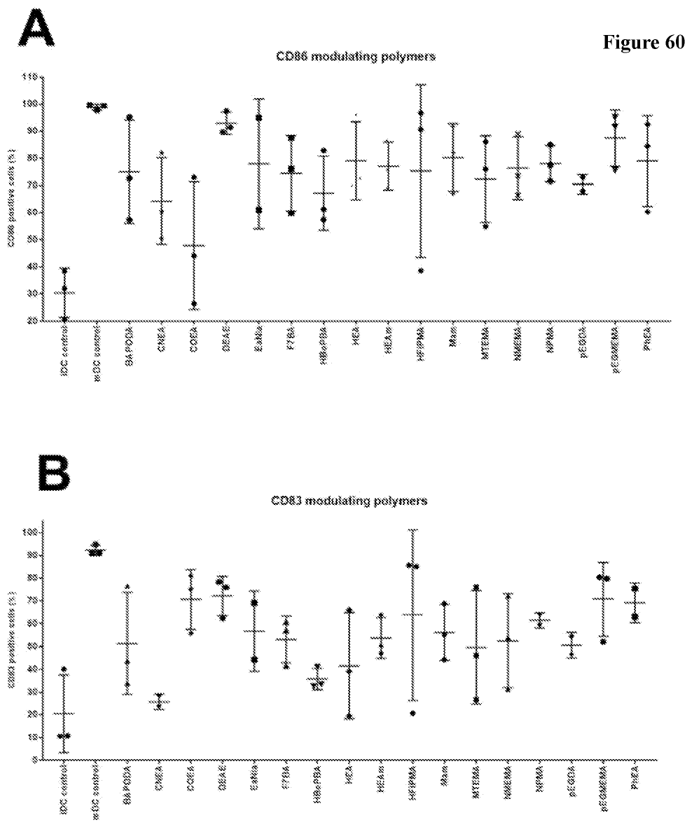

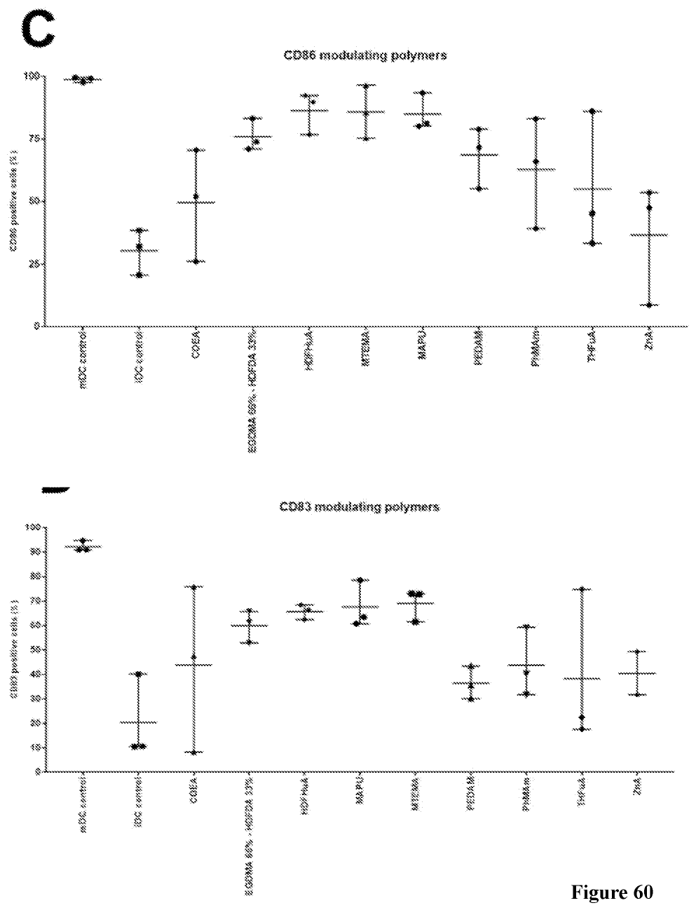

Assessment of DC maturation in response to biomaterials typically involves the treatment of immature DCs (iDCs) with biomaterials pre-placed in wells using immunological assays such as flow cytometry for the expression of DC-specific or maturation surface markers. Assessing whether biomaterials are able to suppress DC maturation, has the same methodology. A total of 120 polymers were shown to induce good viability, which were screened on their ability to either stimulate or suppress dendritic cell maturation. In FIG. 60 we summarized specific polymers, that were shown to modulate key markers (CD83 and CD86), that give us information about the level of activation/maturation the cells possess. CD83 and CD86 are DC activation markers: CD86 binds to surface markers on T cells and CD83 has co-stimulatory effects on T cell activation. As such, they are essential for evoking a T cell effector response and therefore have been chosen to be good markers to assess DC activation level. 17 polymers increased CD86 and CD83 expression levels significantly (close to the levels of the DC control stimulated with LPS; see FIG. 60 A-B). Overall, the polymers presented were elevating the expression levels of CD86 more than of CD83, but a correlation between both shows good fit of the two expression levels (R²=0.931). However, DCs conditioned with the majority of different polymers were still able to fully mature in response to LPS stimulation. A minority of polymers were able to prevent development of a mature phenotype and showed significantly decreased expression levels of CD83 and CD86 (FIG. 60 C-D). Other surface markers that were investigated (such as HLA-DR, CCR7 and PD-L1) did not show relevant changes of expression levels (data not shown).

### Polymer-Induced Immunomodulation of DC Function (Cytokine Secretion)

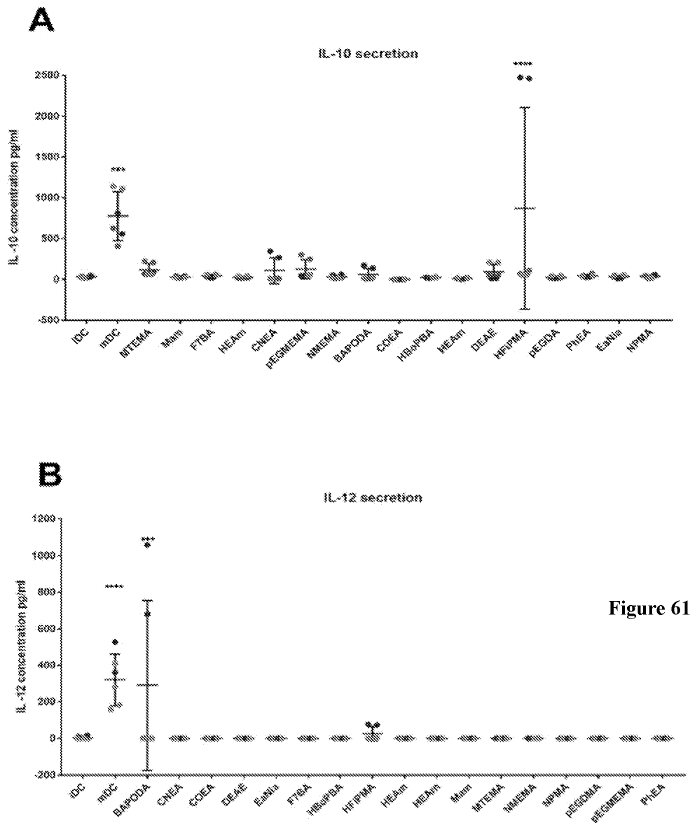

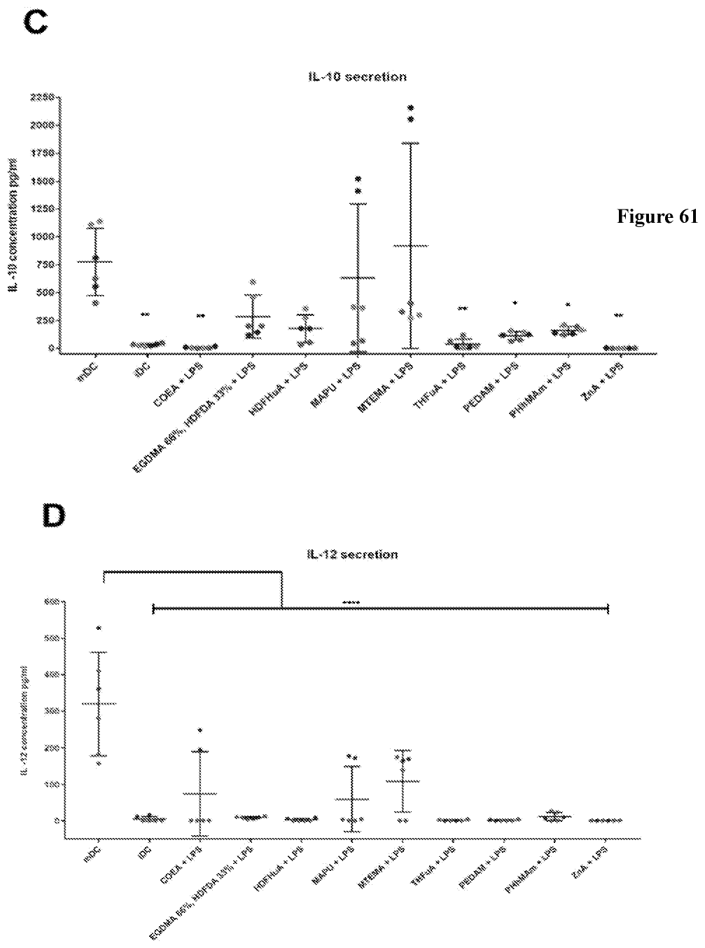

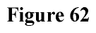

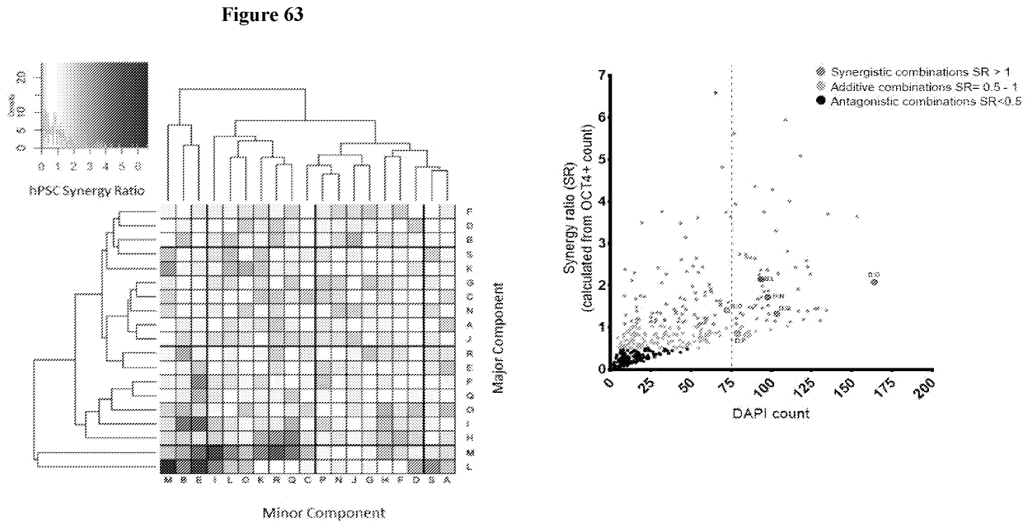

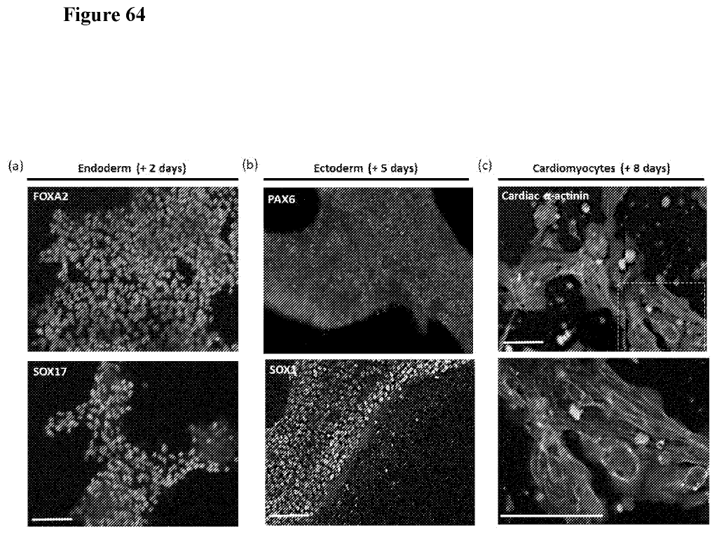

For a proper T cell effector response, T cells need 3 signals: 1. Antigen needs to be presented via MHC I or II; 2. Co-stimulation via CD83, CD86 and other surface markers; 3. Polarizing cytokines secreted by DCs. For this reason, we then went on to evaluate the cytokine secretion of IL-10 and IL-12 (IL-12p70) to investigate for any modulation by polymer culture. DCs can skew the differentiation of naïve T cells towards Th1 cells (responsible for cell-mediated immunity; via IL-12 secretion), Th2 cells (dedicated to humoral response but also responsible for allergic disease; via IL-6, IL-10 secretion but low IL-12), Th17 cells (committed to protecting against extracellular pathogens; via TGF-β, IL-6, IL-23 secretion) and also regulatory T cells (capable of suppressing Th1, Th2, Th17 subsets; via IL-10 secretion) [25]. To evaluate the T cell polarizing capability of the phenotypically modulated DCs, we went on to check the levels of secreted IL-10 and IL-12 after polymer cultures. IL-12p70 and IL-10 secretion levels have been significantly decreased when DC where initially conditioned on the polymers, after which they were stimulated with LPS (see FIG. 61 A-B). IL-10 secretion were slightly elevated when DCs were cultured on polymers, whereas only one polymer increased IL-12 secretion (FIG. 61 C-D).

### Discussion

DCs are crucial immune cells linking the innate and adaptive immunity, playing an important role in the orchestration of the adaptive immune response. Notably, DC phenotype is a powerful indicator of their downstream effector functions. The discovery of the ability to modulate these functions has opened up a new direction of immune system targeted therapies. Biomaterials have in the past been found to modulate host immune responses, as well as on the phenotypic state of DCs. Various biomaterial polymers (alginate, agarose, chitosan, HA, poly(lactid-co-glycolic acid)) have been shown to exert differential effects on DC activation levels [31], [33], [34]. In the past ten years, established libraries of combinatorial polymers in high throughput-screening formats have created opportunities to identify simple polymers with bio-instructive interactions with great success [44]-[46], [54]-[56]. A polymer library previously used to identify materials with the ability to instruct stem cell differentiation or stop bacterial biofilm formation [44], [45], [54] was used as the material source here. As far as we know, there has been no attempt yet to have a directed screen to identify new biomaterials to bring into clinical use for DC modulation.

The results of this study demonstrate that high throughput-screening can be utilised to identify polymers that could be applied in clinical settings, and more specifically, presents synthetic polymer coatings that can be used to engineer the immune response. These three polymers have potently modulated DC phenotype and function—actively driving the ensuing adaptive immune response. Two (stimulatory) polymers were identified, that activate DCs and via this enhance T cell response. In case of the stimulatory polymers, these could induce a tumour-specific immune response that kills tumour cells efficiently. An inhibitory polymer was also identified and was shown to portray several indicators of a tolerance inducing material. Whereas stimulatory polymer effect on DCs may prolong the immune response to biomaterials and delay wound healing, the effect of inhibitory polymers means that DCs are capable to down-regulate the immune cells and resolve inflammation. Thus, induction of tolerogenic DC by designing the surface chemistry appears to be a promising strategy of modulating immune responses to biomaterials to improve biocompatibility.

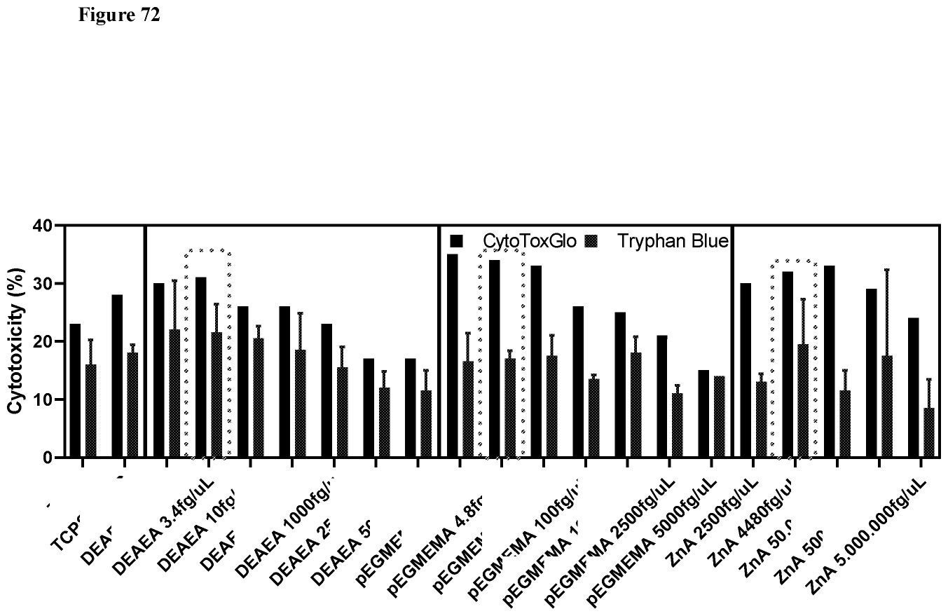

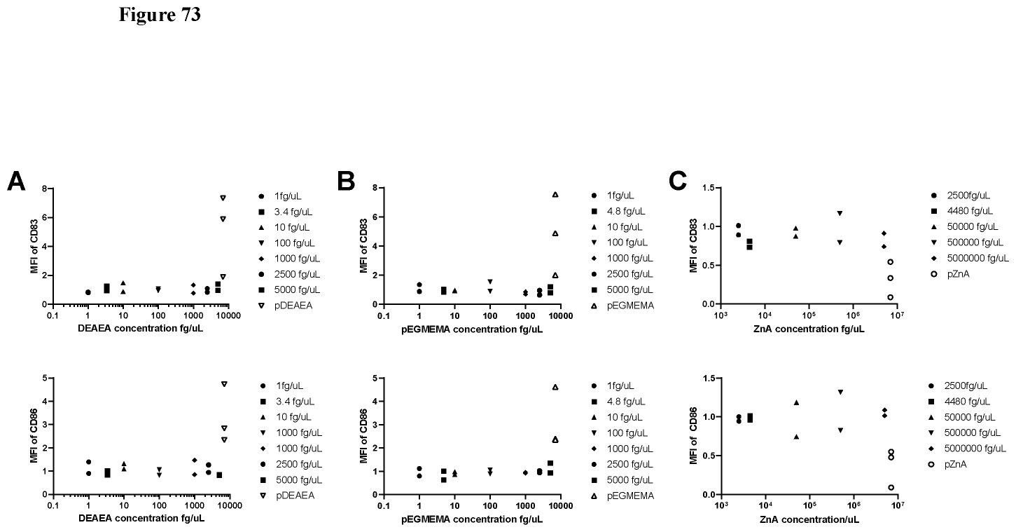

One possibility as the source of the introduced cytotoxicity in the viability were remnants of non-polymerised monomer. Analysis of cell culture supernatant revealed low concentrations of monomer to be present (femtogram range; Table S1). As even low amounts of monomer can have an detrimental or even modulatory effect on cells, raw monomer in the range of the concentration found in cell culture supernatant was added to DC cultures with no significant subsequent cytotoxicity or phenotype modulation observed (FIG. 72-73). Interestingly, 33% of the investigated polymers supported DC survival more efficiently than the tissue culture plastic, (FIG. 71). A possible explanation for this might be that proteins adsorbed on these polymers support more DC viability, than the surface chemistry of the tissue culture plastic polystyrene. Previously, gas permeable hydrophobic culture bags have been found to be superior for viability, yield and function of DCs cultured in plastic tissue culture flasks or well plates. Comparing various well plate formats and plate materials, a study concluded that DCs cultured and assayed under conditions that minimise cell adherence are functionally better, as well as support more viability [57]. These polymers were not further investigated in this setting but could prove to be relevant for ex vivo DC cultures as an alternative culture surface (such as Polyolefin bags) to tissue culture plastic.

In several studies, polymers were investigated for their ability to modulate DC phenotype. Although none of the polymers identified here were used in previous studies, a number of polymethacrylates-polyhydroxyethylmethacrylate (pHEMA) and poly (isobutyl-co-benzyl-co-terahydrofurfuryl) methacrylate (pIBTMA) were shown to induce the least and most mature DC phenotype, respectively [58]. A conclusion is, therefore, that upon culture with synthetic polymers, DCs react differently to polymer chemistry. None of the polymers from the source library used here, have previously been investigated for their potential on modulating DC phenotype and function.

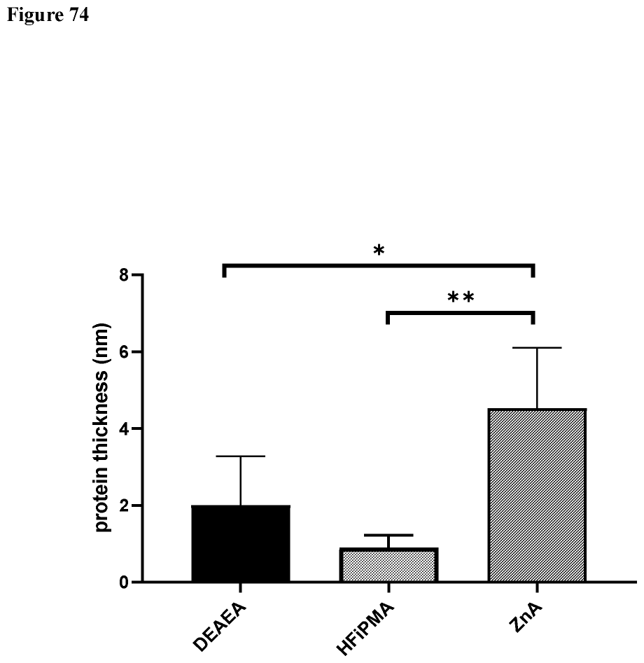

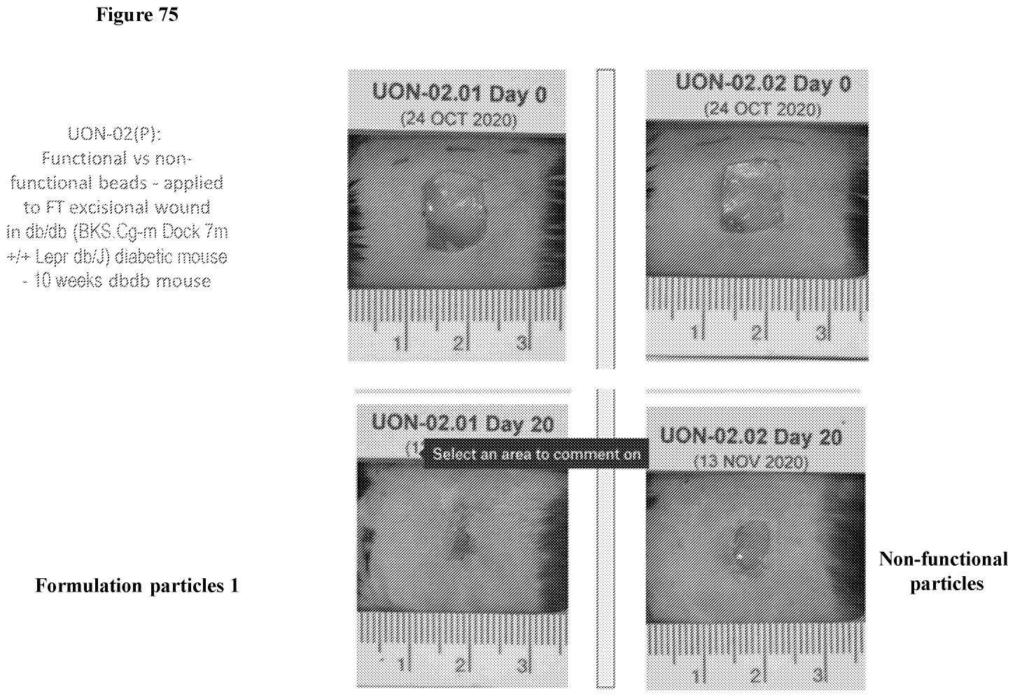

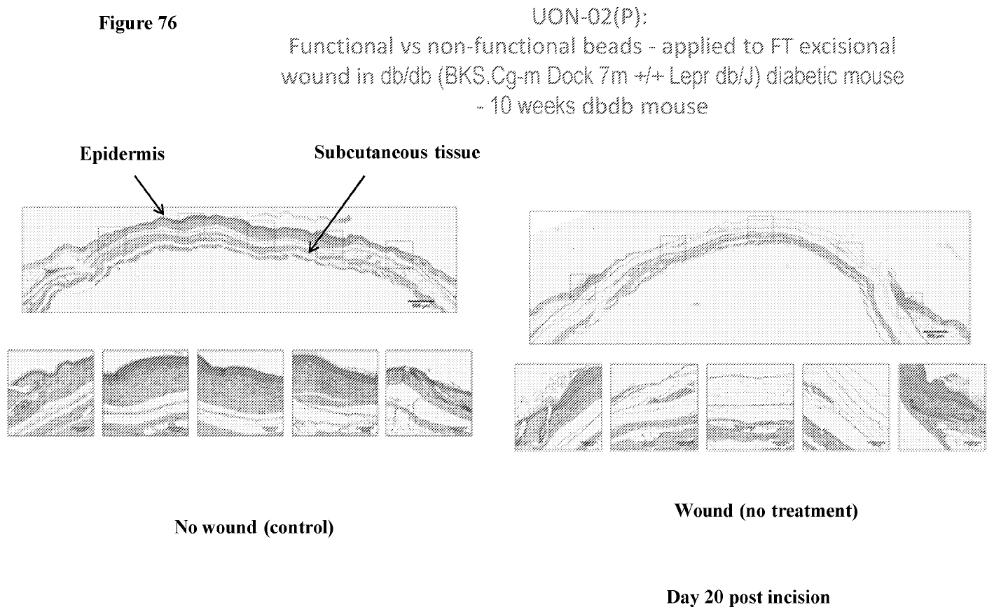

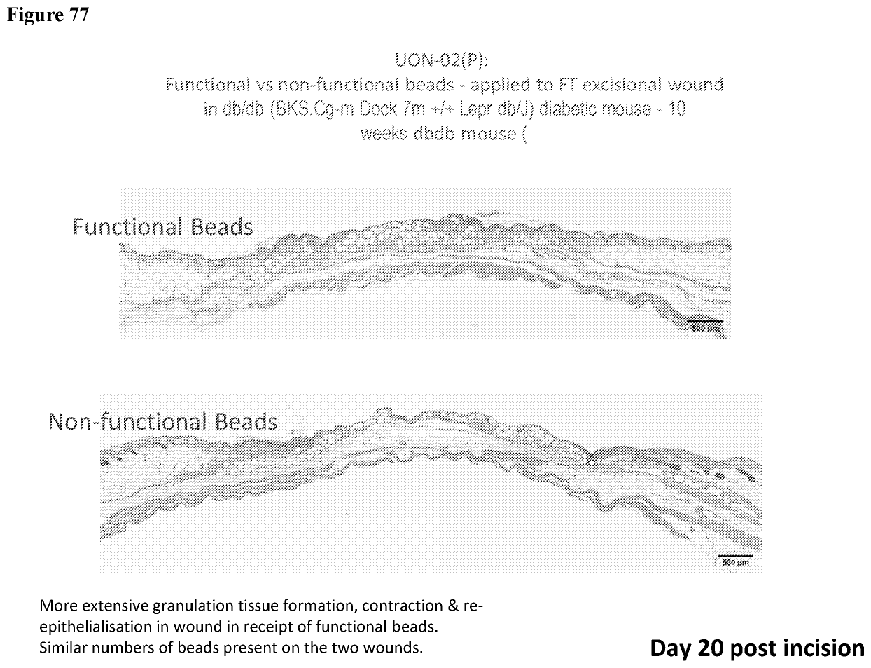

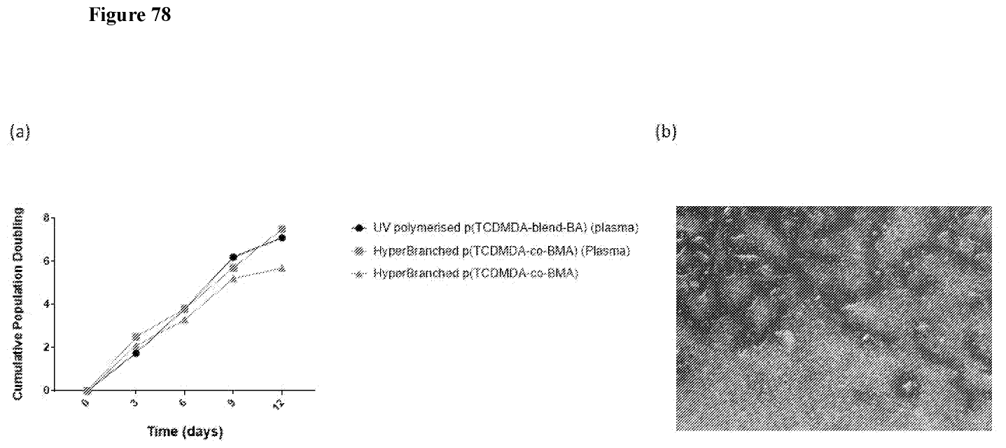

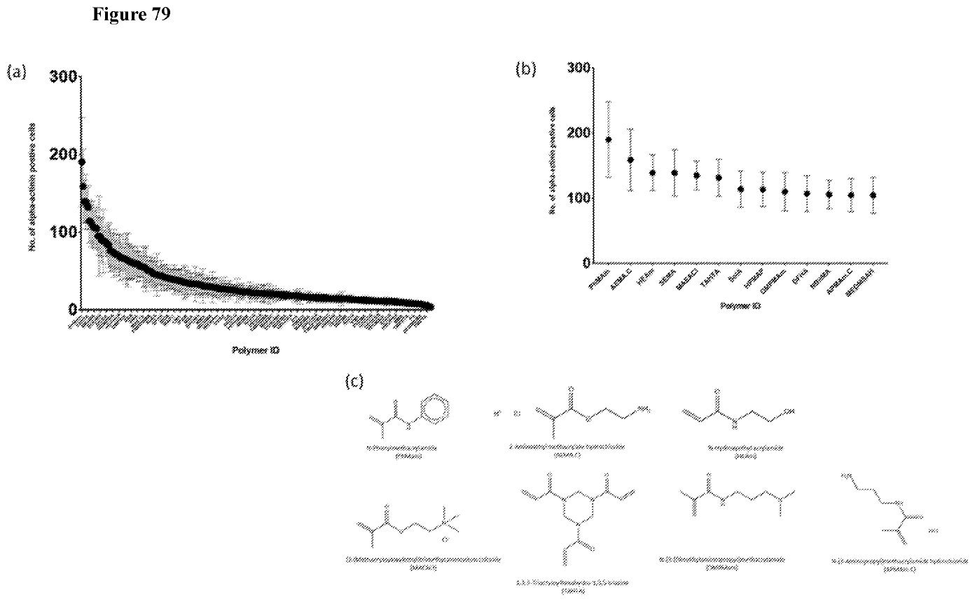

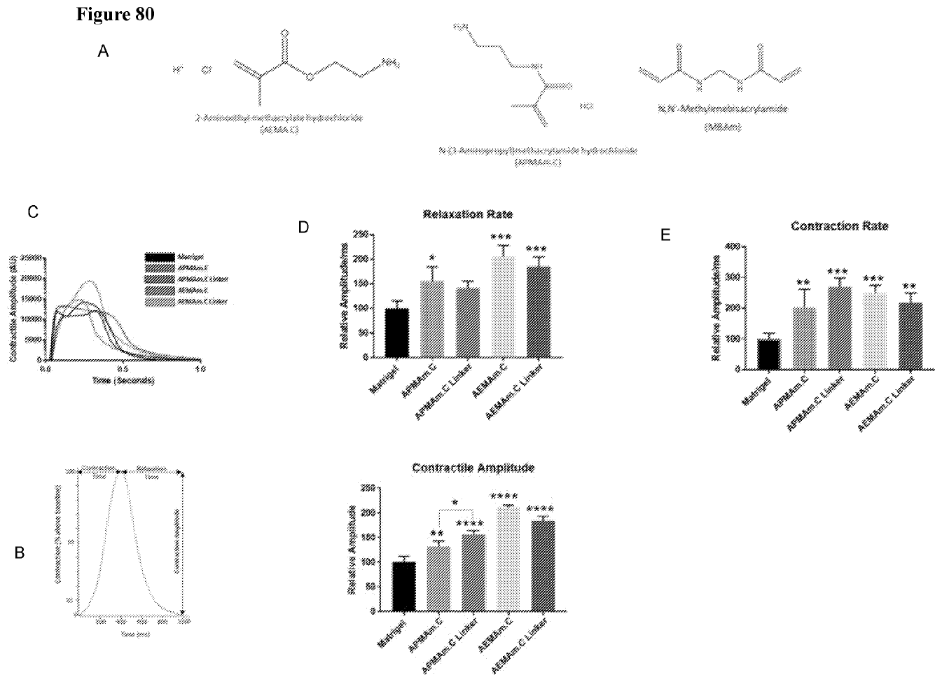

Modulation of CD83 and CD86 leads to specific DC phenotypes that translate into T cell responses—e.g. more proliferation of T cells. Inhibitory polymers identified here limit the activation of DCs and conversely T cells. Kou et al. has previously shown that specific material properties can be used to explain DC response to polymer culture. As such, increases in contact angle (17.2-71.2°) lead to lower DC maturation, as angles close to 71.2° are similar to TCP [58]. Additionally, her work on titanium surfaces presented, that higher surface carbon nitrogen lead to more DC maturation, while more surface oxygen and titanium makes DC remain immature [58]. This contrasts the observation made in this study, where ZnA (inhibitory polymer) had higher surface oxygen and nitrogen (data not shown), and subsequently thicker protein layers adsorbed to the coating (FIG. 74). Both stimulatory polymers also showed higher surface oxygen (while adsorbing less proteins), which contradicts the correlation between higher oxygen equating lower DC maturation. These contradictions can be explained by how these XPS samples were analysed—due to these being solvent cast into well plates (and stated to produce a non-homogenous coating), as well as walls being cut off to perform XPS analysis.

Recent research on MyD88 and TLR knock out mice demonstrated, that DCs use TLR-2, 4 and 6 for the response to a group of chemically and physically diverse biomaterials. Mice missing any of these TLRs or MyD88 showed impaired expression of activation markers and reduced production of pro-inflammatory cytokines relative to the wild-type controls [59]. An explanation for the modulatory ability of polymers would be that their surface presents itself as ligands to either one of the TLRs identified as responsible to react to biomaterials. ECM proteins adsorbed on tissue-culture polystyrene plates have been shown to affect DC morphology, cytokine production, and their allostimulatory capacity. For example, DCs cultured on collagen and vitronectin substrates released higher levels of IL-12p40, whereas DCs treated on albumin and serum-coated substrates generated higher amounts of IL-10 compared to other substrates [38]. Through XPS analysis the inhibitory polymer identified here was shown to significantly adsorb more proteins than both stimulatory polymers (FIG. 74), which could easily be the source of polymer-dependant modulation of DC phenotype and function. To elucidate which proteins specifically are adsorbed on these polymers identified here more assays (such as ToF-SIMS) of single protein controls will have to be performed. As another option, copolymers with higher percentage of hydrophobic monomers were found to have increased adjuvant activity[60]. It is possible that hydrophobic polymers activate DCs in a way that mimics the hydrophobic domains of PAMPs.

Most stimulatory polymers induced slightly elevated secretion levels of IL-10 and IL-12p70, although 2 did so in a statistically significant manner. Other studies looking at pMAs (polymer methacrylates) showed IL-10 to also only be slightly modulated to above TCP levels, which is the same as observed here [58]. The juxtaposition between low cytokine secretion and the efficient interaction with T cell for the stimulatory polymers is remarkable. This could indicate that the cytokines released were not all available to assay (e.g. through adsorption onto the polymer coatings) which gives a false low cytokine secretion.

ZnA treated DCs with LPS stimulation showed the same level of endocytic ability like immature DCs but did not induce T cell proliferation or IFN-γ production after co-culture. These results clearly show the importance of costimulatory surface markers, for DC-T cell crosstalk and underlines the previously presented ‘more immature’ profile of ZnA treated DCs, which is further proven with the high IDO activity and PD-L2 expression.

Strongest effectors for achieving target tumour cell eradication have been thought to be class I MHC-restricted CD8+ CTLs. CD8+ CTLs are regulated by DCs that possess potent cross-presentation capacity. Stimulatory polymers induced a phenotype and functionality in conditioned DCs that is typical for activated/mature DCs, which translates into increased T cell proliferation and therefore adaptive response. Based on these findings, we examined whether DEAEA- and HFiPMA-treated DCs loaded with tumour antigen could prime autologous naïve human CD8+ T cells into tumour-specific CD8+ CTLs.

These results confirmed, this was possible in a class I MHC-restricted manner and that an increased ratio of effector to target cells was more effective than TCP cultured DCs with tumour antigens. The stimulatory polymers were comparatively more effective in % of killed tumour cells than several approaches described in the literature (such as antigen pulsing or conjugation of antigen to virus-like particles) [61], [62]. Additionally, different than in these alternative approaches, DCs do not have to replenished from outside the patient, which will lead to a longer lasting therapeutic intervention without additional external clinical work.

The findings provide rationale for the stimulatory polymers to be tested in vivo for their effect on anti-tumour responses. The next step in this process is the development of a deliverable format of those polymers for application in vivo. Examples of this could be macroporous scaffolds or particulates, which have already found application in acute myeloid leukaemia and breast cancer [19], [63]. Another possible application for these polymers are adjuvants for vaccinations.

Possible applications of the suppressive polymers are autoimmune diseases, allergies. Abnormal high IL-12 levels have been described in animal models of autoimmune diseases, in related studies it was found that IL-12 blocking leads to a stable remission of patients of active Crohn's disease [64]. This could give application to inhibitory polymers usage.

In summary, in this study a number of immune stimulatory and inhibitory polymers have been identified as evidenced by their ability to modulate DC phenotype and function. We have also shown the ability of stimulatory polymers conditioned DCs to prime CD8 T cell tumour killing in an in vitro breast cancer setting. Additionally, it was shown, that inhibitory polymer conditioned DCs show a tolerogenic phenotype and function.

### REFERENCES

### Bibliography

[1] C. S. Verbeke and D. J. Mooney, “Injectable, Pore-Forming Hydrogels for In Vivo Enrichment of Immature Dendritic Cells,” Adv. Healthc. Mater., vol. 4, no. 17, pp. 2677-2687, 2015, doi: 10.1002/adhm.201500618.
[2] C. S. Verbeke et al., “Multicomponent Injectable Hydrogels for Antigen-Specific Tolerogenic Immune Modulation.,” Adv. Healthc. Mater., vol. 42, pp. 1600715-1600773, 2017.
[3] S. K. Wculek, F. J. Cueto, A. M. Mujal, I. Melero, M. F. Krummel, and D. Sancho, “Dendritic cells in cancer immunology and immunotherapy,” Nat. Rev. Immunol., vol. 20, no. 1, pp. 7-24, 2020, doi: 10.1038/s41577-019-0210-z.
[4] J. Calmeiro, M. Carrascal, C. Gomes, A. Falcão, M. T. Cruz, and B. M. Neves, “Biomaterial-based platforms for in situ dendritic cell programming and their use in antitumor immunotherapy,” J. Immunother. Cancer, vol. 7, no. 1, pp. 1-11, 2019, doi: 10.1186/s40425-019-0716-8.
[5] P. Hatfield et al., “Optimization of dendritic cell loading with tumor cell lysates for cancer immunotherapy,” J. Immunother., vol. 31, no. 7, pp. 620-632, 2008, doi: 10.1097/CJI.0b013e31818213df.
[6] A. D. Garg, P. G. Coulie, B. J. Van den Eynde, and P. Agostinis, “Integrating Next-Generation Dendritic Cell Vaccines into the Current Cancer Immunotherapy Landscape,” Trends Immunol., vol. 38, no. 8, pp. 577-593, 2017, doi: 10.1016/j.it.2017.05.006.
[7] A. Huber, F. Dammeijer, J. G. J. V. Aerts, and H. Vroman, “Current State of Dendritic Cell-Based Immunotherapy: Opportunities for in vitro Antigen Loading of Different DC Subsets?,” Frontiers in immunology, vol. 9, no. December. p. 2804, 2018, doi: 10.3389/fimmu.2018.02804.
[8] P. J. Tacken, I. J. M. De Vries, R. Torensma, and C. G. Figdor, “Dendritic-cell immunotherapy: From ex vivo loading to in vivo targeting,” Nat. Rev. Immunol., vol. 7, no. 10, pp. 790-802, 2007, doi: 10.1038/nri2173.
[9] A. G. Thompson and R. Thomas, “Induction of immune tolerance by dendritic cells: Implications for preventative and therapeutic immunotherapy of autoimmune disease,” Immunol. Cell Biol., vol. 80, no. 6, pp. 509-519, 2002, doi: 10.1046/j.1440-1711.2002.01114.x.
[10] G. J. Randolph, K. Inaba, D. F. Robbiani, R. M. Steinman, and W. A. Muller, “Differentiation of phagocytic monocytes into lymph node dendritic cells in vivo,” Immunity, vol. 11, no. 6, pp. 753-761, 1999, doi: 10.1016/S1074-7613(00)80149-1.
[11] P. Blanco, A. K. Palucka, V. Pascual, and J. Banchereau, “Dendritic cells and cytokines in human inflammatory and autoimmune diseases,” Cytokine Growth Factor Rev., vol. 19, no. 1, pp. 41-52, 2008, doi: 10.1016/j.cytogfr.2007.10.004.Dendritic.
[12] J. Banchereau, S. Lebecque, J. Davoust, and W. Liu, “Immunobiology of dendritic cells,” Annu. Rev. Immunol., vol. 18, pp. 767-811, 2000, doi: 10.1146/annurev.immunol.18.1.767.
[13] E. Orsini, A. Guarini, S. Chiaretti, F. R. Mauro, and R. Foa, “The circulating dendritic cell compartment in patients with chronic lymphocytic leukemia is severely defective and unable to stimulate an effective T-cell response,” Cancer Res., vol. 63, no. 15, pp. 4497-4506, 2003, doi: 10.1038/32588.
[14] M. Cella, A. Engering, V. Pinet, J. Pieters, and A. Lanzavecchia, “Inflammatory stimuli induce accumulation of MHC class II complexes on dendritic cells,” Nature, vol. 388, no. 6644, pp. 782-787, 1997, doi: 10.1038/42030.
[15] G. E. Kaiko, J. C. Horvat, K. W. Beagley, and P. M. Hansbro, “Immunological decision-making: How does the immune system decide to mount a helper T-cell response?,” Immunology, vol. 123, no. 3, pp. 326-338, 2008, doi: 10.1111/j.1365-2567.2007.02719.x.
[16] J. Parkin and B. Cohen, “An overview of the immune system,” Lancet, vol. 357, no. 9270, pp. 1777-1789, 2001, doi: 10.1016/S0140-6736(00)04904-7.
[17] A. Waisman, D. Lukas, B. E. Clausen, and N. Yogev, “Dendritic cells as gatekeepers of tolerance,” Semin. Immunopathol., vol. 39, no. 2, pp. 1-11, 2016, doi: 10.1007/s00281-016-0583-z.
[18] J. Kim, W. A. Li, W. Sands, and D. J. Mooney, “Effect of Pore Structure of Macroporous Poly Lactide-co-Glycolide) Scaffolds on the In Vivo Enrichment of Dendritic Cells Effect of Pore Structure of Macroporous Poly (Lactide-co-Glycolide) Scaffolds on the In Vivo Enrichment of Dendritic Cells,” ACS Appl. Mater. Interfaces, vol. 6, no. 11, pp. 8505-8512, 2014, doi: 10.1021/am501376n.
[19] N. J. Shah et al., “A biomaterial-based vaccine eliciting durable tumour-specific responses against acute myeloid leukaemia,” Nat. Biomed. Eng., 2020, doi: 10.1038/s41551-019-0503-3.
[20] M. P. Domogalla, P. V. Rostan, V. K. Raker, and K. Steinbrink, “Tolerance through education: How tolerogenic dendritic cells shape immunity,” Front. Immunol., vol. 8, no. DEC, pp. 1-14, 2017, doi: 10.3389/fimmu.2017.01764.
[21] R. M. Pearson, L. M. Casey, K. R. Hughes, S. D. Miller, and L. D. Shea, “In vivo reprogramming of immune cells: technologies for induction of antigen-specific tolerance,” Adv. Drug Deliv. Rev., vol. 35, no. 114, pp. 240-255, 2017, doi: 10.1177/0333102415576222.ls.
[22] U. Grohmann et al., “Functional Plasticity of Dendritic Cell Subsets as Mediated by CD40 Versus B7 Activation,” J. Immunol., vol. 171, no. 5, pp. 2581-2587, 2003, doi: 10.4049/jimmunol.171.5.2581.
[23] B. Pulendran, H. Tang, and T. L. Denning, “Division of labor between dendritic cell subsets of the lung,” Curr Opin Immunol, vol. 20, no. 1, pp. 61-67, 2008, doi: 10.1038/mi.2008.39.
[24] H. M. Rostam et al., “The impact of surface chemistry modification on macrophage polarisation,” Immunobiology, vol. 221, no. 11, pp. 1-10, 2016, doi: 10.1016/j.imbio.2016.06.010.
[25] J. Doloff et al., “Colony Stimulating Factor-1 Receptor is a central component of the foreign body response to biomaterial implants in rodents and non-human primates,” Nat. Mater., vol. 16, no. 6, pp. 671-680, 2017, doi: doi:10.1038/nmat4866.
[26] K. M. Hotchkiss et al., “Titanium Surface Characteristics, Including Topography and Wettability, Alter Macrophage Activation,” Acta Biomater, vol. 31, pp. 425-434, 2016, doi: 10.1016/j.actbio.2015.12.003.
[27] M. J. Vassey et al., “Immune Modulation by Design: Using Topography to Control Human Monocyte Attachment and Macrophage Differentiation,” Adv. Sci., vol. 1903392, p. 1903392, April 2020, doi: 10.1002/advs.201903392.
[28] L. Burroughs et al., “Synergistic Material-Topography Combinations to Achieve Immunomodulatory Osteogenic Biomaterials,” bioRxiv, p. 2020.04.29.067421, 2020, doi: 10.1101/2020.04.29.067421.
[29] H. M. Rostam et al., “Immune-Instructive Polymers Control Macrophage Phenotype and Modulate the Foreign Body Response In Vivo,” Matter, vol. 2, no. 6, pp. 1564-1581, June 2020, doi: 10.1016/j.matt.2020.03.018.
[30] P. M. Kou, “Elucidation of dendritic cell response-material property relationships using high throughput methodoligies,” pp. 1-239, 2011.
[31] J. E. Babensee, “Interaction of dendritic cells with biomaterials,” Semin. Immunol., vol. 20, no. 2, pp. 101-108, April 2008.
[32] J. Park and J. E. Babensee, “Differential functional effects of biomaterials on dendritic cell maturation,” Acta Biomater., vol. 8, no. 10, pp. 3606-3617, 2012.
[33] J. Babensee and A. Paranjpe, “Differential levels of dendritic cell maturation on different biomaterials used in combination products,” J Biomed Mater Res A., vol. 4, no. 74, pp. 503-10, 2005.
[34] M. Yoshida and J. E. Babensee, “Poly(lactic-co-glycolic acid) enhances maturation of humanmonocyte-derived dendritic cells,” J. Biomed. Mater. Res. Part A, 2004.
[35] D. M. Higgins, R. J. Basaraba, A. C. Hohnbaum, E. J. Lee, D. W. Grainger, and M. Gonzalez-Juarrero, “Localized immunosuppressive environment in the foreign body response to implanted biomaterials.,” Am. J. Pathol., vol. 175, no. 1, pp. 161-170, 2009, doi: 10.2353/ajpath.2009.080962.
[36] B. G. Keselowsky and J. S. Lewis, “Dendritic cells in the host response to implanted materials,” Semin. Immunol., vol. 29, no. November 2016, pp. 33-40, 2016, doi: 10.1016/j.smim.2017.04.002.
[37] S. S. N. E. V. M. R. A. A. M. G. H M Rostam, “Impact of surface chemistry and topography on the function of antigen presenting cells,” Biomater. Sci., vol. 3, no. 3, pp. 424-441, 2015.
[38] B. Shokouhi et al., “The role of multiple toll-like receptor signalling cascades on interactions between biomedical polymers and dendritic cells,” Biomaterials, vol. 31, no. 22, pp. 5759-5771, 2010, doi: 10.1016/j.biomaterials.2010.04.015.
[39] T. H. Rogers and J. E. Babensee, “The role of integrins in the recognition and response of dendritic cells to biomaterials,” Biomaterials, vol. 32, no. 5, pp. 1270-1279, 2011.
[40] F. G. Giancotti and E. Ruoslahti, “Integrin signaling,” Science (80-.)., vol. 285, no. 5430, pp. 1028-1032, August 1999, [Online]. Available: http://science.sciencemag.org/content/285/5430/1028.abstract.
[41] M. F. Maitz, “Applications of synthetic polymers in clinical medicine,” Biosurface and Biotribology, vol. 1, no. 3, pp. 161-176, 2015, doi: 10.1016/j.bsbt.2015.08.002.
[42] J. J. Green and J. H. Elisseeff, “Mimicking biological functionality with polymers for biomedical applications,” Nature, vol. 540, no. 7633, pp. 386-394, 2016, doi: 10.1038/nature21005.
[43] I. Firkowska-Boden, X. Zhang, and K. D. Jandt, “Controlling Protein Adsorption through Nanostructured Polymeric Surfaces,” Adv. Healthc. Mater., vol. 7, no. 1, pp. 1-19, 2018, doi: 10.1002/adhm.201700995.
[44] A. D. Celiz et al., “Discovery of a Novel Polymer for Human Pluripotent Stem Cell Expansion and Multilineage Differentiation,” Adv. Mater., vol. 27, no. 27, pp. 4006-4012, 2015, doi: 10.1002/adma.201501351.
[45] A. K. Patel et al., “A defined synthetic substrate for serum-free culture of human stem cell derived cardiomyocytes with improved functinoal maturity identified using combinatorial materials microarrays,” vol. 61, pp. 257-265, 2016, doi: 10.1016/j.biomaterials.2015.05.019.A.
[46] A. L. Hook et al., “Combinatorial discovery of polymers resistant to bacterial attachment,” Nat. Biotechnol., vol. 30, no. 9, pp. 867-874, 2012, doi: 10.1038/nbt.2316.
[47] D. Awuah, M. Alobaid, A. Latif, F. Salazar, R. D. Emes, and A. M. Ghaemmaghami, “The Cross-Talk between miR-511-3p and C-Type Lectin Receptors on Dendritic Cells Affects Dendritic Cell Function,” J. Immunol., vol. 203, no. 1, pp. 148-157, 2019, doi: 10.4049/jimmunol.1801108.
[48] S. Garcia-Nieto et al., “Laminin and Fibronectin Treatment Leads to Generation of Dendritic Cells with Superior Endocytic Capacity,” PLoS One, vol. 5, no. 4, pp. e10123-10, April 2010, doi: 10.1371/journal.pone.0010123.
[49] H. Harrington et al., “Immunocompetent 3D model of human upper airway for disease modeling and in vitro drug evaluation,” Mol. Pharm., vol. 11, no. 7, pp. 2082-2091, 2014, doi: 10.1021/mp5000295.
[50] F. Salazar, D. Awuah, O. H. Negm, F. Shakib, and A. M. Ghaemmaghami, “The role of indoleamine 2,3-dioxygenase-aryl hydrocarbon receptor pathway in the TLR4-induced tolerogenic phenotype in human DCs,” Sci. Rep., vol. 7, no. March, pp. 1-11, 2017, doi: 10.1038/srep43337.
[51] Y. Tomita, E. Watanabe, M. Shimizu, Y. Negishi, Y. Kondo, and H. Takahashi, “Induction of tumor-specific CD8+ cytotoxic T lymphocytes from naïve human T cells by using Mycobacterium-derived mycolic acid and lipoarabinomannan-stimulated dendritic cells,” Cancer Immunol. Immunother., vol. 68, no. 10, pp. 1605-1619, 2019, doi: 10.1007/s00262-019-02396-8.
[52] A. D. McLellan, A. Heiser, and D. N. J. Hart, “Induction of dendritic cell costimulator molecule expression is suppressed by T cells in the absence of antigen-specific signalling: Role of cluster formation, CD40 and HLA-class II for dendritic cell activation,” Immunology, vol. 98, no. 2, pp. 171-180, 1999, doi: 10.1046/j.1365-2567.1999.00860.x.
[53] C. Aerts-Toegaert et al., “CD83 expression on dendritic cells and T cells: Correlation with effective immune responses,” Eur. J. Immunol., vol. 37, no. 3, pp. 686-695, 2007, doi: 10.1002/eji.200636535.
[54] A. L. Hook, D. G. Anderson, R. Langer, P. Williams, M. C. Davies, and M. R. Alexander, “High throughput methods applied in biomaterial development and discovery,” Biomaterials, vol. 31, no. 2, pp. 187-198, 2010, doi: 10.1016/j.biomaterials.2009.09.037.
[55] J. Yang et al., “Polymer surface functionalities that control human embryoid body cell adhesion revealed by high throughput surface characterization of combinatorial material microarrays,” Biomaterials, vol. 31, no. 34, pp. 8827-8838, 2010.
[56] A. Mant, G. Tourniaire, J. J. Diaz-Mochon, T. J. Elliott, A. P. Williams, and M. Bradley, “Polymer microarrays: Identification of substrates for phagocytosis assays,” Biomaterials, vol. 27, no. 30, pp. 5299-5306, 2006.
[57] C. A. Guyre et al., “Advantages of hydrophobic culture bags over flasks for the generation of monocyte-derived dendritic cells for clinical applications,” J. Immunol. Methods, vol. 262, no. 1-2, pp. 85-94, 2002, doi: 10.1016/S0022-1759(02)00015-7.
[58] P. M. Kou, “Elucidation of dendritic cell response-material property relationships using high-throughput methodologies,” Georgia Institute of Technology, 2011.
[59] A. P. Acharya, N. V Dolgova, M. J. Clare-Salzler, and B. G. Keselowsky, “Adhesive substrate-modulation of adaptive immune responses,” Biomaterials, vol. 29, no. 36, pp. 4736-4750, 2008.
[60] S. T. Reddy, M. A. Swartz, and J. A. Hubbell, “Targeting dendritic cells with biomaterials: developing the next generation of vaccines,” Trends Immunol., vol. 27, no. 12, pp. 573-579, 2006.
[61] S. J. Win et al., “Enhancing the immunogenicity of tumour lysate-loaded dendritic cell vaccines by conjugation to virus-like particles,” Br. J. Cancer, vol. 106, no. 1, pp. 92-98, 2012, doi: 10.1038/bjc.2011.538.
[62] M. Schnurr et al., “Tumor cell lysate-pulsed human dendritic cells induce a T-cell response against pancreatic carcinoma cells: An in vitro model for the assessment of tumor vaccines,” Cancer Res., vol. 61, no. 17, pp. 6445-6450, 2001.
[63] O. A. Ali, E. Doherty, D. J. Mooney, and D. Emerich, “Relationship of vaccine efficacy to the kinetics of DC and T-cell responses induced by PLG-based cancer vaccines.,” Biomatter, vol. 1, no. 1, pp. 66-75, 2011, doi: 10.4161/biom.10.1.16277.
[64] P. J. Mannon et al., “Anti-Interleukin-12 Antibody for Active Crohn's Disease,” N. Engl. J. Med., vol. 351, no. 20, pp. 2069-2079, 2004, doi: 10.1056/NEJMoa033402.

TABLE 12
Names and structures of all polymers tested and referred to herein

| Acronym | Name |
|---|---|
| 13BDDA | Butanediol-1,3 diacrylate |
| AAcAm | Diacetone acrylamide |
| AcAPAm | N-[2-(Acryloylamino)phenyl]acrylamide |
| ACl | Acryloyl Chloride |
| AnMA | Anthracenylmethylacrylate |
| BA | Butyl acrylate |
| BAGDA | Bisphenol A glycerolate diacrylate |
| BMAM | N-Benzylmethacrylamide |
| BnA | Benzyl acrylate |
| BnMA | Benzyl methacrylate |
| BPEODA | Bisphenol A ethoxylate diacrylate |
| BTHPhMA | Benzotriazol-2-yl)-4-hydroxyphenyl]ethyl methacrylate |
| BzHPEA | Benzoyl-3-hydroxy-phenoxy)ethyl acrylate |
| CHMA | Cyclohexyl methacrylate |
| CHPMA | Chloro-2-hydroxy-propyl methacrylate |
| CMAOE | Caprolactone 2-(methacryloyloxy)ethyl ester |
| CNEA | Cyanoethyl acrylate |
| NBMAm | N-(Butoxymethyl) acrylamide |
| DEAEA | Diethylamino ethyl acrylate |
| DEAEMA | Diethylaminoethyl methacrylate |
| DEGDA | Di(ethylene glycol) diacrylate |
| DEGDMA | Diethylene glycol dimethacrylate |
| DEGEEA | Di(ethylene glycol) ethyl ether acrylate |
| DEGEHA | Di(ethylene glycol) 2-ethylhexyl ether acrylate |
| DMCSPMA | Dimethylchlorosilylpropyl methacrylate |
| DPEPHA | Dipentaerythritol penta/hexa-acrylate |
| E3GDA | Triethylene glycol diacrylate |
| EA | Ethyl acrylate |
| EaNIA | Ethyl trans-a-cyano-3-indole-acrylate |
| EbCNA | Ethyl-cis-B-cyano-acrylate |
| ECNTA | Ethyl-2-cyano-3-(2-thienyl)acrylate |
| EG3DMA | Tri(ethylene glycol) dimethacrylate |
| EG4DMA | Tetraethylene glycol dimethacrylate |
| EGDPEA | Ethylene glycol dicyclopentenyl ether acrylate |
| EGDCMA | Ethylene glycol dicyclopentenyl ether methacrylate |
| EGDMA | Ethylene glycol dimethacrylate |
| EGMEA | Ethylene glycol methyl ether acrylate |
| EGMMA | Ethylene glycol methyl ether methacrylate |
| EGPEA | Ethylene glycol phenyl ether acrylate |
| EGPhMA | Ethylene glycol phenyl ether methacrylate |
| EHA | Ethylhexyl acrylate |
| EHMA | Ethylhexyl methacrylate |
| EOEA | Ethoxyethyl acrylate |
| ExA | Epoxidized acrylate |
| FMHPNMA | Trifluoro-2′-(trifluoromethyl)-2′-hydroxy)propyl]-3-norbornyl methacrylate |
| GA | Glycidyl acrylate |
| GPOTA | Glycerol propoxylate triacrylate |
| HA | Hexyl acrylate |
| HDDMA | 1,6-Hexanediol dimethacrylate, |
| HDMPDA | Hydroxy-2,2-dimethylpropyl 3-hydroxy-2,2-dimethylpropionate diacrylate |
| HEA | Hydroxyethyl acrylate |
| HfCEA | Hafnium carboxyethyl acrylate |
| HFiPA | Hexafluoroisopropyl acrylate |
| HFiPMA | Hexafluoroisopropyl methacrylate |
| HMA | Hexyl methacrylate |
| HMDA | Hexamethylene diacrylate |
| HPA | Hydroxypropyl acrylate |
| HPhMA | N-(4-Hydroxyphenyl)methacrylamide |
| iBA | Isobutyl acrylate |
| iBMA | Isobornyl methacrylate |
| iBOA | Isobornyl acrylate |
| iBOMAm | N-(Isobutoxymethyl)acrylamide |
| iDA | Isodecyl acrylate |
| iDMA | Isodecyl methacrylate |
| iOA | Isooctyl acrylate |
| iPAm | N-Isopropylacrylamide |
| LaA | Lauryl acrylate |
| LMA | Lauryl methacrylate |
| MAA | Methyl 2-acetamidoacrylate |
| MAAH | Methacrylic anhydride |
| MAEA | Methacryloyloxy)ethyl acetoacetate |
| MAEACl | [2-(Methacryloyloxy)ethyl]trimethylammonium chloride solution |
| MAL | Methacryloyl-L-Lysine |
| Mam | Methacrylamide |
| MAPtMA | Methacrylamidopropyltrimethylammonium chloride, |
| MBMAm | N,N′-Methylenebismethacrylamide |
| MEDMSAH | [2-(Methacryloyloxy)ethyl]dimethyl-(3-sulfopropyl) ammonium hydroxide |
| MMAm | N-Methylmethacrylamide |
| MPDSAH | Methacryloylamino)propyl]dimethyl(3-sulfopropyl)ammonium hydroxide inner salt |
| MTEMA | Methylthioethyl methacrylate |
| NaPhA | Sodium 3-phenyl-acrylate |
| NBMA | Norbornyl methacrylate |
| NDMAm | N-Dodecylmethacrylamide |
| NGDA | Neopentyl glycol diacrylate |
| NGPDA | Neopentyl glycol propoxylate diacrylate |
| NMEMA | 2-N-Morpholinoethyl methacrylate, 95% |
| NpA | Naphthyl acrylate |
| NpMA | Naphthyl methacrylate |
| ODA | Octadecyl acrylate |
| OFPMA | Octafluoropentyl methacrylate |
| PA | Propargyl acrylate |
| PEDAM | Pentaerythritol diacrylate monostearate |
| pEGMA | Poly(ethylene glycol) methacrylate |
| pEGMEMA | Poly(ethylene glycol) methyl ether methacrylate |
| PETA | Pentaerythritol tetraacrylate |
| PhMA | Phenyl methacrylate |
| PhMAm | N-Phenylmethacrylamide |
| PMAm | N-(Phthalimidomethyl) acrylamide |
| pPGNEA | Poly(propylene glycol) 4-nonylphenyl ether acrylate |
| SMA | Stearyl methacrylate |
| SPAK | Sulfopropyl acrylate potassium salt |
| SPMAK | 3-Sulfopropyl methacrylate potassium salt |
| TAHTA | 1,3,5-Triacryloylhexahydro-1,3,5-triazine |
| TAIC | Tris[2-(acryloyloxy)ethyl] isocyanurate |
| tBA | Tert-butyl acrylate |
| tBAEMA | Tert-butylamino-ethyl methacrylate |
| tBAm | N-tert-Butylacrylamide |
| tBCHA | Tert-butylcyclohexylacrylate |
| tBCHMA | Tertbutylcyclohexyl methacrylate |
| tBMAm | N-tert-Butylmethacrylamide |
| TEGDA | Tetra(ethylene glycol) diacrylate |
| THFuA | Tetrahydrofurfuryl acrylate |
| THFuMA | Tetrahydrofurfuryl methacrylate |
| TMBAm | N-(1,1,3,3-Tetramethylbutyl)acrylamide |
| TMCHMA | Trimethylcyclohexyl methacrylate |
| TMHA | Trimethylhexyl acrylate |
| TMOSPA | Trimethoxysilyl propyl acrylate |
| TMPDAE | Trimethyl propane diallyl ether |
| TMPETA | Trimethylolpropane ethoxylate triacrylate |
| TMPOTA | Trimethylolpropane propoxylate triacrylate |
| TMPTA | Trimethylolpropane triacrylate |
| TMSA | Trimethylsilylacrylate |
| TMSMA | Trimethylsilyl methacrylate |
| TMSOEMA | Trimethylsilyloxy)ethyl methacrylate |
| TMSOSMA | Tris(trimethylsilyloxy)-silyl propyl methacrylate |
| TPEMDA | Trimethylolpropane ethoxylate methyl ether diacrylate |
| TPGDA | Tri(propylene glycol) diacrylate |
| ZnA | Zinc acrylate |
| ZrA | Zirconium acrylate |
| ZrBNCTA | Zirconium bromonorbornanelactone carboxylate triacrylate |
| 13BDDMA | 1,3-Butanediol dimethacrylate, 98% |
| 14BDDMA | 1,4-Butanediol dimethacrylate |
| AA | Allyl acrylate |
| AAm | Acrylamide |
| AEMA.C | 2-Aminoethyl methacrylate hydrochloride, |
| AEMAm.C | N-(2-aminoethyl) methacrylamide hydrochloride |
| AMA | Allyl methacrylate |
| AODMBA | (R)-α-Acryloyloxy-β,β-dimethyl-y-butyrolactone |
| AOHPMA | Acryloyloxy-2-hydroxypropyl methacrylate |
| APMAm.C | N-(3-Aminopropyl)methacrylamide hydrochloride |
| BAC | N,N′-Bis(acryloyl)cystamine |
| BACOEA | Butylamino carbonyl oxy ethyl acrylate |
| BAPA | 1,4-Bis(acryloyl)piperazine |
| BAPODA | Bisphenol A propoxylate diacrylate |
| BDDA | Butanediol diacrylate |
| BFEODA | Bisphenol F ethoxylate diacrylate |
| BHMA | Benzhydryl methacrylate |
| BHMOPhP | 2,2-Bis[4-(2-hydroxy-3-methacryloxypropoxy)phenyl]propane |
| BMA | Butyl methacrylate |
| BMAOEP | Bis[2-(methacryloyloxy)ethyl] phosphate |
| BMENBC | Bis(2-methacryloxyethyl) N,N′-1,9-nonylene biscarbamate |
| BnPA | Benzyl 2-n-propyl acrylate |
| BOEMA | Butoxyethyl methacrylate |
| BOMAm | N-(Butoxymethyl)acrylamide |
| BPAPGDA | Bisphenol A propoxylate glycerolate diacrylate |
| BPDMA | Bisphenol A dimethacrylate |
| CEA | Carboxyethyl acrylate |
| CHA | Cyclohexyl acrylate |
| COEA | 2-Cinnamoyloxyethyl acrylate |
| CzEA | Carbazol-9-yl ethyl acrylate |
| DAAM | N,N-Diallylacrylamide |
| DDDMA | 1,10-Decanediol dimethacrylate |
| DEGMA | Di(ethylene glycol) methyl ether methacrylate |
| DFFMOA | Dodecafluoro-7-(trifluoromethyl)-octyl acrylate |
| DFHA | Dodecafluoroheptyl acrylate |
| DFHNMA | Dodecafluoro-2-hydroxy-8-(trifluoromethyl)nonyl methacrylate |
| DHEBAM | N,N′-(1,2-Dihydroxyethylene)bisacrylamide |
| DiPEMA | 2-Diisopropylaminoethyl methacrylate |
| DMA | Decyl methacrylate |
| DMAEA | Dimethylamino-ethyl acrylate |
| DMAEMA | Dimethylamino-ethyl methacrylate |
| DMAm | N,N′-Dimethylacrylamide |
| DMAPA | Dimethylamino-propyl acrylate |
| DMEMAm | N-[2-(N,N-Dimethylamino)ethyl]methacrylamide |
| DMMAm | N,N-Dimethylmethacrylamide |
| DMPAm | N-[3-(Dimethylamino)propyl]acrylamide |
| DMPMAm | N-[3-(Dimethylamino)propyl]methacrylamide |
| DOAm | Disperse Orange 3 acrylamide |
| DRA | Disperse red 1 acrylate |
| DVAd | Divinyl Adipate |
| DVSeb | Divinyl sebacate |
| DYA | Disperse yellow 7 acrylate |
| EBAM | N,N′-Ethylenebisacrylamide |
| EEA | Ethyl 2-ethylacrylate |
| EEMA | Ethoxyethyl methacrylate |
| EGDA | Ethylene {glycol} diacrylate |
| EMA | Ethyl methacrylate |
| EPA | Ethyl 2-propylacrylate |
| ETMSA | Ethyl 2-(trimethylsilylmethyl)acrylate |
| F6BA | Hexafluorobutyl acrylate |
| F6BMA | Hexafluorobutyl methacrylate |
| F7BA | Heptafluorobutyl acrylate |
| F7BMA | Heptafluorobutyl methacrylate |
| FDA | Fluorescein O,O′-diacrylate |
| FOA | Fluorescein O-acrylate |
| FuMA | Furfuryl methacrylate |
| GDGDA | Glycerol 1,3-diglycerolate diacrylate |
| GDMA | Glycerol dimethacrylate |
| GMA | Glycidyl methacrylate |
| HBA | Hydroxybutyl acrylate |
| HBMA | Hydroxybutyl methacrylate |
| HBOPBA | Hexanediylbis[oxy(2-hydroxy-3,1-propanediyl)] bisacrylate |
| HDFDA | Heptadecafluorodecyl acrylate |
| HDFDMA | Heptadecafluorodecyl methacrylate |
| HDFHUA | Heptadecafluoro-2-hydroxyundecyl acrylate |
| HDMA | 1-Hexadecyl methacrylate |
| HEAm | N-Hydroxyethyl acrylamide |
| HEMA | Hydroxyethyl methacrylate |
| HEODA | Hexanediol ethoxylate diacrylate |
| HFDA | Heneicosafluorododecyl acrylate |
| HFHUMA | Hexadecafluoro-2-hydroxy-10-(trifluoromethyl)undecyl methacrylate |
| HFPDA | Hexafluoropent-1,5-diyl diacrylate |
| HMAm | N-(Hydroxymethyl)acrylamide |
| HMBAM | N,N′-Hexamethylenebisacrylamide |
| HMBMAm | N,N′-Hexamethylenebis(methacrylamide) |
| HPhOPA | Hydroxy-3-phenoxypropyl acrylate |
| HPHPBAH | Hydroxypivalyl hydroxypivalate bis[6-(acryloyloxy)hexanoate] |
| HPMA | Hydroxypropyl methacrylate |
| HPMAm | N-(2-Hydroxypropyl)methacrylamide |
| HPMAP | Hydroxypropyl 2-(methacryloyloxy)ethyl phthalate |
| HTFDA | Hexadecafluoro-9-(trifluoromethyl)decyl acrylate |
| iBuMA | Isobutyl methacrylate |
| iCEMA | Isocyanatoethyl methacrylate |
| MA | Methyl acrylate |
| MAAHS | Methacrylic acid N-hydroxysuccinimide ester |
| MAETA | 4-Methacryloxyethyl trimellitic anhydride |
| MAHBP | 4-Methacryloxy-2-hydroxybenzophenone |
| MAPU | 2-methacryloxyethyl phenyl urethane |
| MBAm | N,N′-Methylenebisacrylamide |
| MHMB | Methyl 3-hydroxy-2-methylenebutyrate |
| MMA | Methyl methacrylate |
| mMAOEM | mono-2-(Methacryloyloxy)ethyl maleate |
| mMAOES | mono-2-(Methacryloyloxy)ethyl succinate |
| MOPAm | N-(3-Methoxypropyl)acrylamide |
| MSPMA | Methyldiethoxysilyl-propyl methacrylate |
| NAM | N-Acryloylmorpholine |
| NAS | N-Acryloxysuccinimide |
| NBnMA | o-Nitrobenzyl methacrylate, . 95% |
| NDDMA | 1,9-Nonanediol dimethacrylate |
| nOcMA | n-Octyl methacrylate, |
| NPhPMA | Nitrophenyl-2-pyrrolidonemethyl acrylate |
| OFHMA | Octafluoro-2-hydroxy-6-(trifluoromethyl)heptyl methacrylate |
| OFPA | Octafluoropentyl acrylate |
| PAHEMA | Phosphoric acid 2-hydroxyethyl methacrylate ester |
| PBBA | Pentabromobenzyl acrylate |
| PDA | 1,4-Phenylene diacrylate |
| PDDMA | 1,5-Pentanediol dimethacrylate |
| pEGDA | Polyethylene glycol diacrylate |
| pEGDMA | Poly(ethylene glycol) (600) dimethacrylate |
| pEGMEA | Poly(ethylene glycol) methyl ether acrylate |
| pEGPhEA | Poly(ethylene glycol) phenyl ether acrylate |
| PhEMA | 2-Phenylethyl methacrylate |
| PETrA | Pentaerythritol triacrylate |
| pFDA | Perfluorodecyl acrylate |
| PFPA | Pentafluoropropyl acrylate |
| PFPhA | Pentafluorophenyl acrylate |
| PFPhMA | Pentafluorophenyl methacrylate, 95% |
| PFPMA | Pentafluoropropyl methacrylate |
| PhA | Phenyl acrylate, 95% |
| PhEA | 2-Phenylethyl acrylate |
| PHPMA | 3-Phenoxy 2 hydroxy propyl methacrylate |
| PMA | Propargyl methacrylate |
| PMMA | 1-Pyrenylmethyl methacrylate |
| pPGA | Poly(propylene glycol) acrylate |
| pPGDA | Poly(propylene glycol) diacrylate |
| pPGDMA | Poly(propylene glycol) (400) dimethacrylate |
| pPGMEA | Poly(propylene glycol) methyl ether acrylate |
| SEMA | 2-Sulfoethyl methacrylate |
| tBMA | Tert-butyl methacrylate |
| TBNpMA | Tribromoneopentyl methacrylate |
| tBOCAPAm | N-(t-BOC-aminopropyl)methacrylamide |
| TBPhA | 2,4,6-Tribromophenyl acrylate |
| TBPMA | Tribromophenyl methacrylate |
| TCDMDA | Tricyclodecane-dimethanol diacrylate |
| TCSPMA | Trichlorosilyl propyl methacrylate |
| TDFOCA | Tridecafluorooctyl acrylate |
| TDFOMA | Tridecafluorooctyl methacrylate |
| TEGMA | Tri(ethylene glycol) methyl ether methacrylate |
| TFCAm | 7-[4-(Trifluoromethyl)coumarin]acrylamide |
| TFPMA | Tetrafluoropropyl methacrylate |
| THMMAm | N-[Tris(hydroxymethyl)methyl]acrylamide |
| TMOBDA | Trimethylolpropane benzoate diacrylate |
| TMOPTMA | 1,1,1-Trimethylolpropane trimethacrylate |
| TMOSPMA | Trimethoxysilyl propyl methacrylate |
| tOcAm | N-tert-Octylacrylamide |
| TPhMAm | N-(Triphenylmethyl)methacrylamide |
| VMA | Vinyl methacrylate |
| ZrCEA | Zirconium carboxyethyl acrylate |
| GMMA | Glycerol monomethacrylate |
| MAEP | Monoacryloxyethyl phosphate |
| MAEPC | 2-Methacryloyloxyethyl phosphorylcholine |
| PBPhA | Pentabromophenyl acrylate |
| PBPhMA | Pentabromophenyl methacrylate |
| PPPDMA | PEO(5800)-b-PPO(3000)-b-PEO(5800) dimethacrylate |
| CIEA | 2-Chloroethyl acrylate |
| MCIMA | Methyl 2-(chloromethyl)acrylate |
| AEAm.C | N-(2-aminoethyl)acrylamide hydrochlide |
| SolA | Solketal acrylate |
| DHPA | 2,3-dihydroxypropyl acrylate |
| PPDDA | 3-phenoxypropane-1,2-diyl diacrylate |
| IBESMA | 1,7,7-trimethylbicyclo[2.2.1]heptan-2-yl 6-(methacryloyloxy)-4-oxohexanoate |
| tBEMAm | N-(3,3-dimethylbutyl)methacrylamide |
| IPBMA | p-isopropylbenzyl methacrylate |
| 2EhMA | 2-ethylhexyl methacrylate |
| MbMA | 4-methylbenzyl methacrylate |
| CyDMA | cyclododecyl methacrylate |
| CeMA | cetyl methacrylate |
| GMA-AD | geranyl methacrylate |
| LMMA | L-menthyl methacrylate |
| CiMA | cinnamyl methacrylate |
| NibMA | 4-nitrobenzyl methacrylate |
| ClbMA | 2-chlorobenzyl methacrylate |
| PiMA | pivaloyl methacrylate |
| DdMA | dodecyl methacrylate |
| MpMA | 4-methylpentan-2-yl methacrylate |

1. A microtopography system for modulating one or more cellular processes on a surface, said microtopography system comprising:
a repeated microtopographic pattern and a polymer coating, said microtopographic pattern comprising:
an array of repeated micropillars applied to a surface of a product, said micropillars being formed of surface structures between 1-100 μm in height, and 1-50 μm in width; and
said polymer coating comprising one of a (meth)acrylate or (meth)acrylamide monomer, or mixture of two (meth)acrylate or (meth)acrylamide monomers;
wherein said microtopographic pattern and said polymer coating act to modulate one or more cellular processes on the surface.

2. The system of claim 1, wherein the micropillars are:
about 1-100 μm in height (vertical), such as about between 5-45 μm, 10-40 μm, 15-35 μm, 20-30 μm, 25 μm, or 50-100 μm; and
about 1-100 μm in width (diameter), such as 2-45 μm, 3-40 μm, 4-35 μm, 5-30 μm, 10-25 μm, 15-20 μm, or 50-100 μm.

3. The system of claim 1 or claim 2, wherein the microtopography of the micropillars protrude above the underlying surface and have a mean area below 50 μm²; and/or the micropillars have an eccentricity of <1, and preferably less than 0.5, preferably between 0.01-0.49, more preferable between 0.1-0.4, and most preferably between 0.2-0.3.

4. The system of any preceding claim, wherein the micropillars are shaped according to a topography determined using a screening technique of possible primitive shape combinations, said primitive combinations comprising one or more of quadrilaterals, circles, triangles or other primitive shapes combined using a computational algorithm to generate a micropillar that does not resemble the original primitives.

5. The system of any preceding claim, wherein the polymers are formed by in situ photopolymerisation of the respective monomer(s) drop cast on top of the topography features.

6. A polymer system for modulating one or more cellular processes on a surface, said polymer system comprising a surface with a polymer coating applied to it, said polymer coating comprising one of a (meth)acrylate or (meth)acrylamide monomer, or mixture of two (meth)acrylate or (meth)acrylamide monomers, and wherein the polymer coating acts to modulate a cellular process on the surface.

7. The system of any preceding claim, wherein the one or more cellular processes are modulated in prokaryotic or eukaryotic cells.

8. The system of any preceding claim, wherein the one or more cellular processes comprises or consists of cell attachment, cell differentiation, cell proliferation, cell viability, cell pluripotency, protein expression and/or immune cell modulation, and wherein the modulation results in an increase or decrease of said one or more cellular processes.

9. The system of claim 8, wherein the cell attachment is one or more of prokaryote or eukaryote attachment, including one or more of Gram positive bacterial cell attachment; Gram negative bacterial cell attachment; fungal cell attachment, Antigen Presenting Cell (APC) attachment such as macrophage or dendritic cell attachment; neutrophil attachment; fibroblast attachment and/or proliferation; stem cell attachment such as human mesenchymal stem cell or embryonic stem cell attachment.

10. The system of claim 8, wherein the cell differentiation is one or more of stem cell differentiation such as mesenchymal stem cell differentiation to an osteoblast, or monocyte differentiation into dendritic cells or macrophages, or differentiation of fibroblasts to myofibroblasts, cell differentiation from stem cells to to cardiomyocytes, neurons, adipocytes, hepatocytes, chondrocytes.

11. The system of claim 8, wherein the immune cell modulation comprises or consists of immune activity such as pro-inflammatory or anti-inflammatory activity.

12. The system of claim 11 wherein the immune activity may be one or more of the activation and/or polarisation of macrophages to an M0, M1 or M2 phenotype; the maturation and/or activation or suppression of dendritic cells; the activation or suppression of neutrophils; the production of cytokines from APCs; the differentiation of monocytes into dendritic cells or macrophages; the attachment of monocytes, macrophages, dendritic cells or neutrophils.

13. The system of any preceding claim, wherein the microtopographic pattern and/or polymer coating may both reduce bacterial cell attachment and increase M2 macrophage polarisation or dendritic cell activation at the surface.

14. The system of any preceding claim, wherein microtopographic pattern and/or polymer coating may both reduce fungal cell attachment and fungal cell proliferation the surface.

15. A product comprising the system of any preceding claim, wherein said surface comprises a surface of the product and wherein said microtopography and polymer coating modulates cell attachment to the surface of said product and/or immune activity of the attached cells.

16. A product according to claim 15, wherein the system is for use in preventing or reducing biofilm formation and/or preventing or treating an infection and/or preventing rust formation, and/or preventing or reducing microorganism colonisation.

17. A product according to claim 15 or claim 16, wherein the product comprises or consists of a wound dressing, a cell culture dish, a food container, packaging or the like, an encasing, a biological catalytic surface for an industrial surface, a food processing product, a water container or treatment product, a beverage container or surface for use in the beverage industry, a food or plant product and/or a surface used in displays or windows.

18. A product according to claim 15 or claim 16, wherein the surface is for use in treating or preventing an immune disease/disorder by modulating the attachment and/or immune activity of an immune cell, such as an APC, in a subject.

19. The product of claim 18, wherein the immune cell is a monocyte, macrophage, neutrophil, or a dendritic cell.

20. The product according to claim 18 or claim 19, wherein the immune disease/disorder is selected from the following group consisting of: transplant rejection, Graft Versus Host Disease (GVHD), psoriasis, eczema, rheumatoid arthritis, a cancer, immunosuppression, systemic lupus erythematosus, inflammatory bowel disease, Crohn's disease, multiple sclerosis, Type I diabetes, Guillain-Barre syndrome, fibrosis, chronic non-healing wounds or medical device rejection.

21. A product comprising a system according to any of claims 1 to 19, for use in treating or preventing an infection in a subject.

22. The product for use according to 21, wherein the microtopography modulates cell attachment to the surface of said product and/or immune activity of the attached cells.

23. The product for use according to claim 21 or claim 22, wherein the disease to be prevented or treated is caused by an infection of one or more of a bacteria, a virus, a fungi, a protozoan, or a mixture thereof.

24. The product use according to claim 23, wherein the one or more bacteria are selected from the group of one or more of Pseudomonas spp., Staphylococcus spp., Bacillus spp., Lactobacillus sp., proteus spp., Enterobacter spp., Escherichia Coli, Klebsiella spp., Salmonella spp., Listeria spp., Yersinia spp., Legionella spp, Clostridium spp., Acinetobacter spp., Pseudomonas aeruginosa, Staphylococcus aureus, Proteus mirabilis, or Acinetobacter baumannii, or one or more fungi selected from the group of one or more of Candida albicans Botrytis cinerea, Zymoseptoria tritici, and/or Aspergillus brasiliensis.

25. The product for use according to any one of claims 15 to 24, wherein the product is one of: an implantable medical device, prosthetic, surgical tool, dental tool; dental device; catheter, dental screw, knee joint replacement, hip joint replacement, heart valve replacement, a stent, pacemaker, glucose sensor, contraceptive implant, breast implant, Implantable Cardioverter Defibrillators, spinal screws/rods/artificial discs, contact lenses, shunts stents or wound care products.

26. A method of screening for a system according to any preceding system claim, wherein the method comprises:
i. Applying at least one microtopography to a surface;
ii. Applying a polymer to at least a substantial portion of the surface;
iii. Culturing one or more of a first set of cells on the surface with said microtopography and said polymer applied to it, and culturing a matching number and type of cells of a second set of cells on a reference surface;
iv. Measuring or detecting the level of one or more cellular processes of the first and second set of cells;
v. Comparing the level of the one or more measured or detected cellular processes of the first and second set of cells; and
vi. Determining whether the level of each of the one or more measured or detected cellular processes between the first and second set of cells is modulated either positively or negatively on the surface with said microtopography and said polymer applied to it compared to the reference surface.

27. A method of screening for a polymer system according to any preceding system claim directly or indirectly dependent on claim 6, wherein the method comprises:
i. Applying a polymer or mixture of polymers to at least a substantial portion of the surface;
ii. Culturing one or more of a first set of cells on the surface with said microtopography and said polymer applied to it, and culturing a matching number and type of cells of a second set of cells on a reference surface;
iii. Measuring or detecting the level of one or more cellular processes of the first and second set of cells;
iv. Comparing the level of the one or more measured or detected cellular processes of the first and second set of cells; and
v. Determining whether the level of each of the one or more measured or detected cellular processes between the first and second set of cells is modulated either positively or negatively on the surface with said polymer or mixture of polymers applied to it compared to the reference surface.

28. A method of claim 26 or claim 27, wherein the polymer or mixture of polymers is formed from a (meth)acrylate or (meth)acrylamide monomer or a mixture of two (meth)acrylate or (meth)acrylamide monomers.

29. A method of modulating one or more cellular processes at a surface of a product, wherein the method comprises applying a microtopography to said surface, and applying a polymer to at least a substantial portion of said surface.

30. A method of modulating one or more cellular processes at a surface of a product, wherein the method comprises applying a polymer to at least a substantial portion of said surface.

31. A product with a surface on which a microtopography has been applied, and on which a polymer has been applied to at least a substantial portion of, for use in modulating one or more cellular processes at said surface.

32. A product with a surface on which a polymer or mixture of polymers has been applied to at least a substantial portion of, for use in modulating one or more cellular processes at said surface.

33. The product of any preceding product claim for use in preventing rust formation, preventing food spoilage, tissue culture and research product coating such as cell culture dishes and plasticsware, glassware, anti-fouling paint, food processing equipment and preparation areas, water systems and containers.

34. A product of the invention according to any preceding product claim, for use in treating or preventing a bone disorder, fibrosis, or wound healing.

35. A method of treating or preventing a disease or disorder in a subject, comprising:
i. Applying a microtopography to the surface of an implantable medical or dental product;
ii. applying a polymer to at least a substantial portion of said surface.
iii. applying said product the subject.

36. A method of treating or preventing a disease or disorder in a subject, comprising:
i. Applying a polymer or mixture of polymers to the surface of an implantable medical or dental product;
ii. applying said product the subject.

37. The method of claim 35 or claim 36, wherein, the disease or disorder may be selected from: a bacterial infection, fungal infection, an inflammatory disease or disorder, a bone disorder, fibrosis, biofilm formation, non-healing/chronic wounds.

38. The system, product, or method of any corresponding claim, wherein the one or more cellular processes comprises or consists of inducing (increasing) cell differentiation.

39. The system, product, or method of claim 38 wherein the cell differentiation may be stem cell differentiation; and optionally or preferably cell differentiation from human mesenchymal stem cells (hMSCs) to osteoblasts.

40. The system, product or method of claim 39, wherein the cell differentiation is inducted by microtopography having features with a radius of about 2-3 μm, preferably 2.5 μm, spacings of about 5-10 μm and wherein the polymer coating is BzHPEA.

41. The system, product or method of claim 39, wherein the cell differentiation is inducted by microtopography having features with a radius of about 2.5-3.5 μm, preferably 3.5 μm, and the polymer coating is mMAOES.

42. The system, product or method of claim 39, wherein the cell differentiation is inducted by microtopography having features with a radius of about 2.5-3.5 μm, preferably 3.5 μm, and the polymer is MAPU.

43. The system, product or method of any one of claims 1 to 37, wherein the one or more cellular processes comprise or consists of immune cell modulation.

44. The system, product or method of claim 43 wherein the immune cell modulation is inducing (increasing) the differentiation of human CD14+ monocytes into APCs.

45. The system, product or method of claim 44, wherein the APCs are macrophages and dendritic cells.

46. The system, product or method of claim 44 wherein the macrophages are polarised to an M2 or M1 phenotype.

47. The system, product or method of claim 46 wherein the microtopography has cylindrical pillars with a mean area below 50 μm², a maximum radii of about 1-3 μm, an eccentricity of below 0.5, preferably between 0.1-0.4, more preferably between 0.15-0.35, and the polymer is DMAm, BzHPEA or DEAEMA.

48. The system, product or method of claim 44, wherein the CD14+ monocytes are differentiated into dendritic cells.

49. The system, product or method of claim 48, wherein the CD14+ monocytes are differentiated into monocyte-derived dendritic cells (MoDCs), which are activated dendritic cells, and wherein the polymer is any one of BADPODA, DEAEA, EaNiA, HFiPMA, COEA, F7BA, pEGMEMA, HEA, pEGDA or PhEA.

50. The system, product or method of claim 48 wherein the CD14+ monocytes are differentiated into monocyte-derived dendritic cells (MoDCs) which are suppressed dendritic cells, and wherein the polymer or mixture of polymers is any one of: COEA, THFuA, ZnA, PEDAM, PhMAm, MAPU, HDFHuA, (EDGMA about 66%+HDFDA about 33%), MTEMA.

51. The system, product or method of claim 48, wherein the longevity and/or viability of monocyte-derived dendritic cells (MoDCs) is increased, and wherein the polymer comprises any one of DFHA, MBMAm, SPAK, SPMAK, THFuMA, NpMA, PhEA, ZrCEA, DEGDMA, TEGDA.

52. The system, product or method of claim 48, wherein the CD14+ monocytes are differentiated to M0 macrophages and wherein the differentiation is induced by a polymer or mixture of polymers selected from (EGDMA about 66%+HDFDMA about 33%), (BOEMA about 66%+DFFMOA about 33%), GPOTA, C398, or C408.

53. The system, product or method of claim 48, wherein the CD14+ monocytes are differentiated to M1 macrophages and wherein the differentiation is induced by a polymer or mixture of polymers selected from (CHMA about 66%+DMAEMA about 33%), tBCHMA, HDDMA, BDDA, DDDMA, TMOPTMA, H126, H98, H135, C176, C170, or C240.

54. The system, product or method of claim 48, wherein the CD14+ monocytes are differentiated to M2 macrophages and wherein the differentiation is induced by a polymer or mixture of polymers selected from (CHMA about 66%+iDMA about 33%), (PhMA about 66%+iDMA about 33%), IDMA, GDGDA, tBMA, TAlC, H47, H37, H9, C255, C140, or C186.

55. The system, product or method of claim 48, wherein the CD14+ monocytes or macrophages are induced to form an attachment to a surface, and wherein the polymer or mixture of polymers comprises one or more of H133, H90, H103, H21, H94, H24, H69, H96, H92, H33, C56, C386, C32, C347, or C295.

56. The system, product or method of claim 48, wherein the CD14+ monocytes or macrophages are induced to deter from an attachment to a surface, and wherein the polymer or mixture of polymers comprises one or more of C358, C209, C434, C94, C48.

57. The system, product or method of claim 48, wherein to induce an increase in CD14+ monocyte attachment to a surface and increase in the differentiation of CD14+ monocytes to M1 macrophages, the polymer or mixture of polymers comprises C170.

58. The system, product or method of claim 48, wherein to induce an increase in CD14+ monocyte attachment to a surface and increase in the differentiation of CD14+ monocytes to M2 macrophages, the polymer or mixture of polymers comprises C162.

59. The system, product or method of claim 48, wherein to induce a decrease in CD14+ monocyte attachment to a surface and increase in the differentiation of CD14+ monocytes to M1 macrophages, the polymer or mixture of polymers comprises C311.

60. The system, product or method of claim 48, wherein to induce a decrease in CD14+ monocyte attachment to a surface and increase in the differentiation of CD14+ monocytes to M1 macrophages, the polymer or mixture of polymers comprises C164.

61. The system, product or method of any preceding claim, wherein the one or more cellular processes comprises or consists of cell proliferation and/or smooth muscle actin (SMA) expression.

62. The system, product or method of claim 61, wherein to induce an increase in SMA expression and increase in cell proliferation, the polymer comprises one or more of PhEA, THFuMA, CzEA or EGDA.

63. The system, product or method of claim 61, wherein to induce a decrease in SMA expression and decrease in cell proliferation, the polymer comprises one or more of PBPhMA, THFuA, pEGPHEA, EGDPEA, LMMA, NibMA, iDA, MAETA, or AODMBA.

64. The system, product or method of claim 61, wherein to induce a decrease in SMA expression and increase in cell proliferation, the polymer comprises one or more of NBnMA, TMPDAE, EGPEA, DMPMAm, THFuA or HFPDA.

65. The system, product or method of claim 61, wherein to induce an increase in SMA expression and decrease in cell proliferation, the polymer comprise one or more of PPDDA, 2EhMA, ClbMA or DVAd.

63. The system, product or method of any preceding claim, wherein the one or more cellular processes comprises or consists of fibroblast attachment to a surface.

64. The system, product or method of claim 63 wherein to induce a decrease in fibroblast attachment, the polymer comprises one or more of HEA, iPAM, AA, iBuMA, PPPDMA, MMaM, MAPU, HMAm or HEAm.

65. The system, product or method of any preceding claim wherein the one or more cellular processes comprises or consists of fungal cell attachment.

66. The system, product or method of claim 65 wherein to induce a decrease in Candida albicans attachment to a surface, the polymer comprising one or more of AODMBA, tBCHMA, tBCHA or IDMA; and/or
to induce a decrease in Botrytis cinerea attachment to a surface, the polymer comprising one or more of mMAOES, DEGEEA or pEGPhEA; and/or
to induce a decrease in both Botrytis cinerea and Candida albicans attachment to a surface, the polymer comprising one or more of DEGMA or TEGMA.

67. The system, product or method of any preceding claim wherein the one or more cellular processes comprises or consists of neutrophil attachment.

68. The system, product or method of claim 67 wherein to induce an increase in neutrophil attachment to a surface, the polymer comprises one or more of DMPAm, AMPAm.C, MAEACI, DMEMAm, EGDA or AEMAm.C.

69. The system, product or method of any preceding claim wherein the one or more cellular processes comprises or consists of retention of stem cell pluripotency after cell proliferation.

70. The system, product or method of any claim 69 wherein to induce an increase in retention of stem cell pluripotency after cell proliferation on a surface, the polymer or mixture of polymers comprises one or more of poly tricyclodecane-dimethanol diacrylate-co-butyl acrylate (poly(TCDMDA-blend-BA)), suitably at a ratio of about 70:30.
